(19) United States
(12) Patent Application Publication
(10) Pub. No.: US 2025/0346636 A1
(43) Pub. Date: Nov. 13, 2025

(54) HUMAN T-CELL LYMPHOTROPIC VIRUS TYPE 1 TARGETING PROTEINS AND METHODS OF USE

(71) Applicant: CITY OF HOPE, Duarte, CA (US)

(72) Inventors: Tristan SCOTT, Duarte, CA (US); Kevin V. MORRIS, Duarte, CA (US)

(21) Appl. No.: 18/854,481

(22) PCT Filed: Apr. 5, 2023
(86) PCT No.: PCT/US2023/065407

§ 371 Date: Oct. 4, 2024

Related U.S. Application Data
(60) Provisional application No. 63/328,108, filed on Apr. 6, 2022.

(51) Int. Cl. C07K 14/005; A61K 35/17; A61K 38/00; A61P 35/02; C07K 14/47; C12N 5/0783

(52) U.S. Cl. CPC C07K 14/005; A61K 35/17; A61P 35/02; C07K 14/4703; C12N 5/0636; A61K 38/00; C07K 2319/09; C12N 2510/00; C12N 2770/00033

(57) ABSTRACT

Provided herein, inter alia, are compositions for treating Human T-cell lymphotropic virus type 1 (HTLV-1) associated diseases. The compositions include a protein having a zinc finger domain capable of binding a sequence within an HTLV-1 long terminal repeat (LTR). Further provided are methods of treating HTLV-1 associated diseases in a subject in need thereof. The methods include administering to the subject the protein including the zinc finger domain, or a nucleic acid encoding the protein.

### CROSS-REFERENCE TO RELATED APPLICATIONS

This application claims priority to U.S. Provisional Application No. 63/328,108, filed Apr. 6, 2022, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under RO1 M113407 awarded by the National Institutes of Health. The government has certain rights in the invention.

### REFERENCE TO A SEQUENCE LISTING, A TABLE OR A COMPUTER PROGRAM LISTING APPENDIX SUBMITTED AS AN ASCII TEXT FILE

The contents of the electronic sequence listing (048440-836001WO_ST26.xml; Size 133,584 bytes; and Date of Creation: Mar. 20, 2023) is hereby incorporated by reference in its entirety.

### BACKGROUND

Human T-lymphotropic virus type I (HTLV-I), a retrovirus, is transmitted by bodily fluids and establishes a life-long infection in patients. The virus infects primarily CD4+ T-cells in which the reverse transcribed genome integrates within the host cell to form a provirus. Viruses are predicted to cause about 15% of known cancers world-wide (1), and HTLV-I is the established etiological agent involved in the development of a group of blood-borne malignances. Through a complex interplay between viral factors over an extended incubation time, the virus has been linked to the transformation of CD4+ T-cells into a tumor state, resulting in acute T-cell leukemia/lymphoma (ATL). In its most aggressive form, acute ATL, the prognosis for the overall survival rate is ˜9 months. There remains no vaccine or treatment for HTLV-I, and, furthermore, ATL is refractory to chemotherapy and radiation therapy with no effective, commercially available alternative cancer treatment. The C-C Motif Chemokine Receptor 4 (CCR4) is upregulated on the surface of most ATLs (2), and a monoclonal antibody, mogamulizumab, has been used in clinical trials in CCR4-positive ATL patients with limited improvement in disease outcomes (3). However, a sub-class of ATLs with gain-of-function CCR4 mutations substantially improved the antibody's treatment response (4). Nonetheless, the overall lack of effective approaches to inhibit ATL urges the development of novel therapeutic strategies.

HTLV-I has ˜9 kb genome flanked by long terminal repeats (LTRs) at the 5′ and 3′ ends that serve as promoters to drive sense and anti-sense expression, respectively. The HTLV-I transactivator protein Tax is expressed from the 5′ LTR, along with other accessory and structural genes involved in productive viral replication, and is a well-established factor in clonal expansion and oncogenic transformation (5). However, Tax is highly immunogenic resulting in cytotoxic CD8+ T-cell clearance of Tax-positive cells, and in ATL is generally lowly expressed or silent as a result of gene mutation, 5′LTR truncation, or promoter epigenetic hypermethylation (6).

Recently, the anti-sense HTLV-1 bZIP factor (HBZ) gene expressed from the 3′LTR has been realized as playing an underappreciated role in oncogenesis as it suppresses apoptosis (7), induces genetic instability (8), and results in T-cell lymphomas in HBZ transgenic mice (9). Importantly, the HBZ RNA and protein have been implicated in various proliferative and pathological roles in ATL (10), such as the up-regulation of CCR4 that augments the tumor's migration and proliferation (11). Furthermore, all primary ATL samples are positive for HBZ expression (12), and the selective inhibition of HBZ reduced proliferation in a range of HTLV-I cell lines (13,14), presenting a potential common molecular target for cancer intervention.

Provided herein, inter alia, are solutions to these and other problems in the art.

### BRIEF SUMMARY

Provided herein, inter alia, are proteins including zinc finger domains capable of binding a sequence within the long terminal repeat (LTR) of Human T-cell lymphotropic virus type 1 (HTLV-I). The proteins provided herein including embodiments thereof are contemplated to be effective for downregulating expression of the HTLV-1 bZIP factor (HBZ) gene. Applicant has further discovered that proteins provided herein including embodiments thereof may be effective for treating and/or preventing HTLV-1 associated diseases (e.g. adult T-cell leukemia, etc.). Thus, in an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:27.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:25.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:28.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:32.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:31.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:30.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:24

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:26

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:29.

In another aspect a nucleic acid encoding the protein provided herein including embodiments thereof is provided.

In an aspect a vector including the nucleic acid provided herein including embodiments thereof is provided.

In another aspect is provided an extracellular vesicle (EV) including a nucleic acid encoding the protein provided herein including embodiments thereof.

In an aspect is provided a pharmaceutical composition including the protein provided herein including embodiments thereof, the nucleic acid provided herein including embodiments thereof, the vector provided herein including embodiments thereof, or the EV provided herein including embodiments thereof.

In another aspect is provided a cell including the protein provided herein including embodiments thereof, the nucleic acid provided herein including embodiments thereof, the vector provided herein including embodiments thereof, or the EV provided herein including embodiments thereof.

In another aspect is provided a method of treating a human T-cell lymphotropic virus type 1 (HTLV-1) associated disease in a subject in need thereof, including administering to the subject an effective amount of the protein provided herein including embodiments thereof, the nucleic acid provided herein including embodiments thereof, the vector provided herein including embodiments thereof, or the EV provided herein including embodiments thereof.

### BRIEF DESCRIPTION OF THE DRAWINGS

FIG. 1: Schematic of the HTLV-I genome and ZFP target sites. The 5′ LTR and 3′ LTRs flank the ˜9 kb integrated HTLV-I genome and the 3′ LTR drives the expression of the anti-sense HBZ gene. The representative target sites of a series of ZFP within the LTR are indicated (arrows, ZFP2 to ZFP10). Transcription factor SpI binding sites, the transcription start site (TSS) in the 3′ LTR, and the HBZ coding sequence are as labeled.

FIGS. 2A-2E: Screening of ZFP repressors that inhibit HTLV-1 LTR expression. (FIG. 2A) HEK293 cells were transfected with a vector that contains a HTLV-1 LTR bidirectionally driving the expression Rluc (anti-sense) and Fluc (sense) luciferase. A mutated Rluc translational start ensures that expression of Rluc only occurs if the 5′ HBZ sequence within the LTR is spliced onto the reporter. A series of HTLV-I ZFP-KRAB repressors (2-10) were transfected with the reporter vector and 48 hrs post-transfection the levels of luciferase were determined. (FIG. 2B) HEK293 cells were transfected with a vector containing the HTLV-1 3′-LTR driving the expression of the HBZ-3×FLAG with the ZFP vectors, and 48 hrs post-transfection the levels of HBZ RNA were assessed. Both spliced (HBZsp) and unspliced (e.g. nascent) HBZ RNA (HBZusp) was detected. For (FIG. 2A) and (FIG. 2B), error bars represent standard deviation from samples treated in triplicate from two independent experiments. The levels of luciferase or HBZ RNA was made relative to a ZFP-HIV-KRAB control, set a 100%. (FIG. 2C) HEK293 cells were transfected as described in (FIG. 2B) and the HBZ-3×FLAG and ZFPs were detected through their Flag and myc tags, respectively. A Rluc expression vector or untreated cells (mock) were included as ZFP and HBZ detection controls, respectively. Alpha-tubulin was detected as a loading control. The RNA levels were determined for (FIG. 2D) spliced (HBZsp) and nascent HBZ RNA (HBZusp), and (FIG. 2E) KRAB, ZFP3, or ZFP5.

FIGS. 3A-3B: Anti-proliferative effects of the anti-HBZ ZFP repressors. TL-Om1 cells were electroporated with an (FIG. 3A) 2 μg ‘low’ dose or (FIG. 3B) 4 μg ‘high’ dose of mRNA expressing the ZFP5-KRAB or ZFP5-KRAB-meCP2 and outgrowth was assessed up to day 21 through proliferation (top panel), viability (middle panel), or cell count (bottom panel). The ZFP-HIV-KRAB or GFP mRNAs were included as negative controls. Error bars represent standard deviation from samples treated in triplicate.

FIGS. 4A-4C: Anti-HTLV-I ZFPs reduce HBZ-induced CCR4 levels. TL-Om1 cells were electroporated with 2 μg of ZFP5-KRAB or ZFP5-KRAB-meCP2 mRNA, and the levels of (FIG. 4A) HBZ spliced RNA, (FIG. 4B) CCR4 RNA, (FIG. 4C) or surface CCR4 receptor was assessed at 24 hrs and 48 hrs post-electroporation. Cells treated with a ZFP-HIV-KRAB mRNA or untreated cells (mock) were included as negative controls. Error bars represent standard deviation from samples treated in triplicate and p-values were determined by one-way ANOVA analysis (Dunnett's post-test) when compared to the ZFP-HIV-control (*p<0.05, **p<0.01 ***p<0.001, ****p<0.0001).

FIGS. 5A-5D: Anti-HBZ ZFPs cause cell cycle arrest and apoptosis. (FIG. 5A) TL-Om1 cells were electroporated with 2 μg of mRNA expressing the ZFP5-KRAB or ZFP5-KRAB-meCP2 and the percentage of cell cycle phase was assessed at 24 hrs post-electroporation. (FIG. 5B) The levels of E2F1 mRNA were assessed at 24 hrs and 48 hrs post-electroporation. Cells treated with a ZFP-HIV-KRAB mRNA or untreated (mock) were included as negative controls. For (FIG. 5C), the samples were made relative to the ZFP-HIV-KRAB set at 100%. To assess the induction of apoptosis, TL-Om1 cells were electroporated with a (FIG. 5C) 2 μg ‘low’ dose or (FIG. 5D) 4 μg ‘high’ dose of mRNA and Annexin V and PI detected at 48 hrs and 72 hrs post-electroporation. ZFP-HIV-KRAB was used as a negative control. For (FIG. 5A) and (FIG. 5B), error bars represent standard deviation from samples treated in triplicate. For (FIG. 5C) and (FIG. 5D), the line represents the mean from samples treated in triplicate. The p-values were determined by one-way ANOVA analysis (Dunnett's post-test) when compared against ZFP-HIV-control (*p<0.05, **p<0.01 ***p<0.001, ****p<0.0001).

FIGS. 6A-6B: Anti-HTLV-I ZFP repressors inhibit the LTRs from multiple HTLV-I genotypes. (FIG. 6A) A schematic of the vector that contains a HTLV-1 LTR bidirectionally driving the expression Rluc (anti-sense) and Fluc (sense) luciferase. The LTR upstream of the HBZ start was replaced with sequences from different HTLV-I genotypes (a-g). The country of origins, accession numbers, genotypes, and ZFP5 target site sequences are indicated. Mismatches are in bold. (FIG. 6B) HEK293 cells were transfected with an LTR(a-g) spliced reporter vector with the ZFP5-KRAB and ZFP5-KRAB-meCP2 vectors, and 48 hrs post-transfection the levels of luciferase was determined. Error bars represent standard deviation from samples treated in triplicate. The levels of luciferase were made relative to a ZFP-HIV-KRAB control set a 100%.

FIGS. 7A-7D: Verification of HTLV-1 ZFP repressor activity and expression. (FIG. 7A) Schematic of the ZFP expression vector. CMV=cytomegalovirus promoter, NLS=nuclear localization signal, KRAB=kruppel-associated box, PA=polyA transcription terminator. Generic (KRAB) or ZFP specific (ZFP3/5) primer binding sites for detection of the expressed ZFP RNA are indicated. (FIG. 7B) HEK293 cells were transfected with a vector that contains a HTLV-1 LTR bidirectionally driving the expression Rluc (anti-sense) and Fluc (sense) luciferase. A series of HTLV-I ZFP-KRAB (2-10) were transfected with the reporter vector and 48 hrs post-transfection the levels of luciferase were determined. (FIG. 7C, FIG. 7D) HEK293 were transfected with a vector containing the HTLV-I 3′-LTR driving the expression of the HBZ-3×FLAG with the ZFP expression vectors, and at 48 hrs post-transfection the levels of HBZ RNA were assessed. (FIG. 7C) Both spliced (HBZsp), unspliced HBZ RNA (HBZusp), (FIG. 7D) KRAB, or ZFP3, ZFP5, RNA was determined. For (FIG. 7B-7D), error bars represent standard deviation from samples treated in triplicate from two independent experiments. For (FIG. 7B), the levels of luciferase or HBZ RNA were made relative to a ZFP-HIV-KRAB control set a 100%.

FIGS. 8A-8C: Assessing anti-HTLV-I DNA vectors for anti-proliferative effects. TL-Om1 cells were electroporated with DNA vectors expressing the ZFP5-KRAB or ZFP6-KRAB and outgrowth measured up to day 24 through (FIG. 8A) proliferation, (FIG. 8B) viability or (FIG. 8C) cell count. The ZFP-HIV-KRAB or GFP vectors were included as negative controls. Error bars represent standard deviation from samples treated in triplicate.

FIGS. 9A-9D: Screening of ZFP repressors with alternative repressor domains. (FIG. 9A) Schematic of the ZFP expression vectors with alternative repressor domains. CMV=cytomegalovirus promoter, NLS=nuclear localization signal, KRAB=kruppel-associated box, ZIM3=KRAB(ZIM3), meCP2=methyl CpG binding protein 2, PA=polyA transcription terminator. (FIG. 9B) HEK293 were transfected with a vector containing the HTLV-1 LTR bi-directional reporter to measure Fluc (sense) or the HBZ(spliced)-Rluc (anti-sense) activity with the ZFP5 variant vectors. At 48 hrs post-transfection the levels of luciferase activity were assessed. The ZFP5 variants were generated by fusing a KRAB, KRAB(ZIM3), KRAB-meCP2, PAM. A ZFP5 without a KRAB domain was also included (−). The levels of ZFP and HBZ (FIG. 9C) RNA or (FIG. 9D) protein were determined after transfecting HEK293 cells with an LTR-HBZ and the ZFP5 variants vectors. For (FIG. 9B) and (FIG. 9C), the ZFP5 variants were made relative to a control ZFP-HIV-KRAB, which was set a 100%. Error bars represent standard deviation from samples treated in triplicate. The levels of luciferase or HBZ RNA were made relative to a ZFP-HIV-KRAB control set a 100%. For (FIG. 9D), the HBZ and ZFPs were detected through a FLAG tag and myc tag, respectively. Untreated cells (mock) were included as ZFP and HBZ detection controls. Alpha-tubulin was detected as a loading control.

FIGS. 10A-10F: The anti-HTLV-I ZFPs do not affect a non-HTLV-I transformed T-cell line. Jurkat cells were electroporated with an (FIG. 10A) 2 μg ‘low’ dose or (FIG. 10B) 4 μg ‘high’ dose of mRNA expressing the ZFP5-KRAB or ZFP5-KRAB-meCP2 and outgrowth measured up to day 21 through proliferation (top panel), viability (middle panel) or cell count (bottom panel). (FIG. 10C) HEK293 cells stably expressing GFP from a LTR from HIV-1 was transfected with the ZFP5-KRAB, ZFP5-KRAB-meCP2 and ZFP-HIV-KRAB expression vectors, and 72 hrs post-transfection the levels of GFP were assessed by flow cytometry. An empty vector (pUC19) was included as a negative control. Short hairpin RNAs (shRNAs) targeted to the HIV-1 promoter (shRNA-362) and GFP (shRNA-GFP) were included as positive controls. ATL55T(+) cells were electroporated with 4 μg of ZFP5-KRAB and the levels of (FIG. 10D) HBZ and TAX RNA was assessed at 24 hrs post-electroporation. (FIG. 10E) ATL55T(+) cell line proliferation and (FIG. 10F) cell counts were assessed at day 3 and 6. The ZFP-HIV-KRAB or GFP mRNAs were included as negative controls. Error bars represent standard deviation from samples treated in triplicate.

FIGS. 11A-11C: Detection of HBZ and anti-HTLV-I ZFP molecules. TL-Om1 cells were electroporated with 2 μg or 4 μg of ZFP mRNA and the (FIG. 11A) RNA (KRAB) or (FIG. 11B) protein (anti-myc) was assessed. Untreated (mock) cells were included as a ZFP detection control. Alpha-tubulin was detected as a loading control. (FIG. 11C) TL-Om1 cells were electroporated with 2 μg of mRNA and the ZFP (KRAB), HBZsp, or HBZusp RNA was detected at 24, 48, and 72 hrs post-electroporation. A ZFP-HIV-KRAB mRNA was included as a negative control. Error bars represent standard deviation from samples treated in triplicate. The levels of HBZ RNA were made relative to a ZFP-HIV-KRAB control set a 100%.

FIGS. 12A-12C: TL-Om1 cells were electroporated with 4 μg (or 2 μg as indicated as ‘low’) of ZFP5-KRAB or ZFP5-KRAB-meCP2 mRNA, and the levels of (FIG. 12A) HBZ spliced RNA, (FIG. 12B) CCR4 RNA (24 hrs only), (FIG. 12C) or surface CCR4 receptor was assessed at 24 hrs and 48 hrs post-electroporation. Cells treated with the ZFP-HIV-KRAB mRNA or untreated cells (mock) were included as negative controls. Error bars represent standard deviation from samples treated in triplicate and p-values were determined by one-way ANOVA analysis (Dunnett's post-test) when compared to the ZFP-HIV-control (*p<0.05, **p<0.01).

FIGS. 13A-13C: ZFP5-KRAB-meCP2 is a more potent inhibitor of the HTLV-I LTR. (FIG. 13A) Jurkat cells were selected to stably express the HBZ gene expressed off a HTLV-I 3′ LTR in-frame with an internal ribosomal entry site (IRES) and a GFP-puromycin fusion protein (GFP-puro). (FIG. 13B) The Jurkat cells containing the LTR-HBZ-IRES-GFP construct were electroporated with 2 μg of ZFP5-KRAB or ZFP5-KRAB-meCP2 mRNA, and the percentage of GFP negative cells was assessed by flow cytometry at day 1, 2 or 4 post-electroporation. (FIG. 13C) Data from FIG. 13B represented as the percentage of GFP positive cells as assessed by flow cytometry at day 1, 2 or 4 post-electroporation. Error bars represent standard deviation from samples treated in triplicate. Cells treated with the ZFP-HIV-KRAB mRNA were included as a control.

FIG. 14: Anti-HTLV-I ZFP induce caspase activity. TL-Om1 cells were electroporated with 2 μg ‘low’ or 4 μg ‘high’ of ZFP5-KRAB or ZFP5-KRAB-meCP2 mRNA, and the levels of caspase 3/7 activity was assessed 24 hrs post-electroporation. Cells treated with the ZFP-HIV-KRAB mRNA or untreated cells (mock) were included as negative controls. Error bars represent standard deviation from samples treated in triplicate.

FIG. 15: Effect of ZFP repressor on the Fluc levels from a vector with an LTR from different HTLV-I genotypes. HEK293 cells were transfected with an LTR(a-g) spliced reporter vector with the ZFP5-KRAB and ZFP5-KRAB-meCP2 vectors, and 48 hrs post-transfection the levels of Fluc luciferase were determined. Error bars represent standard deviation from samples treated in triplicate. The levels of luciferase were made relative to a ZFP-HIV-KRAB control set a 100%.

FIGS. 16A-16B: Schematic for the development of anti-HTLV-1 EV HBZ CCR4 targeted therapy. (FIG. 16A) Stable HEK293 cells are transduced to express the EXOtic EV producer machinery including Connexion (CX43)(7), the HTLV-1 epigenetic repressor, ZFP5-KRAB/meCP2-CD mRNA (ZFP5-KrMe-CD), CD63-L7ae or CD63-anti-CCR4 for CCR4 targeted EVs. Over-expression of ZFP5-KrMe-CD results in expression and de novo packaging of ZFP5-KRAB/meCP2 protein (8). (FIG. 16B) Three different EVs are generated and tested in this proposal containing ZFP5 fused to KRAB and meCP2; the untargeted EV-a (ZFP5-KrMe), and the CCR4 targeted EVs; EV-b which consists of the PTGFRN CCR4 scFV fusion (ZFP5-KrMe-PTGFRN-R4) and EV-c which consists of CD63 fused to CCR4 (ZFP5-KrMe-CD63-R4). The EVs (EV-a-c) become taken up by HTLV-1 infected T-cells and deliver the HTLV-1 HBZ epigenetic repressor (ZFP5-KrMe-CD) mRNA and corresponding proteins (ZFP5-KrMe) both packaged into the EVs. The ZFP5-KrMe protein translocates to the nucleus where it binds and epigenetically inhibits the HBZ promoter which leads to death of the HTLV-1 HBZ driven oncogenic T-cell.

FIG. 17: Receptor targeted exosomes. Schematic of the CD63 receptor and example insertion sites of an scFv or nanobody (Ex1.1, Ex2.2, Ex2.3, or Ex2.4).

FIG. 18: Model for EV treatment of HTLV-1 infected NOD SCID ß2m mouse. Human CD34+ cells from cord blood are injected at day 1 or 2 after birth and following total body irradiation at 100 cGy. After 12 weeks engraftment the mice will be injected with HTLV-1 (MOI=5.0) infected donor matched CD4+ T-cells. The infection with HTLV-1 is monitored on weeks 4 and 8 post-infection for HTLV-1 infection by ELISA (p19) (Ji, 2020 #4460) and qRT-PCR for HBZ and Tax mRNA expression. Following detectable infection ˜week 8, EVs are administered R.O. every week thereafter until week 14. At week 14 and bi-weekly blood draws will be carried out to measure anti-HTLV-1 effects of the EV treatment and HTLV-1 persistence. The mice are euthanized and analysed at 18 weeks post-viral infection for tissue harvest and analysis (˜32 weeks post-transplantation).

FIGS. 19A-19B: LTR-targeted ZFP repressors reduce chromatin accessibility. TL-Om1 cells were electroporated with 4 μg of mRNA expressing the ZFP5-KRAB or ZFP5-KRAB-meCP2 and at 24 hrs the cells were subjected to ATAC-seq to assess chromatin accessibility. (FIG. 19A) Integrated genomic viewer (IGV) of the HTLV-I genome displaying accessibility. (FIG. 19B) Enrichment plot of nucleosome-free regions across HTLV-I's LTR. The read counts are the average of triplicate treated cells.

FIGS. 20A-20B: Specificity of the ZFP-KRAB vectors. (FIG. 20A) HEK293 cells were transfected with the HTLV-I 3′-LTR driving the expression of the HBZ-3×FLAG with the ZFP5-KRAB vector, and 48 hrs post-transfection the levels of HBZ RNA and protein were assessed. (FIG. 20B) Jurkat cells were electroporated with 2 μg of mRNA expressing the ZFP3-KRAB or ZFP-HIV-KRAB and proliferation was assessed at day 3. Error bars represent standard deviation from samples treated in triplicate.

FIGS. 21A-21B: Anti-HTLV-I ZFPs effects in TL-Om1 cells. (FIG. 21A) The levels of HBZ and TAX RNA was determined for MT-2, MT-4, Jurkat and TL-Om1 cells. (FIG. 21B) TL-Om1 cells were electroporated with a 2 μg ‘low’ dose or 4 μg ‘high’ dose of mRNA expressing the ZFP5-KRAB or ZFP5-KRAB-meCP and the number of viable cells per ml was determined using flow cytometry at day 2 and 5 (top panels), and day 3 and 6 (bottom panels). The ZFP-HIV-KRAB was included as negative controls. Error bars represent standard deviation from samples treated in triplicate.

FIGS. 22A-22C: Pathway analysis on a ATL cell line treated with anti-HTLV ZFPs. TL-Om1 cells were electroporated with 4 μg of (FIG. 21A) ZFP5-KRAB, (FIG. 21B) ZFP5-KRAB-meCP2, or (FIG. 21C) ZFP-HIV-KRAB mRNA and subjected to ATAC-seq. KEGG pathway analysis was performed for the ZFPs and each compared to mock treated cells. Dot size corresponds to gene ratio. Moreover, adjusted p values are also indicated.

FIG. 23: Reduced viability with ZFP5-HTLV treatment in ATL55T(+) cells compared to control.

FIG. 24: ATAC-seq reads reduced at a known enhancer site within SRF-ERK1 site in the HTLV ZFP treated samples compared to controls.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. In embodiments, the nucleic acid provided herein may be part of a lentiviral vector, which may be transduced into a cell. Polynucleotides useful in the methods of the disclosure may comprise natural nucleic acid sequences and variants thereof, artificial nucleic acid sequences, or a combination of such sequences.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 75%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

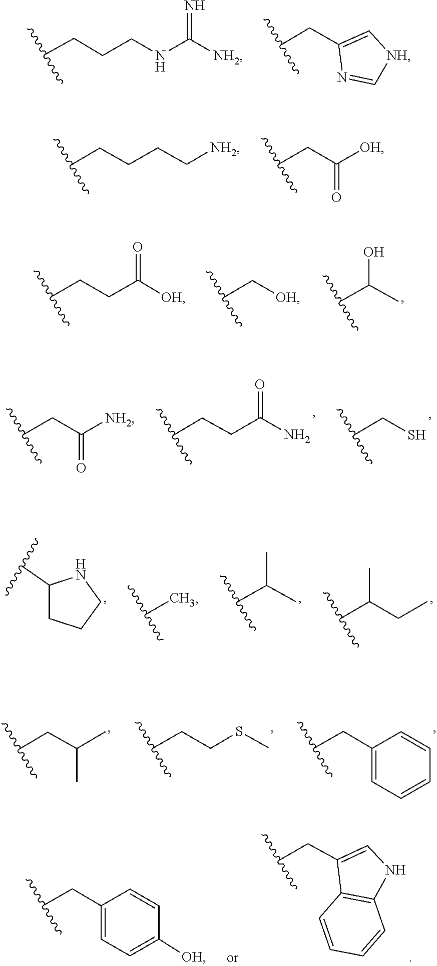

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

A “fusion protein” refers to a chimeric protein encoding two or more separate protein sequences that are recombinantly expressed as a single moiety. Because the different proteins in fusion proteins may affect the functionality of other proteins under certain circumstances, peptide linkers may be used between different proteins within the same fusion protein. These peptide linkers may have a flexible structure and separate the proteins within the fusion protein so that each protein in the fusion proteins substantially retains its function. Peptide linkers are known in the art and described, for example, in Chen et al, Adv Drug Deliv Rev, 65(10); 1357-1369 (2013).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 75%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. The preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

For specific proteins described herein, the named protein includes any of the protein's naturally occurring forms, variants or homologs that maintain activity of the protein (e.g., within at least 50%, 75%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “HBZ protein” or “HBZ” as used herein includes any of the recombinant or naturally-occurring forms of HTLV-1 basic zipper factor (HBZ), or variants or homologs thereof that maintain HBZ activity (e.g. within at least 50%, 80%, 90%, 95%, 96%, 97%, 98%, 99% or 100% activity compared to HBZ). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HBZ protein. In embodiments, the HBZ protein is substantially identical to the protein identified by the UniProt reference number POC746 or a variant or homolog having substantial identity thereto.

The term “meCP2 protein” or “meCP2” as used herein includes any of the recombinant or naturally-occurring forms of methyl CpG binding protein 2 (meCP2), also known as demethylase, DMTase, or variants or homologs thereof that maintain meCP2 activity (e.g. within at least 50%, 80%, 90%, 95%, 96%, 97%, 98%, 99% or 100% activity compared to meCP2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring meCP2 protein. In embodiments, the meCP2 protein is substantially identical to the protein identified by the UniProt reference number Q9UBB5 or a variant or homolog having substantial identity thereto. In embodiments, the meCP2 protein includes a sequence having at least 90%, 95%, 96%, 97%, 98%, 99% or 100% sequence identity to the sequence of SEQ ID NO:125. In embodiments, the meCP2 protein includes a sequence having at least 80% sequence identity to the sequence of SEQ ID NO:125. In embodiments, the meCP2 protein includes a sequence having at least 90% sequence identity to the sequence of SEQ ID NO:125. In embodiments, the meCP2 protein includes a sequence having at least 95% sequence identity to the sequence of SEQ ID NO:125. In embodiments, the meCP2 protein includes a sequence having at least 96% sequence identity to the sequence of SEQ ID NO:125. In embodiments, the meCP2 protein includes a sequence having at least 97% sequence identity to the sequence of SEQ ID NO:125. In embodiments, the meCP2 protein includes a sequence having at least 98% sequence identity to the sequence of SEQ ID NO:125. In embodiments, the meCP2 protein includes a sequence having at least 99% sequence identity to the sequence of SEQ ID NO:125. In embodiments, the meCP2 protein includes the sequence of SEQ ID NO:125. In embodiments, the meCP2 protein is the sequence of SEQ ID NO:125.

The term “DNA methyltransferase” or “DNA methyltransferase protein” as provided herein refers to an enzyme that catalyzes the transfer of a methyl group to DNA. Non-limiting examples of DNA methyltransferases include Dnmt1, Dnmt3A, and Dnmt3B. In aspects, the DNA methyltransferase is mammalian DNA methyltransferase. In aspects, the DNA methyltransferase is human DNA methyltransferase. In aspects, the DNA methyltransferase is mouse DNA methyltransferase. In aspects, the DNA methyltransferase is a bacterial cytosine methyltransferase and/or a bacterial non-cytosine methyltransferase. Depending on the specific DNA methyltransferase, different regions of DNA are methylated. For example, Dnmt3A typically targets CpG dinucleotides for methylation. Through DNA methylation, DNA methyltransferases can modify the activity of a DNA segment (e.g., gene expression) without altering the DNA sequence. In aspects, DNA methylation results in repression of gene transcription and/or modulation of methylation sensitive transcription factors or CTCF. As described herein, fusion proteins may include one or more (e.g., two) DNA methyltransferases. When a DNA methyltransferase is included as part of a fusion protein, the DNA methyltransferase may be referred to as a “DNA methyltransferase domain.”

A “Dnmt3A”, “Dnmt3a,” “DNA (cytosine-5)-methyltransferase 3A” or “DNA methyltransferase 3a” protein as referred to herein includes any of the recombinant or naturally-occurring forms of the Dnmt3A enzyme or variants or homologs thereof that maintain Dnmt3A enzyme activity (e.g. within at least 50%, 55%, 60%, 65%, 70%, 75%, 80%, 85%, 90%, 91%, 92%, 93%, 94%, 95%, 96%, 97%, 98%, 99% or 100% activity compared to Dnmt3A). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Dnmt3A protein. In aspects, the Dnmt3A protein is substantially identical to the protein identified by the UniProt reference number Q9Y6K1 or a variant or homolog having substantial identity thereto.

The term “Kruppel associated box domain” or “KRAB domain” as provided herein refers to a category of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, 2017; Lambert et al. The human transcription factors, Cell 172, 2018; Gilbert et al., Cell (2013); and Gilbert et al., Cell (2014). In embodiments, the KRAB domain includes a sequence having at least 80%, 90%, 95%, 96%, 97%, 98%, 99% or 100% sequence identity to the sequence of SEQ ID NO:123. In embodiments, the KRAB domain includes a sequence having at least 80% sequence identity to the sequence of SEQ ID NO:123. In embodiments, the KRAB domain includes a sequence having at least 90% sequence identity to the sequence of SEQ ID NO:123. In embodiments, the KRAB domain includes a sequence having at least 95% sequence identity to the sequence of SEQ ID NO:123. In embodiments, the KRAB domain includes a sequence having at least 96% sequence identity to the sequence of SEQ ID NO:123. In embodiments, the KRAB domain includes a sequence having at least 97% sequence identity to the sequence of SEQ ID NO:123. In embodiments, the KRAB domain includes a sequence having at least 98% sequence identity to the sequence of SEQ ID NO:123. In embodiments, the KRAB domain includes a sequence having at least 99% sequence identity to the sequence of SEQ ID NO:123. In embodiments, the KRAB domain includes the sequence of SEQ ID NO:123. In embodiments, the KRAB domain is the sequence of SEQ ID NO:123.

The term “CD63 protein” or “CD63” as used herein includes any of the recombinant or naturally-occurring forms of CD63, also known as Granulophysin, Lysosomal-associated membrane protein 3, LAMP-3, Lysosome integral membrane protein 1, Limp1, or variants or homologs thereof that maintain CD63 activity (e.g. within at least 50%, 80%, 90%, 95%, 96%, 97%, 98%, 99% or 100% activity compared to CD63). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD63 protein. In embodiments, the CD63 protein is substantially identical to the protein identified by the UniProt reference number P08962 or a variant or homolog having substantial identity thereto.

The term “PTGFRN protein” or “PTGFRN” as used herein includes any of the recombinant or naturally-occurring forms of Prostaglandin F2 receptor negative regulator (PTGFRN), also known as CD9 partner 1, EWI motif-containing protein F, CD315, or variants or homologs thereof that maintain PTGFRN activity (e.g. within at least 50%, 80%, 90%, 95%, 96%, 97%, 98%, 99% or 100% activity compared to PTGFRN). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PTGFRN protein. In embodiments, the PTGFRN protein is substantially identical to the protein identified by the UniProt reference number Q9P2B2 or a variant or homolog having substantial identity thereto.

The term “CD9 protein” or “CD9” as used herein includes any of the recombinant or naturally-occurring forms of CD9, also known as MIC3, or TSPAN29, or variants or homologs thereof that maintain CD9 activity (e.g. within at least 50%, 80%, 90%, 95%, 96%, 97%, 98%, 99% or 100% activity compared to CD9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD9 protein. In embodiments, the CD9 protein is substantially identical to the protein identified by the UniProt reference number P21926 or a variant or homolog having substantial identity thereto.

The term “CCR4 protein” or “CCR4” as used herein includes any of the recombinant or naturally-occurring forms of C-C chemokine receptor type 4 (CCR4), also known as K5-5, CD194, or variants or homologs thereof that maintain CCR4 activity (e.g. within at least 50%, 80%, 90%, 95%, 96%, 97%, 98%, 99% or 100% activity compared to CCR4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CCR4 protein. In embodiments, the CCR4 protein is substantially identical to the protein identified by the UniProt reference number P51679 or a variant or homolog having substantial identity thereto.

The term “CD4 protein” or “CD4” as used herein includes any of the recombinant or naturally-occurring forms of CD4, also known as T-cell surface glycoprotein CD4, T-cell surface antigen T4/Leu-3 or variants or homologs thereof that maintain CD4 activity (e.g. within at least 50%, 80%, 90%, 95%, 96%, 97%, 98%, 99% or 100% activity compared to CD4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 protein. In embodiments, the CD4 protein is substantially identical to the protein identified by the UniProt reference number P01730 or a variant or homolog having substantial identity thereto.

The term “OX40 protein” or “OX40” as used herein includes any of the recombinant or naturally-occurring forms of OX40, also known as tumor necrosis factor receptor superfamily member 4 (TNFRSF4), ACT35 antigen, TAX transcriptionally-activated glycoprotein 1 receptor, CD134, or variants or homologs thereof that maintain OX40 activity (e.g. within at least 50%, 80%, 90%, 95%, 96%, 97%, 98%, 99% or 100% activity compared to OX40). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring OX40 protein. In embodiments, the OX40 protein is substantially identical to the protein identified by the UniProt reference number P43489 or a variant or homolog having substantial identity thereto.

The term “CD5 protein” or “CD5” as used herein includes any of the recombinant or naturally-occurring forms of CD5, also known as T-cell surface glycoprotein CD5, lymphocyte antigen T1/Leu-1, or variants or homologs thereof that maintain CD5 activity (e.g. within at least 50%, 80%, 90%, 95%, 96%, 97%, 98%, 99% or 100% activity compared to CD5). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD5 protein. In embodiments, the CD5 protein is substantially identical to the protein identified by the UniProt reference number P06127 or a variant or homolog having substantial identity thereto.

The term “CD25 protein” or “CD25” as used herein includes any of the recombinant or naturally-occurring forms of CD25, also known as Interleukin-2 receptor subunit alpha, TAC antigen, p55, IL-2-RA, IL2-RA, or variants or homologs thereof that maintain CD25 activity (e.g. within at least 50%, 80%, 90%, 95%, 96%, 97%, 98%, 99% or 100% activity compared to CD25). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD25 protein. In embodiments, the CD25 protein is substantially identical to the protein identified by the UniProt reference number P01589 or a variant or homolog having substantial identity thereto.

The term “lactadherin protein” or “lactadherin” as used herein includes any of the recombinant or naturally-occurring forms of lactadherin, also known as breast epithelial antigen BA46, IMIFG, MFGM, milk fat globule-EGF factor 8, SED1, or variants or homologs thereof that maintain lactadherin activity (e.g. within at least 50%, 80%, 90%, 95%, 96%, 97%, 98%, 99% or 100% activity compared to lactadherin). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring lactadherin protein. In embodiments, the lactadherin protein is substantially identical to the protein identified by the UniProt reference number Q08431 or a variant or homolog having substantial identity thereto.

The term “CD37 protein” or “CD37” as used herein includes any of the recombinant or naturally-occurring forms of CD37, also known as leukocyte antigen CD37, tetraspanin-26, or variants or homologs thereof that maintain CD37 activity (e.g. within at least 50%, 80%, 90%, 95%, 96%, 97%, 98%, 99% or 100% activity compared to CD37). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD37 protein. In embodiments, the CD37 protein is substantially identical to the protein identified by the UniProt reference number P11049 or a variant or homolog having substantial identity thereto.

The term “LAMP-1 protein” or “LAMP-1” as used herein includes any of the recombinant or naturally-occurring forms of LAMP-1, also known lysosome-associated membrane glycoprotein 1, CD107a, or variants or homologs thereof that maintain LAMP-1 activity (e.g. within at least 50%, 80%, 90%, 95%, 96%, 97%, 98%, 99% or 100% activity compared to LAMP-1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LAMP-1 protein. In embodiments, the LAMP-1 protein is substantially identical to the protein identified by the UniProt reference number P11279 or a variant or homolog having substantial identity thereto.

The term “LAMP-2A protein” or “LAMP-2A” as used herein includes any of the recombinant or naturally-occurring forms of LAMP-2A, also known lysosome-associated membrane glycoprotein 2, CD107b, LGP-96, LAMP-2, or variants or homologs thereof that maintain LAMP-2A activity (e.g. within at least 50%, 80%, 90%, 95%, 96%, 97%, 98%, 99% or 100% activity compared to LAMP-2A). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LAMP-2A protein. In embodiments, the LAMP-2A protein is substantially identical to the protein identified by the UniProt reference number P13473 or a variant or homolog having substantial identity thereto.

The term “CD70 protein” or “CD70” as used herein includes any of the recombinant or naturally-occurring forms of CD70, also known as CD27 ligand, tumor necrosis factor ligand superfamily member 7, or variants or homologs thereof that maintain CD70 activity (e.g. within at least 50%, 80%, 90%, 95%, 96%, 97%, 98%, 99% or 100% activity compared to CD70). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD70 protein. In embodiments, the CD70 protein is substantially identical to the protein identified by the UniProt reference number P32970 or a variant or homolog having substantial identity thereto.

The term “IL15RA protein” or “IL15RA” as used herein includes any of the recombinant or naturally-occurring forms of IL15RA, also known as CD215, soluble interleukin-15 receptor subunit alpha, IL-15 receptor subunit alpha, tumor necrosis factor ligand superfamily member 7, or variants or homologs thereof that maintain IL15RA activity (e.g. within at least 50%, 80%, 90%, 95%, 96%, 97%, 98%, 99% or 100% activity compared to IL15RA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL15RA protein. In embodiments, the IL15RA protein is substantially identical to the protein identified by the UniProt reference number Q13261 or a variant or homolog having substantial identity thereto.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain” or “V_{H},” refers to the variable region of an immunoglobulin heavy chain, including an Fv, scFv, dsFv or Fab; while the terms “variable light chain” or “V_{L}” refers to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., Fundamental Immunology (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A “ligand” refers to an agent, e.g., a polypeptide or other molecule, capable of binding to a receptor or antibody, antibody variant, antibody region or fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

As used herein, the term “construct” is intended to mean any recombinant nucleic acid molecule. In embodiments, a construct includes an expression cassette, plasmid, cosmid, virus, autonomously replicating polynucleotide molecule, phage, or linear or circular, single-stranded or double-stranded, DNA or RNA polynucleotide molecule. A construct may be derived from any source, capable of genomic integration or autonomous replication, including a nucleic acid molecule where one or more nucleic acid sequences has been linked in a functionally operative manner, e.g., operably linked.

The terms “operably linked” or “functionally linked”, are interchangeable and denote a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter, an LTR, a sequence within an LTR) is functional link that allows for expression of the polynucleotide of interest. In this sense, the term “operably linked” refers to the positioning of a regulatory region (e.g. an LTR, a sequence within an LTR) and a coding sequence (e.g. polynucleotide encoding a gene editing agent, etc.) to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, operably linked elements may be contiguous or non-contiguous. In addition, in the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, regions, or domains of the engineered antibodies disclosed herein may be operably linked to retain proper folding, processing, targeting, expression, binding, and other functional properties of the engineered antibodies in the cell. Operably linked regions, domains, and segments of the engineered antibodies of the disclosure may be contiguous or non-contiguous (e.g., linked to one another through a linker).

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a lentiviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Transduce” or “transduction” are used according to their plain ordinary meanings and refer to the process by which one or more foreign nucleic acids (i.e. DNA not naturally found in the cell) are introduced into a cell. Typically, transduction occurs by introduction of a virus or viral vector (e.g. a CMV vector, a lentivirus vector, etc.) into the cell.

As used herein, the term “promoter” refers to a sequence of DNA which proteins bind to initiate gene expression. For example, transcription factors may bind a promoter region of a gene to transcribe RNA from DNA. In embodiments, the HTLV-1 LRT functions as a promoter for the HBZ gene.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be, for example, a nucleic acid as provided herein and a cell. In embodiments contacting includes, for example, allowing a nucleic acid as described herein to interact with a cell. Thus, in embodiments, contacting includes allowing a nucleic acid to interact with a cell, thereby resulting in transduced cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule.

In the context of a virus, the term “replicate” includes the ability of a virus to replicate (duplicate the viral genome and packaging said genome into viral particles) in a host cell and subsequently release progeny viruses from the host cell, which results in the lysis of the host cell.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express proteins that are not found within the native (non-recombinant) form of the cell.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In aspects inhibition means negatively affecting (e.g. decreasing) the concentration or levels of the protein relative to the concentration or level of the protein in the absence of the inhibitor. In aspects inhibition refers to reduction of a disease or symptoms of disease. In aspects, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In aspects, inhibition refers to a reduction of activity of a target protein resulting from a direct interaction (e.g. an inhibitor binds to the target protein). In aspects, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 75%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient”, “subject” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a human T-cell lymphotropic virus type 1 (HTLV-1) associated disease. The HTLV-1 associated disease may be adult T-cell leukemia, adult T-cell lymphoma, HTLV-1 associated myelopathy, tropical spastic paraparesis, or HTLV-1 infection.

The term “associated” or “associated with” in the context of a substance or substance activity or function associated with a disease means that the disease (e.g. adult T-cell leukemia, adult T-cell lymphoma, HTLV-1 Associated Myelopathy, Tropical spastic paraparesis, HTLV-1 infection) is caused by (in whole or in part), or a symptom of the disease is caused by (in whole or in part) the substance or substance activity or function. For example, an HTLV-1 associated disease may be caused by HTVL-1 infection. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things. Thus in the disclosed method, treatment can refer to a 10%, 20%, 30%, 40%, 50%, 60%, 70%, 75%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 75%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 75%, 90% or greater as compared to a control level and such terms can include but do not necessarily include complete elimination.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Zinc Finger Containing Proteins

Provided herein, inter alia, are compositions including a protein having a zinc finger domain where the zinc finger domain binds a sequence within the long terminal repeat (LTR) of Human T-cell lymphotropic virus type 1 (HTLV-1). Applicant has discovered that binding of the zinc finger domain to the sequence within the HTLV-1 LTR potently suppresses HTLV-1 bZIP factor (HBZ) expression. The term “zinc finger domain” refers to a protein, or a domain within a larger protein, that binds DNA in a sequence-specific manner through one or more zinc fingers. Zinc fingers are regions of amino acid sequences whose structure is typically stabilized through coordination of a metal (e.g. a zinc ion). In embodiments, a zinc finger may adopt a structure including an antiparallel β sheet followed by an α helix. In embodiments, a zinc finger includes an antiparallel β sheet including two β strands followed by an α helix. Any of the zinc finger domains described herein may include 1, 2, 3, 4, 5, 6 or more zinc fingers, each zinc finger having a recognition helix region that binds a sequence within the LTR of HTLV-1. In embodiments, the zinc finger domain includes 4, 5 or 6 zinc fingers. In embodiments, the zinc finger domain includes 4 zinc fingers. In embodiments, the zinc finger domain includes 5 zinc fingers. In embodiments, the zinc finger domain includes 6 zinc fingers. In embodiments, the individual zinc fingers include zinc finger recognition helix regions (e.g. recognition helix regions), wherein the zinc finger recognition helix regions are designated F1, F2, F3, F4, F5 and F6, and include the amino acid sequences of the recognition helix regions as shown in Table 4. As used herein, zinc finger recognition helix region (e.g. recognition helix region), refers to a subportion of the zinc finger that makes specific contacts with a target nucleic acid sequence (e.g. a sequence within the HTLV-1 LTR). For example, a zinc finger recognition helix region may be a sequence within an α-helix structure within the zinc finger that makes specific contacts with a target nucleic acid sequence (e.g. a sequence within the HTLV-1 LTR).

In embodiments, the zinc finger domain is non-naturally occurring in that it is engineered to bind to a target site of choice. There is generally a wide range of sequence variation in the amino acids of the known zinc finger domains. In embodiments, a zinc finger domain has a sequence of the form X₃-Cys-X₂-4-Cys-Xu-His-X₃₋₅-His-X₄, wherein X is any amino acid (e.g., X₂₄ indicates an oligopeptide 2-4 amino acids in length). In embodiments, only the two consensus histidine residues and two consensus cysteine residues bound to the central zinc atom are invariant. Of the remaining residues, typically three to five are highly conserved, while there may be significant variation among the other residues. Despite the wide range of sequence variation in zinc finger domains, zinc finger domains of this type generally have a similar three dimensional structure. However, there is a wide range of binding specificities among the different zinc finger domains, i.e., different zinc fingers may bind double stranded polynucleotides having a wide range of nucleotides sequences. In embodiments, the zinc finger domain is the C2H2 type. In embodiments, the zinc finger domain is the CCHC type. In embodiments, the zinc finger domain is the PHD type. In embodiments, the zinc finger domain is the RING type.

In embodiments, the zinc finger domain recognizes with specificity (e.g. specifically binds) about 3 bases, about 4 bases, about 5 bases, about 6 bases, about 7 bases, about 8 bases, about 9 bases, about 10 bases, about 11 bases, about 12 bases, about 13 bases, about 14 bases, about 15 bases, about 16 bases, about 18 bases, about 20 bases, about 22 bases, about 24 bases, about 26 bases, about 28 bases, about 30 bases, about 32 bases, about 34 bases, about 36 bases, about 38 bases, or about 40 bases of a recognized sequence (e.g. a sequence within the HTLV-1 LTR). In embodiments, the zinc finger domain recognizes with specificity about 3 bases of a recognized sequence (e.g. a sequence within the HTLV-1 LTR). In embodiments, the zinc finger domain recognizes with specificity about 4 bases of a recognized sequence (e.g. a sequence within the HTLV-1 LTR). In embodiments, the zinc finger domain recognizes with specificity about 5 bases of a recognized sequence (e.g. a sequence within the HTLV-1 LTR). In embodiments, the zinc finger domain recognizes with specificity about 6 bases of a recognized sequence (e.g. a sequence within the HTLV-1 LTR). In embodiments, the zinc finger domain recognizes with specificity about 7 bases of a recognized sequence (e.g. a sequence within the HTLV-1 LTR). In embodiments, the zinc finger domain recognizes with specificity about 8 bases of a recognized sequence (e.g. a sequence within the HTLV-1 LTR). In embodiments, the zinc finger domain recognizes with specificity about 9 bases of a recognized sequence (e.g. a sequence within the HTLV-1 LTR). In embodiments, the zinc finger domain recognizes with specificity about 10 bases of a recognized sequence (e.g. a sequence within the HTLV-1 LTR). In embodiments, the zinc finger domain recognizes with specificity about 12 bases of a recognized sequence (e.g. a sequence within the HTLV-1 LTR). In embodiments, the zinc finger domain recognizes with specificity about 14 bases of a recognized sequence (e.g. a sequence within the HTLV-1 LTR). In embodiments, the zinc finger domain recognizes with specificity about 16 bases of a recognized sequence (e.g. a sequence within the HTLV-1 LTR). In embodiments, the zinc finger domain recognizes with specificity about 18 bases of a recognized sequence (e.g. a sequence within the HTLV-1 LTR). In embodiments, the zinc finger domain recognizes with specificity about 20 bases of a recognized sequence (e.g. a sequence within the HTLV-1 LTR). In embodiments, the zinc finger domain recognizes with specificity about 22 bases of a recognized sequence (e.g. a sequence within the HTLV-1 LTR). In embodiments, the zinc finger domain recognizes with specificity about 24 bases of a recognized sequence (e.g. a sequence within the HTLV-1 LTR). In embodiments, the zinc finger domain recognizes with specificity about 26 bases of a recognized sequence (e.g. a sequence within the HTLV-1 LTR). In embodiments, the zinc finger domain recognizes with specificity about 28 bases of a recognized sequence (e.g. a sequence within the HTLV-1 LTR). In embodiments, the zinc finger domain recognizes with specificity about 30 bases of a recognized sequence (e.g. a sequence within the HTLV-1 LTR). In embodiments, the zinc finger domain recognizes with specificity about 32 bases of a recognized sequence (e.g. a sequence within the HTLV-1 LTR). In embodiments, the zinc finger domain recognizes with specificity about 34 bases of a recognized sequence (e.g. a sequence within the HTLV-1 LTR). In embodiments, the zinc finger domain recognizes with specificity about 36 bases of a recognized sequence (e.g. a sequence within the HTLV-1 LTR). In embodiments, the zinc finger domain recognizes with specificity about 38 bases of a recognized sequence (e.g. a sequence within the HTLV-1 LTR). In embodiments, the zinc finger domain recognizes with specificity about 40 bases of a recognized sequence (e.g. a sequence within the HTLV-1 LTR). In embodiments, the zinc finger domain recognizes (e.g. binds to) a derivative of the target sequence which has at least 50%, 55%, 60%, 65%, 70%, 75%, 80%, 85%, 90%, 95%, 96%, 97%, 98% or 99% sequence identify to the target sequence (e.g. a sequence within the HTLV-1 LTR).

In embodiments, the zinc finger domain recognizes with specificity (e.g. specifically binds) about 3 bases to about 40 bases of a recognized sequence (e.g. a sequence within the HTLV-1 LTR). In embodiments, the zinc finger domain recognizes with specificity (e.g. specifically binds) about 6 bases to about 40 bases of a recognized sequence (e.g. a sequence within the HTLV-1 LTR). In embodiments, the zinc finger domain recognizes with specificity (e.g. specifically binds) about 9 bases to about 40 bases of a recognized sequence (e.g. a sequence within the HTLV-1 LTR). In embodiments, the zinc finger domain recognizes with specificity (e.g. specifically binds) about 12 bases to about 40 bases of a recognized sequence (e.g. a sequence within the HTLV-1 LTR). In embodiments, the zinc finger domain recognizes with specificity (e.g. specifically binds) about 15 bases to about 40 bases of a recognized sequence (e.g. a sequence within the HTLV-1 LTR). In embodiments, the zinc finger domain recognizes with specificity (e.g. specifically binds) about 18 bases to about 40 bases of a recognized sequence (e.g. a sequence within the HTLV-1 LTR). In embodiments, the zinc finger domain recognizes with specificity (e.g. specifically binds) about 21 bases to about 40 bases of a recognized sequence (e.g. a sequence within the HTLV-1 LTR). In embodiments, the zinc finger domain recognizes with specificity (e.g. specifically binds) about 24 bases to about 40 bases of a recognized sequence (e.g. a sequence within the HTLV-1 LTR). In embodiments, the zinc finger domain recognizes with specificity (e.g. specifically binds) about 27 bases to about 40 bases of a recognized sequence (e.g. a sequence within the HTLV-1 LTR). In embodiments, the zinc finger domain recognizes with specificity (e.g. specifically binds) about 30 bases to about 40 bases of a recognized sequence (e.g. a sequence within the HTLV-1 LTR). In embodiments, the zinc finger domain recognizes with specificity (e.g. specifically binds) about 33 bases to about 40 bases of a recognized sequence (e.g. a sequence within the HTLV-1 LTR). In embodiments, the zinc finger domain recognizes with specificity (e.g. specifically binds) about 36 bases to about 40 bases of a recognized sequence (e.g. a sequence within the HTLV-1 LTR).

In embodiments, the zinc finger domain recognizes with specificity (e.g. specifically binds) about 3 bases to about 36 bases of a recognized sequence (e.g. a sequence within the HTLV-1 LTR). In embodiments, the zinc finger domain recognizes with specificity (e.g. specifically binds) about 3 bases to about 33 bases of a recognized sequence (e.g. a sequence within the HTLV-1 LTR). In embodiments, the zinc finger domain recognizes with specificity (e.g. specifically binds) about 3 bases to about 30 bases of a recognized sequence (e.g. a sequence within the HTLV-1 LTR). In embodiments, the zinc finger domain recognizes with specificity (e.g. specifically binds) about 3 bases to about 27 bases of a recognized sequence (e.g. a sequence within the HTLV-1 LTR). In embodiments, the zinc finger domain recognizes with specificity (e.g. specifically binds) about 3 bases to about 24 bases of a recognized sequence (e.g. a sequence within the HTLV-1 LTR). In embodiments, the zinc finger domain recognizes with specificity (e.g. specifically binds) about 3 bases to about 21 bases of a recognized sequence (e.g. a sequence within the HTLV-1 LTR). In embodiments, the zinc finger domain recognizes with specificity (e.g. specifically binds) about 3 bases to about 18 bases of a recognized sequence (e.g. a sequence within the HTLV-1 LTR). In embodiments, the zinc finger domain recognizes with specificity (e.g. specifically binds) about 3 bases to about 15 bases of a recognized sequence (e.g. a sequence within the HTLV-1 LTR). In embodiments, the zinc finger domain recognizes with specificity (e.g. specifically binds) about 3 bases to about 12 bases of a recognized sequence (e.g. a sequence within the HTLV-1 LTR). In embodiments, the zinc finger domain recognizes with specificity (e.g. specifically binds) about 3 bases to about 9 bases of a recognized sequence (e.g. a sequence within the HTLV-1 LTR). In embodiments, the zinc finger domain recognizes with specificity (e.g. specifically binds) about 3 bases to about 6 bases of a recognized sequence (e.g. a sequence within the HTLV-1 LTR).

Long terminal repeats (LTRs) are used according to their plain and ordinary meaning and the art. Thus, LTR's may contain identical sequences of DNA or RNA that repeat tens, and more often hundreds or thousands of times found at either end of viral retroviral genome or proviral DNA that is formed by reverse transcription of retroviral RNA. LTRs may be used by viruses to insert their genetic material into the host genomes. The LTRs may be partially transcribed into an RNA intermediate, followed by reverse transcription into complementary DNA (cDNA) and ultimately dsDNA (double-stranded DNA) with full LTRs. The LTRs may then mediate integration of the retroviral DNA via an LTR specific integrase into another region of the host chromosome. In the proviral latency, once the provirus has been integrated, the LTR on the 5′ end may serve as the promoter for the entire retroviral genome, while the LTR at the 3′ end may provide for nascent viral RNA polyadenylation and encodes some accessory proteins. In embodiments, the protein provided herein including embodiments thereof targets (or binds to) a sequence within the 5′ LTR, 3′ LTR or both. In embodiments, the protein provided herein including embodiments thereof binds to a sequence within the 3′LTR.

Thus, in an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:27. In embodiments, the sequence has at least 75%, 80%, 85%, 90%, 95%, 96%, 97%, 98% or 99% sequence identity to the sequence of SEQ ID NO:27. In embodiments, the sequence has at least 90%, 95%, 96%, 97%, 98%, 99% or 100% nucleic acid sequence identity across the whole sequence or a portion of the sequence (e.g. a 5, 10, 11, 12, 13, 14, or 15 continuous nucleic acid portion) of SEQ ID NO:27.

In embodiments, the sequence within the HTLV-1 LTR has at least 75% sequence identity to the sequence of SEQ ID NO:27. In embodiments, the sequence within the HTLV-1 LTR has at least 80% sequence identity to the sequence of SEQ ID NO:27. In embodiments, the sequence within the HTLV-1 LTR has at least 85% sequence identity to the sequence of SEQ ID NO:27. In embodiments, the sequence within the HTLV-1 LTR has at least 90% sequence identity to the sequence of SEQ ID NO:27. In embodiments, the sequence within the HTLV-1 LTR has at least 95% sequence identity to the sequence of SEQ ID NO:27. In embodiments, the sequence within the HTLV-1 LTR has at least 98% sequence identity to the sequence of SEQ ID NO:27. In embodiments, the sequence within the HTLV-1 LTR has at least 99% sequence identity to the sequence of SEQ ID NO:27. In embodiments, the sequence within the HTLV-1 LTR includes the sequence of SEQ ID NO:27. In embodiments, the sequence within the HTLV-1 LTR is the sequence of SEQ ID NO:27.

In embodiments, the sequence within the HTLV-1 LTR has about 75% sequence identity to the sequence of SEQ ID NO:27. In embodiments, the sequence within the HTLV-1 LTR has about 80% sequence identity to the sequence of SEQ ID NO:27. In embodiments, the sequence within the HTLV-1 LTR has about 85% sequence identity to the sequence of SEQ ID NO:27. In embodiments, the sequence within the HTLV-1 LTR has about 90% sequence identity to the sequence of SEQ ID NO:27. In embodiments, the sequence within the HTLV-1 LTR has about 95% sequence identity to the sequence of SEQ ID NO:27. In embodiments, the sequence within the HTLV-1 LTR has about 98% sequence identity to the sequence of SEQ ID NO:27. In embodiments, the sequence within the HTLV-1 LTR has about 99% sequence identity to the sequence of SEQ ID NO:27.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:51, F2 includes SEQ ID NO:52, F3 includes SEQ ID NO:53, F4 includes SEQ ID NO:54, F5 includes SEQ ID NO:55 and F6 includes SEQ ID NO:56. In embodiments, the F1 is SEQ ID NO:51, F2 is SEQ ID NO:52, F3 is SEQ ID NO:53, F4 is SEQ ID NO:54, F5 is SEQ ID NO:55 and F6 is SEQ ID NO:56.

In embodiments, the zinc finger domain has at least 75%, 80%, 85%, 90%, 95%, 96%, 97%, 98% or 99% sequence identity to the sequence of SEQ ID NO:4. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:4. In embodiments, the zinc finger domain includes a sequence having at least 75% sequence identity to SEQ ID NO:4. In embodiments, the zinc finger domain includes a sequence having at least 80% sequence identity to SEQ ID NO:4. In embodiments, the zinc finger domain includes a sequence having at least 85% sequence identity to SEQ ID NO:4. In embodiments, the zinc finger domain includes a sequence having at least 90% sequence identity to SEQ ID NO:4. In embodiments, the zinc finger domain includes a sequence having at least 91% sequence identity to SEQ ID NO:4. In embodiments, the zinc finger domain includes a sequence having at least 92% sequence identity to SEQ ID NO:4. In embodiments, the zinc finger domain includes a sequence having at least 93% sequence identity to SEQ ID NO:4. In embodiments, the zinc finger domain includes a sequence having at least 94% sequence identity to SEQ ID NO:4. In embodiments, the zinc finger domain includes a sequence having at least 95% sequence identity to SEQ ID NO:4. In embodiments, the zinc finger domain includes a sequence having at least 96% sequence identity to SEQ ID NO:4. In embodiments, the zinc finger domain includes a sequence having at least 97% sequence identity to SEQ ID NO:4. In embodiments, the zinc finger domain includes a sequence having at least 98% sequence identity to SEQ ID NO:4. In embodiments, the zinc finger domain includes a sequence having at least 99% sequence identity to SEQ ID NO:4. In embodiments, the zinc finger domain includes the sequence of SEQ ID NO:4. In embodiments, the zinc finger domain is the sequence of SEQ ID NO:4.

In embodiments, the zinc finger domain includes a sequence having about 75% sequence identity to SEQ ID NO:4. In embodiments, the zinc finger domain includes a sequence having about 80% sequence identity to SEQ ID NO:4. In embodiments, the zinc finger domain includes a sequence having about 85% sequence identity to SEQ ID NO:4. In embodiments, the zinc finger domain includes a sequence having about 90% sequence identity to SEQ ID NO:4. In embodiments, the zinc finger domain includes a sequence having about 91% sequence identity to SEQ ID NO:4. In embodiments, the zinc finger domain includes a sequence having about 92% sequence identity to SEQ ID NO:4. In embodiments, the zinc finger domain includes a sequence having about 93% sequence identity to SEQ ID NO:4. In embodiments, the zinc finger domain includes a sequence having about 94% sequence identity to SEQ ID NO:4. In embodiments, the zinc finger domain includes a sequence having about 95% sequence identity to SEQ ID NO:4. In embodiments, the zinc finger domain includes a sequence having about 96% sequence identity to SEQ ID NO:4. In embodiments, the zinc finger domain includes a sequence having about 97% sequence identity to SEQ ID NO:4. In embodiments, the zinc finger domain includes a sequence having about 98% sequence identity to SEQ ID NO:4. In embodiments, the zinc finger domain includes a sequence having about 99% sequence identity to SEQ ID NO:4.

In embodiments, the zinc finger domain has a sequence with the percentage sequence identity as disclosed in the above paragraphs, and the sequence having the percentage sequence identity as disclosed above is a non-contiguous sequence. A “non-contiguous sequence” as provided herein refers to a sequence including one or more sequence fragments having no sequence identity to the indicated sequence. In embodiments, the non-contiguous sequence is a sequence including a first sequence fragment having at least the percentage sequence identity as disclosed above to SEQ ID NO:4 connected to a second sequence fragment having at least the percentage sequence identity as disclosed above to SEQ ID NO:4 through a sequence fragment having no sequence identity to SEQ ID NO:4. In embodiments, the non-contiguous sequence is a sequence including a plurality of sequence fragments having at least the percentage sequence identity as disclosed above to SEQ ID NO:4 connected through a plurality of sequence fragments having no sequence identity to SEQ ID NO:4.

In embodiments, the zinc finger domain has the percentage sequence identity as disclosed in the above paragraphs to at least 170 continuous amino acids of SEQ ID NO:4. In embodiments, the zinc finger domain has the percentage sequence identity as disclosed in the above paragraphs to at least 160 continuous amino acids of SEQ ID NO:4. In embodiments, the zinc finger domain has the percentage sequence identity as disclosed in the above paragraphs to at least 150 continuous amino acids of SEQ ID NO:4. In embodiments, the zinc finger domain has the percentage sequence identity as disclosed in the above paragraphs to at least 140 continuous amino acids of SEQ ID NO:4. In embodiments, the zinc finger domain has the percentage sequence identity as disclosed in the above paragraphs to at least 130 continuous amino acids of SEQ ID NO:4. In embodiments, the zinc finger domain has the percentage sequence identity as disclosed in the above paragraphs to at least 120 continuous amino acids of SEQ ID NO:4. In embodiments, the zinc finger domain has the percentage sequence identity as disclosed in the above paragraphs to at least 110 continuous amino acids of SEQ ID NO:4. In embodiments, the zinc finger domain has the percentage sequence identity as disclosed in the above paragraphs to at least 100 continuous amino acids of SEQ ID NO:4. In embodiments, the zinc finger domain has the percentage sequence identity as disclosed in the above paragraphs to at least 90 continuous amino acids of SEQ ID NO:4. In embodiments, the zinc finger domain has the percentage sequence identity as disclosed in the above paragraphs to at least 80 continuous amino acids of SEQ ID NO:4. In embodiments, the zinc finger domain has the percentage sequence identity as disclosed in the above paragraphs to at least 70 continuous amino acids of SEQ ID NO:4. In embodiments, the zinc finger domain has the percentage sequence identity as disclosed in the above paragraphs to at least 60 continuous amino acids of SEQ ID NO:4. In embodiments, the zinc finger domain has the percentage sequence identity as disclosed in the above paragraphs to at least 50 continuous amino acids of SEQ ID NO:4. In embodiments, the zinc finger domain has the percentage sequence identity as disclosed in the above paragraphs to at least 40 continuous amino acids of SEQ ID NO:4. In embodiments, the zinc finger domain has the percentage sequence identity as disclosed in the above paragraphs to at least 30 continuous amino acids of SEQ ID NO:4. In embodiments, the zinc finger domain has the percentage sequence identity as disclosed in the above paragraphs to at least 20 continuous amino acids of SEQ ID NO:4.

The sequence of SEQ ID NO:4 encodes a non-naturally occurring peptide sequence, which may be referred to herein as “HTLV-ZFP-5” or “ZFP-5”.

In embodiments, the protein further includes a transcriptional repressor. The term “transcriptional repressor” refers to a protein that decreases gene transcription of a gene or set of genes. For example, transcriptional repressors may be DNA-binding proteins that bind to promoter-proximal elements, including the HTLV-1 LTR or sequences within the HTLV-1 LTR. The transcriptional repressors used in the fusion proteins described herein include, but are not limited to, Kruppel associated box (KRAB) domains, methyl CpG binding protein 2 (meCP2), DNA methyltransferase (DNMT) domains and derivatives or functional fragments thereof.

In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a DNMT domain. In embodiments, the transcriptional repressor includes a KRAB domain and meCP2 or a fragment thereof.

In embodiments, the protein of the present disclosure includes further components, including, but are not limited to, a cell-penetrating peptide (e.g. a TAT peptide or a derivative thereof) and/or one or more nuclear localization signals. In embodiments, the protein includes a peptide that promotes stabilization of the protein and/or enhances protein isolation (e.g. myc-tag sequence).

Cell-penetrating peptides (CPPs) generally are short peptides that can facilitate cellular intake/uptake of various molecular equipment (e.g. a protein). The cargo is associated with the CPPs either through chemical linkage via covalent bonds or through non-covalent interactions. The function of the CPPs is to deliver the cargo into cells. Any peptide that is known to be capable of facilitating cellular uptake or have cell-penetrating activity can be used in the composition and methods of the disclosure. In embodiments, the CPP is trans-activating transcriptional activator (Tat) or a derivative thereof. In embodiments, Tat enhances the cellular intake/uptake of the protein into the cells. Thus, in embodiments, the protein provided herein further includes Tat. In embodiments, Tat includes a sequence having at least 80% sequence identity to SEQ ID NO:120. In embodiments, Tat includes a sequence having at least 90% sequence identity to SEQ ID NO: 120. In embodiments, Tat includes a sequence having at least 95% sequence identity to SEQ ID NO:120. In embodiments, Tat includes a sequence having at least 98% sequence identity to SEQ ID NO: 120. In embodiments, Tat includes a sequence having at least 99% sequence identity to SEQ ID NO:120. In embodiments, Tat includes the sequence of SEQ ID NO:20. In embodiments, Tat is SEQ ID NO:120.

A nuclear localization signal or sequence (NLS) is an amino acid sequence that tags a protein for import into the cell nucleus by nuclear transport. Any peptides that are known to be capable of nuclear localization activity can be used in the composition and methods provided herein including embodiments thereof. In embodiments, the protein provided herein includes one or more NLSs. In embodiments, the protein provided herein includes 1, 2, 3, 4, 5, 6, 7, 8, 9, 10 or more NLS. In embodiments, the NLS includes the sequence having at least 90% sequence identity to SEQ ID NO:121. In embodiments, the NLS includes the sequence of SEQ ID NO:121. In embodiments, the NLS is the sequence of SEQ ID NO:121. In embodiments, the NLS includes the sequence having at least 80% sequence identity to SEQ ID NO:124. In embodiments, the NLS includes the sequence having at least 90% sequence identity to SEQ ID NO:124. In embodiments, the NLS includes the sequence having at least 95% sequence identity to SEQ ID NO:124. In embodiments, the NLS includes the sequence having at least 98% sequence identity to SEQ ID NO:124. In embodiments, the NLS includes the sequence having at least 99% sequence identity to SEQ ID NO:124. In embodiments, the NLS includes the sequence of SEQ ID NO:124. In embodiments, the NLS is the sequence of SEQ ID NO:124.

In embodiments, the protein provided herein includes one or more additional sequences such as a myc-tag sequence. A myc tag is a polypeptide protein tag derived from the c-myc gene product. In embodiments, the myc tag is used for affinity chromatography (e.g. to isolate the protein provided herein including embodiments thereof from a non-homogenous composition). In embodiments, the Myc tag includes a sequence having at least 80% sequence identity to SEQ ID NO:122. In embodiments, the Myc tag includes a sequence having at least 90% sequence identity to SEQ ID NO:122. In embodiments, the Myc tag includes a sequence having at least 95% sequence identity to SEQ ID NO: 122. In embodiments, the Myc tag includes a sequence having at least 98% sequence identity to SEQ ID NO:122. In embodiments, the Myc tag includes a sequence having at least 99% sequence identity to SEQ ID NO:122. In embodiments, the Myc tag includes SEQ ID NO:122. In embodiments, the Myc tag is the sequence of SEQ ID NO:122.

Thus, in embodiments, the protein further includes a nuclear localization signal, a Tat domain, a Myc tag, or combinations thereof. In embodiments, the protein further includes a nuclear localization signal. In embodiments, the protein further includes a a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 13. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:13. In embodiments, the protein includes a sequence having at least 75% sequence identity to SEQ ID NO:13. In embodiments, the protein includes a sequence having at least 80% sequence identity to SEQ ID NO:13. In embodiments, the protein includes a sequence having at least 85% sequence identity to SEQ ID NO:13. In embodiments, the protein includes a sequence having at least 90% sequence identity to SEQ ID NO:13. In embodiments, the protein includes a sequence having at least 91% sequence identity to SEQ ID NO:13. In embodiments, the protein includes a sequence having at least 92% sequence identity to SEQ ID NO:13. In embodiments, the protein includes a sequence having at least 93% sequence identity to SEQ ID NO:13. In embodiments, the protein includes a sequence having at least 94% sequence identity to SEQ ID NO:13. In embodiments, the protein includes a sequence having at least 95% sequence identity to SEQ ID NO:13. In embodiments, the protein includes a sequence having at least 96% sequence identity to SEQ ID NO: 13. In embodiments, the protein includes a sequence having at least 97% sequence identity to SEQ ID NO:13. In embodiments, the protein includes a sequence having at least 98% sequence identity to SEQ ID NO:13. In embodiments, the protein includes a sequence having at least 99% sequence identity to SEQ ID NO:13. In embodiments, the protein includes the sequence of SEQ ID NO:13. In embodiments, the protein is the sequence of SEQ ID NO:13.

In embodiments, the protein includes a sequence having about 75% sequence identity to SEQ ID NO:13. In embodiments, the protein includes a sequence having about 80% sequence identity to SEQ ID NO:13. In embodiments, the protein includes a sequence having about 85% sequence identity to SEQ ID NO:13. In embodiments, the protein includes a sequence having about 90% sequence identity to SEQ ID NO:13. In embodiments, the protein includes a sequence having about 91% sequence identity to SEQ ID NO:13. In embodiments, the protein includes a sequence having about 92% sequence identity to SEQ ID NO:13. In embodiments, the protein includes a sequence having about 93% sequence identity to SEQ ID NO:13. In embodiments, the protein includes a sequence having about 94% sequence identity to SEQ ID NO:13. In embodiments, the protein includes a sequence having about 95% sequence identity to SEQ ID NO:13. In embodiments, the protein includes a sequence having about 96% sequence identity to SEQ ID NO:13. In embodiments, the protein includes a sequence having about 97% sequence identity to SEQ ID NO:13. In embodiments, the protein includes a sequence having about 98% sequence identity to SEQ ID NO:13. In embodiments, the protein includes a sequence having about 99% sequence identity to SEQ ID NO:13.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:13 connected to a second sequence fragment having at least the percentage sequence identity as disclosed above to SEQ ID NO:13 through a sequence fragment having no sequence identity to SEQ ID NO:13. In embodiments, the non-contiguous sequence is a sequence including a plurality of sequence fragments having at least the percentage sequence identity as disclosed above to SEQ ID NO:13 connected through a plurality of sequence fragments having no sequence identity to SEQ ID NO:13.

In embodiments, the protein has the percentage sequence identity as disclosed in the above paragraphs to at least 300 continuous amino acids of SEQ ID NO:13. In embodiments, the protein has the percentage sequence identity as disclosed in the above paragraphs to at least 290 continuous amino acids of SEQ ID NO:13. In embodiments, the protein has the percentage sequence identity as disclosed in the above paragraphs to at least 280 continuous amino acids of SEQ ID NO:13. In embodiments, the protein has the percentage sequence identity as disclosed in the above paragraphs to at least 270 continuous amino acids of SEQ ID NO:13. In embodiments, the protein has the percentage sequence identity as disclosed in the above paragraphs to at least 260 continuous amino acids of SEQ ID NO:13. In embodiments, the protein has the percentage sequence identity as disclosed in the above paragraphs to at least 250 continuous amino acids of SEQ ID NO:13. In embodiments, the protein has the percentage sequence identity as disclosed in the above paragraphs to at least 240 continuous amino acids of SEQ ID NO:13. In embodiments, the protein has the percentage sequence identity as disclosed in the above paragraphs to at least 230 continuous amino acids of SEQ ID NO:13. In embodiments, the protein has the percentage sequence identity as disclosed in the above paragraphs to at least 220 continuous amino acids of SEQ ID NO:13. In embodiments, the protein has the percentage sequence identity as disclosed in the above paragraphs to at least 210 continuous amino acids of SEQ ID NO:13. In embodiments, the protein has the percentage sequence identity as disclosed in the above paragraphs to at least 200 continuous amino acids of SEQ ID NO:13. In embodiments, the protein has the percentage sequence identity as disclosed in the above paragraphs to at least 190 continuous amino acids of SEQ ID NO:13. In embodiments, the protein has the percentage sequence identity as disclosed in the above paragraphs to at least 180 continuous amino acids of SEQ ID NO:13. In embodiments, the protein has the percentage sequence identity as disclosed in the above paragraphs to at least 170 continuous amino acids of SEQ ID NO:13. In embodiments, the protein has the percentage sequence identity as disclosed in the above paragraphs to at least 160 continuous amino acids of SEQ ID NO:13. In embodiments, the protein has the percentage sequence identity as disclosed in the above paragraphs to at least 150 continuous amino acids of SEQ ID NO:13. In embodiments, the protein has the percentage sequence identity as disclosed in the above paragraphs to at least 140 continuous amino acids of SEQ ID NO:13. In embodiments, the protein has the percentage sequence identity as disclosed in the above paragraphs to at least 130 continuous amino acids of SEQ ID NO:13. In embodiments, the protein has the percentage sequence identity as disclosed in the above paragraphs to at least 120 continuous amino acids of SEQ ID NO:13. In embodiments, the protein has the percentage sequence identity as disclosed in the above paragraphs to at least 110 continuous amino acids of SEQ ID NO:13. In embodiments, the protein has the percentage sequence identity as disclosed in the above paragraphs to at least 100 continuous amino acids of SEQ ID NO:13. In embodiments, the protein has the percentage sequence identity as disclosed in the above paragraphs to at least 90 continuous amino acids of SEQ ID NO:13. In embodiments, the protein has the percentage sequence identity as disclosed in the above paragraphs to at least 80 continuous amino acids of SEQ ID NO:13. In embodiments, the protein has the percentage sequence identity as disclosed in the above paragraphs to at least 70 continuous amino acids of SEQ ID NO:13. In embodiments, the protein has the percentage sequence identity as disclosed in the above paragraphs to at least 60 continuous amino acids of SEQ ID NO:13. In embodiments, the protein has the percentage sequence identity as disclosed in the above paragraphs to at least 50 continuous amino acids of SEQ ID NO:13. In embodiments, the protein has the percentage sequence identity as disclosed in the above paragraphs to at least 40 continuous amino acids of SEQ ID NO:13. In embodiments, the protein has the percentage sequence identity as disclosed in the above paragraphs to at least 30 continuous amino acids of SEQ ID NO:13. In embodiments, the protein has the percentage sequence identity as disclosed in the above paragraphs to at least 20 continuous amino acids of SEQ ID NO:13.

In embodiments, the protein has at least 75%, 80%, 85%, 90%, 95%, 96%, 97%, 98% or 99% sequence identity to the sequence of SEQ ID NO:20. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, or 220 continuous amino acid portion) compared to SEQ ID NO:20. In embodiments, the protein includes a sequence having at least 75% sequence identity to SEQ ID NO:20. In embodiments, the protein includes a sequence having at least 80% sequence identity to SEQ ID NO:20. In embodiments, the protein includes a sequence having at least 85% sequence identity to SEQ ID NO:20. In embodiments, the protein includes a sequence having at least 90% sequence identity to SEQ ID NO:20. In embodiments, the protein includes a sequence having at least 91% sequence identity to SEQ ID NO:20. In embodiments, the protein includes a sequence having at least 92% sequence identity to SEQ ID NO:20. In embodiments, the protein includes a sequence having at least 93% sequence identity to SEQ ID NO:20. In embodiments, the protein includes a sequence having at least 94% sequence identity to SEQ ID NO:20. In embodiments, the protein includes a sequence having at least 95% sequence identity to SEQ ID NO:20. In embodiments, the protein includes a sequence having at least 96% sequence identity to SEQ ID NO:20. In embodiments, the protein includes a sequence having at least 97% sequence identity to SEQ ID NO:20. In embodiments, the protein includes a sequence having at least 98% sequence identity to SEQ ID NO:20. In embodiments, the protein includes a sequence having at least 99% sequence identity to SEQ ID NO:20. In embodiments, the protein includes the sequence of SEQ ID NO:20. In embodiments, the protein is the sequence of SEQ ID NO:20.

In embodiments, the protein includes a sequence having about 75% sequence identity to SEQ ID NO:20. In embodiments, the protein includes a sequence having about 80% sequence identity to SEQ ID NO:20. In embodiments, the protein includes a sequence having about 85% sequence identity to SEQ ID NO:20. In embodiments, the protein includes a sequence having about 90% sequence identity to SEQ ID NO:20. In embodiments, the protein includes a sequence having about 91% sequence identity to SEQ ID NO:20. In embodiments, the protein includes a sequence having about 92% sequence identity to SEQ ID NO:20. In embodiments, the protein includes a sequence having about 93% sequence identity to SEQ ID NO:20. In embodiments, the protein includes a sequence having about 94% sequence identity to SEQ ID NO:20. In embodiments, the protein includes a sequence having about 95% sequence identity to SEQ ID NO:20. In embodiments, the protein includes a sequence having about 96% sequence identity to SEQ ID NO:20. In embodiments, the protein includes a sequence having about 97% sequence identity to SEQ ID NO:20. In embodiments, the protein includes a sequence having about 98% sequence identity to SEQ ID NO:20. In embodiments, the protein includes a sequence having about 99% sequence identity to SEQ ID NO:20.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:20 connected to a second sequence fragment having at least the percentage sequence identity as disclosed above to SEQ ID NO:20 through a sequence fragment having no sequence identity to SEQ ID NO:20. In embodiments, the non-contiguous sequence is a sequence including a plurality of sequence fragments having at least the percentage sequence identity as disclosed above to SEQ ID NO:20 connected through a plurality of sequence fragments having no sequence identity to SEQ ID NO:20.

In embodiments, the protein has the percentage sequence identity as disclosed in the above paragraphs to at least 220 continuous amino acids of SEQ ID NO:20. In embodiments, the protein has the percentage sequence identity as disclosed in the above paragraphs to at least 210 continuous amino acids of SEQ ID NO:20. In embodiments, the protein has the percentage sequence identity as disclosed in the above paragraphs to at least 200 continuous amino acids of SEQ ID NO:20. In embodiments, the protein has the percentage sequence identity as disclosed in the above paragraphs to at least 190 continuous amino acids of SEQ ID NO:20. In embodiments, the protein has the percentage sequence identity as disclosed in the above paragraphs to at least 180 continuous amino acids of SEQ ID NO:20. In embodiments, the protein has the percentage sequence identity as disclosed in the above paragraphs to at least 170 continuous amino acids of SEQ ID NO:20. In embodiments, the protein has the percentage sequence identity as disclosed in the above paragraphs to at least 160 continuous amino acids of SEQ ID NO:20. In embodiments, the protein has the percentage sequence identity as disclosed in the above paragraphs to at least 150 continuous amino acids of SEQ ID NO:20. In embodiments, the protein has the percentage sequence identity as disclosed in the above paragraphs to at least 140 continuous amino acids of SEQ ID NO:20. In embodiments, the protein has the percentage sequence identity as disclosed in the above paragraphs to at least 130 continuous amino acids of SEQ ID NO:20. In embodiments, the protein has the percentage sequence identity as disclosed in the above paragraphs to at least 120 continuous amino acids of SEQ ID NO:20. In embodiments, the protein has the percentage sequence identity as disclosed in the above paragraphs to at least 110 continuous amino acids of SEQ ID NO:20. In embodiments, the protein has the percentage sequence identity as disclosed in the above paragraphs to at least 100 continuous amino acids of SEQ ID NO:20. In embodiments, the protein has the percentage sequence identity as disclosed in the above paragraphs to at least 90 continuous amino acids of SEQ ID NO:20. In embodiments, the protein has the percentage sequence identity as disclosed in the above paragraphs to at least 80 continuous amino acids of SEQ ID NO:20. In embodiments, the protein has the percentage sequence identity as disclosed in the above paragraphs to at least 70 continuous amino acids of SEQ ID NO:20. In embodiments, the protein has the percentage sequence identity as disclosed in the above paragraphs to at least 60 continuous amino acids of SEQ ID NO:20. In embodiments, the protein has the percentage sequence identity as disclosed in the above paragraphs to at least 50 continuous amino acids of SEQ ID NO:20. In embodiments, the protein has the percentage sequence identity as disclosed in the above paragraphs to at least 40 continuous amino acids of SEQ ID NO:20. In embodiments, the protein has the percentage sequence identity as disclosed in the above paragraphs to at least 30 continuous amino acids of SEQ ID NO:20. In embodiments, the protein has the percentage sequence identity as disclosed in the above paragraphs to at least 20 continuous amino acids of SEQ ID NO:20.

In embodiments, the protein has at least 75%, 80%, 85%, 90%, 95%, 96%, 97%, 98% or 99% sequence identity to the sequence of SEQ ID NO:21. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:21. In embodiments, the protein includes a sequence having at least 75% sequence identity to SEQ ID NO:21. In embodiments, the protein includes a sequence having at least 80% sequence identity to SEQ ID NO:21. In embodiments, the protein includes a sequence having at least 85% sequence identity to SEQ ID NO:21. In embodiments, the protein includes a sequence having at least 90% sequence identity to SEQ ID NO:21. In embodiments, the protein includes a sequence having at least 91% sequence identity to SEQ ID NO:21. In embodiments, the protein includes a sequence having at least 92% sequence identity to SEQ ID NO:21. In embodiments, the protein includes a sequence having at least 93% sequence identity to SEQ ID NO:21. In embodiments, the protein includes a sequence having at least 94% sequence identity to SEQ ID NO:21. In embodiments, the protein includes a sequence having at least 95% sequence identity to SEQ ID NO:21. In embodiments, the protein includes a sequence having at least 96% sequence identity to SEQ ID NO:21. In embodiments, the protein includes a sequence having at least 97% sequence identity to SEQ ID NO:21. In embodiments, the protein includes a sequence having at least 98% sequence identity to SEQ ID NO:21. In embodiments, the protein includes a sequence having at least 99% sequence identity to SEQ ID NO:21. In embodiments, the protein includes the sequence of SEQ ID NO:21. In embodiments, the protein is the sequence of SEQ ID NO:21.

In embodiments, the protein includes a sequence having about 75% sequence identity to SEQ ID NO:21. In embodiments, the protein includes a sequence having about 80% sequence identity to SEQ ID NO:21. In embodiments, the protein includes a sequence having about 85% sequence identity to SEQ ID NO:21. In embodiments, the protein includes a sequence having about 90% sequence identity to SEQ ID NO:21. In embodiments, the protein includes a sequence having about 91% sequence identity to SEQ ID NO:21. In embodiments, the protein includes a sequence having about 92% sequence identity to SEQ ID NO:21. In embodiments, the protein includes a sequence having about 93% sequence identity to SEQ ID NO:21. In embodiments, the protein includes a sequence having about 94% sequence identity to SEQ ID NO:21. In embodiments, the protein includes a sequence having about 95% sequence identity to SEQ ID NO:21. In embodiments, the protein includes a sequence having about 96% sequence identity to SEQ ID NO:21. In embodiments, the protein includes a sequence having about 97% sequence identity to SEQ ID NO:21. In embodiments, the protein includes a sequence having about 98% sequence identity to SEQ ID NO:21. In embodiments, the protein includes a sequence having about 99% sequence identity to SEQ ID NO:21.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:21 connected to a second sequence fragment having at least the percentage sequence identity as disclosed above to SEQ ID NO:21 through a sequence fragment having no sequence identity to SEQ ID NO:21. In embodiments, the non-contiguous sequence is a sequence including a plurality of sequence fragments having at least the percentage sequence identity as disclosed above to SEQ ID NO:21 connected through a plurality of sequence fragments having no sequence identity to SEQ ID NO:21.

In embodiments, the protein has the percentage sequence identity as disclosed in the above paragraphs to at least 330 continuous amino acids of SEQ ID NO:21. In embodiments, the protein has the percentage sequence identity as disclosed in the above paragraphs to at least 320 continuous amino acids of SEQ ID NO:21. In embodiments, the protein has the percentage sequence identity as disclosed in the above paragraphs to at least 310 continuous amino acids of SEQ ID NO:21. In embodiments, the protein has the percentage sequence identity as disclosed in the above paragraphs to at least 300 continuous amino acids of SEQ ID NO:21. In embodiments, the protein has the percentage sequence identity as disclosed in the above paragraphs to at least 290 continuous amino acids of SEQ ID NO:21. In embodiments, the protein has the percentage sequence identity as disclosed in the above paragraphs to at least 280 continuous amino acids of SEQ ID NO:21. In embodiments, the protein has the percentage sequence identity as disclosed in the above paragraphs to at least 270 continuous amino acids of SEQ ID NO:21. In embodiments, the protein has the percentage sequence identity as disclosed in the above paragraphs to at least 260 continuous amino acids of SEQ ID NO:21. In embodiments, the protein has the percentage sequence identity as disclosed in the above paragraphs to at least 250 continuous amino acids of SEQ ID NO:21. In embodiments, the protein has the percentage sequence identity as disclosed in the above paragraphs to at least 240 continuous amino acids of SEQ ID NO:21. In embodiments, the protein has the percentage sequence identity as disclosed in the above paragraphs to at least 230 continuous amino acids of SEQ ID NO:21. In embodiments, the protein has the percentage sequence identity as disclosed in the above paragraphs to at least 220 continuous amino acids of SEQ ID NO:21. In embodiments, the protein has the percentage sequence identity as disclosed in the above paragraphs to at least 210 continuous amino acids of SEQ ID NO:21. In embodiments, the protein has the percentage sequence identity as disclosed in the above paragraphs to at least 200 continuous amino acids of SEQ ID NO:21. In embodiments, the protein has the percentage sequence identity as disclosed in the above paragraphs to at least 190 continuous amino acids of SEQ ID NO:21. In embodiments, the protein has the percentage sequence identity as disclosed in the above paragraphs to at least 180 continuous amino acids of SEQ ID NO:21. In embodiments, the protein has the percentage sequence identity as disclosed in the above paragraphs to at least 170 continuous amino acids of SEQ ID NO:21. In embodiments, the protein has the percentage sequence identity as disclosed in the above paragraphs to at least 160 continuous amino acids of SEQ ID NO:21. In embodiments, the protein has the percentage sequence identity as disclosed in the above paragraphs to at least 150 continuous amino acids of SEQ ID NO:21. In embodiments, the protein has the percentage sequence identity as disclosed in the above paragraphs to at least 140 continuous amino acids of SEQ ID NO:21. In embodiments, the protein has the percentage sequence identity as disclosed in the above paragraphs to at least 130 continuous amino acids of SEQ ID NO:21. In embodiments, the protein has the percentage sequence identity as disclosed in the above paragraphs to at least 120 continuous amino acids of SEQ ID NO:21. In embodiments, the protein has the percentage sequence identity as disclosed in the above paragraphs to at least 110 continuous amino acids of SEQ ID NO:21. In embodiments, the protein has the percentage sequence identity as disclosed in the above paragraphs to at least 100 continuous amino acids of SEQ ID NO:21. In embodiments, the protein has the percentage sequence identity as disclosed in the above paragraphs to at least 90 continuous amino acids of SEQ ID NO:21. In embodiments, the protein has the percentage sequence identity as disclosed in the above paragraphs to at least 80 continuous amino acids of SEQ ID NO:21. In embodiments, the protein has the percentage sequence identity as disclosed in the above paragraphs to at least 70 continuous amino acids of SEQ ID NO:21. In embodiments, the protein has the percentage sequence identity as disclosed in the above paragraphs to at least 60 continuous amino acids of SEQ ID NO:21. In embodiments, the protein has the percentage sequence identity as disclosed in the above paragraphs to at least 50 continuous amino acids of SEQ ID NO:21. In embodiments, the protein has the percentage sequence identity as disclosed in the above paragraphs to at least 40 continuous amino acids of SEQ ID NO:21. In embodiments, the protein has the percentage sequence identity as disclosed in the above paragraphs to at least 30 continuous amino acids of SEQ ID NO:21. In embodiments, the protein has the percentage sequence identity as disclosed in the above paragraphs to at least 20 continuous amino acids of SEQ ID NO:21.

In embodiments, the protein has at least 75%, 80%, 85%, 90%, 95%, 96%, 97%, 98% or 99% sequence identity to the sequence of SEQ ID NO:22. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 continuous amino acid portion) compared to SEQ ID NO:22. In embodiments, the protein includes a sequence having at least 75% sequence identity to SEQ ID NO:22. In embodiments, the protein includes a sequence having at least 80% sequence identity to SEQ ID NO:22. In embodiments, the protein includes a sequence having at least 85% sequence identity to SEQ ID NO:22. In embodiments, the protein includes a sequence having at least 90% sequence identity to SEQ ID NO:22. In embodiments, the protein includes a sequence having at least 91% sequence identity to SEQ ID NO:22. In embodiments, the protein includes a sequence having at least 92% sequence identity to SEQ ID NO:22. In embodiments, the protein includes a sequence having at least 93% sequence identity to SEQ ID NO:22. In embodiments, the protein includes a sequence having at least 94% sequence identity to SEQ ID NO:22. In embodiments, the protein includes a sequence having at least 95% sequence identity to SEQ ID NO:22. In embodiments, the protein includes a sequence having at least 96% sequence identity to SEQ ID NO:22. In embodiments, the protein includes a sequence having at least 97% sequence identity to SEQ ID NO:22. In embodiments, the protein includes a sequence having at least 98% sequence identity to SEQ ID NO:22. In embodiments, the protein includes a sequence having at least 99% sequence identity to SEQ ID NO:22. In embodiments, the protein includes the sequence of SEQ ID NO:22. In embodiments, the protein is the sequence of SEQ ID NO:22.

In embodiments, the protein includes a sequence having about 75% sequence identity to SEQ ID NO:22. In embodiments, the protein includes a sequence having about 80% sequence identity to SEQ ID NO:22. In embodiments, the protein includes a sequence having about 85% sequence identity to SEQ ID NO:22. In embodiments, the protein includes a sequence having about 90% sequence identity to SEQ ID NO:22. In embodiments, the protein includes a sequence having about 91% sequence identity to SEQ ID NO:22. In embodiments, the protein includes a sequence having about 92% sequence identity to SEQ ID NO:22. In embodiments, the protein includes a sequence having about 93% sequence identity to SEQ ID NO:22. In embodiments, the protein includes a sequence having about 94% sequence identity to SEQ ID NO:22. In embodiments, the protein includes a sequence having about 95% sequence identity to SEQ ID NO:22. In embodiments, the protein includes a sequence having about 96% sequence identity to SEQ ID NO:22. In embodiments, the protein includes a sequence having about 97% sequence identity to SEQ ID NO:22. In embodiments, the protein includes a sequence having about 98% sequence identity to SEQ ID NO:22. In embodiments, the protein includes a sequence having about 99% sequence identity to SEQ ID NO:22.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:22 connected to a second sequence fragment having at least the percentage sequence identity as disclosed above to SEQ ID NO:22 through a sequence fragment having no sequence identity to SEQ ID NO:22. In embodiments, the non-contiguous sequence is a sequence including a plurality of sequence fragments having at least the percentage sequence identity as disclosed above to SEQ ID NO:22 connected through a plurality of sequence fragments having no sequence identity to SEQ ID NO:22.

In embodiments, the protein has the percentage sequence identity as disclosed in the above paragraphs to at least 600 continuous amino acids of SEQ ID NO:22. In embodiments, the protein has the percentage sequence identity as disclosed in the above paragraphs to at least 590 continuous amino acids of SEQ ID NO:22. In embodiments, the protein has the percentage sequence identity as disclosed in the above paragraphs to at least 580 continuous amino acids of SEQ ID NO:22. In embodiments, the protein has the percentage sequence identity as disclosed in the above paragraphs to at least 570 continuous amino acids of SEQ ID NO:22. In embodiments, the protein has the percentage sequence identity as disclosed in the above paragraphs to at least 560 continuous amino acids of SEQ ID NO:22. In embodiments, the protein has the percentage sequence identity as disclosed in the above paragraphs to at least 550 continuous amino acids of SEQ ID NO:22. In embodiments, the protein has the percentage sequence identity as disclosed in the above paragraphs to at least 540 continuous amino acids of SEQ ID NO:22. In embodiments, the protein has the percentage sequence identity as disclosed in the above paragraphs to at least 530 continuous amino acids of SEQ ID NO:22. In embodiments, the protein has the percentage sequence identity as disclosed in the above paragraphs to at least 520 continuous amino acids of SEQ ID NO:22. In embodiments, the protein has the percentage sequence identity as disclosed in the above paragraphs to at least 510 continuous amino acids of SEQ ID NO:22. In embodiments, the protein has the percentage sequence identity as disclosed in the above paragraphs to at least 500 continuous amino acids of SEQ ID NO:22. In embodiments, the protein has the percentage sequence identity as disclosed in the above paragraphs to at least 490 continuous amino acids of SEQ ID NO:22. In embodiments, the protein has the percentage sequence identity as disclosed in the above paragraphs to at least 480 continuous amino acids of SEQ ID NO:22. In embodiments, the protein has the percentage sequence identity as disclosed in the above paragraphs to at least 470 continuous amino acids of SEQ ID NO:22. In embodiments, the protein has the percentage sequence identity as disclosed in the above paragraphs to at least 460 continuous amino acids of SEQ ID NO:22. In embodiments, the protein has the percentage sequence identity as disclosed in the above paragraphs to at least 450 continuous amino acids of SEQ ID NO:22. In embodiments, the protein has the percentage sequence identity as disclosed in the above paragraphs to at least 440 continuous amino acids of SEQ ID NO:22. In embodiments, the protein has the percentage sequence identity as disclosed in the above paragraphs to at least 430 continuous amino acids of SEQ ID NO:22. In embodiments, the protein has the percentage sequence identity as disclosed in the above paragraphs to at least 420 continuous amino acids of SEQ ID NO:22. In embodiments, the protein has the percentage sequence identity as disclosed in the above paragraphs to at least 410 continuous amino acids of SEQ ID NO:22. In embodiments, the protein has the percentage sequence identity as disclosed in the above paragraphs to at least 400 continuous amino acids of SEQ ID NO:22. In embodiments, the protein has the percentage sequence identity as disclosed in the above paragraphs to at least 390 continuous amino acids of SEQ ID NO:22. In embodiments, the protein has the percentage sequence identity as disclosed in the above paragraphs to at least 380 continuous amino acids of SEQ ID NO:22. In embodiments, the protein has the percentage sequence identity as disclosed in the above paragraphs to at least 370 continuous amino acids of SEQ ID NO:22. In embodiments, the protein has the percentage sequence identity as disclosed in the above paragraphs to at least 360 continuous amino acids of SEQ ID NO:22. In embodiments, the protein has the percentage sequence identity as disclosed in the above paragraphs to at least 350 continuous amino acids of SEQ ID NO:22. In embodiments, the protein has the percentage sequence identity as disclosed in the above paragraphs to at least 340 continuous amino acids of SEQ ID NO:22. In embodiments, the protein has the percentage sequence identity as disclosed in the above paragraphs to at least 330 continuous amino acids of SEQ ID NO:22. In embodiments, the protein has the percentage sequence identity as disclosed in the above paragraphs to at least 320 continuous amino acids of SEQ ID NO:22. In embodiments, the protein has the percentage sequence identity as disclosed in the above paragraphs to at least 310 continuous amino acids of SEQ ID NO:22. In embodiments, the protein has the percentage sequence identity as disclosed in the above paragraphs to at least 300 continuous amino acids of SEQ ID NO:22. In embodiments, the protein has the percentage sequence identity as disclosed in the above paragraphs to at least 290 continuous amino acids of SEQ ID NO:22. In embodiments, the protein has the percentage sequence identity as disclosed in the above paragraphs to at least 280 continuous amino acids of SEQ ID NO:22. In embodiments, the protein has the percentage sequence identity as disclosed in the above paragraphs to at least 270 continuous amino acids of SEQ ID NO:22. In embodiments, the protein has the percentage sequence identity as disclosed in the above paragraphs to at least 260 continuous amino acids of SEQ ID NO:22. In embodiments, the protein has the percentage sequence identity as disclosed in the above paragraphs to at least 250 continuous amino acids of SEQ ID NO:22. In embodiments, the protein has the percentage sequence identity as disclosed in the above paragraphs to at least 240 continuous amino acids of SEQ ID NO:22. In embodiments, the protein has the percentage sequence identity as disclosed in the above paragraphs to at least 230 continuous amino acids of SEQ ID NO:22. In embodiments, the protein has the percentage sequence identity as disclosed in the above paragraphs to at least 220 continuous amino acids of SEQ ID NO:22. In embodiments, the protein has the percentage sequence identity as disclosed in the above paragraphs to at least 210 continuous amino acids of SEQ ID NO:22. In embodiments, the protein has the percentage sequence identity as disclosed in the above paragraphs to at least 200 continuous amino acids of SEQ ID NO:22. In embodiments, the protein has the percentage sequence identity as disclosed in the above paragraphs to at least 190 continuous amino acids of SEQ ID NO:22. In embodiments, the protein has the percentage sequence identity as disclosed in the above paragraphs to at least 180 continuous amino acids of SEQ ID NO:22. In embodiments, the protein has the percentage sequence identity as disclosed in the above paragraphs to at least 170 continuous amino acids of SEQ ID NO:22. In embodiments, the protein has the percentage sequence identity as disclosed in the above paragraphs to at least 160 continuous amino acids of SEQ ID NO:22. In embodiments, the protein has the percentage sequence identity as disclosed in the above paragraphs to at least 150 continuous amino acids of SEQ ID NO:22. In embodiments, the protein has the percentage sequence identity as disclosed in the above paragraphs to at least 140 continuous amino acids of SEQ ID NO:22. In embodiments, the protein has the percentage sequence identity as disclosed in the above paragraphs to at least 130 continuous amino acids of SEQ ID NO:22. In embodiments, the protein has the percentage sequence identity as disclosed in the above paragraphs to at least 120 continuous amino acids of SEQ ID NO:22. In embodiments, the protein has the percentage sequence identity as disclosed in the above paragraphs to at least 110 continuous amino acids of SEQ ID NO:22. In embodiments, the protein has the percentage sequence identity as disclosed in the above paragraphs to at least 100 continuous amino acids of SEQ ID NO:22. In embodiments, the protein has the percentage sequence identity as disclosed in the above paragraphs to at least 90 continuous amino acids of SEQ ID NO:22. In embodiments, the protein has the percentage sequence identity as disclosed in the above paragraphs to at least 80 continuous amino acids of SEQ ID NO:22. In embodiments, the protein has the percentage sequence identity as disclosed in the above paragraphs to at least 70 continuous amino acids of SEQ ID NO:22. In embodiments, the protein has the percentage sequence identity as disclosed in the above paragraphs to at least 60 continuous amino acids of SEQ ID NO:22. In embodiments, the protein has the percentage sequence identity as disclosed in the above paragraphs to at least 50 continuous amino acids of SEQ ID NO:22. In embodiments, the protein has the percentage sequence identity as disclosed in the above paragraphs to at least 40 continuous amino acids of SEQ ID NO:22. In embodiments, the protein has the percentage sequence identity as disclosed in the above paragraphs to at least 30 continuous amino acids of SEQ ID NO:22. In embodiments, the protein has the percentage sequence identity as disclosed in the above paragraphs to at least 40 continuous amino acids of SEQ ID NO:22.

In embodiments, the protein has at least 75%, 80%, 85%, 90%, 95%, 96%, 97%, 98% or 99% sequence identity to the sequence of SEQ ID NO:23. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, or 800 continuous amino acid portion) compared to SEQ ID NO:23. In embodiments, the protein includes a sequence having at least 75% sequence identity to SEQ ID NO:23. In embodiments, the protein includes a sequence having at least 80% sequence identity to SEQ ID NO:23. In embodiments, the protein includes a sequence having at least 85% sequence identity to SEQ ID NO:23. In embodiments, the protein includes a sequence having at least 90% sequence identity to SEQ ID NO:23. In embodiments, the protein includes a sequence having at least 91% sequence identity to SEQ ID NO:23. In embodiments, the protein includes a sequence having at least 92% sequence identity to SEQ ID NO:23. In embodiments, the protein includes a sequence having at least 93% sequence identity to SEQ ID NO:23. In embodiments, the protein includes a sequence having at least 94% sequence identity to SEQ ID NO:23. In embodiments, the protein includes a sequence having at least 95% sequence identity to SEQ ID NO:23. In embodiments, the protein includes a sequence having at least 96% sequence identity to SEQ ID NO:23. In embodiments, the protein includes a sequence having at least 97% sequence identity to SEQ ID NO:23. In embodiments, the protein includes a sequence having at least 98% sequence identity to SEQ ID NO:23. In embodiments, the protein includes a sequence having at least 99% sequence identity to SEQ ID NO:23. In embodiments, the protein includes the sequence of SEQ ID NO:23. In embodiments, the protein is the sequence of SEQ ID NO:23.

In embodiments, the protein includes a sequence having about 75% sequence identity to SEQ ID NO:23. In embodiments, the protein includes a sequence having about 80% sequence identity to SEQ ID NO:23. In embodiments, the protein includes a sequence having about 85% sequence identity to SEQ ID NO:23. In embodiments, the protein includes a sequence having about 90% sequence identity to SEQ ID NO:23. In embodiments, the protein includes a sequence having about 91% sequence identity to SEQ ID NO:23. In embodiments, the protein includes a sequence having about 92% sequence identity to SEQ ID NO:23. In embodiments, the protein includes a sequence having about 93% sequence identity to SEQ ID NO:23. In embodiments, the protein includes a sequence having about 94% sequence identity to SEQ ID NO:23. In embodiments, the protein includes a sequence having about 95% sequence identity to SEQ ID NO:23. In embodiments, the protein includes a sequence having about 96% sequence identity to SEQ ID NO:23. In embodiments, the protein includes a sequence having about 97% sequence identity to SEQ ID NO:23. In embodiments, the protein includes a sequence having about 98% sequence identity to SEQ ID NO:23. In embodiments, the protein includes a sequence having about 99% sequence identity to SEQ ID NO:23.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:23 connected to a second sequence fragment having at least the percentage sequence identity as disclosed above to SEQ ID NO:23 through a sequence fragment having no sequence identity to SEQ ID NO:23. In embodiments, the non-contiguous sequence is a sequence including a plurality of sequence fragments having at least the percentage sequence identity as disclosed above to SEQ ID NO:23 connected through a plurality of sequence fragments having no sequence identity to SEQ ID NO:23.

In embodiments, the protein has the percentage sequence identity as disclosed in the above paragraphs to at least 810 continuous amino acids of SEQ ID NO:23. In embodiments, the protein has the percentage sequence identity as disclosed in the above paragraphs to at least 800 continuous amino acids of SEQ ID NO:23. In embodiments, the protein has the percentage sequence identity as disclosed in the above paragraphs to at least 790 continuous amino acids of SEQ ID NO:23. In embodiments, the protein has the percentage sequence identity as disclosed in the above paragraphs to at least 780 continuous amino acids of SEQ ID NO:23. In embodiments, the protein has the percentage sequence identity as disclosed in the above paragraphs to at least 770 continuous amino acids of SEQ ID NO:23. In embodiments, the protein has the percentage sequence identity as disclosed in the above paragraphs to at least 760 continuous amino acids of SEQ ID NO:23. In embodiments, the protein has the percentage sequence identity as disclosed in the above paragraphs to at least 750 continuous amino acids of SEQ ID NO:23. In embodiments, the protein has the percentage sequence identity as disclosed in the above paragraphs to at least 740 continuous amino acids of SEQ ID NO:23. In embodiments, the protein has the percentage sequence identity as disclosed in the above paragraphs to at least 730 continuous amino acids of SEQ ID NO:23. In embodiments, the protein has the percentage sequence identity as disclosed in the above paragraphs to at least 720 continuous amino acids of SEQ ID NO:23. In embodiments, the protein has the percentage sequence identity as disclosed in the above paragraphs to at least 710 continuous amino acids of SEQ ID NO:23. In embodiments, the protein has the percentage sequence identity as disclosed in the above paragraphs to at least 700 continuous amino acids of SEQ ID NO:23. In embodiments, the protein has the percentage sequence identity as disclosed in the above paragraphs to at least 690 continuous amino acids of SEQ ID NO:23. In embodiments, the protein has the percentage sequence identity as disclosed in the above paragraphs to at least 680 continuous amino acids of SEQ ID NO:23. In embodiments, the protein has the percentage sequence identity as disclosed in the above paragraphs to at least 670 continuous amino acids of SEQ ID NO:23. In embodiments, the protein has the percentage sequence identity as disclosed in the above paragraphs to at least 660 continuous amino acids of SEQ ID NO:23. In embodiments, the protein has the percentage sequence identity as disclosed in the above paragraphs to at least 650 continuous amino acids of SEQ ID NO:23. In embodiments, the protein has the percentage sequence identity as disclosed in the above paragraphs to at least 640 continuous amino acids of SEQ ID NO:23. In embodiments, the protein has the percentage sequence identity as disclosed in the above paragraphs to at least 630 continuous amino acids of SEQ ID NO:23. In embodiments, the protein has the percentage sequence identity as disclosed in the above paragraphs to at least 620 continuous amino acids of SEQ ID NO:23. In embodiments, the protein has the percentage sequence identity as disclosed in the above paragraphs to at least 610 continuous amino acids of SEQ ID NO:23. In embodiments, the protein has the percentage sequence identity as disclosed in the above paragraphs to at least 600 continuous amino acids of SEQ ID NO:23. In embodiments, the protein has the percentage sequence identity as disclosed in the above paragraphs to at least 590 continuous amino acids of SEQ ID NO:23. In embodiments, the protein has the percentage sequence identity as disclosed in the above paragraphs to at least 580 continuous amino acids of SEQ ID NO:23. In embodiments, the protein has the percentage sequence identity as disclosed in the above paragraphs to at least 570 continuous amino acids of SEQ ID NO:23. In embodiments, the protein has the percentage sequence identity as disclosed in the above paragraphs to at least 560 continuous amino acids of SEQ ID NO:23. In embodiments, the protein has the percentage sequence identity as disclosed in the above paragraphs to at least 550 continuous amino acids of SEQ ID NO:23. In embodiments, the protein has the percentage sequence identity as disclosed in the above paragraphs to at least 540 continuous amino acids of SEQ ID NO:23. In embodiments, the protein has the percentage sequence identity as disclosed in the above paragraphs to at least 530 continuous amino acids of SEQ ID NO:23. In embodiments, the protein has the percentage sequence identity as disclosed in the above paragraphs to at least 520 continuous amino acids of SEQ ID NO:23. In embodiments, the protein has the percentage sequence identity as disclosed in the above paragraphs to at least 510 continuous amino acids of SEQ ID NO:23. In embodiments, the protein has the percentage sequence identity as disclosed in the above paragraphs to at least 500 continuous amino acids of SEQ ID NO:23. In embodiments, the protein has the percentage sequence identity as disclosed in the above paragraphs to at least 490 continuous amino acids of SEQ ID NO:23. In embodiments, the protein has the percentage sequence identity as disclosed in the above paragraphs to at least 480 continuous amino acids of SEQ ID NO:23. In embodiments, the protein has the percentage sequence identity as disclosed in the above paragraphs to at least 470 continuous amino acids of SEQ ID NO:23. In embodiments, the protein has the percentage sequence identity as disclosed in the above paragraphs to at least 460 continuous amino acids of SEQ ID NO:23. In embodiments, the protein has the percentage sequence identity as disclosed in the above paragraphs to at least 450 continuous amino acids of SEQ ID NO:23. In embodiments, the protein has the percentage sequence identity as disclosed in the above paragraphs to at least 440 continuous amino acids of SEQ ID NO:23. In embodiments, the protein has the percentage sequence identity as disclosed in the above paragraphs to at least 430 continuous amino acids of SEQ ID NO:23. In embodiments, the protein has the percentage sequence identity as disclosed in the above paragraphs to at least 420 continuous amino acids of SEQ ID NO:23. In embodiments, the protein has the percentage sequence identity as disclosed in the above paragraphs to at least 410 continuous amino acids of SEQ ID NO:23. In embodiments, the protein has the percentage sequence identity as disclosed in the above paragraphs to at least 400 continuous amino acids of SEQ ID NO:23. In embodiments, the protein has the percentage sequence identity as disclosed in the above paragraphs to at least 390 continuous amino acids of SEQ ID NO:23. In embodiments, the protein has the percentage sequence identity as disclosed in the above paragraphs to at least 380 continuous amino acids of SEQ ID NO:23. In embodiments, the protein has the percentage sequence identity as disclosed in the above paragraphs to at least 370 continuous amino acids of SEQ ID NO:23. In embodiments, the protein has the percentage sequence identity as disclosed in the above paragraphs to at least 360 continuous amino acids of SEQ ID NO:23. In embodiments, the protein has the percentage sequence identity as disclosed in the above paragraphs to at least 350 continuous amino acids of SEQ ID NO:23. In embodiments, the protein has the percentage sequence identity as disclosed in the above paragraphs to at least 340 continuous amino acids of SEQ ID NO:23. In embodiments, the protein has the percentage sequence identity as disclosed in the above paragraphs to at least 330 continuous amino acids of SEQ ID NO:23. In embodiments, the protein has the percentage sequence identity as disclosed in the above paragraphs to at least 320 continuous amino acids of SEQ ID NO:23. In embodiments, the protein has the percentage sequence identity as disclosed in the above paragraphs to at least 310 continuous amino acids of SEQ ID NO:23. In embodiments, the protein has the percentage sequence identity as disclosed in the above paragraphs to at least 300 continuous amino acids of SEQ ID NO:23. In embodiments, the protein has the percentage sequence identity as disclosed in the above paragraphs to at least 290 continuous amino acids of SEQ ID NO:23. In embodiments, the protein has the percentage sequence identity as disclosed in the above paragraphs to at least 280 continuous amino acids of SEQ ID NO:23. In embodiments, the protein has the percentage sequence identity as disclosed in the above paragraphs to at least 270 continuous amino acids of SEQ ID NO:23. In embodiments, the protein has the percentage sequence identity as disclosed in the above paragraphs to at least 260 continuous amino acids of SEQ ID NO:23. In embodiments, the protein has the percentage sequence identity as disclosed in the above paragraphs to at least 250 continuous amino acids of SEQ ID NO:23. In embodiments, the protein has the percentage sequence identity as disclosed in the above paragraphs to at least 240 continuous amino acids of SEQ ID NO:23. In embodiments, the protein has the percentage sequence identity as disclosed in the above paragraphs to at least 230 continuous amino acids of SEQ ID NO:23. In embodiments, the protein has the percentage sequence identity as disclosed in the above paragraphs to at least 220 continuous amino acids of SEQ ID NO:23. In embodiments, the protein has the percentage sequence identity as disclosed in the above paragraphs to at least 210 continuous amino acids of SEQ ID NO:23. In embodiments, the protein has the percentage sequence identity as disclosed in the above paragraphs to at least 200 continuous amino acids of SEQ ID NO:23. In embodiments, the protein has the percentage sequence identity as disclosed in the above paragraphs to at least 190 continuous amino acids of SEQ ID NO:23. In embodiments, the protein has the percentage sequence identity as disclosed in the above paragraphs to at least 180 continuous amino acids of SEQ ID NO:23. In embodiments, the protein has the percentage sequence identity as disclosed in the above paragraphs to at least 170 continuous amino acids of SEQ ID NO:23. In embodiments, the protein has the percentage sequence identity as disclosed in the above paragraphs to at least 160 continuous amino acids of SEQ ID NO:23. In embodiments, the protein has the percentage sequence identity as disclosed in the above paragraphs to at least 150 continuous amino acids of SEQ ID NO:23. In embodiments, the protein has the percentage sequence identity as disclosed in the above paragraphs to at least 140 continuous amino acids of SEQ ID NO:23. In embodiments, the protein has the percentage sequence identity as disclosed in the above paragraphs to at least 130 continuous amino acids of SEQ ID NO:23. In embodiments, the protein has the percentage sequence identity as disclosed in the above paragraphs to at least 120 continuous amino acids of SEQ ID NO:23. In embodiments, the protein has the percentage sequence identity as disclosed in the above paragraphs to at least 110 continuous amino acids of SEQ ID NO:23. In embodiments, the protein has the percentage sequence identity as disclosed in the above paragraphs to at least 100 continuous amino acids of SEQ ID NO:23. In embodiments, the protein has the percentage sequence identity as disclosed in the above paragraphs to at least 90 continuous amino acids of SEQ ID NO:23. In embodiments, the protein has the percentage sequence identity as disclosed in the above paragraphs to at least 80 continuous amino acids of SEQ ID NO:23. In embodiments, the protein has the percentage sequence identity as disclosed in the above paragraphs to at least 70 continuous amino acids of SEQ ID NO:23. In embodiments, the protein has the percentage sequence identity as disclosed in the above paragraphs to at least 60 continuous amino acids of SEQ ID NO:23. In embodiments, the protein has the percentage sequence identity as disclosed in the above paragraphs to at least 50 continuous amino acids of SEQ ID NO:23. In embodiments, the protein has the percentage sequence identity as disclosed in the above paragraphs to at least 40 continuous amino acids of SEQ ID NO:23. In embodiments, the protein has the percentage sequence identity as disclosed in the above paragraphs to at least 30 continuous amino acids of SEQ ID NO:23. In embodiments, the protein has the percentage sequence identity as disclosed in the above paragraphs to at least 20 continuous amino acids of SEQ ID NO:23.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:25. In embodiments, the sequence has at least 75%, 80%, 85%, 90%, 95%, 96%, 97%, 98% or 99% sequence identity to the sequence of SEQ ID NO:25. In embodiments, the sequence has at least 90%, 95%, 96%, 97%, 98%, 99% or 100% nucleic acid sequence identity across the whole sequence or a portion of the sequence (e.g. a 5, 10, 11, 12, 13, 14, or 15 continuous nucleic acid portion) of SEQ ID NO:25.

In embodiments, the sequence within the HTLV-1 LTR has at least 75% sequence identity to the sequence of SEQ ID NO:25. In embodiments, the sequence within the HTLV-1 LTR has at least 80% sequence identity to the sequence of SEQ ID NO:25. In embodiments, the sequence within the HTLV-1 LTR has at least 85% sequence identity to the sequence of SEQ ID NO:25. In embodiments, the sequence within the HTLV-1 LTR has at least 90% sequence identity to the sequence of SEQ ID NO:25. In embodiments, the sequence within the HTLV-1 LTR has at least 95% sequence identity to the sequence of SEQ ID NO:25. In embodiments, the sequence within the HTLV-1 LTR has at least 98% sequence identity to the sequence of SEQ ID NO:25. In embodiments, the sequence within the HTLV-1 LTR has at least 99% sequence identity to the sequence of SEQ ID NO:25. In embodiments, the sequence within the HTLV-1 LTR includes the sequence of SEQ ID NO:25. In embodiments, the sequence within the HTLV-1 LTR is the sequence of SEQ ID NO:25.

In embodiments, the sequence within the HTLV-1 LTR has about 75% sequence identity to the sequence of SEQ ID NO:25. In embodiments, the sequence within the HTLV-1 LTR has about 80% sequence identity to the sequence of SEQ ID NO:25. In embodiments, the sequence within the HTLV-1 LTR has about 85% sequence identity to the sequence of SEQ ID NO:25. In embodiments, the sequence within the HTLV-1 LTR has about 90% sequence identity to the sequence of SEQ ID NO:25. In embodiments, the sequence within the HTLV-1 LTR has about 95% sequence identity to the sequence of SEQ ID NO:25. In embodiments, the sequence within the HTLV-1 LTR has about 98% sequence identity to the sequence of SEQ ID NO:25. In embodiments, the sequence within the HTLV-1 LTR has about 99% sequence identity to the sequence of SEQ ID NO:25.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:39, F2 includes SEQ ID NO:40, F3 includes SEQ ID NO:41, F4 includes SEQ ID NO:42, F5 includes SEQ ID NO:43 and F6 includes SEQ ID NO:44. In embodiments, F1 is SEQ ID NO:39, F2 is SEQ ID NO:40, F3 is SEQ ID NO:41, F4 is SEQ ID NO:42, F5 is SEQ ID NO:43 and F6 is SEQ ID NO:44.

In embodiments, the zinc finger domain has at least 75%, 80%, 85%, 90%, 95%, 96%, 97%, 98% or 99% sequence identity to the sequence of SEQ ID NO:2. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:2. In embodiments, the zinc finger domain includes a sequence having at least 75% sequence identity to SEQ ID NO:2. In embodiments, the zinc finger domain has includes a sequence having at least 80% sequence identity to SEQ ID NO:2. In embodiments, the zinc finger domain has includes a sequence having at least 85% sequence identity to SEQ ID NO:2. In embodiments, the zinc finger domain has includes a sequence having at least 90% sequence identity to SEQ ID NO:2. In embodiments, the zinc finger domain includes a sequence having at least 91% sequence identity to SEQ ID NO:2. In embodiments, the zinc finger domain includes a sequence having at least 92% sequence identity to SEQ ID NO:2. In embodiments, the zinc finger domain includes a sequence having at least 93% sequence identity to SEQ ID NO:2. In embodiments, the zinc finger domain includes a sequence having at least 94% sequence identity to SEQ ID NO:2. In embodiments, the zinc finger domain has includes a sequence having at least 95% sequence identity to SEQ ID NO:2. In embodiments, the zinc finger domain includes a sequence having at least 96% sequence identity to SEQ ID NO:2. In embodiments, the zinc finger domain includes a sequence having at least 97% sequence identity to SEQ ID NO:2. In embodiments, the zinc finger domain includes a sequence having at least 98% sequence identity to SEQ ID NO:2. In embodiments, the zinc finger domain includes a sequence having at least 99% sequence identity to SEQ ID NO:2. In embodiments, the zinc finger domain includes the sequence of SEQ ID NO:2. In embodiments, the zinc finger domain is the sequence of SEQ ID NO:2.

In embodiments, the zinc finger domain includes a sequence having about 75% sequence identity to SEQ ID NO:2. In embodiments, the zinc finger domain includes a sequence having about 80% sequence identity to SEQ ID NO:2. In embodiments, the zinc finger domain includes a sequence having about 85% sequence identity to SEQ ID NO:2. In embodiments, the zinc finger domain includes a sequence having about 90% sequence identity to SEQ ID NO:2. In embodiments, the zinc finger domain includes a sequence having about 91% sequence identity to SEQ ID NO:2. In embodiments, the zinc finger domain includes a sequence having about 92% sequence identity to SEQ ID NO:2. In embodiments, the zinc finger domain includes a sequence having about 93% sequence identity to SEQ ID NO:2. In embodiments, the zinc finger domain includes a sequence having about 94% sequence identity to SEQ ID NO:2. In embodiments, the zinc finger domain includes a sequence having about 95% sequence identity to SEQ ID NO:2. In embodiments, the zinc finger domain includes a sequence having about 96% sequence identity to SEQ ID NO:2. In embodiments, the zinc finger domain includes a sequence having about 97% sequence identity to SEQ ID NO:2. In embodiments, the zinc finger domain includes a sequence having about 98% sequence identity to SEQ ID NO:2. In embodiments, the zinc finger domain includes a sequence having about 99% sequence identity to SEQ ID NO:2.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:2 connected to a second sequence fragment having at least the percentage sequence identity as disclosed above to SEQ ID NO:2 through a sequence fragment having no sequence identity to SEQ ID NO:2. In embodiments, the non-contiguous sequence is a sequence including a plurality of sequence fragments having at least the percentage sequence identity as disclosed above to SEQ ID NO:2 connected through a plurality of sequence fragments having no sequence identity to SEQ ID NO:2.

In embodiments, the zinc finger domain has the percentage sequence identity as disclosed in the above paragraphs to at least 170 continuous amino acids of SEQ ID NO:2. In embodiments, the zinc finger domain has the percentage sequence identity as disclosed in the above paragraphs to at least 160 continuous amino acids of SEQ ID NO:2. In embodiments, the zinc finger domain has the percentage sequence identity as disclosed in the above paragraphs to at least 150 continuous amino acids of SEQ ID NO:2. In embodiments, the zinc finger domain has the percentage sequence identity as disclosed in the above paragraphs to at least 140 continuous amino acids of SEQ ID NO:2. In embodiments, the zinc finger domain has the percentage sequence identity as disclosed in the above paragraphs to at least 130 continuous amino acids of SEQ ID NO:2. In embodiments, the zinc finger domain has the percentage sequence identity as disclosed in the above paragraphs to at least 120 continuous amino acids of SEQ ID NO:2. In embodiments, the zinc finger domain has the percentage sequence identity as disclosed in the above paragraphs to at least 110 continuous amino acids of SEQ ID NO:2. In embodiments, the zinc finger domain has the percentage sequence identity as disclosed in the above paragraphs to at least 100 continuous amino acids of SEQ ID NO:2. In embodiments, the zinc finger domain has the percentage sequence identity as disclosed in the above paragraphs to at least 90 continuous amino acids of SEQ ID NO:2. In embodiments, the zinc finger domain has the percentage sequence identity as disclosed in the above paragraphs to at least 80 continuous amino acids of SEQ ID NO:2. In embodiments, the zinc finger domain has the percentage sequence identity as disclosed in the above paragraphs to at least 70 continuous amino acids of SEQ ID NO:2. In embodiments, the zinc finger domain has the percentage sequence identity as disclosed in the above paragraphs to at least 60 continuous amino acids of SEQ ID NO:2. In embodiments, the zinc finger domain has the percentage sequence identity as disclosed in the above paragraphs to at least 50 continuous amino acids of SEQ ID NO:2. In embodiments, the zinc finger domain has the percentage sequence identity as disclosed in the above paragraphs to at least 40 continuous amino acids of SEQ ID NO:2. In embodiments, the zinc finger domain has the percentage sequence identity as disclosed in the above paragraphs to at least 30 continuous amino acids of SEQ ID NO:2. In embodiments, the zinc finger domain has the percentage sequence identity as disclosed in the above paragraphs to at least 20 continuous amino acids of SEQ ID NO:2.

The sequence of SEQ ID NO:2 encodes a non-naturally occurring peptide sequence, which may be referred to herein as “HTLV-ZFP-3” or “ZFP-3”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a DNMT domain. In embodiments, the transcriptional repressor includes a KRAB domain and meCP2 or a fragment thereof.

In embodiments, the protein further includes a nuclear localization signal, a Tat domain, a Myc tag, or combinations thereof. In embodiments, the protein further includes a nuclear localization signal. In embodiments, the protein further includes a a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 11. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:11. In embodiments, the protein includes a sequence having at least 75% sequence identity to SEQ ID NO:11. In embodiments, the protein includes a sequence having at least 80% sequence identity to SEQ ID NO:11. In embodiments, the protein includes a sequence having at least 85% sequence identity to SEQ ID NO:11. In embodiments, the protein includes a sequence having at least 90% sequence identity to SEQ ID NO:11. In embodiments, the protein includes a sequence having at least 91% sequence identity to SEQ ID NO:11. In embodiments, the protein includes a sequence having at least 92% sequence identity to SEQ ID NO:11. In embodiments, the protein includes a sequence having at least 93% sequence identity to SEQ ID NO:11. In embodiments, the protein includes a sequence having at least 94% sequence identity to SEQ ID NO:11. In embodiments, the protein includes a sequence having at least 95% sequence identity to SEQ ID NO:11. In embodiments, the protein includes a sequence having at least 96% sequence identity to SEQ ID NO: 11. In embodiments, the protein includes a sequence having at least 97% sequence identity to SEQ ID NO:11. In embodiments, the protein includes a sequence having at least 98% sequence identity to SEQ ID NO:11. In embodiments, the protein includes a sequence having at least 99% sequence identity to SEQ ID NO:11. In embodiments, the protein includes the sequence of SEQ ID NO:11. In embodiments, the protein is the sequence of SEQ ID NO:11.

In embodiments, the protein includes a sequence having about 75% sequence identity to SEQ ID NO:11. In embodiments, the protein includes a sequence having about 80% sequence identity to SEQ ID NO:11. In embodiments, the protein includes a sequence having about 85% sequence identity to SEQ ID NO:11. In embodiments, the protein includes a sequence having about 90% sequence identity to SEQ ID NO:11. In embodiments, the protein includes a sequence having about 91% sequence identity to SEQ ID NO:11. In embodiments, the protein includes a sequence having about 92% sequence identity to SEQ ID NO:11. In embodiments, the protein includes a sequence having about 93% sequence identity to SEQ ID NO:11. In embodiments, the protein includes a sequence having about 94% sequence identity to SEQ ID NO:11. In embodiments, the protein includes a sequence having about 95% sequence identity to SEQ ID NO:11. In embodiments, the protein includes a sequence having about 96% sequence identity to SEQ ID NO:11. In embodiments, the protein includes a sequence having about 97% sequence identity to SEQ ID NO:11. In embodiments, the protein includes a sequence having about 98% sequence identity to SEQ ID NO:11. In embodiments, the protein includes a sequence having about 99% sequence identity to SEQ ID NO:11.

In embodiments, the protein has a sequence with the percentage sequence identity as disclosed above, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:11 connected to a second sequence fragment having at least the percentage sequence identity as disclosed above to SEQ ID NO:11 through a sequence fragment having no sequence identity to SEQ ID NO: 11. In embodiments, the non-contiguous sequence is a sequence including a plurality of sequence fragments having at least the percentage sequence identity as disclosed above to SEQ ID NO:11 connected through a plurality of sequence fragments having no sequence identity to SEQ ID NO:11.

In embodiments, the protein has the percentage sequence identity as disclosed in the above paragraphs to at least 300 continuous amino acids of SEQ ID NO:11. In embodiments, the protein has the percentage sequence identity as disclosed in the above paragraphs to at least 290 continuous amino acids of SEQ ID NO:11. In embodiments, the protein has the percentage sequence identity as disclosed in the above paragraphs to at least 280 continuous amino acids of SEQ ID NO:11. In embodiments, the protein has the percentage sequence identity as disclosed in the above paragraphs to at least 270 continuous amino acids of SEQ ID NO:11. In embodiments, the protein has the percentage sequence identity as disclosed in the above paragraphs to at least 260 continuous amino acids of SEQ ID NO:11. In embodiments, the protein has the percentage sequence identity as disclosed in the above paragraphs to at least 250 continuous amino acids of SEQ ID NO:11. In embodiments, the protein has the percentage sequence identity as disclosed in the above paragraphs to at least 240 continuous amino acids of SEQ ID NO:11. In embodiments, the protein has the percentage sequence identity as disclosed in the above paragraphs to at least 230 continuous amino acids of SEQ ID NO:11. In embodiments, the protein has the percentage sequence identity as disclosed in the above paragraphs to at least 220 continuous amino acids of SEQ ID NO:11. In embodiments, the protein has the percentage sequence identity as disclosed in the above paragraphs to at least 210 continuous amino acids of SEQ ID NO:11. In embodiments, the protein has the percentage sequence identity as disclosed in the above paragraphs to at least 200 continuous amino acids of SEQ ID NO:11. In embodiments, the protein has the percentage sequence identity as disclosed in the above paragraphs to at least 190 continuous amino acids of SEQ ID NO:11. In embodiments, the protein has the percentage sequence identity as disclosed in the above paragraphs to at least 180 continuous amino acids of SEQ ID NO:11. In embodiments, the protein has the percentage sequence identity as disclosed in the above paragraphs to at least 170 continuous amino acids of SEQ ID NO:11. In embodiments, the protein has the percentage sequence identity as disclosed in the above paragraphs to at least 160 continuous amino acids of SEQ ID NO:11. In embodiments, the protein has the percentage sequence identity as disclosed in the above paragraphs to at least 150 continuous amino acids of SEQ ID NO:11. In embodiments, the protein has the percentage sequence identity as disclosed in the above paragraphs to at least 140 continuous amino acids of SEQ ID NO:11. In embodiments, the protein has the percentage sequence identity as disclosed in the above paragraphs to at least 130 continuous amino acids of SEQ ID NO:11. In embodiments, the protein has the percentage sequence identity as disclosed in the above paragraphs to at least 120 continuous amino acids of SEQ ID NO:11. In embodiments, the protein has the percentage sequence identity as disclosed in the above paragraphs to at least 110 continuous amino acids of SEQ ID NO:11. In embodiments, the protein has the percentage sequence identity as disclosed in the above paragraphs to at least 100 continuous amino acids of SEQ ID NO:11. In embodiments, the protein has the percentage sequence identity as disclosed in the above paragraphs to at least 90 continuous amino acids of SEQ ID NO:11. In embodiments, the protein has the percentage sequence identity as disclosed in the above paragraphs to at least 80 continuous amino acids of SEQ ID NO:11. In embodiments, the protein has the percentage sequence identity as disclosed in the above paragraphs to at least 70 continuous amino acids of SEQ ID NO:11. In embodiments, the protein has the percentage sequence identity as disclosed in the above paragraphs to at least 60 continuous amino acids of SEQ ID NO:11. In embodiments, the protein has the percentage sequence identity as disclosed in the above paragraphs to at least 50 continuous amino acids of SEQ ID NO:11. In embodiments, the protein has the percentage sequence identity as disclosed in the above paragraphs to at least 40 continuous amino acids of SEQ ID NO:11. In embodiments, the protein has the percentage sequence identity as disclosed in the above paragraphs to at least 30 continuous amino acids of SEQ ID NO:11. In embodiments, the protein has the percentage sequence identity as disclosed in the above paragraphs to at least 20 continuous amino acids of SEQ ID NO:11.

In embodiments, the protein has at least 75%, 80%, 85%, 90%, 95%, 96%, 97%, 98% or 99% sequence identity to the sequence of SEQ ID NO: 19. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, or 220 continuous amino acid portion) compared to SEQ ID NO:19. In embodiments, the protein includes a sequence having at least 75% sequence identity to SEQ ID NO:19. In embodiments, the protein includes a sequence having at least 80% sequence identity to SEQ ID NO:19. In embodiments, the protein includes a sequence having at least 85% sequence identity to SEQ ID NO:19. In embodiments, the protein includes a sequence having at least 90% sequence identity to SEQ ID NO:19. In embodiments, the protein includes a sequence having at least 91% sequence identity to SEQ ID NO:19. In embodiments, the protein includes a sequence having at least 92% sequence identity to SEQ ID NO: 19. In embodiments, the protein includes a sequence having at least 93% sequence identity to SEQ ID NO:19. In embodiments, the protein includes a sequence having at least 94% sequence identity to SEQ ID NO:19. In embodiments, the protein includes a sequence having at least 95% sequence identity to SEQ ID NO:19. In embodiments, the protein includes a sequence having at least 96% sequence identity to SEQ ID NO:19. In embodiments, the protein includes a sequence having at least 97% sequence identity to SEQ ID NO: 19. In embodiments, the protein includes a sequence having at least 98% sequence identity to SEQ ID NO:19. In embodiments, the protein includes a sequence having at least 99% sequence identity to SEQ ID NO:19. In embodiments, the protein includes the sequence of SEQ ID NO:19. In embodiments, the protein is the sequence of SEQ ID NO:19.

In embodiments, the protein includes a sequence having about 75% sequence identity to SEQ ID NO:19. In embodiments, the protein includes a sequence having about 80% sequence identity to SEQ ID NO:19. In embodiments, the protein includes a sequence having about 85% sequence identity to SEQ ID NO:19. In embodiments, the protein includes a sequence having about 90% sequence identity to SEQ ID NO:19. In embodiments, the protein includes a sequence having about 91% sequence identity to SEQ ID NO:19. In embodiments, the protein includes a sequence having about 92% sequence identity to SEQ ID NO:19. In embodiments, the protein includes a sequence having about 93% sequence identity to SEQ ID NO:19. In embodiments, the protein includes a sequence having about 94% sequence identity to SEQ ID NO:19. In embodiments, the protein includes a sequence having about 95% sequence identity to SEQ ID NO:19. In embodiments, the protein includes a sequence having about 96% sequence identity to SEQ ID NO:19. In embodiments, the protein includes a sequence having about 97% sequence identity to SEQ ID NO:19. In embodiments, the protein includes a sequence having about 98% sequence identity to SEQ ID NO:19. In embodiments, the protein includes a sequence having about 99% sequence identity to SEQ ID NO:19.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:19 connected to a second sequence fragment having at least the percentage sequence identity as disclosed above to SEQ ID NO:19 through a sequence fragment having no sequence identity to SEQ ID NO:19. In embodiments, the non-contiguous sequence is a sequence including a plurality of sequence fragments having at least the percentage sequence identity as disclosed above to SEQ ID NO:19 connected through a plurality of sequence fragments having no sequence identity to SEQ ID NO:19.

In embodiments, the protein has the percentage sequence identity as disclosed in the above paragraphs to at least 220 continuous amino acids of SEQ ID NO:19. In embodiments, the protein has the percentage sequence identity as disclosed in the above paragraphs to at least 210 continuous amino acids of SEQ ID NO:19. In embodiments, the protein has the percentage sequence identity as disclosed in the above paragraphs to at least 200 continuous amino acids of SEQ ID NO:19. In embodiments, the protein has the percentage sequence identity as disclosed in the above paragraphs to at least 190 continuous amino acids of SEQ ID NO:19. In embodiments, the protein has the percentage sequence identity as disclosed in the above paragraphs to at least 180 continuous amino acids of SEQ ID NO:19. In embodiments, the protein has the percentage sequence identity as disclosed in the above paragraphs to at least 170 continuous amino acids of SEQ ID NO:19. In embodiments, the protein has the percentage sequence identity as disclosed in the above paragraphs to at least 160 continuous amino acids of SEQ ID NO:19. In embodiments, the protein has the percentage sequence identity as disclosed in the above paragraphs to at least 150 continuous amino acids of SEQ ID NO:19. In embodiments, the protein has the percentage sequence identity as disclosed in the above paragraphs to at least 140 continuous amino acids of SEQ ID NO:19. In embodiments, the protein has the percentage sequence identity as disclosed in the above paragraphs to at least 130 continuous amino acids of SEQ ID NO:19. In embodiments, the protein has the percentage sequence identity as disclosed in the above paragraphs to at least 120 continuous amino acids of SEQ ID NO:19. In embodiments, the protein has the percentage sequence identity as disclosed in the above paragraphs to at least 110 continuous amino acids of SEQ ID NO:19. In embodiments, the protein has the percentage sequence identity as disclosed in the above paragraphs to at least 100 continuous amino acids of SEQ ID NO:19. In embodiments, the protein has the percentage sequence identity as disclosed in the above paragraphs to at least 90 continuous amino acids of SEQ ID NO:19. In embodiments, the protein has the percentage sequence identity as disclosed in the above paragraphs to at least 80 continuous amino acids of SEQ ID NO:19. In embodiments, the protein has the percentage sequence identity as disclosed in the above paragraphs to at least 70 continuous amino acids of SEQ ID NO:19. In embodiments, the protein has the percentage sequence identity as disclosed in the above paragraphs to at least 60 continuous amino acids of SEQ ID NO:19. In embodiments, the protein has the percentage sequence identity as disclosed in the above paragraphs to at least 50 continuous amino acids of SEQ ID NO:19. In embodiments, the protein has the percentage sequence identity as disclosed in the above paragraphs to at least 40 continuous amino acids of SEQ ID NO:19. In embodiments, the protein has the percentage sequence identity as disclosed in the above paragraphs to at least 30 continuous amino acids of SEQ ID NO:19. In embodiments, the protein has the percentage sequence identity as disclosed in the above paragraphs to at least 20 continuous amino acids of SEQ ID NO:19.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:28. In embodiments, the sequence has at least 75%, 80%, 85%, 90%, 95%, 96%, 97%, 98% or 99% sequence identity to the sequence of SEQ ID NO:28. In embodiments, the sequence has at least 90%, 95%, 96%, 97%, 98%, 99% or 100% nucleic acid sequence identity across the whole sequence or a portion of the sequence (e.g. a 5, 10, 15 or 20 continuous nucleic acid portion) of SEQ ID NO:28.

In embodiments, the sequence within the HTLV-1 LTR has at least 75% sequence identity to the sequence of SEQ ID NO:28. In embodiments, the sequence within the HTLV-1 LTR has at least 80% sequence identity to the sequence of SEQ ID NO:28. In embodiments, the sequence within the HTLV-1 LTR has at least 85% sequence identity to the sequence of SEQ ID NO:28. In embodiments, the sequence within the HTLV-1 LTR has at least 90% sequence identity to the sequence of SEQ ID NO:28. In embodiments, the sequence within the HTLV-1 LTR has at least 95% sequence identity to the sequence of SEQ ID NO:28. In embodiments, the sequence within the HTLV-1 LTR has at least 98% sequence identity to the sequence of SEQ ID NO:28. In embodiments, the sequence within the HTLV-1 LTR has at least 99% sequence identity to the sequence of SEQ ID NO:28. In embodiments, the sequence within the HTLV-1 LTR includes the sequence of SEQ ID NO:28. In embodiments, the sequence within the HTLV-1 LTR is the sequence of SEQ ID NO:28.

In embodiments, the sequence within the HTLV-1 LTR has about 75% sequence identity to the sequence of SEQ ID NO:28. In embodiments, the sequence within the HTLV-1 LTR has about 80% sequence identity to the sequence of SEQ ID NO:28. In embodiments, the sequence within the HTLV-1 LTR has about 85% sequence identity to the sequence of SEQ ID NO:28. In embodiments, the sequence within the HTLV-1 LTR has about 90% sequence identity to the sequence of SEQ ID NO:28. In embodiments, the sequence within the HTLV-1 LTR has about 95% sequence identity to the sequence of SEQ ID NO:28. In embodiments, the sequence within the HTLV-1 LTR has about 98% sequence identity to the sequence of SEQ ID NO:28. In embodiments, the sequence within the HTLV-1 LTR has about 99% sequence identity to the sequence of SEQ ID NO:28.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:57, F2 includes SEQ ID NO:58, F3 includes SEQ ID NO:59, F4 includes SEQ ID NO:60, F5 includes SEQ ID NO:61 and F6 includes SEQ ID NO:62. In embodiments, F1 is SEQ ID NO:57, F2 is SEQ ID NO:58, F3 is SEQ ID NO:59, F4 is SEQ ID NO:60, F5 is SEQ ID NO:61 and F6 is SEQ ID NO:62.

In embodiments, the zinc finger domain has at least 75%, 80%, 85%, 90%, 95%, 96%, 97%, 98% or 99% sequence identity to the sequence of SEQ ID NO:5. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, 160, or 170 continuous amino acid portion) of SEQ ID NO: 5. In embodiments, the zinc finger domain has at least 75% sequence identity to SEQ ID NO: 5. In embodiments, the zinc finger domain has at least 80% sequence identity to SEQ ID NO:5. In embodiments, the zinc finger domain has at least 85% sequence identity to SEQ ID NO:5. In embodiments, the zinc finger domain has at least 90% sequence identity to SEQ ID NO:5. In embodiments, the zinc finger domain includes a sequence having at least 91% sequence identity to SEQ ID NO:5. In embodiments, the zinc finger domain includes a sequence having at least 92% sequence identity to SEQ ID NO:5. In embodiments, the zinc finger domain includes a sequence having at least 93% sequence identity to SEQ ID NO:5. In embodiments, the zinc finger domain includes a sequence having at least 94% sequence identity to SEQ ID NO:5. In embodiments, the zinc finger domain has at least 95% sequence identity to SEQ ID NO: 5. In embodiments, the zinc finger domain has at least 96% sequence identity to SEQ ID NO:5. In embodiments, the zinc finger domain includes a sequence having at least 97% sequence identity to SEQ ID NO:5. In embodiments, the zinc finger domain has at least 98% sequence identity to SEQ ID NO:5. In embodiments, the zinc finger domain has at least 99% sequence identity to SEQ ID NO:5. In embodiments, the zinc finger domain includes the sequence of SEQ ID NO:5. In embodiments, the zinc finger domain is the sequence of SEQ ID NO:5.

In embodiments, the zinc finger domain includes a sequence having about 75% sequence identity to SEQ ID NO:5. In embodiments, the zinc finger domain includes a sequence having about 80% sequence identity to SEQ ID NO:5. In embodiments, the zinc finger domain includes a sequence having about 85% sequence identity to SEQ ID NO:5. In embodiments, the zinc finger domain includes a sequence having about 90% sequence identity to SEQ ID NO:5. In embodiments, the zinc finger domain includes a sequence having about 91% sequence identity to SEQ ID NO:5. In embodiments, the zinc finger domain includes a sequence having about 92% sequence identity to SEQ ID NO:5. In embodiments, the zinc finger domain includes a sequence having about 93% sequence identity to SEQ ID NO:5. In embodiments, the zinc finger domain includes a sequence having about 94% sequence identity to SEQ ID NO:5. In embodiments, the zinc finger domain includes a sequence having about 95% sequence identity to SEQ ID NO:5. In embodiments, the zinc finger domain includes a sequence having about 96% sequence identity to SEQ ID NO:5. In embodiments, the zinc finger domain includes a sequence having about 97% sequence identity to SEQ ID NO:5. In embodiments, the zinc finger domain includes a sequence having about 98% sequence identity to SEQ ID NO:5. In embodiments, the zinc finger domain includes a sequence having about 99% sequence identity to SEQ ID NO:5.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:5 connected to a second sequence fragment having at least the percentage sequence identity as disclosed above to SEQ ID NO:5 through a sequence fragment having no sequence identity to SEQ ID NO:5. In embodiments, the non-contiguous sequence is a sequence including a plurality of sequence fragments having at least the percentage sequence identity as disclosed above to SEQ ID NO:5 connected through a plurality of sequence fragments having no sequence identity to SEQ ID NO:5.

In embodiments, the zinc finger domain has the percentage sequence identity as disclosed in the above paragraphs to at least 170 continuous amino acids of SEQ ID NO:5. In embodiments, the zinc finger domain has the percentage sequence identity as disclosed in the above paragraphs to at least 160 continuous amino acids of SEQ ID NO:5. In embodiments, the zinc finger domain has the percentage sequence identity as disclosed in the above paragraphs to at least 150 continuous amino acids of SEQ ID NO:5. In embodiments, the zinc finger domain has the percentage sequence identity as disclosed in the above paragraphs to at least 140 continuous amino acids of SEQ ID NO:5. In embodiments, the zinc finger domain has the percentage sequence identity as disclosed in the above paragraphs to at least 130 continuous amino acids of SEQ ID NO:5. In embodiments, the zinc finger domain has the percentage sequence identity as disclosed in the above paragraphs to at least 120 continuous amino acids of SEQ ID NO:5. In embodiments, the zinc finger domain has the percentage sequence identity as disclosed in the above paragraphs to at least 110 continuous amino acids of SEQ ID NO:5. In embodiments, the zinc finger domain has the percentage sequence identity as disclosed in the above paragraphs to at least 100 continuous amino acids of SEQ ID NO:5. In embodiments, the zinc finger domain has the percentage sequence identity as disclosed in the above paragraphs to at least 90 continuous amino acids of SEQ ID NO:5. In embodiments, the zinc finger domain has the percentage sequence identity as disclosed in the above paragraphs to at least 80 continuous amino acids of SEQ ID NO:5. In embodiments, the zinc finger domain has the percentage sequence identity as disclosed in the above paragraphs to at least 70 continuous amino acids of SEQ ID NO:5. In embodiments, the zinc finger domain has the percentage sequence identity as disclosed in the above paragraphs to at least 60 continuous amino acids of SEQ ID NO:5. In embodiments, the zinc finger domain has the percentage sequence identity as disclosed in the above paragraphs to at least 50 continuous amino acids of SEQ ID NO:5. In embodiments, the zinc finger domain has the percentage sequence identity as disclosed in the above paragraphs to at least 40 continuous amino acids of SEQ ID NO:5. In embodiments, the zinc finger domain has the percentage sequence identity as disclosed in the above paragraphs to at least 30 continuous amino acids of SEQ ID NO:5. In embodiments, the zinc finger domain has the percentage sequence identity as disclosed in the above paragraphs to at least 20 continuous amino acids of SEQ ID NO:5.

The sequence of SEQ ID NO:5 encodes a non-naturally occurring peptide sequence, which may be referred to herein as “HTLV-ZFP-6” or “ZFP-6”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a DNMT domain.

In embodiments, the protein includes a nuclear localization signal, a Tat domain, a Myc tag, or combinations thereof. In embodiments, the protein further includes a nuclear localization signal. In embodiments, the protein further includes a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 14. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:14. In embodiments, the protein includes a sequence having at least 75% sequence identity to SEQ ID NO:14. In embodiments, the protein includes a sequence having at least 80% sequence identity to SEQ ID NO:14. In embodiments, the protein includes a sequence having at least 85% sequence identity to SEQ ID NO:14. In embodiments, the protein includes a sequence having at least 90% sequence identity to SEQ ID NO:14. In embodiments, the protein includes a sequence having at least 91% sequence identity to SEQ ID NO:14. In embodiments, the protein includes a sequence having at least 92% sequence identity to SEQ ID NO:14. In embodiments, the protein includes a sequence having at least 93% sequence identity to SEQ ID NO:14. In embodiments, the protein includes a sequence having at least 94% sequence identity to SEQ ID NO:14. In embodiments, the protein includes a sequence having at least 95% sequence identity to SEQ ID NO:14. In embodiments, the protein includes a sequence having at least 96% sequence identity to SEQ ID NO: 14. In embodiments, the protein includes a sequence having at least 97% sequence identity to SEQ ID NO:14. In embodiments, the protein includes a sequence having at least 98% sequence identity to SEQ ID NO:14. In embodiments, the protein includes a sequence having at least 99% sequence identity to SEQ ID NO:14. In embodiments, the protein includes the sequence of SEQ ID NO:14. In embodiments, the protein is the sequence of SEQ ID NO:14.

In embodiments, the protein includes a sequence having about 75% sequence identity to SEQ ID NO:14. In embodiments, the protein includes a sequence having about 80% sequence identity to SEQ ID NO:14. In embodiments, the protein includes a sequence having about 85% sequence identity to SEQ ID NO:14. In embodiments, the protein includes a sequence having about 90% sequence identity to SEQ ID NO:14. In embodiments, the protein includes a sequence having about 91% sequence identity to SEQ ID NO:14. In embodiments, the protein includes a sequence having about 92% sequence identity to SEQ ID NO:14. In embodiments, the protein includes a sequence having about 93% sequence identity to SEQ ID NO:14. In embodiments, the protein includes a sequence having about 94% sequence identity to SEQ ID NO:14. In embodiments, the protein includes a sequence having about 95% sequence identity to SEQ ID NO:14. In embodiments, the protein includes a sequence having about 96% sequence identity to SEQ ID NO:14. In embodiments, the protein includes a sequence having about 97% sequence identity to SEQ ID NO:14. In embodiments, the protein includes a sequence having about 98% sequence identity to SEQ ID NO:14. In embodiments, the protein includes a sequence having about 99% sequence identity to SEQ ID NO:14.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:14 connected to a second sequence fragment having at least the percentage sequence identity as disclosed above to SEQ ID NO:14 through a sequence fragment having no sequence identity to SEQ ID NO:14. In embodiments, the non-contiguous sequence is a sequence including a plurality of sequence fragments having at least the percentage sequence identity as disclosed above to SEQ ID NO:14 connected through a plurality of sequence fragments having no sequence identity to SEQ ID NO:14.

In embodiments, the protein has the percentage sequence identity as disclosed in the above paragraphs to at least 300 continuous amino acids of SEQ ID NO:14. In embodiments, the protein has the percentage sequence identity as disclosed in the above paragraphs to at least 290 continuous amino acids of SEQ ID NO:14. In embodiments, the protein has the percentage sequence identity as disclosed in the above paragraphs to at least 280 continuous amino acids of SEQ ID NO:14. In embodiments, the protein has the percentage sequence identity as disclosed in the above paragraphs to at least 270 continuous amino acids of SEQ ID NO:14. In embodiments, the protein has the percentage sequence identity as disclosed in the above paragraphs to at least 260 continuous amino acids of SEQ ID NO:14. In embodiments, the protein has the percentage sequence identity as disclosed in the above paragraphs to at least 250 continuous amino acids of SEQ ID NO:14. In embodiments, the protein has the percentage sequence identity as disclosed in the above paragraphs to at least 240 continuous amino acids of SEQ ID NO:14. In embodiments, the protein has the percentage sequence identity as disclosed in the above paragraphs to at least 230 continuous amino acids of SEQ ID NO:14. In embodiments, the protein has the percentage sequence identity as disclosed in the above paragraphs to at least 220 continuous amino acids of SEQ ID NO:14. In embodiments, the protein has the percentage sequence identity as disclosed in the above paragraphs to at least 210 continuous amino acids of SEQ ID NO:14. In embodiments, the protein has the percentage sequence identity as disclosed in the above paragraphs to at least 200 continuous amino acids of SEQ ID NO:14. In embodiments, the protein has the percentage sequence identity as disclosed in the above paragraphs to at least 190 continuous amino acids of SEQ ID NO:14. In embodiments, the protein has the percentage sequence identity as disclosed in the above paragraphs to at least 180 continuous amino acids of SEQ ID NO:14. In embodiments, the protein has the percentage sequence identity as disclosed in the above paragraphs to at least 170 continuous amino acids of SEQ ID NO:14. In embodiments, the protein has the percentage sequence identity as disclosed in the above paragraphs to at least 160 continuous amino acids of SEQ ID NO:14. In embodiments, the protein has the percentage sequence identity as disclosed in the above paragraphs to at least 150 continuous amino acids of SEQ ID NO:14. In embodiments, the protein has the percentage sequence identity as disclosed in the above paragraphs to at least 140 continuous amino acids of SEQ ID NO:14. In embodiments, the protein has the percentage sequence identity as disclosed in the above paragraphs to at least 130 continuous amino acids of SEQ ID NO:14. In embodiments, the protein has the percentage sequence identity as disclosed in the above paragraphs to at least 120 continuous amino acids of SEQ ID NO:14. In embodiments, the protein has the percentage sequence identity as disclosed in the above paragraphs to at least 110 continuous amino acids of SEQ ID NO:14. In embodiments, the protein has the percentage sequence identity as disclosed in the above paragraphs to at least 100 continuous amino acids of SEQ ID NO:14. In embodiments, the protein has the percentage sequence identity as disclosed in the above paragraphs to at least 90 continuous amino acids of SEQ ID NO:14. In embodiments, the protein has the percentage sequence identity as disclosed in the above paragraphs to at least 80 continuous amino acids of SEQ ID NO:14. In embodiments, the protein has the percentage sequence identity as disclosed in the above paragraphs to at least 70 continuous amino acids of SEQ ID NO:14. In embodiments, the protein has the percentage sequence identity as disclosed in the above paragraphs to at least 60 continuous amino acids of SEQ ID NO:14. In embodiments, the protein has the percentage sequence identity as disclosed in the above paragraphs to at least 50 continuous amino acids of SEQ ID NO:14. In embodiments, the protein has the percentage sequence identity as disclosed in the above paragraphs to at least 40 continuous amino acids of SEQ ID NO:14. In embodiments, the protein has the percentage sequence identity as disclosed in the above paragraphs to at least 30 continuous amino acids of SEQ ID NO:14. In embodiments, the protein has the percentage sequence identity as disclosed in the above paragraphs to at least 20 continuous amino acids of SEQ ID NO:14.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:32. In embodiments, the sequence has at least 75%, 80%, 85%, 90%, 95%, 96%, 97%, 98% or 99% sequence identity to the sequence of SEQ ID NO:32. In embodiments, the sequence has at least 90%, 95%, 96%, 97%, 98%, 99% or 100% nucleic acid sequence identity across the whole sequence or a portion of the sequence (e.g. a 5, 10, 15 or 20 continuous nucleic acid portion) of SEQ ID NO:32.

In embodiments, the sequence within the HTLV-1 LTR has at least 75% sequence identity to the sequence of SEQ ID NO:32. In embodiments, the sequence within the HTLV-1 LTR has at least 80% sequence identity to the sequence of SEQ ID NO:32. In embodiments, the sequence within the HTLV-1 LTR has at least 85% sequence identity to the sequence of SEQ ID NO:32. In embodiments, the sequence within the HTLV-1 LTR has at least 90% sequence identity to the sequence of SEQ ID NO:32. In embodiments, the sequence within the HTLV-1 LTR has at least 95% sequence identity to the sequence of SEQ ID NO:32. In embodiments, the sequence within the HTLV-1 LTR has at least 98% sequence identity to the sequence of SEQ ID NO:32. In embodiments, the sequence within the HTLV-1 LTR has at least 99% sequence identity to the sequence of SEQ ID NO:32. In embodiments, the sequence within the HTLV-1 LTR includes the sequence of SEQ ID NO:32. In embodiments, the sequence within the HTLV-1 LTR is the sequence of SEQ ID NO:32.

In embodiments, the sequence within the HTLV-1 LTR has about 75% sequence identity to the sequence of SEQ ID NO:32. In embodiments, the sequence within the HTLV-1 LTR has about 80% sequence identity to the sequence of SEQ ID NO:32. In embodiments, the sequence within the HTLV-1 LTR has about 85% sequence identity to the sequence of SEQ ID NO:32. In embodiments, the sequence within the HTLV-1 LTR has about 90% sequence identity to the sequence of SEQ ID NO:32. In embodiments, the sequence within the HTLV-1 LTR has about 95% sequence identity to the sequence of SEQ ID NO:32. In embodiments, the sequence within the HTLV-1 LTR has about 98% sequence identity to the sequence of SEQ ID NO:32. In embodiments, the sequence within the HTLV-1 LTR has about 99% sequence identity to the sequence of SEQ ID NO:32.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:81, F2 includes SEQ ID NO:82, F3 includes SEQ ID NO:83, F4 includes SEQ ID NO:84, F5 includes SEQ ID NO:85 and F6 includes SEQ ID NO:86. In embodiments, F1 is SEQ ID NO:81, F2 is SEQ ID NO:82, F3 is SEQ ID NO:83, F4 is SEQ ID NO:84, F5 is SEQ ID NO:85 and F6 is SEQ ID NO:86.

In embodiments, the zinc finger domain has at least 75%, 80%, 85%, 90%, 95%, 96%, 97%, 98% or 99% sequence identity to the sequence of SEQ ID NO:9. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:9. In embodiments, the zinc finger domain has at least 75% sequence identity to SEQ ID NO:9. In embodiments, the zinc finger domain has at least 80% sequence identity to SEQ ID NO:9. In embodiments, the zinc finger domain has at least 85% sequence identity to SEQ ID NO:9. In embodiments, the zinc finger domain has at least 90% sequence identity to SEQ ID NO:9. In embodiments, the zinc finger domain includes a sequence having at least 91% sequence identity to SEQ ID NO:9. In embodiments, the zinc finger domain includes a sequence having at least 92% sequence identity to SEQ ID NO:9. In embodiments, the zinc finger domain includes a sequence having at least 93% sequence identity to SEQ ID NO:9. In embodiments, the zinc finger domain includes a sequence having at least 94% sequence identity to SEQ ID NO:9. In embodiments, the zinc finger domain has at least 95% sequence identity to SEQ ID NO:9. In embodiments, the zinc finger domain has at least 96% sequence identity to SEQ ID NO:9. In embodiments, the zinc finger domain includes a sequence having at least 97% sequence identity to SEQ ID NO:9. In embodiments, the zinc finger domain has at least 98% sequence identity to SEQ ID NO:9. In embodiments, the zinc finger domain has at least 99% sequence identity to SEQ ID NO:9. In embodiments, the zinc finger domain includes the sequence of SEQ ID NO:9. In embodiments, the zinc finger domain is the sequence of SEQ ID NO:9.

In embodiments, the zinc finger domain includes a sequence having about 75% sequence identity to SEQ ID NO:9. In embodiments, the zinc finger domain includes a sequence having about 80% sequence identity to SEQ ID NO:9. In embodiments, the zinc finger domain includes a sequence having about 85% sequence identity to SEQ ID NO:9. In embodiments, the zinc finger domain includes a sequence having about 90% sequence identity to SEQ ID NO:9. In embodiments, the zinc finger domain includes a sequence having about 91% sequence identity to SEQ ID NO:9. In embodiments, the zinc finger domain includes a sequence having about 92% sequence identity to SEQ ID NO:9. In embodiments, the zinc finger domain includes a sequence having about 93% sequence identity to SEQ ID NO:9. In embodiments, the zinc finger domain includes a sequence having about 94% sequence identity to SEQ ID NO:9. In embodiments, the zinc finger domain includes a sequence having about 95% sequence identity to SEQ ID NO:9. In embodiments, the zinc finger domain includes a sequence having about 96% sequence identity to SEQ ID NO:9. In embodiments, the zinc finger domain includes a sequence having about 97% sequence identity to SEQ ID NO:9. In embodiments, the zinc finger domain includes a sequence having about 98% sequence identity to SEQ ID NO:9. In embodiments, the zinc finger domain includes a sequence having about 99% sequence identity to SEQ ID NO:9.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:9 connected to a second sequence fragment having at least the percentage sequence identity as disclosed above to SEQ ID NO:9 through a sequence fragment having no sequence identity to SEQ ID NO:9. In embodiments, the non-contiguous sequence is a sequence including a plurality of sequence fragments having at least the percentage sequence identity as disclosed above to SEQ ID NO:9 connected through a plurality of sequence fragments having no sequence identity to SEQ ID NO:9.

In embodiments, the zinc finger domain has the percentage sequence identity as disclosed in the above paragraphs to at least 170 continuous amino acids of SEQ ID NO:9. In embodiments, the zinc finger domain has the percentage sequence identity as disclosed in the above paragraphs to at least 160 continuous amino acids of SEQ ID NO:9. In embodiments, the zinc finger domain has the percentage sequence identity as disclosed in the above paragraphs to at least 150 continuous amino acids of SEQ ID NO:9. In embodiments, the zinc finger domain has the percentage sequence identity as disclosed in the above paragraphs to at least 140 continuous amino acids of SEQ ID NO:9. In embodiments, the zinc finger domain has the percentage sequence identity as disclosed in the above paragraphs to at least 130 continuous amino acids of SEQ ID NO:9. In embodiments, the zinc finger domain has the percentage sequence identity as disclosed in the above paragraphs to at least 120 continuous amino acids of SEQ ID NO:9. In embodiments, the zinc finger domain has the percentage sequence identity as disclosed in the above paragraphs to at least 110 continuous amino acids of SEQ ID NO:9. In embodiments, the zinc finger domain has the percentage sequence identity as disclosed in the above paragraphs to at least 100 continuous amino acids of SEQ ID NO:9. In embodiments, the zinc finger domain has the percentage sequence identity as disclosed in the above paragraphs to at least 90 continuous amino acids of SEQ ID NO:9. In embodiments, the zinc finger domain has the percentage sequence identity as disclosed in the above paragraphs to at least 80 continuous amino acids of SEQ ID NO:9. In embodiments, the zinc finger domain has the percentage sequence identity as disclosed in the above paragraphs to at least 70 continuous amino acids of SEQ ID NO:9. In embodiments, the zinc finger domain has the percentage sequence identity as disclosed in the above paragraphs to at least 60 continuous amino acids of SEQ ID NO:9. In embodiments, the zinc finger domain has the percentage sequence identity as disclosed in the above paragraphs to at least 50 continuous amino acids of SEQ ID NO:9. In embodiments, the zinc finger domain has the percentage sequence identity as disclosed in the above paragraphs to at least 40 continuous amino acids of SEQ ID NO:9. In embodiments, the zinc finger domain has the percentage sequence identity as disclosed in the above paragraphs to at least 30 continuous amino acids of SEQ ID NO:9. In embodiments, the zinc finger domain has the percentage sequence identity as disclosed in the above paragraphs to at least 20 continuous amino acids of SEQ ID NO:9.

The sequence of SEQ ID NO:9 encodes a non-naturally occurring peptide sequence, which may be referred to herein as “HTLV-ZFP-10” or “ZFP-10”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a DNMT domain. In embodiments, the transcriptional repressor includes a KRAB domain and meCP2 or a fragment thereof.

In embodiments, the protein further includes a nuclear localization signal, a Tat domain, a Myc tag, or combinations thereof. In embodiments, the protein further includes a nuclear localization signal. In embodiments, the protein further includes a Tat domain. In embodiments, the protein further includes a Myc tag.

In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:18. In embodiments, the protein includes a sequence having at least 75% sequence identity to SEQ ID NO:18. In embodiments, the protein includes a sequence having at least 80% sequence identity to SEQ ID NO:18. In embodiments, the protein includes a sequence having at least 85% sequence identity to SEQ ID NO: 18. In embodiments, the protein includes a sequence having at least 90% sequence identity to SEQ ID NO:18. In embodiments, the protein includes a sequence having at least 91% sequence identity to SEQ ID NO:18. In embodiments, the protein includes a sequence having at least 92% sequence identity to SEQ ID NO:18. In embodiments, the protein includes a sequence having at least 93% sequence identity to SEQ ID NO:18. In embodiments, the protein includes a sequence having at least 94% sequence identity to SEQ ID NO: 18. In embodiments, the protein includes a sequence having at least 95% sequence identity to SEQ ID NO:18. In embodiments, the protein includes a sequence having at least 96% sequence identity to SEQ ID NO:18. In embodiments, the protein includes a sequence having at least 97% sequence identity to SEQ ID NO:18. In embodiments, the protein includes a sequence having at least 98% sequence identity to SEQ ID NO:18. In embodiments, the protein includes a sequence having at least 99% sequence identity to SEQ ID NO: 18. In embodiments, the protein includes the sequence of SEQ ID NO:18. In embodiments, the protein is the sequence of SEQ ID NO:18.

In embodiments, the protein includes a sequence having about 75% sequence identity to SEQ ID NO:18. In embodiments, the protein includes a sequence having about 80% sequence identity to SEQ ID NO:18. In embodiments, the protein includes a sequence having about 85% sequence identity to SEQ ID NO:18. In embodiments, the protein includes a sequence having about 90% sequence identity to SEQ ID NO:18. In embodiments, the protein includes a sequence having about 91% sequence identity to SEQ ID NO:18. In embodiments, the protein includes a sequence having about 92% sequence identity to SEQ ID NO:18. In embodiments, the protein includes a sequence having about 93% sequence identity to SEQ ID NO:18. In embodiments, the protein includes a sequence having about 94% sequence identity to SEQ ID NO:18. In embodiments, the protein includes a sequence having about 95% sequence identity to SEQ ID NO:18. In embodiments, the protein includes a sequence having about 96% sequence identity to SEQ ID NO:18. In embodiments, the protein includes a sequence having about 97% sequence identity to SEQ ID NO:18. In embodiments, the protein includes a sequence having about 98% sequence identity to SEQ ID NO:18. In embodiments, the protein includes a sequence having about 99% sequence identity to SEQ ID NO:18.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:18 connected to a second sequence fragment having at least the percentage sequence identity as disclosed above to SEQ ID NO:18 through a sequence fragment having no sequence identity to SEQ ID NO:18. In embodiments, the non-contiguous sequence is a sequence including a plurality of sequence fragments having at least the percentage sequence identity as disclosed above to SEQ ID NO:18 connected through a plurality of sequence fragments having no sequence identity to SEQ ID NO:18.

In embodiments, the protein has the percentage sequence identity as disclosed in the above paragraphs to at least 300 continuous amino acids of SEQ ID NO:18. In embodiments, the protein has the percentage sequence identity as disclosed in the above paragraphs to at least 290 continuous amino acids of SEQ ID NO:18. In embodiments, the protein has the percentage sequence identity as disclosed in the above paragraphs to at least 280 continuous amino acids of SEQ ID NO:18. In embodiments, the protein has the percentage sequence identity as disclosed in the above paragraphs to at least 270 continuous amino acids of SEQ ID NO:18. In embodiments, the protein has the percentage sequence identity as disclosed in the above paragraphs to at least 260 continuous amino acids of SEQ ID NO:18. In embodiments, the protein has the percentage sequence identity as disclosed in the above paragraphs to at least 250 continuous amino acids of SEQ ID NO:18. In embodiments, the protein has the percentage sequence identity as disclosed in the above paragraphs to at least 240 continuous amino acids of SEQ ID NO:18. In embodiments, the protein has the percentage sequence identity as disclosed in the above paragraphs to at least 230 continuous amino acids of SEQ ID NO:18. In embodiments, the protein has the percentage sequence identity as disclosed in the above paragraphs to at least 220 continuous amino acids of SEQ ID NO:18. In embodiments, the protein has the percentage sequence identity as disclosed in the above paragraphs to at least 210 continuous amino acids of SEQ ID NO:18. In embodiments, the protein has the percentage sequence identity as disclosed in the above paragraphs to at least 200 continuous amino acids of SEQ ID NO:18. In embodiments, the protein has the percentage sequence identity as disclosed in the above paragraphs to at least 190 continuous amino acids of SEQ ID NO:18. In embodiments, the protein has the percentage sequence identity as disclosed in the above paragraphs to at least 180 continuous amino acids of SEQ ID NO:18. In embodiments, the protein has the percentage sequence identity as disclosed in the above paragraphs to at least 170 continuous amino acids of SEQ ID NO:18. In embodiments, the protein has the percentage sequence identity as disclosed in the above paragraphs to at least 160 continuous amino acids of SEQ ID NO:18. In embodiments, the protein has the percentage sequence identity as disclosed in the above paragraphs to at least 150 continuous amino acids of SEQ ID NO:18. In embodiments, the protein has the percentage sequence identity as disclosed in the above paragraphs to at least 140 continuous amino acids of SEQ ID NO:18. In embodiments, the protein has the percentage sequence identity as disclosed in the above paragraphs to at least 130 continuous amino acids of SEQ ID NO:18. In embodiments, the protein has the percentage sequence identity as disclosed in the above paragraphs to at least 120 continuous amino acids of SEQ ID NO:18. In embodiments, the protein has the percentage sequence identity as disclosed in the above paragraphs to at least 110 continuous amino acids of SEQ ID NO:18. In embodiments, the protein has the percentage sequence identity as disclosed in the above paragraphs to at least 100 continuous amino acids of SEQ ID NO:18. In embodiments, the protein has the percentage sequence identity as disclosed in the above paragraphs to at least 90 continuous amino acids of SEQ ID NO:18. In embodiments, the protein has the percentage sequence identity as disclosed in the above paragraphs to at least 80 continuous amino acids of SEQ ID NO:18. In embodiments, the protein has the percentage sequence identity as disclosed in the above paragraphs to at least 70 continuous amino acids of SEQ ID NO:18. In embodiments, the protein has the percentage sequence identity as disclosed in the above paragraphs to at least 60 continuous amino acids of SEQ ID NO:18. In embodiments, the protein has the percentage sequence identity as disclosed in the above paragraphs to at least 50 continuous amino acids of SEQ ID NO:18. In embodiments, the protein has the percentage sequence identity as disclosed in the above paragraphs to at least 40 continuous amino acids of SEQ ID NO:18. In embodiments, the protein has the percentage sequence identity as disclosed in the above paragraphs to at least 30 continuous amino acids of SEQ ID NO:18. In embodiments, the protein has the percentage sequence identity as disclosed in the above paragraphs to at least 20 continuous amino acids of SEQ ID NO:18.

In another aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:31. In embodiments, the sequence has at least 75%, 80%, 85%, 90%, 95%, 96%, 97%, 98% or 99% sequence identity to the sequence of SEQ ID NO:31. In embodiments, the sequence has at least 90%, 95%, 96%, 97%, 98%, 99% or 100% nucleic acid sequence identity across the whole sequence or a portion of the sequence (e.g. a 5, 10, 11, 12, 13, 14, or 15 continuous nucleic acid portion) of SEQ ID NO:31.

In embodiments, the sequence within the HTLV-1 LTR has at least 75% sequence identity to the sequence of SEQ ID NO:31. In embodiments, the sequence within the HTLV-1 LTR has at least 80% sequence identity to the sequence of SEQ ID NO:31. In embodiments, the sequence within the HTLV-1 LTR has at least 85% sequence identity to the sequence of SEQ ID NO:31. In embodiments, the sequence within the HTLV-1 LTR has at least 90% sequence identity to the sequence of SEQ ID NO:31. In embodiments, the sequence within the HTLV-1 LTR has at least 95% sequence identity to the sequence of SEQ ID NO:31. In embodiments, the sequence within the HTLV-1 LTR has at least 98% sequence identity to the sequence of SEQ ID NO:31. In embodiments, the sequence within the HTLV-1 LTR has at least 99% sequence identity to the sequence of SEQ ID NO:31. In embodiments, the sequence within the HTLV-1 LTR includes the sequence of SEQ ID NO:31. In embodiments, the sequence within the HTLV-1 LTR is the sequence of SEQ ID NO:31.

In embodiments, the sequence within the HTLV-1 LTR has about 75% sequence identity to the sequence of SEQ ID NO:31. In embodiments, the sequence within the HTLV-1 LTR has about 80% sequence identity to the sequence of SEQ ID NO:31. In embodiments, the sequence within the HTLV-1 LTR has about 85% sequence identity to the sequence of SEQ ID NO:31. In embodiments, the sequence within the HTLV-1 LTR has about 90% sequence identity to the sequence of SEQ ID NO:31. In embodiments, the sequence within the HTLV-1 LTR has about 95% sequence identity to the sequence of SEQ ID NO:31. In embodiments, the sequence within the HTLV-1 LTR has about 98% sequence identity to the sequence of SEQ ID NO:31. In embodiments, the sequence within the HTLV-1 LTR has about 99% sequence identity to the sequence of SEQ ID NO:31.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:75, F2 includes SEQ ID NO:76, F3 includes SEQ ID NO:77, F4 includes SEQ ID NO:78, F5 includes SEQ ID NO:79 and F6 includes SEQ ID NO:80. In embodiments, the F1 is SEQ ID NO:75, F2 is SEQ ID NO:76, F3 is SEQ ID NO:77, F4 is SEQ ID NO:78, F5 is SEQ ID NO:79 and F6 is SEQ ID NO:80.

In embodiments, the zinc finger domain has at least 75%, 80%, 85%, 90%, 95%, 96%, 97%, 98% or 99% sequence identity to the sequence of SEQ ID NO:8. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:8. In embodiments, the zinc finger domain includes a sequence having at least 75% sequence identity to SEQ ID NO:8. In embodiments, the zinc finger domain includes a sequence having at least 80% sequence identity to SEQ ID NO:8. In embodiments, the zinc finger domain includes a sequence having at least 85% sequence identity to SEQ ID NO:8. In embodiments, the zinc finger domain includes a sequence having at least 90% sequence identity to SEQ ID NO:8. In embodiments, the zinc finger domain includes a sequence having at least 91% sequence identity to SEQ ID NO:8. In embodiments, the zinc finger domain includes a sequence having at least 92% sequence identity to SEQ ID NO:8. In embodiments, the zinc finger domain includes a sequence having at least 93% sequence identity to SEQ ID NO:8. In embodiments, the zinc finger domain includes a sequence having at least 94% sequence identity to SEQ ID NO:8. In embodiments, the zinc finger domain includes a sequence having at least 95% sequence identity to SEQ ID NO:8. In embodiments, the zinc finger domain includes a sequence having at least 96% sequence identity to SEQ ID NO:8. In embodiments, the zinc finger domain includes a sequence having at least 97% sequence identity to SEQ ID NO:8. In embodiments, the zinc finger domain includes a sequence having at least 98% sequence identity to SEQ ID NO:8. In embodiments, the zinc finger domain includes a sequence having at least 99% sequence identity to SEQ ID NO:8. In embodiments, the zinc finger domain includes the sequence of SEQ ID NO: 8. In embodiments, the zinc finger domain is the sequence of SEQ ID NO:8.

In embodiments, the zinc finger domain includes a sequence having about 75% sequence identity to SEQ ID NO:8. In embodiments, the zinc finger domain includes a sequence having about 80% sequence identity to SEQ ID NO:8. In embodiments, the zinc finger domain includes a sequence having about 85% sequence identity to SEQ ID NO:8. In embodiments, the zinc finger domain includes a sequence having about 90% sequence identity to SEQ ID NO:8. In embodiments, the zinc finger domain includes a sequence having about 91% sequence identity to SEQ ID NO:8. In embodiments, the zinc finger domain includes a sequence having about 92% sequence identity to SEQ ID NO:8. In embodiments, the zinc finger domain includes a sequence having about 93% sequence identity to SEQ ID NO:8. In embodiments, the zinc finger domain includes a sequence having about 94% sequence identity to SEQ ID NO:8. In embodiments, the zinc finger domain includes a sequence having about 95% sequence identity to SEQ ID NO:8. In embodiments, the zinc finger domain includes a sequence having about 96% sequence identity to SEQ ID NO:8. In embodiments, the zinc finger domain includes a sequence having about 97% sequence identity to SEQ ID NO:8. In embodiments, the zinc finger domain includes a sequence having about 98% sequence identity to SEQ ID NO:8. In embodiments, the zinc finger domain includes a sequence having about 99% sequence identity to SEQ ID NO:8.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:8 connected to a second sequence fragment having at least the percentage sequence identity as disclosed above to SEQ ID NO:8 through a sequence fragment having no sequence identity to SEQ ID NO:8. In embodiments, the non-contiguous sequence is a sequence including a plurality of sequence fragments having at least the percentage sequence identity as disclosed above to SEQ ID NO:8 connected through a plurality of sequence fragments having no sequence identity to SEQ ID NO:8.

In embodiments, the zinc finger domain has the percentage sequence identity as disclosed in the above paragraphs to at least 170 continuous amino acids of SEQ ID NO:8. In embodiments, the zinc finger domain has the percentage sequence identity as disclosed in the above paragraphs to at least 160 continuous amino acids of SEQ ID NO:8. In embodiments, the zinc finger domain has the percentage sequence identity as disclosed in the above paragraphs to at least 150 continuous amino acids of SEQ ID NO:8. In embodiments, the zinc finger domain has the percentage sequence identity as disclosed in the above paragraphs to at least 140 continuous amino acids of SEQ ID NO:8. In embodiments, the zinc finger domain has the percentage sequence identity as disclosed in the above paragraphs to at least 130 continuous amino acids of SEQ ID NO:8. In embodiments, the zinc finger domain has the percentage sequence identity as disclosed in the above paragraphs to at least 120 continuous amino acids of SEQ ID NO:8. In embodiments, the zinc finger domain has the percentage sequence identity as disclosed in the above paragraphs to at least 110 continuous amino acids of SEQ ID NO:8. In embodiments, the zinc finger domain has the percentage sequence identity as disclosed in the above paragraphs to at least 100 continuous amino acids of SEQ ID NO:8. In embodiments, the zinc finger domain has the percentage sequence identity as disclosed in the above paragraphs to at least 90 continuous amino acids of SEQ ID NO:8. In embodiments, the zinc finger domain has the percentage sequence identity as disclosed in the above paragraphs to at least 80 continuous amino acids of SEQ ID NO:8. In embodiments, the zinc finger domain has the percentage sequence identity as disclosed in the above paragraphs to at least 70 continuous amino acids of SEQ ID NO:8. In embodiments, the zinc finger domain has the percentage sequence identity as disclosed in the above paragraphs to at least 60 continuous amino acids of SEQ ID NO:8. In embodiments, the zinc finger domain has the percentage sequence identity as disclosed in the above paragraphs to at least 50 continuous amino acids of SEQ ID NO:8. In embodiments, the zinc finger domain has the percentage sequence identity as disclosed in the above paragraphs to at least 40 continuous amino acids of SEQ ID NO:8. In embodiments, the zinc finger domain has the percentage sequence identity as disclosed in the above paragraphs to at least 30 continuous amino acids of SEQ ID NO:8. In embodiments, the zinc finger domain has the percentage sequence identity as disclosed in the above paragraphs to at least 20 continuous amino acids of SEQ ID NO:8.

The sequence of SEQ ID NO:8 encodes a non-naturally occurring peptide sequence, which may be referred to herein as “HTLV-ZFP-9” or “ZFP-9”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a KRAB domain and meCP2 or a fragment thereof.

In embodiments, the protein further includes a nuclear localization signal, a Tat domain, a Myc tag, or combinations thereof. In embodiments, the protein further includes a nuclear localization signal. In embodiments, the protein further includes a a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 17. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:17. In embodiments, the protein includes a sequence having at least 75% sequence identity to SEQ ID NO:17. In embodiments, the protein includes a sequence having at least 80% sequence identity to SEQ ID NO:17. In embodiments, the protein includes a sequence having at least 85% sequence identity to SEQ ID NO:17. In embodiments, the protein includes a sequence having at least 90% sequence identity to SEQ ID NO:17. In embodiments, the protein includes a sequence having at least 91% sequence identity to SEQ ID NO:17. In embodiments, the protein includes a sequence having at least 92% sequence identity to SEQ ID NO:17. In embodiments, the protein includes a sequence having at least 93% sequence identity to SEQ ID NO:17. In embodiments, the protein includes a sequence having at least 94% sequence identity to SEQ ID NO:17. In embodiments, the protein includes a sequence having at least 95% sequence identity to SEQ ID NO:17. In embodiments, the protein includes a sequence having at least 96% sequence identity to SEQ ID NO: 17. In embodiments, the protein includes a sequence having at least 97% sequence identity to SEQ ID NO:17. In embodiments, the protein includes a sequence having at least 98% sequence identity to SEQ ID NO:17. In embodiments, the protein includes a sequence having at least 99% sequence identity to SEQ ID NO:17. In embodiments, the protein includes the sequence of SEQ ID NO:17. In embodiments, the protein is the sequence of SEQ ID NO:17.

In embodiments, the protein includes a sequence having about 75% sequence identity to SEQ ID NO:17. In embodiments, the protein includes a sequence having about 80% sequence identity to SEQ ID NO:17. In embodiments, the protein includes a sequence having about 85% sequence identity to SEQ ID NO:17. In embodiments, the protein includes a sequence having about 90% sequence identity to SEQ ID NO:17. In embodiments, the protein includes a sequence having about 91% sequence identity to SEQ ID NO:17. In embodiments, the protein includes a sequence having about 92% sequence identity to SEQ ID NO:17. In embodiments, the protein includes a sequence having about 93% sequence identity to SEQ ID NO:17. In embodiments, the protein includes a sequence having about 94% sequence identity to SEQ ID NO:17. In embodiments, the protein includes a sequence having about 95% sequence identity to SEQ ID NO:17. In embodiments, the protein includes a sequence having about 96% sequence identity to SEQ ID NO:17. In embodiments, the protein includes a sequence having about 97% sequence identity to SEQ ID NO:17. In embodiments, the protein includes a sequence having about 98% sequence identity to SEQ ID NO:17. In embodiments, the protein includes a sequence having about 99% sequence identity to SEQ ID NO:17.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:17 connected to a second sequence fragment having at least the percentage sequence identity as disclosed above to SEQ ID NO:17 through a sequence fragment having no sequence identity to SEQ ID NO:17. In embodiments, the non-contiguous sequence is a sequence including a plurality of sequence fragments having at least the percentage sequence identity as disclosed above to SEQ ID NO:17 connected through a plurality of sequence fragments having no sequence identity to SEQ ID NO:17.

In embodiments, the protein has the percentage sequence identity as disclosed in the above paragraphs to at least 300 continuous amino acids of SEQ ID NO:17. In embodiments, the protein has the percentage sequence identity as disclosed in the above paragraphs to at least 290 continuous amino acids of SEQ ID NO:17. In embodiments, the protein has the percentage sequence identity as disclosed in the above paragraphs to at least 280 continuous amino acids of SEQ ID NO:17. In embodiments, the protein has the percentage sequence identity as disclosed in the above paragraphs to at least 270 continuous amino acids of SEQ ID NO:17. In embodiments, the protein has the percentage sequence identity as disclosed in the above paragraphs to at least 260 continuous amino acids of SEQ ID NO:17. In embodiments, the protein has the percentage sequence identity as disclosed in the above paragraphs to at least 250 continuous amino acids of SEQ ID NO:17. In embodiments, the protein has the percentage sequence identity as disclosed in the above paragraphs to at least 240 continuous amino acids of SEQ ID NO:17. In embodiments, the protein has the percentage sequence identity as disclosed in the above paragraphs to at least 230 continuous amino acids of SEQ ID NO:17. In embodiments, the protein has the percentage sequence identity as disclosed in the above paragraphs to at least 220 continuous amino acids of SEQ ID NO:17. In embodiments, the protein has the percentage sequence identity as disclosed in the above paragraphs to at least 210 continuous amino acids of SEQ ID NO:17. In embodiments, the protein has the percentage sequence identity as disclosed in the above paragraphs to at least 200 continuous amino acids of SEQ ID NO:17. In embodiments, the protein has the percentage sequence identity as disclosed in the above paragraphs to at least 190 continuous amino acids of SEQ ID NO:17. In embodiments, the protein has the percentage sequence identity as disclosed in the above paragraphs to at least 170 continuous amino acids of SEQ ID NO:17. In embodiments, the protein has the percentage sequence identity as disclosed in the above paragraphs to at least 170 continuous amino acids of SEQ ID NO:17. In embodiments, the protein has the percentage sequence identity as disclosed in the above paragraphs to at least 160 continuous amino acids of SEQ ID NO:17. In embodiments, the protein has the percentage sequence identity as disclosed in the above paragraphs to at least 150 continuous amino acids of SEQ ID NO:17. In embodiments, the protein has the percentage sequence identity as disclosed in the above paragraphs to at least 170 continuous amino acids of SEQ ID NO:17. In embodiments, the protein has the percentage sequence identity as disclosed in the above paragraphs to at least 130 continuous amino acids of SEQ ID NO:17. In embodiments, the protein has the percentage sequence identity as disclosed in the above paragraphs to at least 120 continuous amino acids of SEQ ID NO:17. In embodiments, the protein has the percentage sequence identity as disclosed in the above paragraphs to at least 110 continuous amino acids of SEQ ID NO:17. In embodiments, the protein has the percentage sequence identity as disclosed in the above paragraphs to at least 100 continuous amino acids of SEQ ID NO:17. In embodiments, the protein has the percentage sequence identity as disclosed in the above paragraphs to at least 90 continuous amino acids of SEQ ID NO:17. In embodiments, the protein has the percentage sequence identity as disclosed in the above paragraphs to at least 80 continuous amino acids of SEQ ID NO:17. In embodiments, the protein has the percentage sequence identity as disclosed in the above paragraphs to at least 70 continuous amino acids of SEQ ID NO:17. In embodiments, the protein has the percentage sequence identity as disclosed in the above paragraphs to at least 60 continuous amino acids of SEQ ID NO:17. In embodiments, the protein has the percentage sequence identity as disclosed in the above paragraphs to at least 50 continuous amino acids of SEQ ID NO:17. In embodiments, the protein has the percentage sequence identity as disclosed in the above paragraphs to at least 40 continuous amino acids of SEQ ID NO:17.

In another aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:30. In embodiments, the sequence has at least 75%, 80%, 85%, 90%, 95%, 96%, 97%, 98% or 99% sequence identity to the sequence of SEQ ID NO:30. In embodiments, the sequence has at least 90%, 95%, 96%, 97%, 98%, 99% or 100% nucleic acid sequence identity across the whole sequence or a portion of the sequence (e.g. a 5, 10, 11, 12, 13, 14, or 15 continuous nucleic acid portion) of SEQ ID NO:30.

In embodiments, the sequence within the HTLV-1 LTR has at least 75% sequence identity to the sequence of SEQ ID NO:30. In embodiments, the sequence within the HTLV-1 LTR has at least 80% sequence identity to the sequence of SEQ ID NO:30. In embodiments, the sequence within the HTLV-1 LTR has at least 85% sequence identity to the sequence of SEQ ID NO:30. In embodiments, the sequence within the HTLV-1 LTR has at least 90% sequence identity to the sequence of SEQ ID NO:30. In embodiments, the sequence within the HTLV-1 LTR has at least 95% sequence identity to the sequence of SEQ ID NO:30. In embodiments, the sequence within the HTLV-1 LTR has at least 98% sequence identity to the sequence of SEQ ID NO:30. In embodiments, the sequence within the HTLV-1 LTR has at least 99% sequence identity to the sequence of SEQ ID NO:30. In embodiments, the sequence within the HTLV-1 LTR includes the sequence of SEQ ID NO:30. In embodiments, the sequence within the HTLV-1 LTR is the sequence of SEQ ID NO:30.

In embodiments, the sequence within the HTLV-1 LTR has about 75% sequence identity to the sequence of SEQ ID NO:30. In embodiments, the sequence within the HTLV-1 LTR has about 80% sequence identity to the sequence of SEQ ID NO:30. In embodiments, the sequence within the HTLV-1 LTR has about 85% sequence identity to the sequence of SEQ ID NO:30. In embodiments, the sequence within the HTLV-1 LTR has about 90% sequence identity to the sequence of SEQ ID NO:30. In embodiments, the sequence within the HTLV-1 LTR has about 95% sequence identity to the sequence of SEQ ID NO:30. In embodiments, the sequence within the HTLV-1 LTR has about 98% sequence identity to the sequence of SEQ ID NO:30. In embodiments, the sequence within the HTLV-1 LTR has about 99% sequence identity to the sequence of SEQ ID NO:30.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:69, F2 includes SEQ ID NO:70, F3 includes SEQ ID NO:71, F4 includes SEQ ID NO:72, F5 includes SEQ ID NO:73 and F6 includes SEQ ID NO:74. In embodiments, the F1 is SEQ ID NO:69, F2 is SEQ ID NO:70, F3 is SEQ ID NO:71, F4 is SEQ ID NO:72, F5 is SEQ ID NO:73 and F6 is SEQ ID NO:74.

In embodiments, the zinc finger domain has at least 75%, 80%, 85%, 90%, 95%, 96%, 97%, 98% or 99% sequence identity to the sequence of SEQ ID NO:7. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:7. In embodiments, the zinc finger domain includes a sequence having at least 75% sequence identity to SEQ ID NO:7. In embodiments, the zinc finger domain includes a sequence having at least 80% sequence identity to SEQ ID NO:7. In embodiments, the zinc finger domain includes a sequence having at least 85% sequence identity to SEQ ID NO:7. In embodiments, the zinc finger domain includes a sequence having at least 90% sequence identity to SEQ ID NO:7. In embodiments, the zinc finger domain includes a sequence having at least 91% sequence identity to SEQ ID NO:7. In embodiments, the zinc finger domain includes a sequence having at least 92% sequence identity to SEQ ID NO:7. In embodiments, the zinc finger domain includes a sequence having at least 93% sequence identity to SEQ ID NO:7. In embodiments, the zinc finger domain includes a sequence having at least 94% sequence identity to SEQ ID NO:7. In embodiments, the zinc finger domain includes a sequence having at least 95% sequence identity to SEQ ID NO:7. In embodiments, the zinc finger domain includes a sequence having at least 96% sequence identity to SEQ ID NO:7. In embodiments, the zinc finger domain includes a sequence having at least 97% sequence identity to SEQ ID NO:7. In embodiments, the zinc finger domain includes a sequence having at least 98% sequence identity to SEQ ID NO:7. In embodiments, the zinc finger domain includes a sequence having at least 99% sequence identity to SEQ ID NO:7. In embodiments, the zinc finger domain includes the sequence of SEQ ID NO:7. In embodiments, the zinc finger domain is the sequence of SEQ ID NO:7.

In embodiments, the zinc finger domain includes a sequence having about 75% sequence identity to SEQ ID NO:7. In embodiments, the zinc finger domain includes a sequence having about 80% sequence identity to SEQ ID NO:7. In embodiments, the zinc finger domain includes a sequence having about 85% sequence identity to SEQ ID NO:7. In embodiments, the zinc finger domain includes a sequence having about 90% sequence identity to SEQ ID NO:7. In embodiments, the zinc finger domain includes a sequence having about 91% sequence identity to SEQ ID NO:7. In embodiments, the zinc finger domain includes a sequence having about 92% sequence identity to SEQ ID NO:7. In embodiments, the zinc finger domain includes a sequence having about 93% sequence identity to SEQ ID NO:7. In embodiments, the zinc finger domain includes a sequence having about 94% sequence identity to SEQ ID NO:7. In embodiments, the zinc finger domain includes a sequence having about 95% sequence identity to SEQ ID NO:7. In embodiments, the zinc finger domain includes a sequence having about 96% sequence identity to SEQ ID NO:7. In embodiments, the zinc finger domain includes a sequence having about 97% sequence identity to SEQ ID NO:7. In embodiments, the zinc finger domain includes a sequence having about 98% sequence identity to SEQ ID NO:7. In embodiments, the zinc finger domain includes a sequence having about 99% sequence identity to SEQ ID NO:7.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:7 connected to a second sequence fragment having at least the percentage sequence identity as disclosed above to SEQ ID NO:7 through a sequence fragment having no sequence identity to SEQ ID NO:7. In embodiments, the non-contiguous sequence is a sequence including a plurality of sequence fragments having at least the percentage sequence identity as disclosed above to SEQ ID NO:7 connected through a plurality of sequence fragments having no sequence identity to SEQ ID NO:7.

In embodiments, the zinc finger domain has the percentage sequence identity as disclosed in the above paragraphs to at least 170 continuous amino acids of SEQ ID NO:7. In embodiments, the zinc finger domain has the percentage sequence identity as disclosed in the above paragraphs to at least 160 continuous amino acids of SEQ ID NO:7. In embodiments, the zinc finger domain has the percentage sequence identity as disclosed in the above paragraphs to at least 150 continuous amino acids of SEQ ID NO:7. In embodiments, the zinc finger domain has the percentage sequence identity as disclosed in the above paragraphs to at least 140 continuous amino acids of SEQ ID NO:7. In embodiments, the zinc finger domain has the percentage sequence identity as disclosed in the above paragraphs to at least 130 continuous amino acids of SEQ ID NO:7. In embodiments, the zinc finger domain has the percentage sequence identity as disclosed in the above paragraphs to at least 120 continuous amino acids of SEQ ID NO:7. In embodiments, the zinc finger domain has the percentage sequence identity as disclosed in the above paragraphs to at least 110 continuous amino acids of SEQ ID NO:7. In embodiments, the zinc finger domain has the percentage sequence identity as disclosed in the above paragraphs to at least 100 continuous amino acids of SEQ ID NO:7. In embodiments, the zinc finger domain has the percentage sequence identity as disclosed in the above paragraphs to at least 90 continuous amino acids of SEQ ID NO:7. In embodiments, the zinc finger domain has the percentage sequence identity as disclosed in the above paragraphs to at least 80 continuous amino acids of SEQ ID NO:7. In embodiments, the zinc finger domain has the percentage sequence identity as disclosed in the above paragraphs to at least 70 continuous amino acids of SEQ ID NO:7. In embodiments, the zinc finger domain has the percentage sequence identity as disclosed in the above paragraphs to at least 60 continuous amino acids of SEQ ID NO:7. In embodiments, the zinc finger domain has the percentage sequence identity as disclosed in the above paragraphs to at least 50 continuous amino acids of SEQ ID NO:7. In embodiments, the zinc finger domain has the percentage sequence identity as disclosed in the above paragraphs to at least 40 continuous amino acids of SEQ ID NO:7. In embodiments, the zinc finger domain has the percentage sequence identity as disclosed in the above paragraphs to at least 30 continuous amino acids of SEQ ID NO:7. In embodiments, the zinc finger domain has the percentage sequence identity as disclosed in the above paragraphs to at least 20 continuous amino acids of SEQ ID NO:7.

The sequence of SEQ ID NO:7 encodes a non-naturally occurring peptide sequence, which may be referred to herein as “HTLV-ZFP-8” or “ZFP-8”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a KRAB domain and meCP2 or a fragment thereof.

In embodiments, the protein further includes a nuclear localization signal, a Tat domain, a Myc tag, or combinations thereof. In embodiments, the protein further includes a nuclear localization signal. In embodiments, the protein further includes a a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 16. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:16. In embodiments, the protein includes a sequence having at least 75% sequence identity to SEQ ID NO:16. In embodiments, the protein includes a sequence having at least 80% sequence identity to SEQ ID NO:16. In embodiments, the protein includes a sequence having at least 85% sequence identity to SEQ ID NO:16. In embodiments, the protein includes a sequence having at least 90% sequence identity to SEQ ID NO:16. In embodiments, the protein includes a sequence having at least 91% sequence identity to SEQ ID NO:16. In embodiments, the protein includes a sequence having at least 92% sequence identity to SEQ ID NO:16. In embodiments, the protein includes a sequence having at least 93% sequence identity to SEQ ID NO:16. In embodiments, the protein includes a sequence having at least 94% sequence identity to SEQ ID NO:16. In embodiments, the protein includes a sequence having at least 95% sequence identity to SEQ ID NO:16. In embodiments, the protein includes a sequence having at least 96% sequence identity to SEQ ID NO: 16. In embodiments, the protein includes a sequence having at least 97% sequence identity to SEQ ID NO:16. In embodiments, the protein includes a sequence having at least 98% sequence identity to SEQ ID NO:16. In embodiments, the protein includes a sequence having at least 99% sequence identity to SEQ ID NO:16. In embodiments, the protein includes the sequence of SEQ ID NO:16. In embodiments, the protein is the sequence of SEQ ID NO:16.

In embodiments, the protein includes a sequence having about 75% sequence identity to SEQ ID NO:16. In embodiments, the protein includes a sequence having about 80% sequence identity to SEQ ID NO:16. In embodiments, the protein includes a sequence having about 85% sequence identity to SEQ ID NO:16. In embodiments, the protein includes a sequence having about 90% sequence identity to SEQ ID NO:16. In embodiments, the protein includes a sequence having about 91% sequence identity to SEQ ID NO:16. In embodiments, the protein includes a sequence having about 92% sequence identity to SEQ ID NO:16. In embodiments, the protein includes a sequence having about 93% sequence identity to SEQ ID NO:16. In embodiments, the protein includes a sequence having about 94% sequence identity to SEQ ID NO:16. In embodiments, the protein includes a sequence having about 95% sequence identity to SEQ ID NO:16. In embodiments, the protein includes a sequence having about 96% sequence identity to SEQ ID NO:16. In embodiments, the protein includes a sequence having about 97% sequence identity to SEQ ID NO:16. In embodiments, the protein includes a sequence having about 98% sequence identity to SEQ ID NO:16. In embodiments, the protein includes a sequence having about 99% sequence identity to SEQ ID NO:16.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:16 connected to a second sequence fragment having at least the percentage sequence identity as disclosed above to SEQ ID NO:16 through a sequence fragment having no sequence identity to SEQ ID NO:16. In embodiments, the non-contiguous sequence is a sequence including a plurality of sequence fragments having at least the percentage sequence identity as disclosed above to SEQ ID NO:16 connected through a plurality of sequence fragments having no sequence identity to SEQ ID NO:16.

In embodiments, the protein has the percentage sequence identity as disclosed in the above paragraphs to at least 300 continuous amino acids of SEQ ID NO:16. In embodiments, the protein has the percentage sequence identity as disclosed in the above paragraphs to at least 290 continuous amino acids of SEQ ID NO:16. In embodiments, the protein has the percentage sequence identity as disclosed in the above paragraphs to at least 280 continuous amino acids of SEQ ID NO:16. In embodiments, the protein has the percentage sequence identity as disclosed in the above paragraphs to at least 270 continuous amino acids of SEQ ID NO:16. In embodiments, the protein has the percentage sequence identity as disclosed in the above paragraphs to at least 260 continuous amino acids of SEQ ID NO:16. In embodiments, the protein has the percentage sequence identity as disclosed in the above paragraphs to at least 250 continuous amino acids of SEQ ID NO:16. In embodiments, the protein has the percentage sequence identity as disclosed in the above paragraphs to at least 240 continuous amino acids of SEQ ID NO:16. In embodiments, the protein has the percentage sequence identity as disclosed in the above paragraphs to at least 230 continuous amino acids of SEQ ID NO:16. In embodiments, the protein has the percentage sequence identity as disclosed in the above paragraphs to at least 220 continuous amino acids of SEQ ID NO:16. In embodiments, the protein has the percentage sequence identity as disclosed in the above paragraphs to at least 210 continuous amino acids of SEQ ID NO:16. In embodiments, the protein has the percentage sequence identity as disclosed in the above paragraphs to at least 200 continuous amino acids of SEQ ID NO:16. In embodiments, the protein has the percentage sequence identity as disclosed in the above paragraphs to at least 190 continuous amino acids of SEQ ID NO:16. In embodiments, the protein has the percentage sequence identity as disclosed in the above paragraphs to at least 180 continuous amino acids of SEQ ID NO:16. In embodiments, the protein has the percentage sequence identity as disclosed in the above paragraphs to at least 170 continuous amino acids of SEQ ID NO:16. In embodiments, the protein has the percentage sequence identity as disclosed in the above paragraphs to at least 160 continuous amino acids of SEQ ID NO:16. In embodiments, the protein has the percentage sequence identity as disclosed in the above paragraphs to at least 150 continuous amino acids of SEQ ID NO:16. In embodiments, the protein has the percentage sequence identity as disclosed in the above paragraphs to at least 140 continuous amino acids of SEQ ID NO:16. In embodiments, the protein has the percentage sequence identity as disclosed in the above paragraphs to at least 130 continuous amino acids of SEQ ID NO:16. In embodiments, the protein has the percentage sequence identity as disclosed in the above paragraphs to at least 120 continuous amino acids of SEQ ID NO:16. In embodiments, the protein has the percentage sequence identity as disclosed in the above paragraphs to at least 110 continuous amino acids of SEQ ID NO:16. In embodiments, the protein has the percentage sequence identity as disclosed in the above paragraphs to at least 100 continuous amino acids of SEQ ID NO:16. In embodiments, the protein has the percentage sequence identity as disclosed in the above paragraphs to at least 90 continuous amino acids of SEQ ID NO:16. In embodiments, the protein has the percentage sequence identity as disclosed in the above paragraphs to at least 80 continuous amino acids of SEQ ID NO:16. In embodiments, the protein has the percentage sequence identity as disclosed in the above paragraphs to at least 70 continuous amino acids of SEQ ID NO:16. In embodiments, the protein has the percentage sequence identity as disclosed in the above paragraphs to at least 60 continuous amino acids of SEQ ID NO:16. In embodiments, the protein has the percentage sequence identity as disclosed in the above paragraphs to at least 50 continuous amino acids of SEQ ID NO:16. In embodiments, the protein has the percentage sequence identity as disclosed in the above paragraphs to at least 40 continuous amino acids of SEQ ID NO:16. In embodiments, the protein has the percentage sequence identity as disclosed in the above paragraphs to at least 30 continuous amino acids of SEQ ID NO:16. In embodiments, the protein has the percentage sequence identity as disclosed in the above paragraphs to at least 20 continuous amino acids of SEQ ID NO:16.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:24. In embodiments, the sequence has at least 75%, 80%, 85%, 90%, 95%, 96%, 97%, 98% or 99% sequence identity to the sequence of SEQ ID NO:24. In embodiments, the sequence has at least 90%, 95%, 96%, 97%, 98%, 99% or 100% nucleic acid sequence identity across the whole sequence or a portion of the sequence (e.g. a 5, 10, 11, 12, 13, 14, or 15 continuous nucleic acid portion) of SEQ ID NO:24.

In embodiments, the sequence within the HTLV-1 LTR has at least 75% sequence identity to the sequence of SEQ ID NO:24. In embodiments, the sequence within the HTLV-1 LTR has at least 80% sequence identity to the sequence of SEQ ID NO:24. In embodiments, the sequence within the HTLV-1 LTR has at least 85% sequence identity to the sequence of SEQ ID NO:24. In embodiments, the sequence within the HTLV-1 LTR has at least 90% sequence identity to the sequence of SEQ ID NO:24. In embodiments, the sequence within the HTLV-1 LTR has at least 95% sequence identity to the sequence of SEQ ID NO:24. In embodiments, the sequence within the HTLV-1 LTR has at least 98% sequence identity to the sequence of SEQ ID NO:24. In embodiments, the sequence within the HTLV-1 LTR has at least 99% sequence identity to the sequence of SEQ ID NO:24. In embodiments, the sequence within the HTLV-1 LTR includes the sequence of SEQ ID NO:24. In embodiments, the sequence within the HTLV-1 LTR is the sequence of SEQ ID NO:24.

In embodiments, the sequence within the HTLV-1 LTR has about 75% sequence identity to the sequence of SEQ ID NO:24. In embodiments, the sequence within the HTLV-1 LTR has about 80% sequence identity to the sequence of SEQ ID NO:24. In embodiments, the sequence within the HTLV-1 LTR has about 85% sequence identity to the sequence of SEQ ID NO:24. In embodiments, the sequence within the HTLV-1 LTR has about 90% sequence identity to the sequence of SEQ ID NO:24. In embodiments, the sequence within the HTLV-1 LTR has about 95% sequence identity to the sequence of SEQ ID NO:24. In embodiments, the sequence within the HTLV-1 LTR has about 98% sequence identity to the sequence of SEQ ID NO:24. In embodiments, the sequence within the HTLV-1 LTR has about 99% sequence identity to the sequence of SEQ ID NO:24.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:33, F2 includes SEQ ID NO:34, F3 includes SEQ ID NO:35, F4 includes SEQ ID NO:36, F5 includes SEQ ID NO:37 and F6 includes SEQ ID NO:38. In embodiments, the F1 is SEQ ID NO:33, F2 is SEQ ID NO:34, F3 is SEQ ID NO:35, F4 is SEQ ID NO:36, F5 is SEQ ID NO:37 and F6 is SEQ ID NO:38.

In embodiments, the zinc finger domain has at least 75%, 80%, 85%, 90%, 95%, 96%, 97%, 98% or 99% sequence identity to the sequence of SEQ ID NO:1. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:1. In embodiments, the zinc finger domain includes a sequence having at least 75% sequence identity to SEQ ID NO:1. In embodiments, the zinc finger domain includes a sequence having at least 80% sequence identity to SEQ ID NO:1. In embodiments, the zinc finger domain includes a sequence having at least 85% sequence identity to SEQ ID NO:1. In embodiments, the zinc finger domain includes a sequence having at least 90% sequence identity to SEQ ID NO:1. In embodiments, the zinc finger domain includes a sequence having at least 91% sequence identity to SEQ ID NO:1. In embodiments, the zinc finger domain includes a sequence having at least 92% sequence identity to SEQ ID NO:1. In embodiments, the zinc finger domain includes a sequence having at least 93% sequence identity to SEQ ID NO:1. In embodiments, the zinc finger domain includes a sequence having at least 94% sequence identity to SEQ ID NO:1. In embodiments, the zinc finger domain includes a sequence having at least 95% sequence identity to SEQ ID NO:1. In embodiments, the zinc finger domain includes a sequence having at least 96% sequence identity to SEQ ID NO:1. In embodiments, the zinc finger domain includes a sequence having at least 97% sequence identity to SEQ ID NO:1. In embodiments, the zinc finger domain includes a sequence having at least 98% sequence identity to SEQ ID NO:1. In embodiments, the zinc finger domain includes a sequence having at least 99% sequence identity to SEQ ID NO:1. In embodiments, the zinc finger domain includes the sequence of SEQ ID NO: 1. In embodiments, the zinc finger domain is the sequence of SEQ ID NO:1.

In embodiments, the zinc finger domain includes a sequence having about 75% sequence identity to SEQ ID NO:1. In embodiments, the zinc finger domain includes a sequence having about 80% sequence identity to SEQ ID NO:1. In embodiments, the zinc finger domain includes a sequence having about 85% sequence identity to SEQ ID NO:1. In embodiments, the zinc finger domain includes a sequence having about 90% sequence identity to SEQ ID NO:1. In embodiments, the zinc finger domain includes a sequence having about 91% sequence identity to SEQ ID NO:1. In embodiments, the zinc finger domain includes a sequence having about 92% sequence identity to SEQ ID NO:1. In embodiments, the zinc finger domain includes a sequence having about 93% sequence identity to SEQ ID NO:1. In embodiments, the zinc finger domain includes a sequence having about 94% sequence identity to SEQ ID NO:1. In embodiments, the zinc finger domain includes a sequence having about 95% sequence identity to SEQ ID NO:1. In embodiments, the zinc finger domain includes a sequence having about 96% sequence identity to SEQ ID NO:1. In embodiments, the zinc finger domain includes a sequence having about 97% sequence identity to SEQ ID NO:1. In embodiments, the zinc finger domain includes a sequence having about 98% sequence identity to SEQ ID NO:1. In embodiments, the zinc finger domain includes a sequence having about 99% sequence identity to SEQ ID NO:1.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:1 connected to a second sequence fragment having at least the percentage sequence identity as disclosed above to SEQ ID NO:1 through a sequence fragment having no sequence identity to SEQ ID NO:1. In embodiments, the non-contiguous sequence is a sequence including a plurality of sequence fragments having at least the percentage sequence identity as disclosed above to SEQ ID NO:1 connected through a plurality of sequence fragments having no sequence identity to SEQ ID NO:1.

In embodiments, the zinc finger domain has the percentage sequence identity as disclosed in the above paragraphs to at least 170 continuous amino acids of SEQ ID NO:1. In embodiments, the zinc finger domain has the percentage sequence identity as disclosed in the above paragraphs to at least 160 continuous amino acids of SEQ ID NO:1. In embodiments, the zinc finger domain has the percentage sequence identity as disclosed in the above paragraphs to at least 150 continuous amino acids of SEQ ID NO:1. In embodiments, the zinc finger domain has the percentage sequence identity as disclosed in the above paragraphs to at least 140 continuous amino acids of SEQ ID NO:1. In embodiments, the zinc finger domain has the percentage sequence identity as disclosed in the above paragraphs to at least 130 continuous amino acids of SEQ ID NO:1. In embodiments, the zinc finger domain has the percentage sequence identity as disclosed in the above paragraphs to at least 120 continuous amino acids of SEQ ID NO:1. In embodiments, the zinc finger domain has the percentage sequence identity as disclosed in the above paragraphs to at least 110 continuous amino acids of SEQ ID NO:1. In embodiments, the zinc finger domain has the percentage sequence identity as disclosed in the above paragraphs to at least 100 continuous amino acids of SEQ ID NO:1. In embodiments, the zinc finger domain has the percentage sequence identity as disclosed in the above paragraphs to at least 90 continuous amino acids of SEQ ID NO:1. In embodiments, the zinc finger domain has the percentage sequence identity as disclosed in the above paragraphs to at least 80 continuous amino acids of SEQ ID NO:1. In embodiments, the zinc finger domain has the percentage sequence identity as disclosed in the above paragraphs to at least 70 continuous amino acids of SEQ ID NO:1. In embodiments, the zinc finger domain has the percentage sequence identity as disclosed in the above paragraphs to at least 60 continuous amino acids of SEQ ID NO:1. In embodiments, the zinc finger domain has the percentage sequence identity as disclosed in the above paragraphs to at least 50 continuous amino acids of SEQ ID NO:1. In embodiments, the zinc finger domain has the percentage sequence identity as disclosed in the above paragraphs to at least 40 continuous amino acids of SEQ ID NO:1.

The sequence of SEQ ID NO:1 encodes a non-naturally occurring peptide sequence, which may be referred to herein as “HTLV-ZFP-1” or “ZFP-1”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a KRAB domain and meCP2 or a fragment thereof.

In embodiments, the protein further includes a nuclear localization signal, a Tat domain, a Myc tag, or combinations thereof. In embodiments, the protein further includes a nuclear localization signal. In embodiments, the protein further includes a a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 10. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:10. In embodiments, the protein includes a sequence having at least 75% sequence identity to SEQ ID NO:10. In embodiments, the protein includes a sequence having at least 80% sequence identity to SEQ ID NO:10. In embodiments, the protein includes a sequence having at least 85% sequence identity to SEQ ID NO:10. In embodiments, the protein includes a sequence having at least 90% sequence identity to SEQ ID NO:10. In embodiments, the protein includes a sequence having at least 91% sequence identity to SEQ ID NO:10. In embodiments, the protein includes a sequence having at least 92% sequence identity to SEQ ID NO:10. In embodiments, the protein includes a sequence having at least 93% sequence identity to SEQ ID NO:10. In embodiments, the protein includes a sequence having at least 94% sequence identity to SEQ ID NO:10. In embodiments, the protein includes a sequence having at least 95% sequence identity to SEQ ID NO:10. In embodiments, the protein includes a sequence having at least 96% sequence identity to SEQ ID NO: 10. In embodiments, the protein includes a sequence having at least 97% sequence identity to SEQ ID NO:10. In embodiments, the protein includes a sequence having at least 98% sequence identity to SEQ ID NO:10. In embodiments, the protein includes a sequence having at least 99% sequence identity to SEQ ID NO:10. In embodiments, the protein includes the sequence of SEQ ID NO:10. In embodiments, the protein is the sequence of SEQ ID NO:10.

In embodiments, the protein includes a sequence having about 75% sequence identity to SEQ ID NO:10. In embodiments, the protein includes a sequence having about 80% sequence identity to SEQ ID NO:10. In embodiments, the protein includes a sequence having about 85% sequence identity to SEQ ID NO:10. In embodiments, the protein includes a sequence having about 90% sequence identity to SEQ ID NO:10. In embodiments, the protein includes a sequence having about 91% sequence identity to SEQ ID NO:10. In embodiments, the protein includes a sequence having about 92% sequence identity to SEQ ID NO:10. In embodiments, the protein includes a sequence having about 93% sequence identity to SEQ ID NO:10. In embodiments, the protein includes a sequence having about 94% sequence identity to SEQ ID NO:10. In embodiments, the protein includes a sequence having about 95% sequence identity to SEQ ID NO:10. In embodiments, the protein includes a sequence having about 96% sequence identity to SEQ ID NO:10. In embodiments, the protein includes a sequence having about 97% sequence identity to SEQ ID NO:10. In embodiments, the protein includes a sequence having about 98% sequence identity to SEQ ID NO:10. In embodiments, the protein includes a sequence having about 99% sequence identity to SEQ ID NO:10.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:10 connected to a second sequence fragment having at least the percentage sequence identity as disclosed above to SEQ ID NO:10 through a sequence fragment having no sequence identity to SEQ ID NO:10. In embodiments, the non-contiguous sequence is a sequence including a plurality of sequence fragments having at least the percentage sequence identity as disclosed above to SEQ ID NO:10 connected through a plurality of sequence fragments having no sequence identity to SEQ ID NO:10.

In embodiments, the protein has the percentage sequence identity as disclosed in the above paragraphs to at least 300 continuous amino acids of SEQ ID NO:10. In embodiments, the protein has the percentage sequence identity as disclosed in the above paragraphs to at least 290 continuous amino acids of SEQ ID NO:10. In embodiments, the protein has the percentage sequence identity as disclosed in the above paragraphs to at least 280 continuous amino acids of SEQ ID NO:10. In embodiments, the protein has the percentage sequence identity as disclosed in the above paragraphs to at least 270 continuous amino acids of SEQ ID NO:10. In embodiments, the protein has the percentage sequence identity as disclosed in the above paragraphs to at least 260 continuous amino acids of SEQ ID NO:10. In embodiments, the protein has the percentage sequence identity as disclosed in the above paragraphs to at least 250 continuous amino acids of SEQ ID NO:10. In embodiments, the protein has the percentage sequence identity as disclosed in the above paragraphs to at least 240 continuous amino acids of SEQ ID NO:10. In embodiments, the protein has the percentage sequence identity as disclosed in the above paragraphs to at least 230 continuous amino acids of SEQ ID NO:10. In embodiments, the protein has the percentage sequence identity as disclosed in the above paragraphs to at least 220 continuous amino acids of SEQ ID NO:10. In embodiments, the protein has the percentage sequence identity as disclosed in the above paragraphs to at least 210 continuous amino acids of SEQ ID NO:10. In embodiments, the protein has the percentage sequence identity as disclosed in the above paragraphs to at least 200 continuous amino acids of SEQ ID NO:10. In embodiments, the protein has the percentage sequence identity as disclosed in the above paragraphs to at least 190 continuous amino acids of SEQ ID NO:10. In embodiments, the protein has the percentage sequence identity as disclosed in the above paragraphs to at least 180 continuous amino acids of SEQ ID NO:10. In embodiments, the protein has the percentage sequence identity as disclosed in the above paragraphs to at least 170 continuous amino acids of SEQ ID NO:10. In embodiments, the protein has the percentage sequence identity as disclosed in the above paragraphs to at least 160 continuous amino acids of SEQ ID NO:10. In embodiments, the protein has the percentage sequence identity as disclosed in the above paragraphs to at least 150 continuous amino acids of SEQ ID NO:10. In embodiments, the protein has the percentage sequence identity as disclosed in the above paragraphs to at least 140 continuous amino acids of SEQ ID NO:10. In embodiments, the protein has the percentage sequence identity as disclosed in the above paragraphs to at least 130 continuous amino acids of SEQ ID NO:10. In embodiments, the protein has the percentage sequence identity as disclosed in the above paragraphs to at least 120 continuous amino acids of SEQ ID NO:10. In embodiments, the protein has the percentage sequence identity as disclosed in the above paragraphs to at least 110 continuous amino acids of SEQ ID NO:10. In embodiments, the protein has the percentage sequence identity as disclosed in the above paragraphs to at least 100 continuous amino acids of SEQ ID NO:10. In embodiments, the protein has the percentage sequence identity as disclosed in the above paragraphs to at least 90 continuous amino acids of SEQ ID NO:10. In embodiments, the protein has the percentage sequence identity as disclosed in the above paragraphs to at least 80 continuous amino acids of SEQ ID NO:10. In embodiments, the protein has the percentage sequence identity as disclosed in the above paragraphs to at least 70 continuous amino acids of SEQ ID NO:10. In embodiments, the protein has the percentage sequence identity as disclosed in the above paragraphs to at least 60 continuous amino acids of SEQ ID NO:10. In embodiments, the protein has the percentage sequence identity as disclosed in the above paragraphs to at least 50 continuous amino acids of SEQ ID NO:10. In embodiments, the protein has the percentage sequence identity as disclosed in the above paragraphs to at least 40 continuous amino acids of SEQ ID NO:10.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:26. In embodiments, the sequence has at least 75%, 80%, 85%, 90%, 95%, 96%, 97%, 98% or 99% sequence identity to the sequence of SEQ ID NO:26. In embodiments, the sequence has at least 90%, 95%, 96%, 97%, 98%, 99% or 100% nucleic acid sequence identity across the whole sequence or a portion of the sequence (e.g. a 5, 10, 11, 12, 13, 14, or 15 continuous nucleic acid portion) of SEQ ID NO:26.

In embodiments, the sequence within the HTLV-1 LTR has at least 75% sequence identity to the sequence of SEQ ID NO:26. In embodiments, the sequence within the HTLV-1 LTR has at least 80% sequence identity to the sequence of SEQ ID NO:26. In embodiments, the sequence within the HTLV-1 LTR has at least 85% sequence identity to the sequence of SEQ ID NO:26. In embodiments, the sequence within the HTLV-1 LTR has at least 90% sequence identity to the sequence of SEQ ID NO:26. In embodiments, the sequence within the HTLV-1 LTR has at least 95% sequence identity to the sequence of SEQ ID NO:26. In embodiments, the sequence within the HTLV-1 LTR has at least 98% sequence identity to the sequence of SEQ ID NO:26. In embodiments, the sequence within the HTLV-1 LTR has at least 99% sequence identity to the sequence of SEQ ID NO:26. In embodiments, the sequence within the HTLV-1 LTR includes the sequence of SEQ ID NO:26. In embodiments, the sequence within the HTLV-1 LTR is the sequence of SEQ ID NO:26.

In embodiments, the sequence within the HTLV-1 LTR has about 75% sequence identity to the sequence of SEQ ID NO:26. In embodiments, the sequence within the HTLV-1 LTR has about 80% sequence identity to the sequence of SEQ ID NO:26. In embodiments, the sequence within the HTLV-1 LTR has about 85% sequence identity to the sequence of SEQ ID NO:26. In embodiments, the sequence within the HTLV-1 LTR has about 90% sequence identity to the sequence of SEQ ID NO:26. In embodiments, the sequence within the HTLV-1 LTR has about 95% sequence identity to the sequence of SEQ ID NO:26. In embodiments, the sequence within the HTLV-1 LTR has about 98% sequence identity to the sequence of SEQ ID NO:26. In embodiments, the sequence within the HTLV-1 LTR has about 99% sequence identity to the sequence of SEQ ID NO:26.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:45, F2 includes SEQ ID NO:46, F3 includes SEQ ID NO:47, F4 includes SEQ ID NO:48, F5 includes SEQ ID NO:49 and F6 includes SEQ ID NO:50. In embodiments, the F1 is SEQ ID NO:45, F2 is SEQ ID NO:46, F3 is SEQ ID NO:47, F4 is SEQ ID NO:48, F5 is SEQ ID NO:49 and F6 is SEQ ID NO:50.

In embodiments, the zinc finger domain has at least 75%, 80%, 85%, 90%, 95%, 96%, 97%, 98% or 99% sequence identity to the sequence of SEQ ID NO:3. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:3. In embodiments, the zinc finger domain includes a sequence having at least 75% sequence identity to SEQ ID NO:3. In embodiments, the zinc finger domain includes a sequence having at least 80% sequence identity to SEQ ID NO:3. In embodiments, the zinc finger domain includes a sequence having at least 85% sequence identity to SEQ ID NO:3. In embodiments, the zinc finger domain includes a sequence having at least 90% sequence identity to SEQ ID NO:3. In embodiments, the zinc finger domain includes a sequence having at least 91% sequence identity to SEQ ID NO:3. In embodiments, the zinc finger domain includes a sequence having at least 92% sequence identity to SEQ ID NO:3. In embodiments, the zinc finger domain includes a sequence having at least 93% sequence identity to SEQ ID NO:3. In embodiments, the zinc finger domain includes a sequence having at least 94% sequence identity to SEQ ID NO:3. In embodiments, the zinc finger domain includes a sequence having at least 95% sequence identity to SEQ ID NO:3. In embodiments, the zinc finger domain includes a sequence having at least 96% sequence identity to SEQ ID NO:3. In embodiments, the zinc finger domain includes a sequence having at least 97% sequence identity to SEQ ID NO:3. In embodiments, the zinc finger domain includes a sequence having at least 98% sequence identity to SEQ ID NO:3. In embodiments, the zinc finger domain includes a sequence having at least 99% sequence identity to SEQ ID NO:3. In embodiments, the zinc finger domain includes the sequence of SEQ ID NO:3. In embodiments, the zinc finger domain is the sequence of SEQ ID NO:3.

In embodiments, the zinc finger domain includes a sequence having about 75% sequence identity to SEQ ID NO:3. In embodiments, the zinc finger domain includes a sequence having about 80% sequence identity to SEQ ID NO:3. In embodiments, the zinc finger domain includes a sequence having about 85% sequence identity to SEQ ID NO:3. In embodiments, the zinc finger domain includes a sequence having about 90% sequence identity to SEQ ID NO:3. In embodiments, the zinc finger domain includes a sequence having about 91% sequence identity to SEQ ID NO:3. In embodiments, the zinc finger domain includes a sequence having about 92% sequence identity to SEQ ID NO:3. In embodiments, the zinc finger domain includes a sequence having about 93% sequence identity to SEQ ID NO:3. In embodiments, the zinc finger domain includes a sequence having about 94% sequence identity to SEQ ID NO:3. In embodiments, the zinc finger domain includes a sequence having about 95% sequence identity to SEQ ID NO:3. In embodiments, the zinc finger domain includes a sequence having about 96% sequence identity to SEQ ID NO:3. In embodiments, the zinc finger domain includes a sequence having about 97% sequence identity to SEQ ID NO:3. In embodiments, the zinc finger domain includes a sequence having about 98% sequence identity to SEQ ID NO:3. In embodiments, the zinc finger domain includes a sequence having about 99% sequence identity to SEQ ID NO:3.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:3 connected to a second sequence fragment having at least the percentage sequence identity as disclosed above to SEQ ID NO:3 through a sequence fragment having no sequence identity to SEQ ID NO:3. In embodiments, the non-contiguous sequence is a sequence including a plurality of sequence fragments having at least the percentage sequence identity as disclosed above to SEQ ID NO:3 connected through a plurality of sequence fragments having no sequence identity to SEQ ID NO:3.

In embodiments, the zinc finger domain has the percentage sequence identity as disclosed in the above paragraphs to at least 170 continuous amino acids of SEQ ID NO:3. In embodiments, the zinc finger domain has the percentage sequence identity as disclosed in the above paragraphs to at least 160 continuous amino acids of SEQ ID NO:3. In embodiments, the zinc finger domain has the percentage sequence identity as disclosed in the above paragraphs to at least 150 continuous amino acids of SEQ ID NO:3. In embodiments, the zinc finger domain has the percentage sequence identity as disclosed in the above paragraphs to at least 140 continuous amino acids of SEQ ID NO:3. In embodiments, the zinc finger domain has the percentage sequence identity as disclosed in the above paragraphs to at least 130 continuous amino acids of SEQ ID NO:3. In embodiments, the zinc finger domain has the percentage sequence identity as disclosed in the above paragraphs to at least 120 continuous amino acids of SEQ ID NO:3. In embodiments, the zinc finger domain has the percentage sequence identity as disclosed in the above paragraphs to at least 110 continuous amino acids of SEQ ID NO:3. In embodiments, the zinc finger domain has the percentage sequence identity as disclosed in the above paragraphs to at least 100 continuous amino acids of SEQ ID NO:3. In embodiments, the zinc finger domain has the percentage sequence identity as disclosed in the above paragraphs to at least 90 continuous amino acids of SEQ ID NO:3. In embodiments, the zinc finger domain has the percentage sequence identity as disclosed in the above paragraphs to at least 80 continuous amino acids of SEQ ID NO:3. In embodiments, the zinc finger domain has the percentage sequence identity as disclosed in the above paragraphs to at least 70 continuous amino acids of SEQ ID NO:3. In embodiments, the zinc finger domain has the percentage sequence identity as disclosed in the above paragraphs to at least 60 continuous amino acids of SEQ ID NO:3. In embodiments, the zinc finger domain has the percentage sequence identity as disclosed in the above paragraphs to at least 50 continuous amino acids of SEQ ID NO:3. In embodiments, the zinc finger domain has the percentage sequence identity as disclosed in the above paragraphs to at least 40 continuous amino acids of SEQ ID NO:3. In embodiments, the zinc finger domain has the percentage sequence identity as disclosed in the above paragraphs to at least 30 continuous amino acids of SEQ ID NO:3. In embodiments, the zinc finger domain has the percentage sequence identity as disclosed in the above paragraphs to at least 20 continuous amino acids of SEQ ID NO:3.

The sequence of SEQ ID NO:3 encodes a non-naturally occurring peptide sequence, which may be referred to herein as “HTLV-ZFP-4” or “ZFP-4”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a KRAB domain and meCP2 or a fragment thereof.

In embodiments, the protein further includes a nuclear localization signal, a Tat domain, a Myc tag, or combinations thereof. In embodiments, the protein further includes a nuclear localization signal. In embodiments, the protein further includes a a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 12. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:12. In embodiments, the protein includes a sequence having at least 75% sequence identity to SEQ ID NO:12. In embodiments, the protein includes a sequence having at least 80% sequence identity to SEQ ID NO:12. In embodiments, the protein includes a sequence having at least 85% sequence identity to SEQ ID NO:12. In embodiments, the protein includes a sequence having at least 90% sequence identity to SEQ ID NO:12. In embodiments, the protein includes a sequence having at least 91% sequence identity to SEQ ID NO:12. In embodiments, the protein includes a sequence having at least 92% sequence identity to SEQ ID NO:12. In embodiments, the protein includes a sequence having at least 93% sequence identity to SEQ ID NO:12. In embodiments, the protein includes a sequence having at least 94% sequence identity to SEQ ID NO:12. In embodiments, the protein includes a sequence having at least 95% sequence identity to SEQ ID NO:12. In embodiments, the protein includes a sequence having at least 96% sequence identity to SEQ ID NO: 12. In embodiments, the protein includes a sequence having at least 97% sequence identity to SEQ ID NO:12. In embodiments, the protein includes a sequence having at least 98% sequence identity to SEQ ID NO:12. In embodiments, the protein includes a sequence having at least 99% sequence identity to SEQ ID NO:12. In embodiments, the protein includes the sequence of SEQ ID NO:12. In embodiments, the protein is the sequence of SEQ ID NO:12.

In embodiments, the protein includes a sequence having about 75% sequence identity to SEQ ID NO:12. In embodiments, the protein includes a sequence having about 80% sequence identity to SEQ ID NO:12. In embodiments, the protein includes a sequence having about 85% sequence identity to SEQ ID NO:12. In embodiments, the protein includes a sequence having about 90% sequence identity to SEQ ID NO:12. In embodiments, the protein includes a sequence having about 91% sequence identity to SEQ ID NO:12. In embodiments, the protein includes a sequence having about 92% sequence identity to SEQ ID NO:12. In embodiments, the protein includes a sequence having about 93% sequence identity to SEQ ID NO:12. In embodiments, the protein includes a sequence having about 94% sequence identity to SEQ ID NO:12. In embodiments, the protein includes a sequence having about 95% sequence identity to SEQ ID NO:12. In embodiments, the protein includes a sequence having about 96% sequence identity to SEQ ID NO:12. In embodiments, the protein includes a sequence having about 97% sequence identity to SEQ ID NO:12. In embodiments, the protein includes a sequence having about 98% sequence identity to SEQ ID NO:12. In embodiments, the protein includes a sequence having about 99% sequence identity to SEQ ID NO:12.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:12 connected to a second sequence fragment having at least the percentage sequence identity as disclosed above to SEQ ID NO:12 through a sequence fragment having no sequence identity to SEQ ID NO:12. In embodiments, the non-contiguous sequence is a sequence including a plurality of sequence fragments having at least the percentage sequence identity as disclosed above to SEQ ID NO:12 connected through a plurality of sequence fragments having no sequence identity to SEQ ID NO:12

In embodiments, the protein has the percentage sequence identity as disclosed in the above paragraphs to at least 300 continuous amino acids of SEQ ID NO:12. In embodiments, the protein has the percentage sequence identity as disclosed in the above paragraphs to at least 290 continuous amino acids of SEQ ID NO:12. In embodiments, the protein has the percentage sequence identity as disclosed in the above paragraphs to at least 280 continuous amino acids of SEQ ID NO:12. In embodiments, the protein has the percentage sequence identity as disclosed in the above paragraphs to at least 270 continuous amino acids of SEQ ID NO:12. In embodiments, the protein has the percentage sequence identity as disclosed in the above paragraphs to at least 260 continuous amino acids of SEQ ID NO:12. In embodiments, the protein has the percentage sequence identity as disclosed in the above paragraphs to at least 250 continuous amino acids of SEQ ID NO:12. In embodiments, the protein has the percentage sequence identity as disclosed in the above paragraphs to at least 240 continuous amino acids of SEQ ID NO:12. In embodiments, the protein has the percentage sequence identity as disclosed in the above paragraphs to at least 230 continuous amino acids of SEQ ID NO:12. In embodiments, the protein has the percentage sequence identity as disclosed in the above paragraphs to at least 220 continuous amino acids of SEQ ID NO:12. In embodiments, the protein has the percentage sequence identity as disclosed in the above paragraphs to at least 210 continuous amino acids of SEQ ID NO:12. In embodiments, the protein has the percentage sequence identity as disclosed in the above paragraphs to at least 200 continuous amino acids of SEQ ID NO:12. In embodiments, the protein has the percentage sequence identity as disclosed in the above paragraphs to at least 190 continuous amino acids of SEQ ID NO:12. In embodiments, the protein has the percentage sequence identity as disclosed in the above paragraphs to at least 180 continuous amino acids of SEQ ID NO:12. In embodiments, the protein has the percentage sequence identity as disclosed in the above paragraphs to at least 170 continuous amino acids of SEQ ID NO:12. In embodiments, the protein has the percentage sequence identity as disclosed in the above paragraphs to at least 160 continuous amino acids of SEQ ID NO:12. In embodiments, the protein has the percentage sequence identity as disclosed in the above paragraphs to at least 150 continuous amino acids of SEQ ID NO:12. In embodiments, the protein has the percentage sequence identity as disclosed in the above paragraphs to at least 140 continuous amino acids of SEQ ID NO:12. In embodiments, the protein has the percentage sequence identity as disclosed in the above paragraphs to at least 130 continuous amino acids of SEQ ID NO:12. In embodiments, the protein has the percentage sequence identity as disclosed in the above paragraphs to at least 120 continuous amino acids of SEQ ID NO:12. In embodiments, the protein has the percentage sequence identity as disclosed in the above paragraphs to at least 110 continuous amino acids of SEQ ID NO:12. In embodiments, the protein has the percentage sequence identity as disclosed in the above paragraphs to at least 100 continuous amino acids of SEQ ID NO:12. In embodiments, the protein has the percentage sequence identity as disclosed in the above paragraphs to at least 90 continuous amino acids of SEQ ID NO:12. In embodiments, the protein has the percentage sequence identity as disclosed in the above paragraphs to at least 80 continuous amino acids of SEQ ID NO:12. In embodiments, the protein has the percentage sequence identity as disclosed in the above paragraphs to at least 70 continuous amino acids of SEQ ID NO:12. In embodiments, the protein has the percentage sequence identity as disclosed in the above paragraphs to at least 60 continuous amino acids of SEQ ID NO:12. In embodiments, the protein has the percentage sequence identity as disclosed in the above paragraphs to at least 50 continuous amino acids of SEQ ID NO:12. In embodiments, the protein has the percentage sequence identity as disclosed in the above paragraphs to at least 40 continuous amino acids of SEQ ID NO:12. In embodiments, the protein has the percentage sequence identity as disclosed in the above paragraphs to at least 30 continuous amino acids of SEQ ID NO:12. In embodiments, the protein has the percentage sequence identity as disclosed in the above paragraphs to at least 20 continuous amino acids of SEQ ID NO:12.

In an aspect is provided a protein including a zinc finger domain capable of binding a sequence within a long terminal repeat (LTR) of Human T-cell lymphotropic virus type 1 (HTLV-1), wherein the sequence has at least 75% sequence identity to SEQ ID NO:29. In embodiments, the sequence has at least 75%, 80%, 85%, 90%, 95%, 96%, 97%, 98% or 99% sequence identity to the sequence of SEQ ID NO:29. In embodiments, the sequence has at least 90%, 95%, 96%, 97%, 98%, 99% or 100% nucleic acid sequence identity across the whole sequence or a portion of the sequence (e.g. a 5, 10, 11, 12, 13, 14, or 15 continuous nucleic acid portion) of SEQ ID NO:29.

In embodiments, the sequence within the HTLV-1 LTR has at least 75% sequence identity to the sequence of SEQ ID NO:29. In embodiments, the sequence within the HTLV-1 LTR has at least 80% sequence identity to the sequence of SEQ ID NO:29. In embodiments, the sequence within the HTLV-1 LTR has at least 85% sequence identity to the sequence of SEQ ID NO:29. In embodiments, the sequence within the HTLV-1 LTR has at least 90% sequence identity to the sequence of SEQ ID NO:29. In embodiments, the sequence within the HTLV-1 LTR has at least 95% sequence identity to the sequence of SEQ ID NO:29. In embodiments, the sequence within the HTLV-1 LTR has at least 98% sequence identity to the sequence of SEQ ID NO:29. In embodiments, the sequence within the HTLV-1 LTR has at least 99% sequence identity to the sequence of SEQ ID NO:29. In embodiments, the sequence within the HTLV-1 LTR includes the sequence of SEQ ID NO:29. In embodiments, the sequence within the HTLV-1 LTR is the sequence of SEQ ID NO:29.

In embodiments, the sequence within the HTLV-1 LTR has about 75% sequence identity to the sequence of SEQ ID NO:29. In embodiments, the sequence within the HTLV-1 LTR has about 80% sequence identity to the sequence of SEQ ID NO:29. In embodiments, the sequence within the HTLV-1 LTR has about 85% sequence identity to the sequence of SEQ ID NO:29. In embodiments, the sequence within the HTLV-1 LTR has about 90% sequence identity to the sequence of SEQ ID NO:29. In embodiments, the sequence within the HTLV-1 LTR has about 95% sequence identity to the sequence of SEQ ID NO:29. In embodiments, the sequence within the HTLV-1 LTR has about 98% sequence identity to the sequence of SEQ ID NO:29. In embodiments, the sequence within the HTLV-1 LTR has about 99% sequence identity to the sequence of SEQ ID NO:29.

In embodiments, the sequence within the HTLV-1 LTR is operably linked to a nucleic acid encoding HTLV-1 bZIP factor (HBZ).

In embodiments, the zinc finger domain includes six zinc finger recognition helix regions designated F1 to F6, wherein F1 includes SEQ ID NO:63, F2 includes SEQ ID NO:64, F3 includes SEQ ID NO:65, F4 includes SEQ ID NO:66, F5 includes SEQ ID NO:67 and F6 includes SEQ ID NO:68. In embodiments, the F1 is SEQ ID NO:63, F2 is SEQ ID NO:64, F3 is SEQ ID NO:65, F4 is SEQ ID NO:66, F5 is SEQ ID NO:67 and F6 is SEQ ID NO:68.

In embodiments, the zinc finger domain has at least 75%, 80%, 85%, 90%, 95%, 96%, 97%, 98% or 99% sequence identity to the sequence of SEQ ID NO:6. In embodiments, the zinc finger domain has at least 90%, 95%, 96%, 97%, 98%, 99% or 100% sequence identity across the whole sequence or a portion of the sequence (e.g. a 20, 40, 60, 80, 100, 120, 140, or 160 continuous amino acid portion) of SEQ ID NO:6. In embodiments, the zinc finger domain includes a sequence having at least 75% sequence identity to SEQ ID NO:6. In embodiments, the zinc finger domain includes a sequence having at least 80% sequence identity to SEQ ID NO:6. In embodiments, the zinc finger domain includes a sequence having at least 85% sequence identity to SEQ ID NO:6. In embodiments, the zinc finger domain includes a sequence having at least 90% sequence identity to SEQ ID NO:6. In embodiments, the zinc finger domain includes a sequence having at least 91% sequence identity to SEQ ID NO:6. In embodiments, the zinc finger domain includes a sequence having at least 92% sequence identity to SEQ ID NO:6. In embodiments, the zinc finger domain includes a sequence having at least 93% sequence identity to SEQ ID NO:6. In embodiments, the zinc finger domain includes a sequence having at least 94% sequence identity to SEQ ID NO:6. In embodiments, the zinc finger domain includes a sequence having at least 95% sequence identity to SEQ ID NO:6. In embodiments, the zinc finger domain includes a sequence having at least 96% sequence identity to SEQ ID NO:6. In embodiments, the zinc finger domain includes a sequence having at least 97% sequence identity to SEQ ID NO:6. In embodiments, the zinc finger domain includes a sequence having at least 98% sequence identity to SEQ ID NO:6. In embodiments, the zinc finger domain includes a sequence having at least 99% sequence identity to SEQ ID NO:6. In embodiments, the zinc finger domain includes the sequence of SEQ ID NO:6. In embodiments, the zinc finger domain is the sequence of SEQ ID NO:6.

In embodiments, the zinc finger domain includes a sequence having about 75% sequence identity to SEQ ID NO:6. In embodiments, the zinc finger domain includes a sequence having about 80% sequence identity to SEQ ID NO:6. In embodiments, the zinc finger domain includes a sequence having about 85% sequence identity to SEQ ID NO:6. In embodiments, the zinc finger domain includes a sequence having about 90% sequence identity to SEQ ID NO:6. In embodiments, the zinc finger domain includes a sequence having about 91% sequence identity to SEQ ID NO:6. In embodiments, the zinc finger domain includes a sequence having about 92% sequence identity to SEQ ID NO:6. In embodiments, the zinc finger domain includes a sequence having about 93% sequence identity to SEQ ID NO:6. In embodiments, the zinc finger domain includes a sequence having about 94% sequence identity to SEQ ID NO:6. In embodiments, the zinc finger domain includes a sequence having about 95% sequence identity to SEQ ID NO:6. In embodiments, the zinc finger domain includes a sequence having about 96% sequence identity to SEQ ID NO:6. In embodiments, the zinc finger domain includes a sequence having about 97% sequence identity to SEQ ID NO:6. In embodiments, the zinc finger domain includes a sequence having about 98% sequence identity to SEQ ID NO:6. In embodiments, the zinc finger domain includes a sequence having about 99% sequence identity to SEQ ID NO:6.

In embodiments, the zinc finger domain has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:6 connected to a second sequence fragment having at least the percentage sequence identity as disclosed above to SEQ ID NO:6 through a sequence fragment having no sequence identity to SEQ ID NO:6. In embodiments, the non-contiguous sequence is a sequence including a plurality of sequence fragments having at least the percentage sequence identity as disclosed above to SEQ ID NO:6 connected through a plurality of sequence fragments having no sequence identity to SEQ ID NO:6.

In embodiments, the zinc finger domain has the percentage sequence identity as disclosed in the above paragraphs to at least 170 continuous amino acids of SEQ ID NO:6. In embodiments, the zinc finger domain has the percentage sequence identity as disclosed in the above paragraphs to at least 160 continuous amino acids of SEQ ID NO:6. In embodiments, the zinc finger domain has the percentage sequence identity as disclosed in the above paragraphs to at least 150 continuous amino acids of SEQ ID NO:6. In embodiments, the zinc finger domain has the percentage sequence identity as disclosed in the above paragraphs to at least 140 continuous amino acids of SEQ ID NO:6. In embodiments, the zinc finger domain has the percentage sequence identity as disclosed in the above paragraphs to at least 130 continuous amino acids of SEQ ID NO:6. In embodiments, the zinc finger domain has the percentage sequence identity as disclosed in the above paragraphs to at least 120 continuous amino acids of SEQ ID NO:6. In embodiments, the zinc finger domain has the percentage sequence identity as disclosed in the above paragraphs to at least 110 continuous amino acids of SEQ ID NO:6. In embodiments, the zinc finger domain has the percentage sequence identity as disclosed in the above paragraphs to at least 100 continuous amino acids of SEQ ID NO:6. In embodiments, the zinc finger domain has the percentage sequence identity as disclosed in the above paragraphs to at least 90 continuous amino acids of SEQ ID NO:6. In embodiments, the zinc finger domain has the percentage sequence identity as disclosed in the above paragraphs to at least 80 continuous amino acids of SEQ ID NO:6. In embodiments, the zinc finger domain has the percentage sequence identity as disclosed in the above paragraphs to at least 70 continuous amino acids of SEQ ID NO:6. In embodiments, the zinc finger domain has the percentage sequence identity as disclosed in the above paragraphs to at least 60 continuous amino acids of SEQ ID NO:6. In embodiments, the zinc finger domain has the percentage sequence identity as disclosed in the above paragraphs to at least 50 continuous amino acids of SEQ ID NO:6. In embodiments, the zinc finger domain has the percentage sequence identity as disclosed in the above paragraphs to at least 40 continuous amino acids of SEQ ID NO:6. In embodiments, the zinc finger domain has the percentage sequence identity as disclosed in the above paragraphs to at least 30 continuous amino acids of SEQ ID NO:6. In embodiments, the zinc finger domain has the percentage sequence identity as disclosed in the above paragraphs to at least 20 continuous amino acids of SEQ ID NO:6.

The sequence of SEQ ID NO:6 encodes a non-naturally occurring peptide sequence, which may be referred to herein as “HTLV-ZFP-7” or “ZFP-7”.

In embodiments, the protein further includes a transcriptional repressor. In embodiments, the transcriptional repressor includes a Kruppel associated box (KRAB) domain, methyl CpG binding protein 2 (meCP2), a DNA methyltransferase (DNMT) domain, or combinations thereof. In embodiments, the transcriptional repressor includes a KRAB domain. In embodiments, the transcriptional repressor includes meCP2 or a fragment thereof. In embodiments, the transcriptional repressor includes a KRAB domain and meCP2 or a fragment thereof.

In embodiments, the protein further includes a nuclear localization signal, a Tat domain, a Myc tag, or combinations thereof. In embodiments, the protein further includes a nuclear localization signal. In embodiments, the protein further includes a a Tat domain. In embodiments, the protein further includes a Myc tag.

In embodiments, the protein has at least 75%, 80%, 85%, 90%, 95%, 96%, 97%, 98% or 99% sequence identity to the sequence of SEQ ID NO: 15. In embodiments, the protein has at least 90%, 95%, 96%, 97%, 98%, 99% or 100% sequence identity across the whole sequence or a portion of the sequence (e.g. a 20, 40, 60, 80, 100, 120, 140, 160, 180, 200, 220, 240, 260, 280, or 300 continuous amino acid portion) compared to SEQ ID NO:15. In embodiments, the protein includes a sequence having at least 75% sequence identity to SEQ ID NO:15. In embodiments, the protein includes a sequence having at least 80% sequence identity to SEQ ID NO:15. In embodiments, the protein includes a sequence having at least 85% sequence identity to SEQ ID NO:15. In embodiments, the protein includes a sequence having at least 90% sequence identity to SEQ ID NO:15. In embodiments, the protein includes a sequence having at least 91% sequence identity to SEQ ID NO:15. In embodiments, the protein includes a sequence having at least 92% sequence identity to SEQ ID NO:15. In embodiments, the protein includes a sequence having at least 93% sequence identity to SEQ ID NO:15. In embodiments, the protein includes a sequence having at least 94% sequence identity to SEQ ID NO:15. In embodiments, the protein includes a sequence having at least 95% sequence identity to SEQ ID NO:15. In embodiments, the protein includes a sequence having at least 96% sequence identity to SEQ ID NO: 15. In embodiments, the protein includes a sequence having at least 97% sequence identity to SEQ ID NO:15. In embodiments, the protein includes a sequence having at least 98% sequence identity to SEQ ID NO:15. In embodiments, the protein includes a sequence having at least 99% sequence identity to SEQ ID NO:15. In embodiments, the protein includes the sequence of SEQ ID NO:15. In embodiments, the protein is the sequence of SEQ ID NO:15.

In embodiments, the protein includes a sequence having about 75% sequence identity to SEQ ID NO:15. In embodiments, the protein includes a sequence having about 80% sequence identity to SEQ ID NO:15. In embodiments, the protein includes a sequence having about 85% sequence identity to SEQ ID NO:15. In embodiments, the protein includes a sequence having about 90% sequence identity to SEQ ID NO:15. In embodiments, the protein includes a sequence having about 91% sequence identity to SEQ ID NO:15. In embodiments, the protein includes a sequence having about 92% sequence identity to SEQ ID NO:15. In embodiments, the protein includes a sequence having about 93% sequence identity to SEQ ID NO:15. In embodiments, the protein includes a sequence having about 94% sequence identity to SEQ ID NO:15. In embodiments, the protein includes a sequence having about 95% sequence identity to SEQ ID NO:15. In embodiments, the protein includes a sequence having about 96% sequence identity to SEQ ID NO:15. In embodiments, the protein includes a sequence having about 97% sequence identity to SEQ ID NO:15. In embodiments, the protein includes a sequence having about 98% sequence identity to SEQ ID NO:15. In embodiments, the protein includes a sequence having about 99% sequence identity to SEQ ID NO:15.

In embodiments, the protein has a sequence with the percentage sequence identity as disclosed in the above paragraphs, and the sequence having the percentage sequence identity as disclosed above is a non-contiguous sequence. In embodiments, the non-contiguous sequence is a sequence including a first sequence fragment having at least the percentage sequence identity as disclosed above to SEQ ID NO:15 connected to a second sequence fragment having at least the percentage sequence identity as disclosed above to SEQ ID NO:15 through a sequence fragment having no sequence identity to SEQ ID NO:15. In embodiments, the non-contiguous sequence is a sequence including a plurality of sequence fragments having at least the percentage sequence identity as disclosed above to SEQ ID NO:15 connected through a plurality of sequence fragments having no sequence identity to SEQ ID NO:15.

In embodiments, the protein has the percentage sequence identity as disclosed in the above paragraphs to at least 300 continuous amino acids of SEQ ID NO:15. In embodiments, the protein has the percentage sequence identity as disclosed in the above paragraphs to at least 290 continuous amino acids of SEQ ID NO:15. In embodiments, the protein has the percentage sequence identity as disclosed in the above paragraphs to at least 280 continuous amino acids of SEQ ID NO:15. In embodiments, the protein has the percentage sequence identity as disclosed in the above paragraphs to at least 270 continuous amino acids of SEQ ID NO:15. In embodiments, the protein has the percentage sequence identity as disclosed in the above paragraphs to at least 260 continuous amino acids of SEQ ID NO:15. In embodiments, the protein has the percentage sequence identity as disclosed in the above paragraphs to at least 250 continuous amino acids of SEQ ID NO:15. In embodiments, the protein has the percentage sequence identity as disclosed in the above paragraphs to at least 240 continuous amino acids of SEQ ID NO:15. In embodiments, the protein has the percentage sequence identity as disclosed in the above paragraphs to at least 230 continuous amino acids of SEQ ID NO:15. In embodiments, the protein has the percentage sequence identity as disclosed in the above paragraphs to at least 220 continuous amino acids of SEQ ID NO:15. In embodiments, the protein has the percentage sequence identity as disclosed in the above paragraphs to at least 210 continuous amino acids of SEQ ID NO:15. In embodiments, the protein has the percentage sequence identity as disclosed in the above paragraphs to at least 200 continuous amino acids of SEQ ID NO:15. In embodiments, the protein has the percentage sequence identity as disclosed in the above paragraphs to at least 190 continuous amino acids of SEQ ID NO:15. In embodiments, the protein has the percentage sequence identity as disclosed in the above paragraphs to at least 180 continuous amino acids of SEQ ID NO:15. In embodiments, the protein has the percentage sequence identity as disclosed in the above paragraphs to at least 170 continuous amino acids of SEQ ID NO:15. In embodiments, the protein has the percentage sequence identity as disclosed in the above paragraphs to at least 160 continuous amino acids of SEQ ID NO:15. In embodiments, the protein has the percentage sequence identity as disclosed in the above paragraphs to at least 150 continuous amino acids of SEQ ID NO:15. In embodiments, the protein has the percentage sequence identity as disclosed in the above paragraphs to at least 140 continuous amino acids of SEQ ID NO:15. In embodiments, the protein has the percentage sequence identity as disclosed in the above paragraphs to at least 130 continuous amino acids of SEQ ID NO:15. In embodiments, the protein has the percentage sequence identity as disclosed in the above paragraphs to at least 120 continuous amino acids of SEQ ID NO:15. In embodiments, the protein has the percentage sequence identity as disclosed in the above paragraphs to at least 110 continuous amino acids of SEQ ID NO:15. In embodiments, the protein has the percentage sequence identity as disclosed in the above paragraphs to at least 100 continuous amino acids of SEQ ID NO:15. In embodiments, the protein has the percentage sequence identity as disclosed in the above paragraphs to at least 90 continuous amino acids of SEQ ID NO:15. In embodiments, the protein has the percentage sequence identity as disclosed in the above paragraphs to at least 80 continuous amino acids of SEQ ID NO:15. In embodiments, the protein has the percentage sequence identity as disclosed in the above paragraphs to at least 70 continuous amino acids of SEQ ID NO:15. In embodiments, the protein has the percentage sequence identity as disclosed in the above paragraphs to at least 60 continuous amino acids of SEQ ID NO:15. In embodiments, the protein has the percentage sequence identity as disclosed in the above paragraphs to at least 50 continuous amino acids of SEQ ID NO:15. In embodiments, the protein has the percentage sequence identity as disclosed in the above paragraphs to at least 40 continuous amino acids of SEQ ID NO:15. In embodiments, the protein has the percentage sequence identity as disclosed in the above paragraphs to at least 30 continuous amino acids of SEQ ID NO:15. In embodiments, the protein has the percentage sequence identity as disclosed in the above paragraphs to at least 20 continuous amino acids of SEQ ID NO:15.

### Nucleic Acids

In an aspect is provided a nucleic acid encoding the protein provided herein including embodiments thereof. The nucleic acid may be provided in a vector, such as an expression vector. Thus, in another aspect a vector including the nucleic acid provided herein including embodiments thereof is provided.

In embodiments, the vector is an expression vector capable of directing the expression of nucleic acids to which they are operatively linked. The term “operably linked” means that the nucleotide sequence of interest is linked to regulatory sequence(s) in a manner that allows for expression of the nucleotide sequence. The regulatory sequence may include, for example, promoters, enhancers, and other expression control elements (e.g., polyadenylation signals). Such regulatory sequences are well known in the art and are described, for example, in Goeddel; Gene Expression Technology: Methods in Enzymology 185, Academic Press, San Diego, CA (1990), which is incorporated herein in its entirety and for all purposes. Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like. Any vector can be used so long as it is compatible with the desired or intended target cell.

Expression vectors contemplated to include, but are not limited to, viral vectors based on various viral sequences as well as those contemplated for eukaryotic target cells or prokaryotic target cells. The “target cells” may refer to the cells where the expression vector is transfected and the nucleotide sequence encoding the protein is expressed. In embodiments, the target cells are oncogenic T-cells.

In embodiments, a vector has one or more transcription and/or translation control elements. Depending on the target/vector system utilized, any of a number of suitable transcription and translation control elements, including constitutive and inducible promoters, transcription enhancer elements, transcription terminators, etc. can be used in the expression vector.

In embodiments, the vector is plasmid, a viral vector, a cosmid, or an artificial chromosome. In embodiments, the vector is a plasmid. In embodiments, the vector is a viral vector. In embodiments, the vector is a lentiviral vector. In embodiments, the vector is a adenoviral vector. In embodiments, the vector is a CMV vector.

Non-limiting examples of suitable eukaryotic promoters (i.e., promoters functional in a eukaryotic cell) include those from cytomegalovirus (CMV) immediate early, H1, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I. The promoter can be a constitutive promoter (e.g., CMV promoter, UBC promoter). In embodiments, the promoter can be a spatially restricted and/or temporally restricted promoter (e.g., a tissue specific promoter, a cell type specific promoter, etc.). EXTRACELLULAR VESICLES

Extracellular vesicles, including exosomes, may be used to deliver the proteins, nucleic acids, and vectors provided herein, including embodiments thereof. The term “extracellular vesicle” refers to a cell-derived vesicle including a membrane that encloses an internal space. Extracellular vesicles include all membrane-bound vesicles that typically have a smaller diameter than the cell from which they are derived. Generally, extracellular vesicles range in diameter from 20 nm to 1000 nm, and can include various macromolecular cargo either within the internal space, displayed on the external surface of the extracellular vesicle, and/or spanning the membrane. The cargo can include nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and cultured cells. Further description and methods for making extracellular vesicles are described, e.g., in Kojima, R., Bojar, D., Rizzi, G. et al. Designer exosomes produced by implanted cells intracerebrally deliver therapeutic cargo for Parkinson's disease treatment. Nat Commun 9, 1305 (2018). https://doi.org/10.1038/s41467-018-03733-8, which is incorporated herein in its entirety and for all purposes.

The term “exosome” refers to a cell-derived small (between 20-300 nm in diameter) vesicle comprising a membrane that encloses an internal space, and which is generated from the cell by direct plasma membrane budding or by fusion of the late endosome with the plasma membrane. The exosome includes lipid and/or fatty acid and optionally includes a payload (e.g., a therapeutic agent), a receiver (e.g., a targeting peptide), a polynucleotide (e.g., a nucleic acid, RNA, or DNA), a sugar (e.g., a simple sugar, polysaccharide, or glycan) or other molecules or drugs. The exosome can be derived from a producer cell, and isolated from the producer cell based on its size, density, biochemical parameters, or a combination thereof. An exosome is a species of extracellular vesicle.

In an aspect is provided an extracellular vesicle (EV) including the protein, nucleic acid, or vector provided herein, including embodiments thereof. In embodiments, the EV includes the protein provided herein, including embodiments thereof. In embodiments, the EV includes the nucleic acid provided herein, including embodiments thereof. In embodiments, the EV includes the vector provided herein, including embodiments thereof. In embodiments, the EV includes a nucleic acid encoding the protein provided herein including embodiments thereof. In embodiments, the EV further includes an EV membrane-associated protein and an oncogenic T-cell targeting protein.

An “EV membrane-associated protein” refers to a membrane protein on the EV, such as a transmembrane protein, an integral protein, or a peripheral protein. EV membrane-associated protein includes various CD proteins, transporters, integrins, lectins and cadherins. Exemplary membrane-associated proteins include CD9, CD37, CD53, CD63, CD68, CD81, CD82, LAMP-1, LAMP-2A, LAMP-2B, LAMP-2C, lactadherin, PTGFRN, BSG, IGSF3, IGSF8, ITGB1, ITGA4, SLC3A2, IGSF2, and ATP transporter proteins (ATP1A1, ATP1A2, ATP1A3, ATP1A4, ATP1B3, ATP2B1, ATP2B2, ATP2B3, ATP2B4). In embodiments, the membrane-associated protein is CD9. In embodiments, the membrane-associated protein is CD37. In embodiments, the membrane-associated protein is CD53. In embodiments, the membrane-associated protein is CD63. In embodiments, the membrane-associated protein is CD68. In embodiments, the membrane-associated protein is CD81. In embodiments, the membrane-associated protein is CD82. In embodiments, the membrane-associated protein is LAMP-1. In embodiments, the membrane-associated protein is LAMP-2A. In embodiments, the membrane-associated protein is LAMP-2B. In embodiments, the membrane-associated protein is LAMP-2C. In embodiments, the membrane-associated protein is lactadherin. In embodiments, the membrane-associated protein is PTGFRN. In embodiments, the membrane-associated protein is BSG. In embodiments, the membrane-associated protein is IGSF3. In embodiments, the membrane-associated protein is IGSF8. In embodiments, the membrane-associated protein is ITGB1. In embodiments, the membrane-associated protein is ITGA4. In embodiments, the membrane-associated protein is SLC3A2. In embodiments, the membrane-associated protein is IGSF2. In embodiments, the membrane-associated protein is an ATP transporter protein.

An “oncogenic T-cell targeting protein” refers to a protein (e.g. oncogenic T-cell protein) that can be used to target the EV to an oncogenic T-cell for a treatment using the EV described herein. In embodiments, the oncogenic T-cell targeting protein binds to or is capable of binding to a protein expressed on the surface of the oncogenic T-cell (e.g. oncogenic T-cell protein). In embodiments, the oncogenic T-cell protein targeted by the oncogenic T-cell targeting protein is expressed in higher levels on the surface of the oncogenic T-cell compared to a standard control (e.g. a non-cancer cell, non-oncogenic T-cell). In embodiments, the oncogenic T-cell protein targeted by the oncogenic T-cell targeting protein is expressed in higher levels on the surface of the oncogenic T-cell compared to a normal or non-oncogenic T-cell.

In embodiments, the expression level of an oncogenic T-cell protein on a oncogenic T-cell is 1.5, 5, 10, 20, 25, 50, 100, 500 or 1000 times higher than the expression level of a standard control (e.g. a non-cancer cell, non-oncogenic T-cell). Detection levels of an oncogenic T-cell protein may be assessed using conventional methods known in the art (e.g., immunofluorescent detection, protein biochemistry, RNA expression level). In embodiments, the oncogenic T-cell protein targeted by the oncogenic T-cell targeting protein is CD4, CD5, CD6, CD45RO, CD25 (IL2Rα), IL2RG (CD132; common 7 chain), IL15RA, CD29, CCR4, TCRαβ, OX40 (CD137; TNFRSF4), CD70 (TNFSF7), GITR (TNFRSF18), CADM1 (TSCL1; IGSF4), or MHC II. In embodiments, the oncogenic T-cell protein is CD4. In embodiments, the oncogenic T-cell protein is CD5. In embodiments, the oncogenic T-cell protein is CD6. In embodiments, the oncogenic T-cell protein is CD45RO. In embodiments, the oncogenic T-cell protein is CD25. In embodiments, the oncogenic T-cell protein is IL2RG. In embodiments, the oncogenic T-cell protein is IL15RA. In embodiments, the oncogenic T-cell protein is CD29. In embodiments, the oncogenic T-cell protein is CCR4. In embodiments, the oncogenic T-cell protein is TCRαβ. In embodiments, the oncogenic T-cell protein is OX40. In embodiments, the oncogenic T-cell protein is CD70. In embodiments, the oncogenic T-cell protein is GITR. In embodiments, the oncogenic T-cell protein is CADM1. In embodiments, the oncogenic T-cell protein is MHC II.

In embodiments, the oncogenic T-cell targeting protein is an antibody or antigen-binding fragment thereof. Antibodies and antigen-binding fragments thereof include whole antibodies, polyclonal, monoclonal and recombinant antibodies, fragments thereof, and further include single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(abl)₂, Fv, dAb, and Fd fragments, diabodies, nanobodies, and antibody-related polypeptides. In embodiments, the antibody is an scFv. Antibodies and antigen-binding fragments thereof also includes bispecific antibodies and multispecific antibodies so long as they exhibit the desired biological activity or function. In embodiments, the oncogenic T-cell targeting protein is a darpin. In embodiments, the oncogenic T-cell targeting protein is a peptide. In embodiments, the oncogenic T-cell targeting protein is an endogenous ligand.

In embodiments, the EV membrane-associated protein is CD63 or PTGFRN. In embodiments, the EV membrane-associated protein is CD63. In embodiments, the EV membrane-associated protein is PTGFRN. In embodiments, the oncogenic T-cell targeting protein is an anti-CCR4 antibody or fragment thereof. In embodiments, the anti-CCR4 antibody is a scFv. In embodiments, the oncogenic T-cell targeting protein is fused to an extracellular portion of the EV membrane-associated protein.

### Pharmaceutical Compositions

In an aspect is provided a pharmaceutical composition including the protein provided herein including embodiments thereof, the nucleic acid provided herein including embodiments thereof, the expression vector (e.g. vector) provided herein including embodiments thereof, or the extracellular vesicle (EV) provided herein including embodiments thereof. In embodiments, the pharmaceutical composition includes a protein provided herein including embodiments thereof. In embodiments, the pharmaceutical composition includes a nucleic acid provided herein including embodiments thereof. In embodiments, the pharmaceutical composition includes a vector provided herein including embodiments thereof. In embodiments, the pharmaceutical composition includes a extracellular vesicle (EV) provided herein including embodiments thereof. In embodiments, the pharmaceutical composition includes a nucleic acid encoding the protein provided herein including embodiments thereof.

The compositions are suitable for formulation and administration in vitro or in vivo. In embodiments, the pharmaceutical composition further includes a pharmaceutically acceptable carrier or excipient. Suitable carriers and excipients and their formulations are known in the art and described, e.g., in Remington: The Science and Practice of Pharmacy, 21st Edition, David B. Troy, ed., Lippicott Williams & Wilkins (2005), which is incorporated herein in its entirety and for all purposes.

### Cells

In an aspect is provided a cell including the protein provided herein including embodiments thereof, the expression vector (e.g. vector) provided herein including embodiments thereof, or the EV provided herein including embodiments thereof. In embodiments, the cell includes a protein provided herein including embodiments thereof. In embodiments, the cell includes a nucleic acid provided herein including embodiments thereof. In embodiments, the cell includes a vector provided herein including embodiments thereof. In embodiments, the cell includes an extracellular vesicle (EV) provided herein including embodiments thereof. In embodiments, the cell includes a nucleic acid encoding the protein provided herein including embodiments thereof.

In embodiments, the cell is an oncogenic T-cell. In embodiments, the oncogenic T-cell is an adult T-cell leukemia cell or an adult T-cell lymphoma cell. In embodiments, the oncogenic T-cell is an adult T-cell leukemia cell. In embodiments, the oncogenic T-cell is an adult T-cell lymphoma cell.

### Methods of Treatment

The protein provided herein including embodiments thereof is contemplated to be effective for the treatment of human T-cell lymphotropic virus type 1 (HTLV-1) associated diseases. A “human T-cell lymphotropic virus type 1 associated disease” or “HTLV-1 associated disease” refers to a condition caused directly or indirectly by infection of a subject's cell (e.g. a T cell, etc.) by HTLV-1. For example, infection of a host cell (e.g. a T-cell) by the virus may cause pro-oncogenic effects, for example, due to incorporation of viral RNA incorporated into the genome of the host cell. In another example, infection of a host cell by HTLV-1 may cause inflammation resulting in damage to the subject's cells. In another example, infection of a host cell may activate immunosuppresive cytokines, causing the subject to become susceptible to pathogens.

Applicant has demonstrated that the protein provided herein, including embodiments thereof, is a potent therapeutic for treatment of HTLV-I associated diseases, including HTLV-1 associated malignancies. For example, Applicant discovered that the protein provided, herein including embodiments thereof, is capable of reducing proliferation and viability of acute T-cell leukemia cells. Thus, in an aspect is provided a method of treating an HTLV-1 infection or an HTLV-1 associated disease in a subject in need thereof, including administering to the subject an effective amount of the protein provided herein including embodiments thereof, the nucleic acid provided herein including embodiments thereof, the vector provided herein including embodiments thereof, or the EV provided herein including embodiments thereof. In embodiments, the method includes treating an HTLV-1 infection. In embodiments, the method includes treating an HTLV-1 associated disease. In embodiments, the HTLV-1 associated disease is adult T-cell leukemia, adult T-cell lymphoma, HTLV-1 associated myelopathy, tropical spastic paraparesis, or HTLV-1 infection. In embodiments, the HTLV-1 associated disease is adult T-cell leukemia. In embodiments, the HTLV-1 associated disease is adult T-cell lymphoma. In embodiments, the HTLV-1 associated disease is HTLV-1 associated myelopathy. In embodiments, the HTLV-1 associated disease is tropical spastic paraparesis. In embodiments, the HTLV-1 associated disease is HTLV-1 infection.

In embodiments, the adult T-cell leukemia is acute, lymphomatous, chronic, or smoldering adult T-cell leukemia. In embodiments, the adult T-cell leukemia is acute adult T-cell leukemia. In embodiments, the adult T-cell leukemia is lymphomatous adult T-cell leukemia. In embodiments, the adult T-cell leukemia is chronic adult T-cell leukemia. In embodiments, the adult T-cell leukemia is smoldering adult T-cell leukemia. In embodiments, the adult T-cell lymphoma is acute, lymphomatous, chronic, or smoldering adult T-cell lymphoma. In embodiments, the adult T-cell lymphoma is acute adult T-cell lymphoma. In embodiments, the adult T-cell lymphoma is lymphomatous adult T-cell leukemia. In embodiments, the adult T-cell lymphoma is chronic adult T-cell leukemia. In embodiments, the adult T-cell lymphoma is smoldering adult T-cell leukemia.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Embodiments

Embodiment 1. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:27.

Embodiment 2. The protein of embodiment 1, wherein the sequence within the HTLV-1 LTR comprises the sequence of SEQ ID NO:27.

Embodiment 3. The protein of embodiment 1 or 2, wherein the sequence within the HTLV-1 LTR is operably linked to a nucleic acid encoding HTLV-1 bZIP factor (HBZ).

Embodiment 4. The protein of any one of embodiments 1-3, wherein the zinc finger domain comprises six zinc finger recognition helix regions designated F1 to F6, wherein F1 comprises SEQ ID NO:51, F2 comprises SEQ ID NO:52, F3 comprises SEQ ID NO:53, F4 comprises SEQ ID NO:54, F5 comprises SEQ ID NO:55 and F6 comprises SEQ ID NO:56.

Embodiment 5. The protein of any one of embodiments 1-4, wherein the zinc finger domain comprises a sequence having at least 75% sequence identity to SEQ ID NO:4.

Embodiment 6. The protein of embodiment 5, wherein the zinc finger domain comprises the sequence of SEQ ID NO:4.

Embodiment 7. The protein of any one of embodiments 1-6, wherein the protein further comprises a transcriptional repressor.

Embodiment 8. The protein of embodiment 7, wherein the transcriptional repressor comprises a Kruppel associated box (KRAB) domain, methyl CpG binding protein 2 (meCP2), a DNA methyltransferase (DNMT) domain, or combinations thereof.

Embodiment 9. The protein of embodiment 8, wherein the transcriptional repressor comprises a KRAB domain.

Embodiment 10. The protein of embodiment 8, wherein the transcriptional repressor comprises a KRAB domain and meCP2.

Embodiment 11. The protein of any one of embodiments 1-10, further comprising a nuclear localization signal, a Tat domain, a Myc tag, or combinations thereof.

Embodiment 12. The protein of any one of embodiments 1-11, comprising a sequence having at least 75% sequence identity to SEQ ID NO:13, 20, 21, 22, or 23.

Embodiment 13. The protein of embodiment 12, comprising the sequence of SEQ ID NO:13, 20, 21, 22, or 23.

Embodiment 14. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:25.

Embodiment 15. The protein of embodiment 14, wherein the sequence within the HTLV-1 LTR comprises the sequence of SEQ ID NO:25.

Embodiment 16. The protein of embodiment 14 or 15, wherein the sequence within the HTLV-1 LTR is operably linked to a nucleic acid encoding HTLV-1 bZIP factor (HBZ).

Embodiment 17. The protein of any one of embodiments 14-16, wherein the zinc finger domain comprises six zinc finger recognition helix regions designated F1 to F6, wherein F1 comprises SEQ ID NO:39, F2 comprises SEQ ID NO:40, F3 comprises SEQ ID NO:41, F4 comprises SEQ ID NO:42, F5 comprises SEQ ID NO:43 and F6 comprises SEQ ID NO:44.

Embodiment 18. The protein of any one of embodiments 14-17, wherein the zinc finger domain comprises a sequence having at least 75% sequence identity to SEQ ID NO:2.

Embodiment 19. The protein of embodiment 18, wherein the zinc finger domain comprises the sequence of SEQ ID NO:2.

Embodiment 20. The protein of any one of embodiments 14-19, wherein the protein further comprises a transcriptional repressor.

Embodiment 21. The protein of embodiment 20, wherein the transcriptional repressor comprises a Kruppel associated box (KRAB) domain, methyl CpG binding protein 2 (meCP2), a DNA methyltransferase (DNMT) domain, or combinations thereof.

Embodiment 22. The protein of embodiment 21, wherein the transcriptional repressor comprises a KRAB domain.

Embodiment 23. The protein of embodiment 21, wherein the transcriptional repressor comprises a KRAB domain and mcCP2.

Embodiment 24. The protein of any one of embodiments 14-23, further comprising a nuclear localization signal, a Tat domain, a Myc tag, or combinations thereof.

Embodiment 25. The protein of any one of embodiments 14-24, comprising a sequence having at least 75% sequence identity to SEQ ID NO:11 or 19.

Embodiment 26. The protein of embodiment 25, comprising the sequence of SEQ ID NO:11 or 19.

Embodiment 27. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:28.

Embodiment 28. The protein of embodiment 27, wherein the sequence within the HTLV-1 LTR comprises SEQ ID NO:28.

Embodiment 29. The protein of embodiment 27, wherein the sequence within the HTLV-1 LTR is operably linked to a nucleic acid encoding HTLV-1 bZIP factor (HBZ).

Embodiment 30. The protein of any one of embodiments 27-29, wherein the zinc finger domain comprises six zinc finger recognition helix regions designated F1 to F6, wherein F1 comprises SEQ ID NO:57, F2 comprises SEQ ID NO:58, F3 comprises SEQ ID NO:59, F4 comprises SEQ ID NO:60, F5 comprises SEQ ID NO:61 and F6 comprises SEQ ID NO:62.

Embodiment 31. The protein of any one of embodiments 27-30, wherein the zinc finger domain comprises a sequence having at least 75% sequence identity to SEQ ID NO:5.

Embodiment 32. The protein of embodiment 31, wherein the zinc finger domain comprises the sequence of SEQ ID NO:5.

Embodiment 33. The protein of any one of embodiments 27-32, wherein the protein further comprises a transcriptional repressor.

Embodiment 34. The protein of embodiment 33, wherein the transcriptional repressor comprises a Kruppel associated box (KRAB) domain, methyl CpG binding protein 2 (meCP2), a DNA methyltransferase (DNMT) domain, or combinations thereof.

Embodiment 35. The protein of embodiment 34, wherein the transcriptional repressor comprises a KRAB domain.

Embodiment 36. The protein of embodiment 34, wherein the transcriptional repressor comprises a KRAB domain and meCP2.

Embodiment 37. The protein of any one of embodiments 27-36, further comprising a nuclear localization signal, a Tat domain, a Myc tag, or combinations thereof.

Embodiment 38. The protein of any one of embodiments 27-37, comprising a sequence having at least 75% sequence identity to SEQ ID NO:14.

Embodiment 39. The protein of embodiment 38, comprising the sequence of SEQ ID NO:14.

Embodiment 40. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:32.

Embodiment 41. The protein of embodiment 40, wherein the sequence within the HTLV-1 LTR comprises the sequence of SEQ ID NO:32.

Embodiment 42. The protein of embodiment 40 or 41, wherein the sequence within the HTLV-1 LTR is operably linked to a nucleic acid encoding HTLV-1 bZIP factor (HBZ).

Embodiment 43. The protein of any one of embodiments 40-42, wherein the zinc finger domain comprises six zinc finger recognition helix regions designated F1 to F6, wherein F1 comprises SEQ ID NO:81, F2 comprises SEQ ID NO:82, F3 comprises SEQ ID NO:83, F4 comprises SEQ ID NO:84, F5 comprises SEQ ID NO:85 and F6 comprises SEQ ID NO:86.

Embodiment 44. The protein of any one of embodiments 40-43, wherein the zinc finger domain comprises a sequence having at least 75% sequence identity to SEQ ID NO:9.

Embodiment 45. The protein of embodiment 44, wherein the zinc finger domain comprises the sequence of SEQ ID NO:9.

Embodiment 46. The protein of any one of embodiments 40-45, wherein the protein further comprises a transcriptional repressor.

Embodiment 47. The protein of embodiment 46, wherein the transcriptional repressor comprises a Kruppel associated box (KRAB) domain, methyl CpG binding protein 2 (meCP2), a DNA methyltransferase (DNMT) domain, or combinations thereof.

Embodiment 48. The protein of embodiment 47, wherein the transcriptional repressor comprises a KRAB domain.

Embodiment 49. The protein of embodiment 47, wherein the transcriptional repressor comprises a KRAB domain and meCP2.

Embodiment 50. The protein of any one of embodiments 40-49, further comprising a nuclear localization signal, a Tat domain, a Myc tag, or combinations thereof.

Embodiment 51. The protein of any one of embodiments 40-50 comprising a sequence having at least 75% sequence identity to SEQ ID NO:18.

Embodiment 52. The protein of embodiment 51, comprising the sequence of SEQ ID NO:18.

Embodiment 53. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:31.

Embodiment 54. The protein of embodiment 53, wherein the sequence within the HTLV-1 LTR comprises the sequence of SEQ ID NO:31.

Embodiment 55. The protein of embodiment 53 or 54, wherein the sequence within the HTLV-1 LTR is operably linked to a nucleic acid encoding HTLV-1 bZIP factor (HBZ).

Embodiment 56. The protein of any one of embodiments 53-55, wherein the zinc finger domain comprises six zinc finger recognition helix regions designated F1 to F6, wherein F1 comprises SEQ ID NO:75, F2 comprises SEQ ID NO:76, F3 comprises SEQ ID NO:77, F4 comprises SEQ ID NO:78, F5 comprises SEQ ID NO:79 and F6 comprises SEQ ID NO:80.

Embodiment 57. The protein of any one of embodiments 53-56, wherein the zinc finger domain comprises a sequence having at least 75% sequence identity to SEQ ID NO:8.

Embodiment 58. The protein of embodiment 57, wherein the zinc finger domain comprises the sequence of SEQ ID NO:8.

Embodiment 59. The protein of any one of embodiments 53-58, wherein the protein further comprises a transcriptional repressor.

Embodiment 60. The protein of embodiment 59, wherein the transcriptional repressor comprises a Kruppel associated box (KRAB) domain, methyl CpG binding protein 2 (meCP2), a DNA methyltransferase (DNMT) domain, or combinations thereof.

Embodiment 61. The protein of embodiment 60, wherein the transcriptional repressor comprises a KRAB domain.

Embodiment 62. The protein of embodiment 60, wherein the transcriptional repressor comprises a KRAB domain and meCP2.

Embodiment 63. The protein of any one of embodiments 53-62, further comprising a nuclear localization signal, a Tat domain, a Myc tag, or combinations thereof.

Embodiment 64. The protein of any one of embodiments 53-63, comprising a sequence having at least 75% sequence identity to SEQ ID NO:17.

Embodiment 65. The protein of any one of embodiment 64, comprising the sequence of SEQ ID NO:17.

Embodiment 66. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:30.

Embodiment 67. The protein of embodiment 66, wherein the sequence within the HTLV-1 LTR comprises the sequence of SEQ ID NO:30.

Embodiment 68. The protein of embodiment 66 or 67, wherein the sequence within the HTLV-1 LTR is operably linked to a nucleic acid encoding HTLV-1 bZIP factor (HBZ).

Embodiment 69. The protein of any one of embodiments 66-68, wherein the zinc finger domain comprises six zinc finger recognition helix regions designated F1 to F6, wherein F1 comprises SEQ ID NO:69, F2 comprises SEQ ID NO:70, F3 comprises SEQ ID NO:71, F4 comprises SEQ ID NO:72, F5 comprises SEQ ID NO:73 and F6 comprises SEQ ID NO:74.

Embodiment 70. The protein of any one of embodiments 66-69, wherein the zinc finger domain comprises a sequence having at least 75% sequence identity to SEQ ID NO:7.

Embodiment 71. The protein of embodiment 70, wherein the zinc finger domain comprises the sequence of SEQ ID NO:7.

Embodiment 72. The protein of any one of embodiments 66-71, wherein the protein further comprises a transcriptional repressor.

Embodiment 73. The protein of embodiment 72, wherein the transcriptional repressor comprises a Kruppel associated box (KRAB) domain, methyl CpG binding protein 2 (meCP2), a DNA methyltransferase (DNMT) domain, or combinations thereof.

Embodiment 74. The protein of embodiment 73, wherein the transcriptional repressor comprises a KRAB domain.

Embodiment 75. The protein of embodiment 73, wherein the transcriptional repressor comprises a KRAB domain and meCP2.

Embodiment 76. The protein of any one of embodiments 66-75, further comprising a nuclear localization signal, a Tat domain, a Myc tag, or combinations thereof.

Embodiment 77. The protein of any one of embodiments 66-76, comprising a sequence having at least 75% sequence identity to SEQ ID NO:16.

Embodiment 78. The protein of embodiment 77, comprising the sequence of SEQ ID NO:16.

Embodiment 79. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:24.

Embodiment 80. The protein of embodiment 79, wherein the sequence within the HTLV-1 LTR comprises the sequence of SEQ ID NO:24.

Embodiment 81. The protein of embodiment 79 or 80, wherein the sequence within the HTLV-1 LTR is operably linked to a nucleic acid encoding HTLV-1 bZIP factor (HBZ).

Embodiment 82. The protein of any one of embodiments 79-81, wherein the zinc finger domain comprises six zinc finger recognition helix regions designated F1 to F6, wherein F1 comprises SEQ ID NO:33, F2 comprises SEQ ID NO:34, F3 comprises SEQ ID NO:35, F4 comprises SEQ ID NO:36, F5 comprises SEQ ID NO:37 and F6 comprises SEQ ID NO:38.

Embodiment 83. The protein of any one of embodiments 79-82, wherein the zinc finger domain comprises a sequence having at least 75% sequence identity to SEQ ID NO:1.

Embodiment 84. The protein of embodiments 83, wherein the zinc finger domain comprises the sequence of SEQ ID NO:1.

Embodiment 85. Embodiment 8. The protein of any one of embodiments 79-84, wherein the protein further comprises a transcriptional repressor.

Embodiment 86. The protein of embodiment 85, wherein the transcriptional repressor comprises a Kruppel associated box (KRAB) domain, methyl CpG binding protein 2 (meCP2), a DNA methyltransferase (DNMT) domain, or combinations thereof.

Embodiment 87. The protein of embodiment 86, wherein the transcriptional repressor comprises a KRAB domain.

Embodiment 88. The protein of embodiment 86, wherein the transcriptional repressor comprises a KRAB domain and meCP2.

Embodiment 89. The protein of any one of embodiments 79-88, further comprising a nuclear localization signal, a Tat domain, a Myc tag, or combinations thereof.

Embodiment 90. The protein of any one of embodiments 79-89, comprising a sequence having at least 75% sequence identity to SEQ ID NO:10.

Embodiment 91. The protein of embodiment 90, comprising the sequence of SEQ ID NO:10.

Embodiment 92. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:26.

Embodiment 93. The protein of embodiment 92, wherein the sequence within the HTLV-1 LTR comprises the sequence of SEQ ID NO:26.

Embodiment 94. The protein of embodiment 92 or 93, wherein the sequence within the HTLV-1 LTR is operably linked to a nucleic acid encoding HTLV-1 bZIP factor (HBZ).

Embodiment 95. The protein of any one of embodiments 92-94, wherein the zinc finger domain comprises six zinc finger recognition helix regions designated F1 to F6, wherein F1 comprises SEQ ID NO:45, F2 comprises SEQ ID NO:46, F3 comprises SEQ ID NO:47, F4 comprises SEQ ID NO:48, F5 comprises SEQ ID NO:49 and F6 comprises SEQ ID NO:50.

Embodiment 96. The protein of any one of embodiments 92-95, wherein the zinc finger domain comprises a sequence having at least 75% sequence identity to SEQ ID NO:3.

Embodiment 97. The protein of embodiments 96, wherein the zinc finger domain comprises the sequence of SEQ ID NO:3.

Embodiment 98. The protein of any one of embodiments 92-97, wherein the protein further comprises a transcriptional repressor.

Embodiment 99. The protein of embodiment 98, wherein the transcriptional repressor comprises a Kruppel associated box (KRAB) domain, methyl CpG binding protein 2 (meCP2), a DNA methyltransferase (DNMT) domain, or combinations thereof.

Embodiment 100. The protein of embodiment 99, wherein the transcriptional repressor comprises a KRAB domain.

Embodiment 101. The protein of embodiment 99, wherein the transcriptional repressor comprises a KRAB domain and meCP2.

Embodiment 102. The protein of any one of embodiments 92-101, further comprising a nuclear localization signal, a Tat domain, a Myc tag, or combinations thereof.

Embodiment 103. The protein of any one of embodiments 92-102, comprising a sequence having at least 75% sequence identity to SEQ ID NO:12.

Embodiment 104. The protein of embodiment 103, comprising the sequence of SEQ ID NO:12.

Embodiment 105. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:29.

Embodiment 106. The protein of embodiment 105, wherein the sequence within the HTLV-1 LTR comprises the sequence of SEQ ID NO:29.

Embodiment 107. The protein of embodiment 105 or 106, wherein the sequence within the HTLV-1 LTR is operably linked to a nucleic acid encoding HTLV-1 bZIP factor (HBZ).

Embodiment 108. The protein of any one of embodiments 105-107, wherein the zinc finger domain comprises six zinc finger recognition helix regions designated F1 to F6, wherein F1 comprises SEQ ID NO:63, F2 comprises SEQ ID NO:64, F3 comprises SEQ ID NO:65, F4 comprises SEQ ID NO:66, F5 comprises SEQ ID NO:67 and F6 comprises SEQ ID NO:68.

Embodiment 109. The protein of any one of embodiments 105-108, wherein the zinc finger domain comprises a sequence having at least 75% sequence identity to SEQ ID NO:6.

Embodiment 110. The protein of embodiment 109, wherein the zinc finger domain comprises the sequence of SEQ ID NO:6.

Embodiment 111. The protein of any one of embodiments 105-110, wherein the protein further comprises a transcriptional repressor.

Embodiment 112. The protein of embodiment 111, wherein the transcriptional repressor comprises a Kruppel associated box (KRAB) domain, methyl CpG binding protein 2 (meCP2), a DNA methyltransferase (DNMT) domain, or combinations thereof.

Embodiment 113. The protein of embodiment 112, wherein the transcriptional repressor comprises a KRAB domain.

Embodiment 114. The protein of embodiment 112, wherein the transcriptional repressor comprises a KRAB domain and meCP2.

Embodiment 115. The protein of any one of embodiments 105-114, further comprising a nuclear localization signal, a Tat domain, a Myc tag, or combinations thereof.

Embodiment 116. The protein of any one of embodiments 105-115, comprising a sequence having at least 75% sequence identity to SEQ ID NO:15.

Embodiment 117. The protein of embodiment 116, comprising the sequence of SEQ ID NO:15.

Embodiment 118. A nucleic acid encoding the protein of any one of embodiments 1-117.

Embodiment 119. A vector comprising the nucleic acid of embodiment 118.

Embodiment 120. An extracellular vesicle (EV) comprising a nucleic acid encoding the protein of any one of embodiments 1-117.

Embodiment 121. The EV of embodiment 120, wherein the EV further comprises an EV membrane-associated protein and an oncogenic T-cell targeting protein.

Embodiment 122. The EV of embodiment 121, wherein the membrane associated protein is CD63 or PTGFRN.

Embodiment 123. The EV of embodiment 121 or 122, wherein the oncogenic T-cell targeting protein is an anti-CCR4 antibody or fragment thereof.

Embodiment 124. The EV of any one of embodiments 121-123, wherein the oncogenic T-cell targeting protein is fused to an extracellular portion of the EV membrane-associated protein.

Embodiment 125. A pharmaceutical composition comprising the protein of any one of embodiments 1-117, the nucleic acid of embodiment 118, the vector of embodiment 119, or the EV of any one of embodiments 120-124

Embodiment 126. A cell comprising the protein of any one of embodiments 1-117, the nucleic acid of embodiment 118, the vector of embodiment 119, or the EV of any one of embodiments 120-124.

Embodiment 127. The cell of embodiment 126, wherein the cell is an oncogenic T-cell.

Embodiment 128. The cell of embodiment 127, wherein the oncogenic T-cell is an adult T-cell leukemia cell or an adult T-cell lymphoma cell.

Embodiment 129. A method of treating a human T-cell lymphotropic virus type 1 (HTLV-1) associated disease in a subject in need thereof, comprising administering to the subject an effective amount of the protein of any one of embodiments 1-117, the nucleic acid of embodiment 118, the vector of embodiment 119, or the EV of any one of embodiments 120-124.

Embodiment 130. The method of embodiment 129, wherein the HTLV-1 associated disease is adult T-cell leukemia, adult T-cell lymphoma, HTLV-1 associated myelopathy, tropical spastic paraparesis, or HTLV-1 infection.

Embodiment 131. The method of embodiment 130, wherein the HTLV-1 associated disease is adult T-cell leukemia.

Embodiment 132. The method of embodiment 130, wherein the HTLV-1 associated disease is adult T-cell lymphoma.

### EXAMPLES

### Example 1: Targeted Zinc-Finger Repressors to the Oncogenic HBZ Gene Inhibit Acute T-Cell Leukeamia (ATL) Proliferation

### Introduction

Human T-lymphotropic virus type I (HTLV-I) largely infects CD4+ T-cells resulting in a latent, life-long infection in patients. Crosstalk between oncogenic viral factors results in the transformation of the host cell into an aggressive cancer, acute T-cell leukemia/lymphoma (ATL). ATL has a very poor prognosis with no currently available effective treatments, urging the development of novel therapeutic strategies. Recent evidence exploring the mechanisms contributing to ATL highlights the viral anti-sense gene HTLV-1 bZIP factor (HBZ) as a tumor driver and a potential therapeutic target. The cys2his2 zinc-finger proteins (ZFPs) are abundant endogenous regulatory proteins that bind specific DNA motifs to control gene expression. As a result of well-characterized rules for DNA motif recognition, custom zinc-finger arrays can be generated to target unique sequences and artificially regulate a gene of interest (15). In this work, a series of Zinc-finger protein (ZFP) repressors were designed to target within the HTLV-I promoter that drives HBZ expression at highly conserved sites covering a wide range of HTLV-I genotypes. ZFPs were identified that potently suppressed HBZ expression, and, furthermore, these anti-HBZ ZFPs resulted in a significant reduction in the proliferation and viability of a patient-derived ATL cell line with the induction of cell cycle arrest and apoptosis. This study demonstrates the utility of this novel ZFP strategy as a targeted modality to inhibit the molecular driver of ATL, a next-generation therapeutic for aggressive HTLV-I associated malignancies.

### Materials and Methods

### Cell Lines

The MT-2 cells (ARP-237) and MT-4 cells (ARP-120) were obtained through the NIH HIV Reagent Program, Division of AIDS, NIAID, NIH: Human T-Lymphotropic Virus (HTLV-1)-Infected, contributed by Dr. Douglas Richman. The patient-derived IL-2 dependent ATL55T(+) cell line¹ was kindly provided by Dr Ye and Dr Maeda. The cells were maintained in RPMI media supplemented with 10% fetal bovine serum, except ATL55T(+) which had an additional 100 U/ml of IL-2 (Gibco Inc, MA, USA), and cultured at 37° C. and 5% CO₂. The HEK293 cell lines expressing GFP were generated and maintained as previously described (²).

### Vectors

The HTLV-I ZFP 2-10 amino acid sequences were identified using the ZF Tools Ver 3.0 (16). The ZFP sequences were designed to be fused to the repressor KRAB domain with an myc tag and NLS and ordered as gBLOCKs™ (IDT, MA, USA) (Tables 2, 6). The DNA fragments were cloned in a NheI and KpnI digested pcDNA3.1 by Gibson assembly using the NEBuilder® HiFi DNA assembly Master mix as instructed (NEB, MA, USA).

For the ZFP5 without a repressor domain, the ZFP5 sequence was amplified from its respective ZFP5-KRAB vector by a PCR with Myc-F and ZFP5-R primers using the Q5@Hot Start High-Fidelity 2× Master Mix (NEB, MA, USA). The ZFP5 amplicon was then inserted into a AflII and KpnI digested HTLV-I ZFP vector by Gibson assembly, which removed the KRAB domain.

The KRAB(ZIM3) and meCP2 sequences were ordered as gBLOCKs™ (Tables 2, 6) and inserted into a ZFP5 vector digested with AfeI with KpnI or Acc65I, respectively. To generate the ZFP5-PAM vector, the PAM repressor domain was amplified from a ZFP vector targeted to HIV (17) using ZFP5-PAM-F and ZFP5-PAM-R primers and inserted into a AfeI and KpnI digested ZFP5-KRAB vector (Table 5). The generation of the ZFP362-KRAB targeting HIV (ZFP-HIV-KRAB) has been described elsewhere (17).

To generate the luciferase HTLV-1 LTR vector, an in-house generated vector with a HERV-K HML-2 (HK2) LTR bi-directionally expressing Rluc and Flue was used as a cloning backbone (Rluc-HK2-LTR-Fluc). The 5′ LTR sequence from HTLV-I (accession number LC192515.1) was ordered as a gBLOCK™ (IDT, MA, USA) (Table 6). The DNA fragment was used to replace the HK-2 LTR sequence by cloning into a MluI and NheI digested Rluc-HK2-LTR-Fluc vector by Gibson assembly to generate the Rluc-HTLV-1-LTR-Fluc vector.

To generate the HTLV-1 LTR reporter with the HBZ spliced Rluc, the remaining intron sequence was ordered as a gBLOCK™ (Table 6). This DNA fragment was cloned into a MluI and EcoRV digested Rluc-HTLV-1-LTR-Fluc vector by Gibson assembly to generate the Rluc(splice)-HTLV-1-LTR-Fluc vector. The translation start of Rluc was mutated and expression only occurs with the correct splicing of the internal HBZ LTR ORF onto the Rluc sequence. To generate the luciferase vectors for the different HTLV-1 genotypes, the 3′LTR promoter sequence upstream of the HBZ start site from subtypes a-g were ordered as a gBLOCKs™ and inserted into a NdeI and NheI digested Rluc(splice)-HTLV-1-LTR-Fluc vector (Table 6).

To generate the pcDNA-LTR-HBZ-3×FLAG vector, the complete TL-Om1 HTLV-I LTR with the HBZ gene was amplified with the pcDNA-HBZ-F and pcDNA-HBZ-R primers (Table 5) using the Q5@Hot Start High-Fidelity 2× Master Mix (NEB, MA, USA) from a genomic DNA template extracted from TL-Om1 cells using the QIAamp DNA Mini Kit (Qiagen, Hilden, Germany). The PCR fragment of the correct size was gel purified using QIAquick® Gel Extraction Kit (Qiagen, Hilden, Germany) and cloned into a MfeI and XhoI digested pcDNA3.1 by Gibson assembly using NEBuilder® HiFi DNA assembly Master mix (NEB, MA, USA). The cloning procedure removed the CMV promoter from the pcDNA3.1 vector. A 3×FLAG tag was ordered as gBLOCK™ and inserted into a pcDNA-LTR-HBZ vector digested with SacII and XhoI using Gibson assembly. To generate the LTR-HBZ-IRES-GFP-Puro vector, an IRES-GFP-PURO was ordered as a gBLOCK™ (IDT, MA, USA) and using Gibson assembly inserted into a pcDNA-LTR-HBZ-3×FLAG digested with EcoRI and XhoI. For the pcDNA-CMV-HBZ-3×FLAG vector, the vector was generated by VectorBuilder (CA, USA). The shRNA-362 targeted to the HIV promoter been previously described 5.

### Flow Cytometry for Cell Count:

At the described time points, 100 μl of the cell suspension was placed into 1.7 mL microfuge tubes. Thereafter, 10 μL of a 1 μg/mL solution of DAPI (in 1×PBS) was added to each sample. Samples were briefly vortexed and incubated in the dark for 10 minutes. Cell count and viability data were acquired on a Nxt Attune Cytometer (ThermoFisher Scientific) using a flow rate of 100 uL/min. Samples were first gated by size and granularity (SSC-A vs FSC-A), followed by single cell gating (FSC-H vs FSC-A). Upon single cell selection, samples were gated for viability using the VL1 (DAPI) channel. A set volume of 50 uL was used so that viable cells/ml could be calculated for each sample.

### Cell Culture

The HEK293 cells were cultured in Dulbecco's modified Eagle's medium (DMEM) supplemented with 10% fetal bovine serum (FBS, Thermo Fisher Scientific, MA, USA). The TL-Om1, Jurkat cells, or Jurkat-LTR-HBZ-IRES-GFP-Puro cells were maintained in Roswell Park Memorial Institute Medium (RPMI) supplemented with 10% fetal bovine serum. The TL-Om1 cells were kindly provided by Prof. Kazuo Sugamura (18). All cell lines were cultured at 37° C. and 5% CO₂.

To generate the Jurkat-LTR-HBZ-IRES-GFP-Puro cell line, the LTR-HBZ-IRES-GFP-puro vector was linearized, purified, and 1 μg of DNA was electroporated using the Neon® transfection system into a Jurkat cell line using the electroporation conditions below. The media was then supplemented with 1.5 μg/ml puromycin (Gibco, Thermo Fisher Scientific, MA, USA).

In vitro mRNA synthesis and electroporation:

The ZFP templates were linearized by digestion with XbaI and purified with the Zymo DNA Clean & Concentrator-25 kit (Zymo Research, CA, USA) and 1 μg of template was used for mRNA production with the T7 mScript™ Standard mRNA Production System according to instructions (Cellscript, WI, USA). The integrity and molecular weight of the mRNA was confirmed using PAGE loaded on to 6% Novex™ TBE-Urea Gels (Thermo Fisher Scientific, MA, USA), and visualised with ethidium bromide staining.

For the proliferation assays, a total of 5×10⁴ TL-Om1 or Jurkat cells were electroporated with 2 μg or 4 μg of mRNA using the 10 μl Neon® transfection system. The electroporation conditions were as follows: ATL55T(+) and TL-Om1 cells: 1325 V, 10 ms, 3 pulse; Jurkat cells: 1450 V, 10 ms, 3 pulse. For the experiments using DNA vectors, 1 μg of expression vector was electroporated into 2×10⁵ TL-Om1 cells with the same described conditions. For the qPCR, western blot, and apoptosis assays, 1×10⁶ TL-Om1 cells were electroporated with the described amount of mRNA. For the LTR-GFP knockdown assays, 1×10⁶ Jurkat-LTR-HBZ-IRES-GFP-Puro cells were electroporated with the described amount of mRNA. For the cell cycle arrest assays, 2×10⁶ TL-Om1 cells were electroporated with 4 μg of mRNA. The electroporated cells were added to 1 ml of pre-warmed complete media in a 48-well plate and processed for further analysis at the described timepoints.

### Transfections and Luciferase Assays:

For the reporter assays, HEK293 cells were seeded at 1.2×10⁵ cells per well, and 24 hrs later were transfected using Lipofectamine 3000@(Thermo fisher scientific, MA, USA) with 250 ng of HBZ luciferase reporter vector (Rluc-HTLV-1-LTR-Fluc or Rluc(splice)-HTLV-1-LTR-Fluc) and 250 ng of the ZFP expression vector. At 48 hrs post-transfection, the levels of Rluc and Fluc were assessed using a Dual-Luciferase® Reporter Assay and activity detected on the Glomax® Explorer system (Promega, WI, USA). For the detection of HBZ RNA and protein, transfections were performed with the pcDNA-LTR-HBZ-3×FLAG vector as described above. At 48 hrs post-transfection, the samples were processed for either the RT-qPCR or western blot assays as described below.

### RT-GPCR Assay:

After treatment, at the specific time points, RNA was extracted from the HEK293 or TL-Om1 cells using the Promega Maxwell™ RSC simplyRNA Tissue Kit (Promega, WI, USA). One-microgram of HEK293 RNA or 200 ng of TL-Om1 or ATL55T(+) RNA was reverse transcribed using the QuantiTect® Reverse Transcription Kit (Qiagen, Hilden, Germany), and 4 μl of RT-template was amplified in a LightCycler® 96 (Roche, Basel, Switzerland) using the KAPA Sybr® Fast qPCR Master Mix (Sigma-aldrich, MO, USA) with the following conditions: initial denaturation: 95° C. for 3 min, denaturation 95° C. for 5 s, annealing/extension at 60° C. for 20 sec. The data was analysed with the LightCycler® 96 software (V1.1.0.1320). The primers used to detect the various expressed and endogenous targets are described in Table 5.

### Western Blots:

After treatment, the cells were lysed in M-PER™ Mammalian Protein Extraction Reagent supplemented with Halt™ Protease Inhibitor Cocktail and the protein concentration determined by Pierce™ BCA Protein Assay Kit according to manufacturer protocols (Thermo Fisher Scientific, MA, USA). Equal amounts of protein from each sample was loaded onto a 4-20% Mini-PROTEAN TGX Precast Protein Gels (Bio-Rad, CA, USA) and transferred using the Trans-Blot® Turbo™ Transfer System with Trans-Blot® Turbo™ Mini Nitrocellulose Transfer Packs (Bio-Rad, CA, USA). The membrane was blocked with 3% BSA TBS-T and subsequently probed with the following antibodies: α-FLAG mouse mAb Anti-Flag® M2 (Cat. No. F1804; Milliporesigma, CA, USA), α-myc mouse mAb 9B11 (Cat. No. 2276; Cell Signalling Technology, MA, USA), or α-alpha tubulin rabbit polyclonal (Cat. No. 4074; Abcam, Cambridge, United Kingdom). Secondary antibodies used were the HIRP-conjugated α-Mouse IgG goat antibody (Cat. No. 1705047; Bio-Rad, CA, USA) or Immun-Star™ Goat Anti-Rabbit (GAR)-IRP Conjugate (Cat. No. 170546; Bio-Rad, CA, USA), and exposed using a Pierce™ SuperSignal™ West Pico PLUS Chemiluminescent Substrate (Thermo Fisher Scientific, MA, USA). The Bio-Rad Chemidoc™ Touch Gel-Imaging System was used to detect the signal and analysed using the Bio-rad Image Lab™ Software V6.1.0. All antibodies were diluted in blocking buffer.

### Cell Proliferation Assays:

After treatment of the TL-Om1 cells or Jurkat cells and at the designated time points, 700 μl of media was removed, the cells resuspended in the remaining 300 μl, and 100 μl transferred to a 96-well plate. The alamarBlue™ Cell Viability Reagent was added (Thermo Fisher Scientific, MA, USA) and the levels of fluorescence was measured at 3 hrs post-addition on the Glomax® Explorer system (Promega, WI, USA). To measure cell viability and counts, 10 μl of resuspend cells was added to 10 μl of trypan blue stain and assessed on the Countess® II Automated Cell Counter (Thermo Fisher Scientific, MA, USA). Then, 810 μl of media was replaced.

### Cell Cycle Arrest Assay:

At 24 hrs post-treatment, the TL-Om1 cells were collected, washed twice with PBS, and the fixed with ice-cold 70% ethanol for 30 min at 4° C. The cells were pelleted by centrifugation at 850 g for 5 min, washed twice with PBS, and resuspended in FxCycle™ PI/RNase Staining Solution (Thermo Fisher Scientific, MA, USA). Single cells were then counted to 10000 events on a BD Accuri™ C6 and cell cycle phase analysed using the FlowJo vX5.0 software.

### Apoptosis Assays:

To assess apoptosis, Annexin V and propidium iodide (PI) staining was performed. One-hundred thousand TL-Om1 cells were electroporated with the described amount of ZFP mRNA and the cells were harvested at 24 or 48 hrs. The cells were washed twice with ice-cold PBS, the pellet resuspend in 100 μl of 1× Annexin V Binding Buffer (Cat. No. 51-66121E; BD Biosciences, NJ, USA), and then 1 μl of anti-Annexin V-FITC (Cat. No. 556419; BD Biosciences, NJ, USA) and 2 μl of PI stain was added (Cat. No. P3566; Thermo Fisher Scientific, MA, USA) and incubated for 15 min in the dark at RT. Four-hundred microliters of 1× Annexin V Binding Buffer was added and 10000 events were assessed on a BD Accuri™ C6 flow cytometer and analysed using the FlowJo vX5.0 software

To assess Caspase 3/7 activity, the TL-Om1 cells electroporated with mRNA as described above were assessed using the Caspase-Glo® 3/7 Assay System according to manufacturer instructions (Promega, WI, USA) and the signal detected on the Glomax® Explorer system (Promega, WI, USA).

### FACS Analysis of CCR4 Surface Expression:

For the detection of the CCR4 receptor, TL-Om1 cells after electroporation were centrifuged at 1000 rpm for 5 min and resuspended in 45 μl of PBS with 1% bovine serum albumin (BSA) and incubated with 5 μl of a mouse PE anti-human CD194 L291H4 (Cat. No. 359411; Biolegend, CA, USA) for 30 min at RT in the dark. Five-hundred microliters of PBS with 1% BSA was added, the cells washed, and resuspended in 100 μl of PBS with 1% BSA. Single cells were counted to a total of 10000 events using the BD Accuri™ C6 and analysed on the FlowJo vX5.0 software.

### ATAC-Seg and Analysis

ATAC-seq analysis was performed by the City of Hope integrative genomic core. A previously published OMNI ATAC-Seq protocol (17) was used for cell lysis, tagmentation, and DNA purification. The Tn5 treated DNA was amplified with 10 cycles of PCR in 50 μl reaction volumes. 1.8× AmpurXP beads purification was used for the PCR product clean-up. The libraries were validated with Agilent Bioanalyzer DNA High Sensitivity Kit, and quantified with qPCR. ATAC-seq libraries were sequenced on Illumina NovaSeq6000 with S4 Reagent v1.5 kit (Illumina, Cat 20028312) at Tgen with the sequencing length of 2×101. Real-time analysis (RTA) 3.4.4 software was used to process the image analysis. Raw sequencing reads were filtered using the fastp (https://github.com/OpenGene/fastp) (18) and aligned against a reference genome with HTLV sequence in chromosome 1 into the hg38 genome using HISAT2 V2.1.0 (19) aligner with its very-sensitive default parameters. Furthermore, aligned reads with a mapping quality less than 20 along with PCR duplicates were filtered out using samtools v1.6 (20). Detection of open chromatin areas was performed with the MACS2 v2.2.5 peak calling tool using the paired-end alignment information setup (-BAMPE parameter), after which the peaks detected within the promoter regions of protein coding genes defined as 3 kb upstream from the Transcription Start Site (TSS) were selected for analysis. The peaks are annotated using ChTPseeker (https://bioconductor.org/packages/release/bioc/html/ChIPseeker.html) and UCSC genome hg38 with default settings. The pathway enrichments were done using ReactomePA package (https://bioconductor.org/packages/release/bioc/html/ReactomePA.html), including 3 canonical pathway databases, KEGG (https://www.genome.jp/kegg/), Reactome (https://reactome.org/), and Biocarta (https://maayanlab.cloud/Harmonizome/resource/Biocarta). The node sizes represent the number of genes overlapped with the pathway genes while the heatmap represent the statistical significance. The peaks are reannotated with narrower genomic regions (tssRegion=c(−1000, 1000)). The R/Bioconductor package csaw (21) was used to detect differential accessibility among groups.

### Statistical Analysis

Graphing and statistical analyses was performed using GraphPad Prism version 8 (V8.1.2).

### Results

### Screening of Potent ZFP Repressors of the HTLV-I LTR Promoter

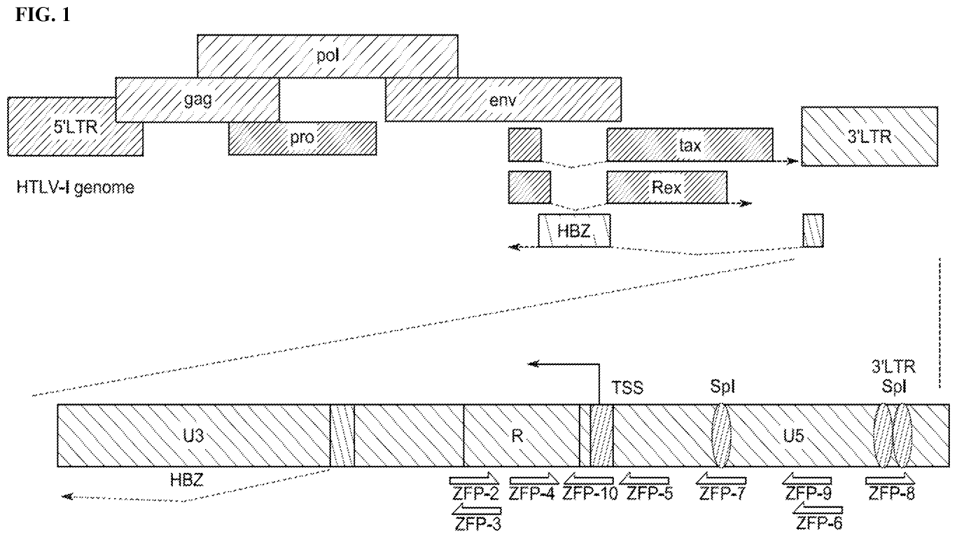

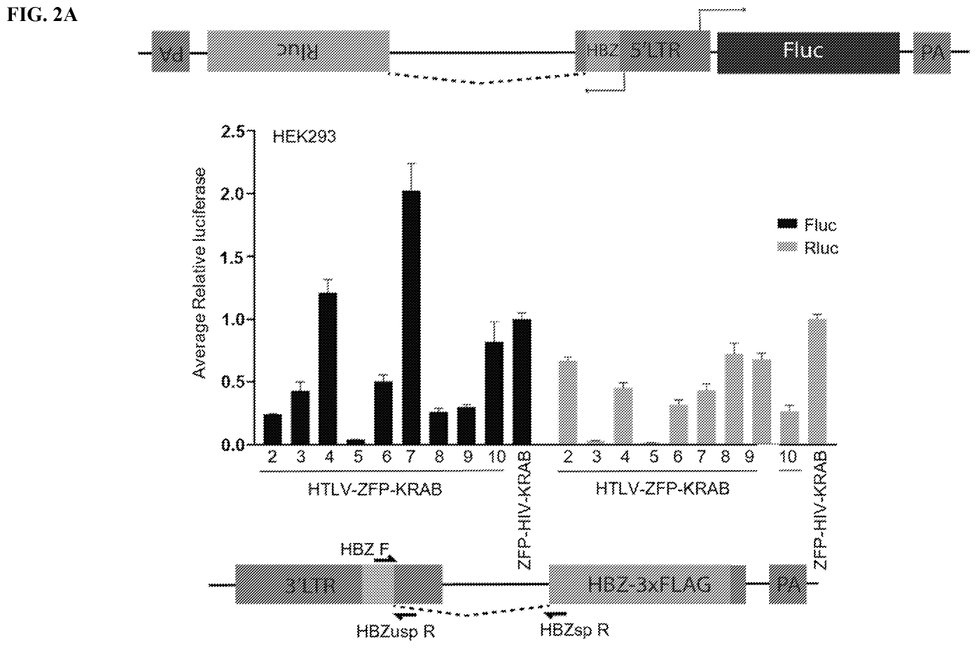

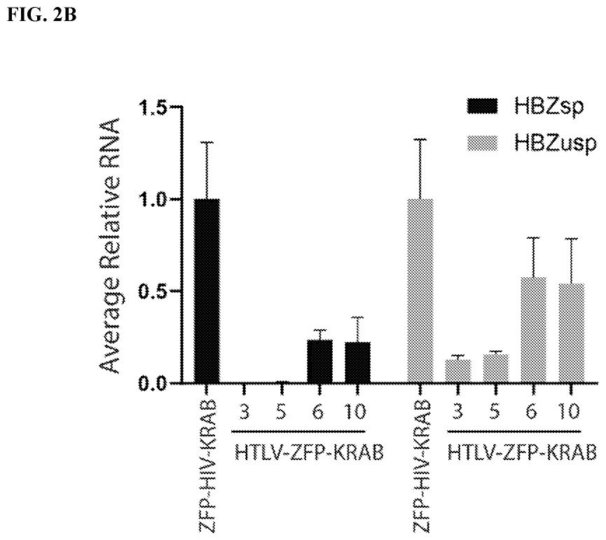

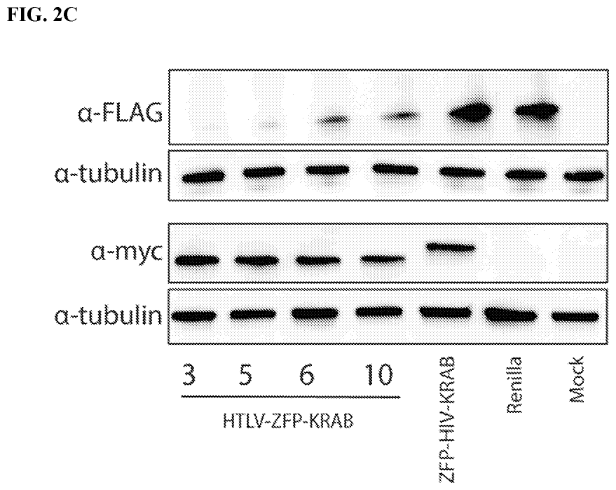

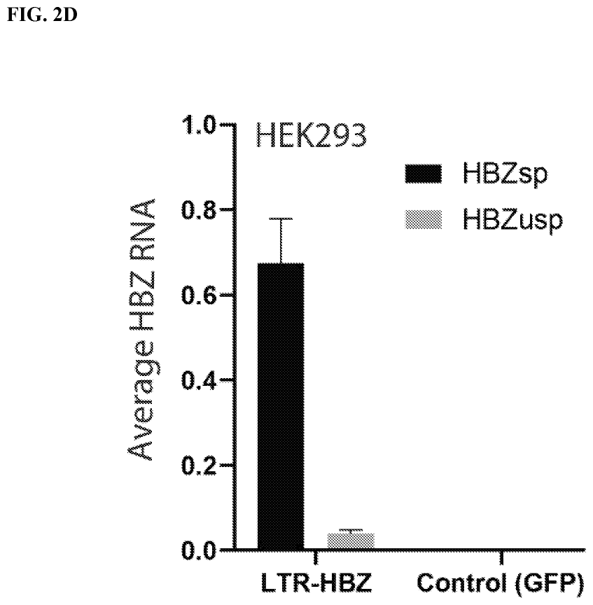

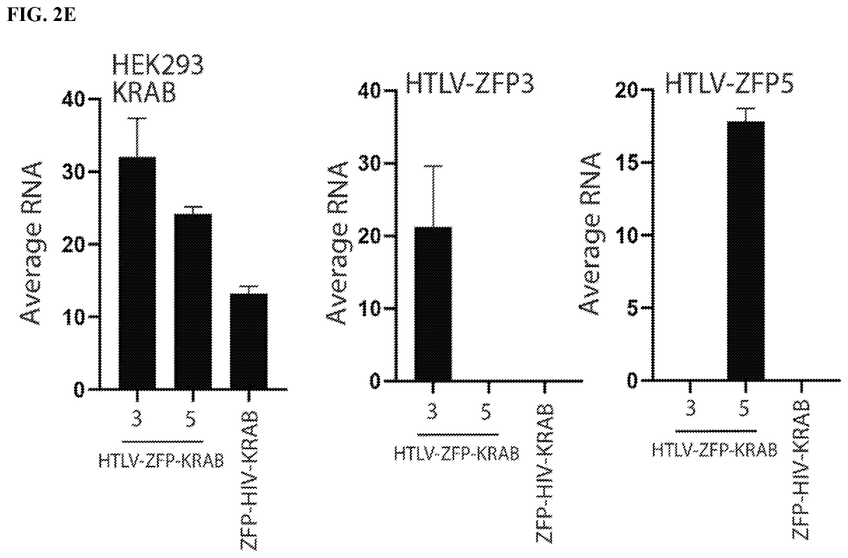

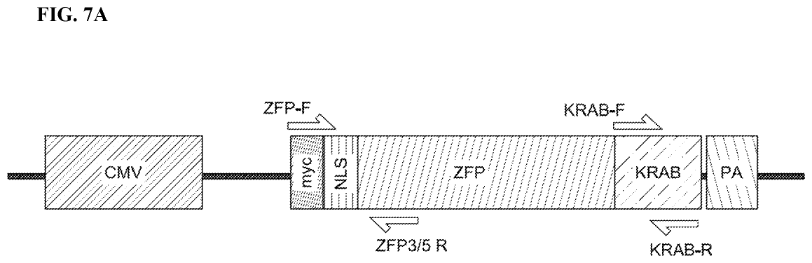

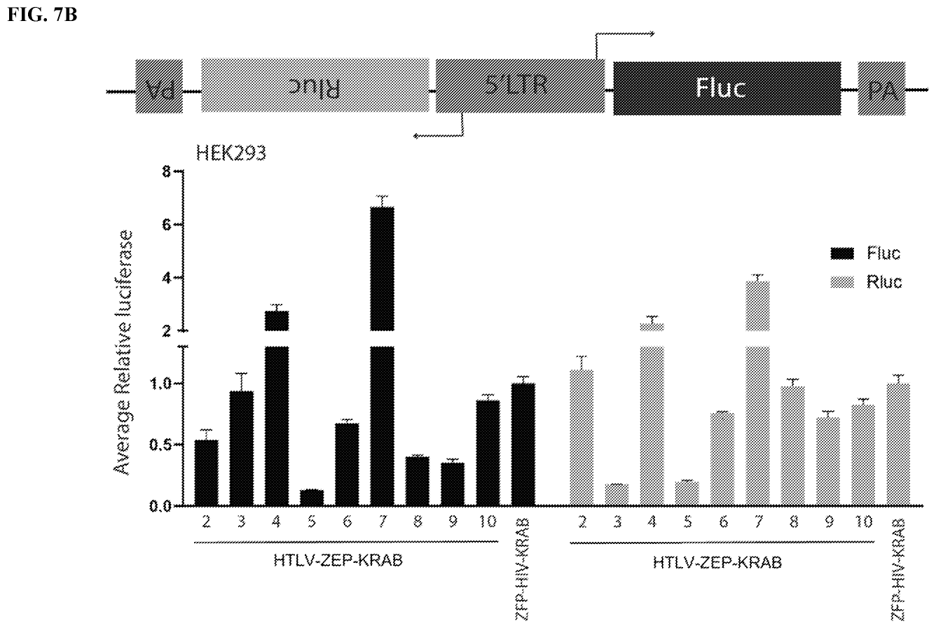

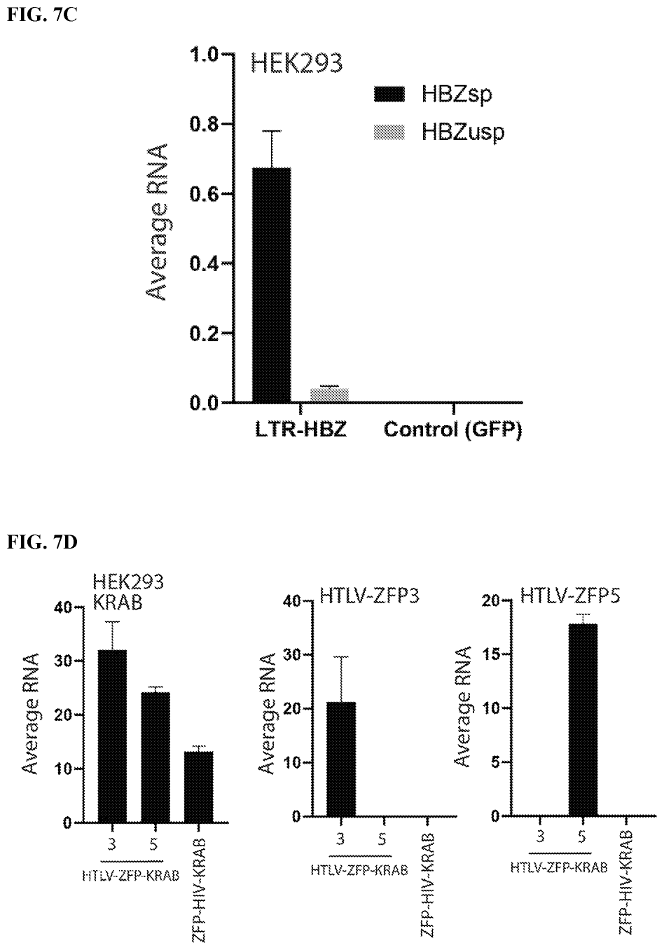

The 3′ LTR of the HTLV-I drives the expression of the anti-sense HBZ RNA and protein, implicated in ATL proliferation and pathology (FIG. 1). Using the ZF Tools Ver 3.0 software (19), a series of nine ZFPs were generated to target the LTR of HTLV-I, each recognizing a unique 18 nt DNA motif (FIG. 1 and Table 1). The ZFP coding sequence was inserted into a cytomegalovirus (CMV) expression vector and fused to a nuclear localization signal (NLS) and well-known kruppel-associated box (KRAB) repressor domain derived ZFP10/KOX1 (20) (FIG. 7A). To assess if the ZFPs affected HTLV promoter expression of the HBZ transcript, the ZFPs were co-transfected with a bi-directional expression vector containing the HTLV-LTR driving Firefly (Fluc) and Renilla (Rluc) luciferase in the sense and anti-sense direction, respectively (FIG. 2A). The HBZ intron was maintained so that the 5′ HBZ sequence located within the LTR spliced onto Rluc, and luciferase activity an indicator of spliced HBZ transcript expression. At 48 hrs post-transfection, the HTLV-ZFP3 and ZFP5 demonstrated a strong reduction in Rluc levels (>99%) compared to a control ZFP known to target the LTR of human immunodeficiency virus (ZFP-HIV-KRAB) (FIG. 2A) (21). The ZFP6-KRAB and 10-KRAB were found to be the next best HBZ repressors and resulted in ˜60% inhibition of Rluc levels. Furthermore, the ZFP5-KRAB was able to potently inhibit sense Fluc activity, while ZFP3-KRAB demonstrated ˜50% inhibition. To assess if the ZFP affected basal LTR promoter activity, the ZFP expression vectors were transfected into HEK293 cells with a bi-directional expression vector without the spliced intron and, likewise, ZFP3-KRAB and ZFP5-KRAB showed a comparable level of luciferase suppression to their activity against the spliced vector, suggesting the ZFPs functionally augment promoter activity and affect HBZ reporter expression (FIG. 7B). As ZFP-HTLV-ZFP-3, 5, 6, and 10-KRAB were the most effective suppressors of anti-sense promoter activity, they were selected for further characterization.

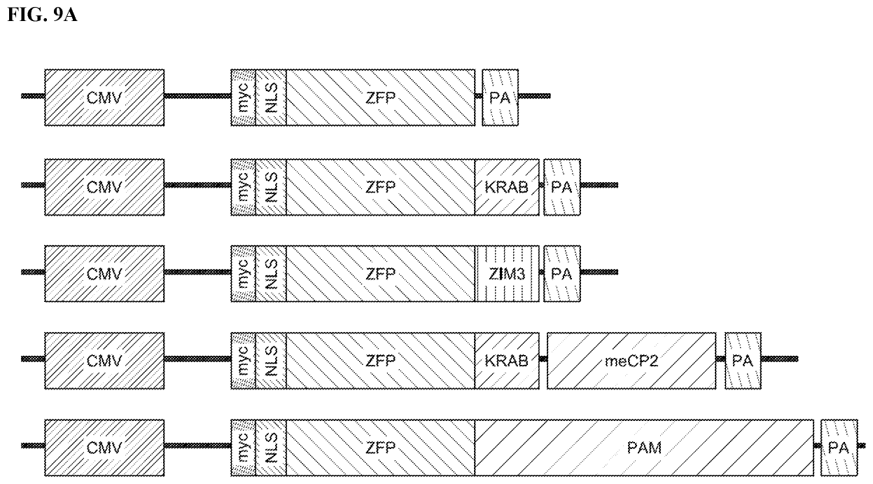

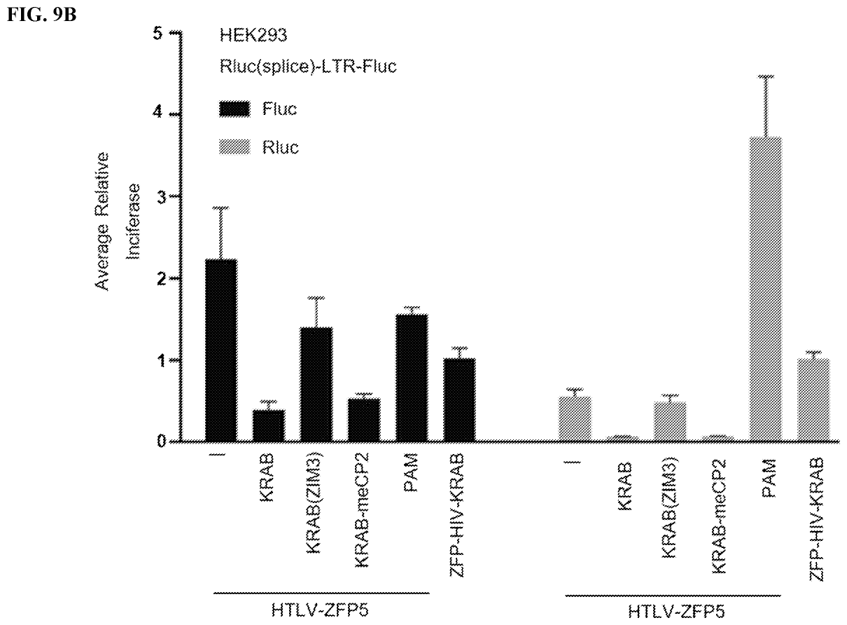

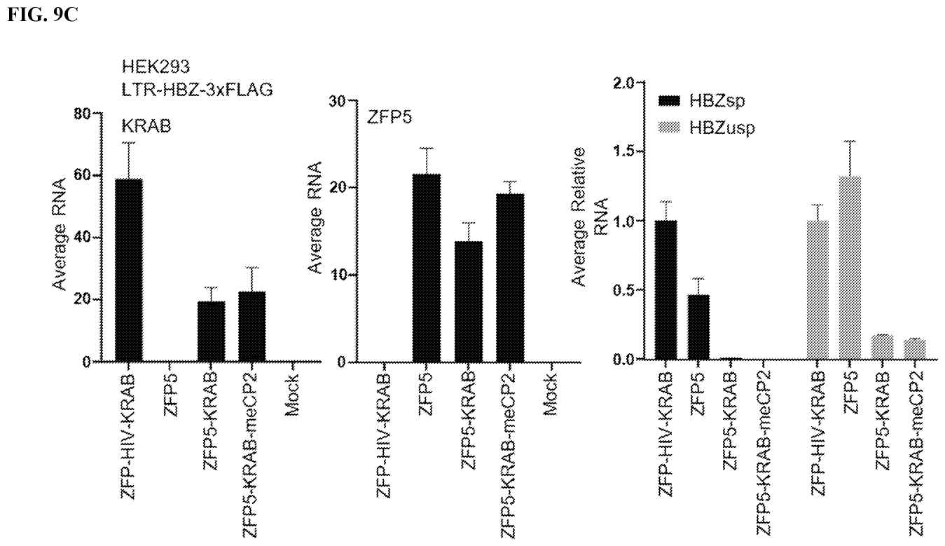

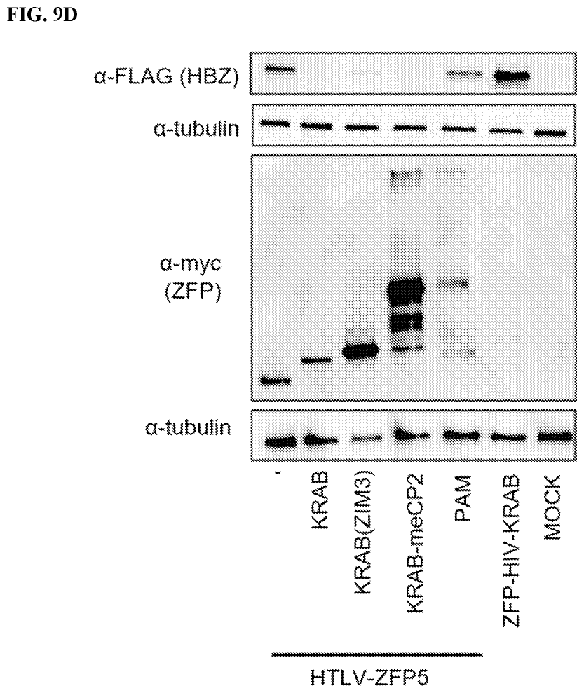

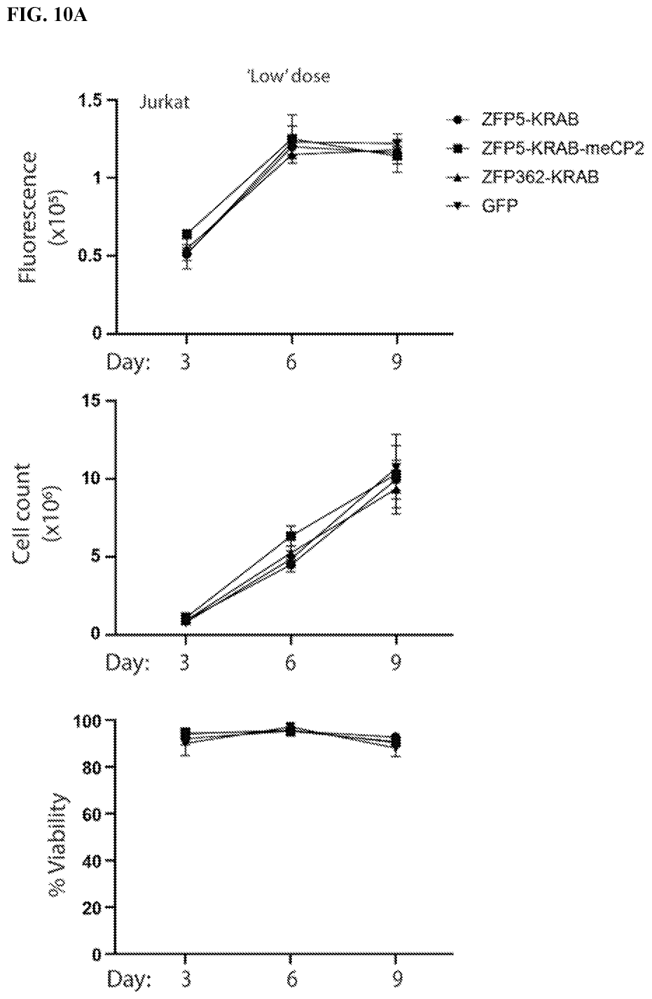

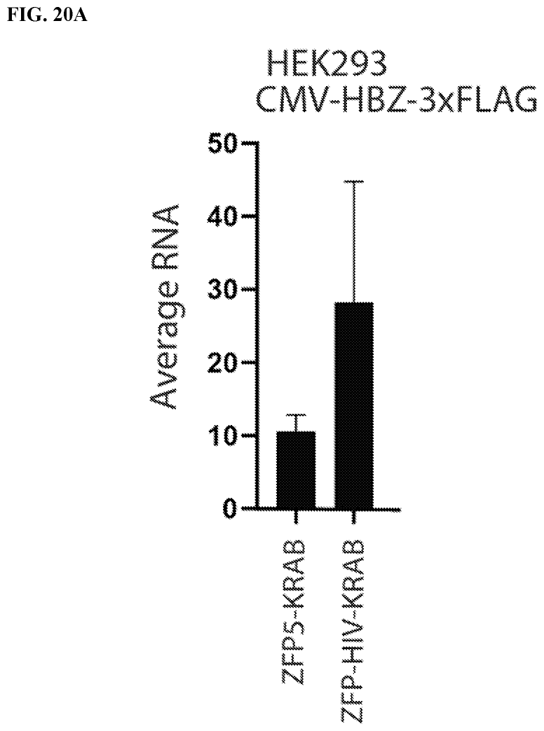

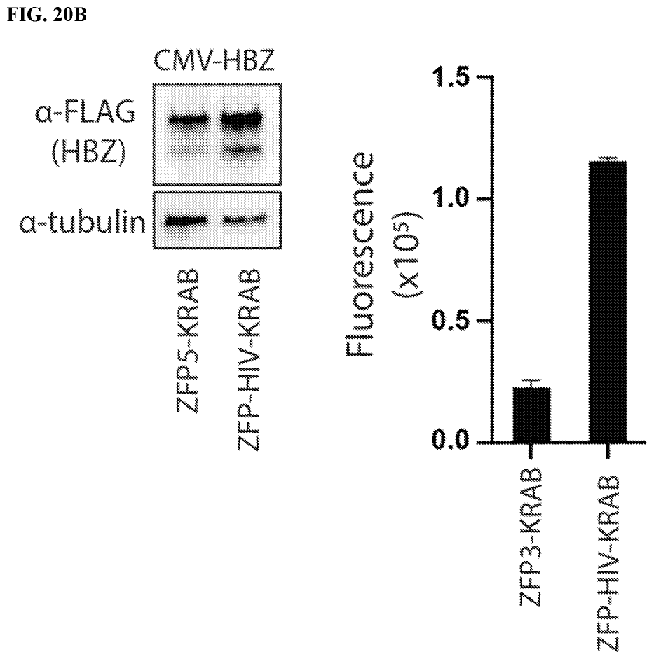

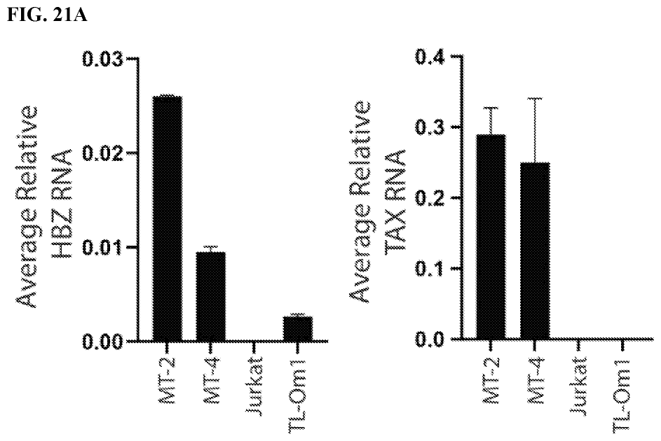

To assess if the ZFP repressors reduced HBZ RNA and protein expression, an exogenous vector containing the 3′LTR driving the expression of the HBZ transcript was generated (LTR-HBZ), cloned out of the genome of the patient-derived TL-Om1 ATL cell line (FIG. 2B) (22). The ZFP repressors were transfected into HEK293 cells with the LTR-HBZ vector, and the expression of the spliced and nascent HBZ RNAs and ZFP mRNAs were readily detectable by RT-qPCR (FIG. 7C and FIG. 9C) as well as the FLAG-tagged or Myc-tagged HBZ and ZFP proteins, respectively (FIG. 2C). When compared to the ZFP-HIV-KRAB control, potent suppression of the spliced HBZ RNA was observed with HTLV-ZFP-3-KRAB and 5-KRAB (>99%) (FIG. 2B) and HBZ protein (FIG. 2C), which corroborated the luciferase reporter data (FIG. 2A). The ZFP5-KRAB had no significant effect on the levels of HBZ from a CMV-HBZ vector (FIG. 20A). However, upon electroporation into a Jurkat cell line, the ZFP3-KRAB had a non-specific restrictive effect on growth (FIG. 20B) that was not observed with the ZFP5-KRAB and, as a result, the ZFP5-KRAB was selected for further characterization.

### Transient HBZ Suppression by a ZFP Repressor Reduces ATL Cell Line Proliferation

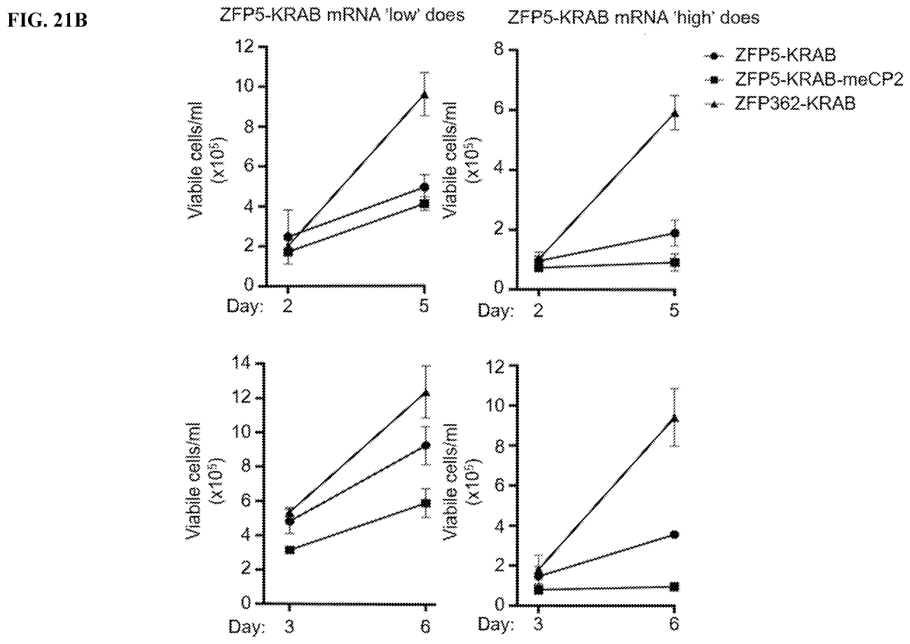

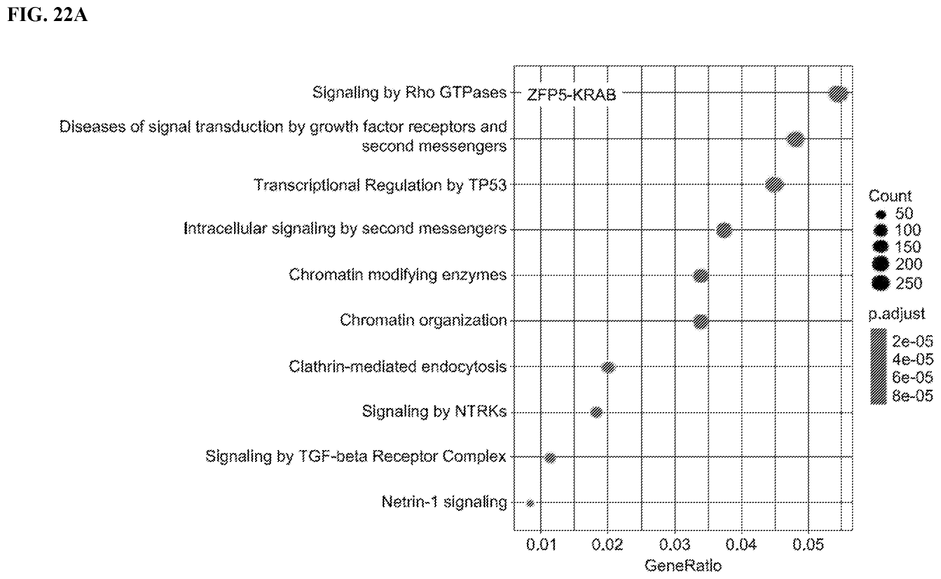

To determine if the ZFP repressors inhibited the proliferation of ATL, the ZFP vectors were tested in a patient-derived cell line, TL-Om1 cells. These cells have been well-characterized to have a single HTLV-I proviral integrant (18), and positive for HBZ but negative for Tax expression (23) (FIG. 21A) as a result of hypermethylation of the 5′LTR (24). As all primary ATLs express HBZ (12), these features make the TL-Om1 cells an ideal representative model for studying the anti-proliferative effects of the identified anti-HBZ ZFP repressors.

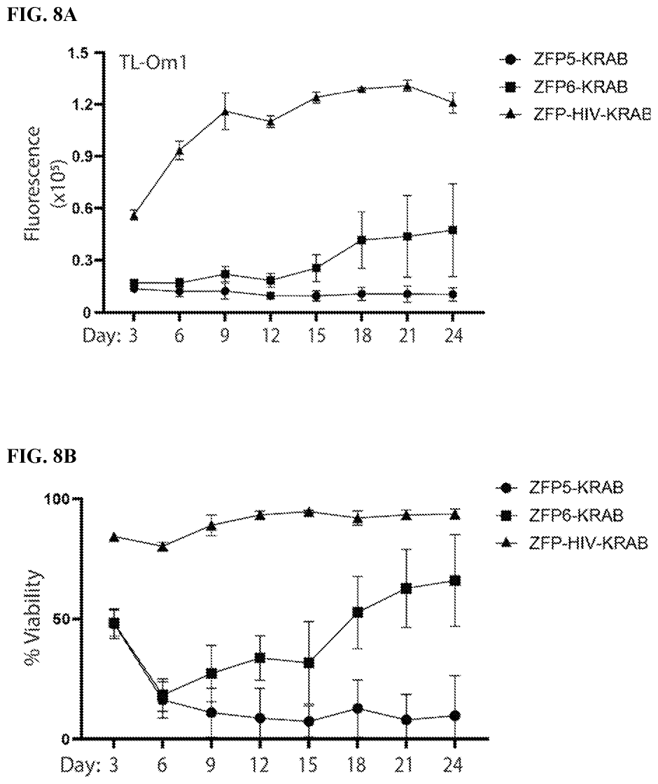

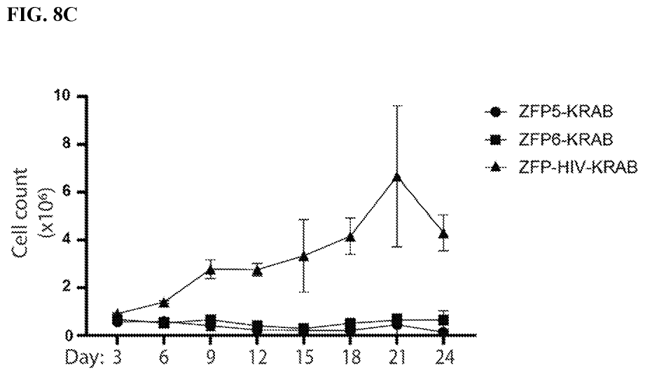

The ‘potent’ ZFP5-KRAB repressor was compared to the ‘weak’ ZFP6-KRAB for anti-proliferative effects. The expression vectors were electroporated into the TL-Om1 cells, and ZFP5-KRAB caused a significant reduction in proliferation, viability and cell counts when measured over 24 days compared to ZFP-HIV-KRAB (FIGS. 8A-8C). Although ZFP6-KRAB initially reduced proliferation and viability, the TL-Om1 cells recovered, providing evidence that the level of HBZ suppression could determine anti-proliferative effects.

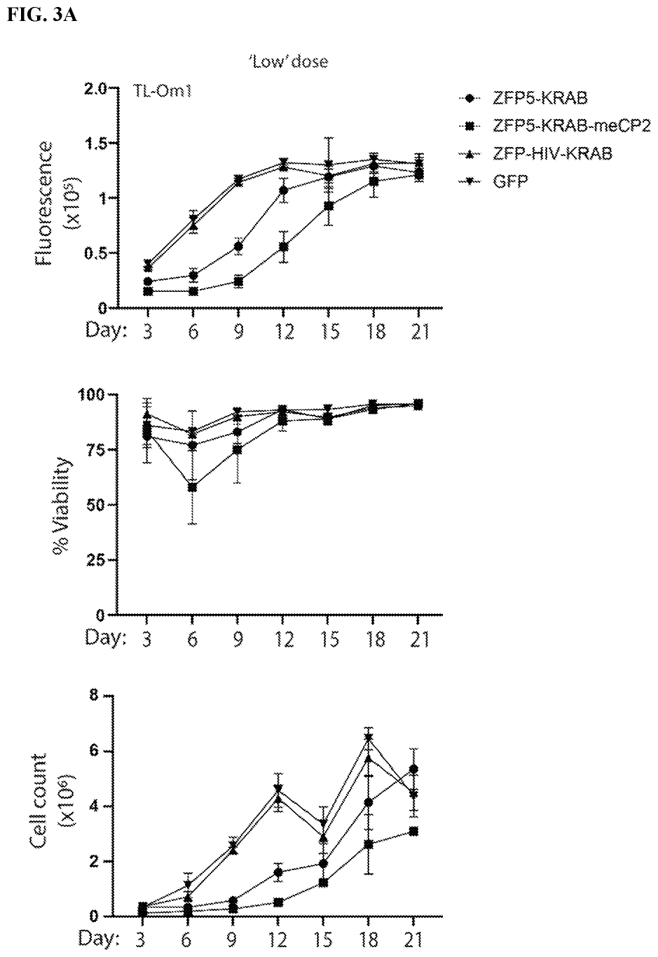

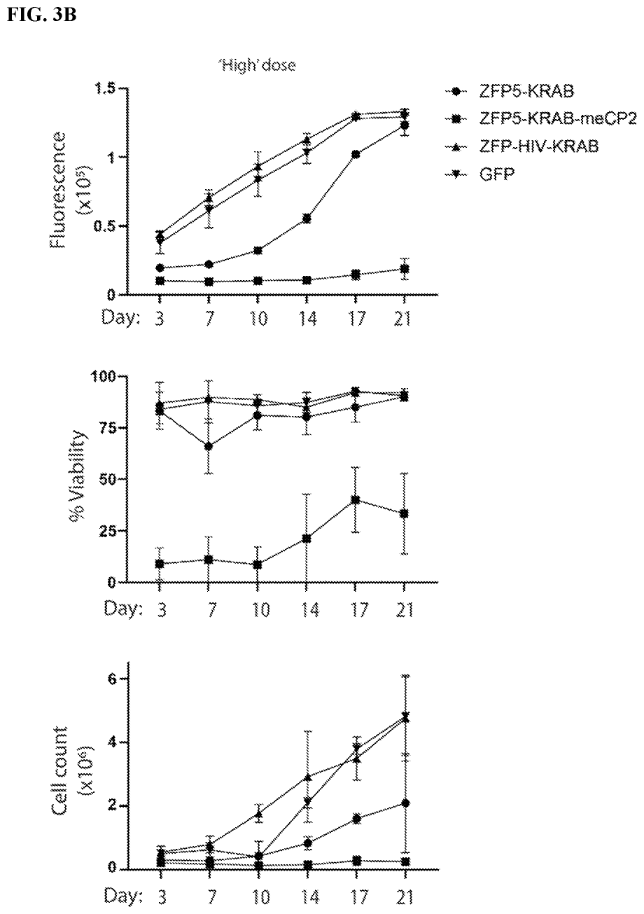

However, the TL-Om1 cells were generally negatively affected by the electroporation of DNA vectors into the cells (data not shown), which prevented further downstream analysis. Furthermore, transient expression of the ZFPs would be preferable for therapeutic development and mRNA is emerging as the nucleic acid of choice for such applications. Accordingly, the ZFP5-KRAB was generated as mRNA and electroporated in the TL-Om1 cells, which was efficiently delivered and well-tolerated (>90% GFP expression; data not shown). In the cells electroporated with ZFP5-KRAB mRNA, a clear reduction in TL-Om1 proliferation was observed compared to controls, although with no effect on cell viability over the 21 days study (FIG. 3A, FIG. 21B). Increasing the amount ZFP5-KRAB mRNA to a ‘high’ dose electroporated into the TL-Om1 cells did slightly elongate the suppressive effect, and some of the treated samples had reduced viability at day 7 (FIG. 3B). Based on the reduced viability observed in this study using DNA vectors (FIG. 8B) and the fluctuations in viability with the ‘high’ dose ZFP5-KRAB mRNA (FIG. 3B), it was thought that the potency or duration of HBZ suppression might be important to observe strong anti-proliferative effects.

### Potent ZFP Repressors Significantly and Specifically Reduced ATL Cell Line Proliferation

With this in mind, we designed several new versions of the ZFP5 with alternative repressor domains described to have more potent activity. These domains included a novel KRAB repressor ZIM3 (25), the current KOX1 KRAB fused to a methyl CpG binding protein 2 (meCP2) (26), or replacing the KRAB with a recently described fusion repressor, PAM (17) (FIG. 9A). The ZFP5 variants were transfected into HEK293 cells with the HBZ spliced Rluc reporter or LTR-HBZ vectors, and the ZFP5-KRAB-meCP2 showed comparable suppressive activity to the ZFP5-KRAB when detecting HBZ spliced Rluc levels (FIG. 9B), HBZ RNA levels (FIG. 9C), and HBZ protein levels (FIG. 9D). A ZFP5 without a KRAB domain was also tested to determine if steric hinderance at the promoter was causing HBZ suppression. The ZFP5 alone slightly suppressed promoter activity by ˜50%, and potent suppression was achieved by the KRAB domain, demonstrating a domain-specific effect (FIGS. 9B-9D). THE ZFP5-KRAB(ZIM3) suppressed activity by ˜50%, suggesting the ZIM3 KRAB was not contributing to suppression. The ZFP5-PAM was ineffective, likely from poor expression of the fusion protein. Overall, in these assays, the ZFP5-KRAB-meCP2 showed comparable activity to the ZFP5-KRAB and was selected for further characterization of its anti-proliferative effects.

The ZFP5-KRAB-meCP2 mRNA was electroporated at a ‘low’ dose into TL-Om1 cells and increased suppression of proliferation and cell counts compared to the ZFP5-KRAB (FIG. 3A). There was no significant effect on viability between the treated groups; however, there were fluctuations at the ‘low’ dose in viability in the ZFP5-KRAB-meCP2 treated cells at day 6. When the amount of electroporated ZFP5-KRAB-meCP2 mRNA was increased to a ‘high’ dose, there was a potent anti-proliferative effect and marked reduction in viability of the TL-Om1 cells compared to the ZFP5-KRAB or controls over the 21-day study (FIG. 3B).

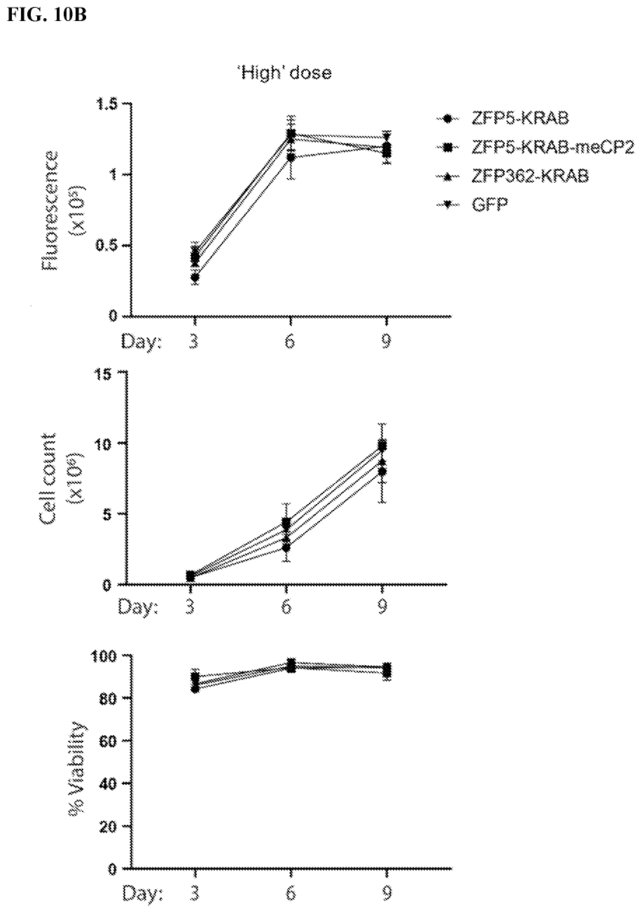

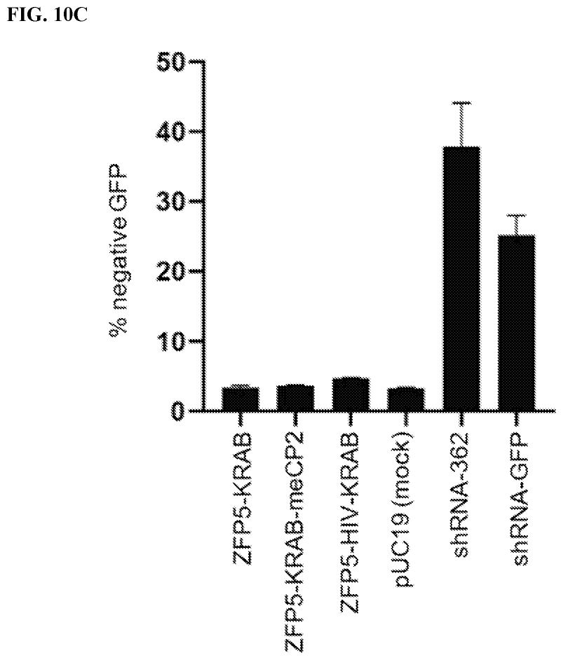

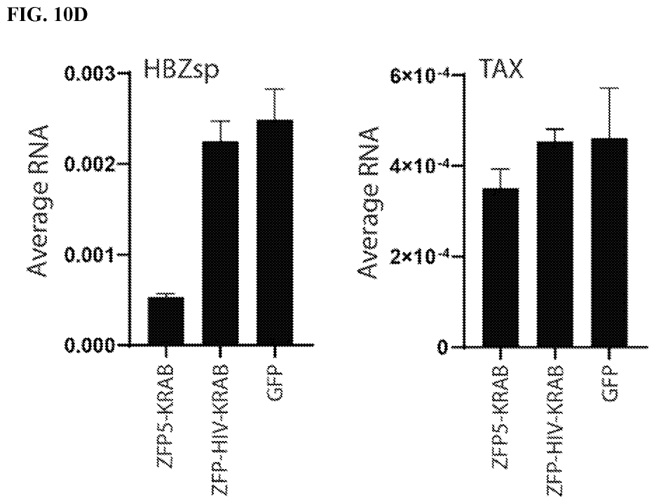

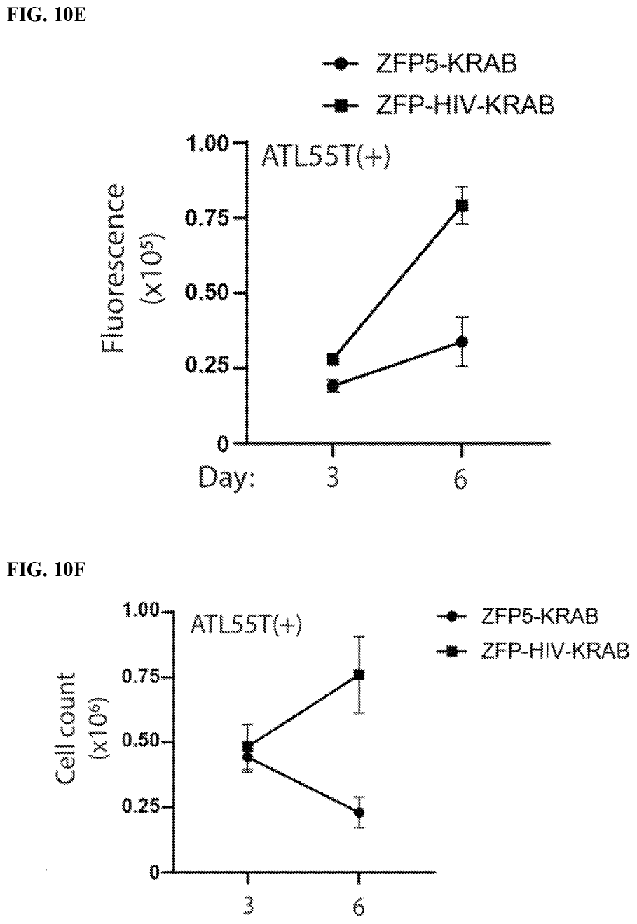

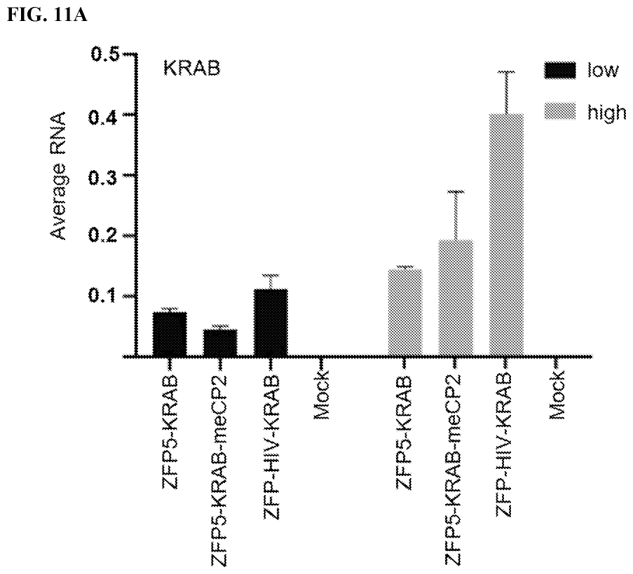

To determine if these effects were specific to a HTLV-I leukaemia cell line, these conditions were repeated in Jurkats cells, a non-HTLV-I leukaemia cell line. The ZFP5 repressors had no effect on the proliferation, cell count, or viability of these cells (FIGS. 10A-10B). Furthermore, to assess if the ZFP could affect LTRs from other retroviral vectors, the HTLV-targeted ZFPs were transfected into a reporter cell line with the HIV-1 LTR driving the expression of GFP and no effect on reporter levels was observed (FIG. 10C). Furthermore, suppression of the HTLV-I LTR in another ATL cell lines, ATL55T(+), likewise resulted in a reduction in HBZ RNA levels and proliferation (FIGS. 10D-10F). These data demonstrate that the anti-proliferative effects from the ZFP5 repressors were specific to an HTLV-I transformed cell line.

### The ZFP Repressors Affected HBZ Levels and Reduce HBZ-Induced CCR4

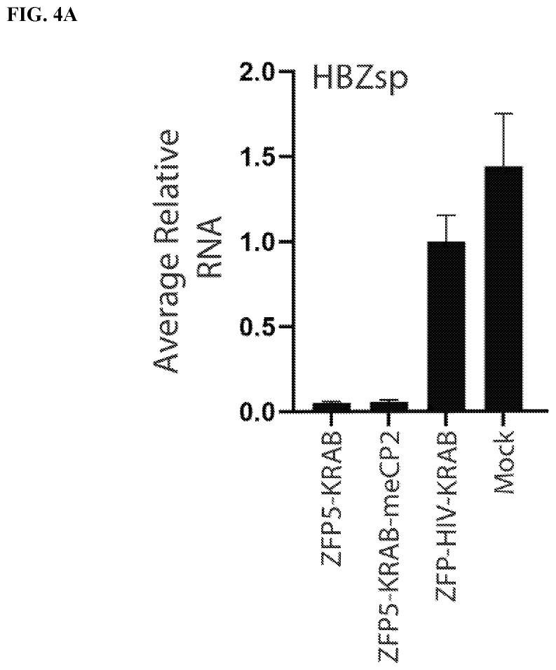

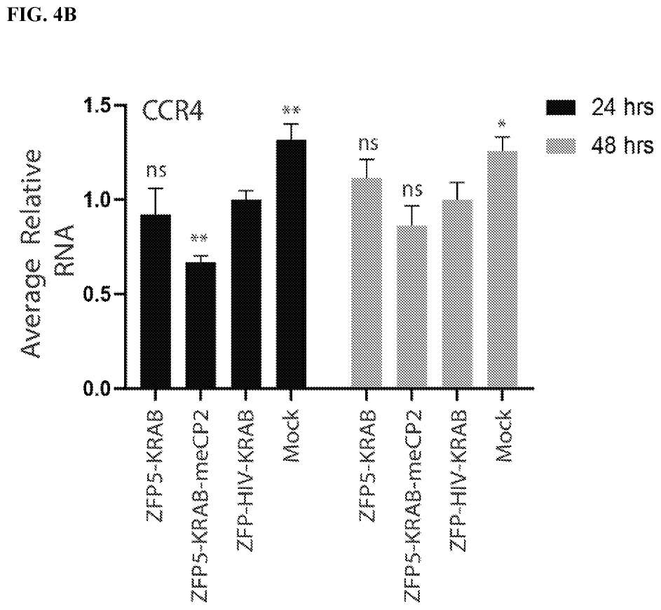

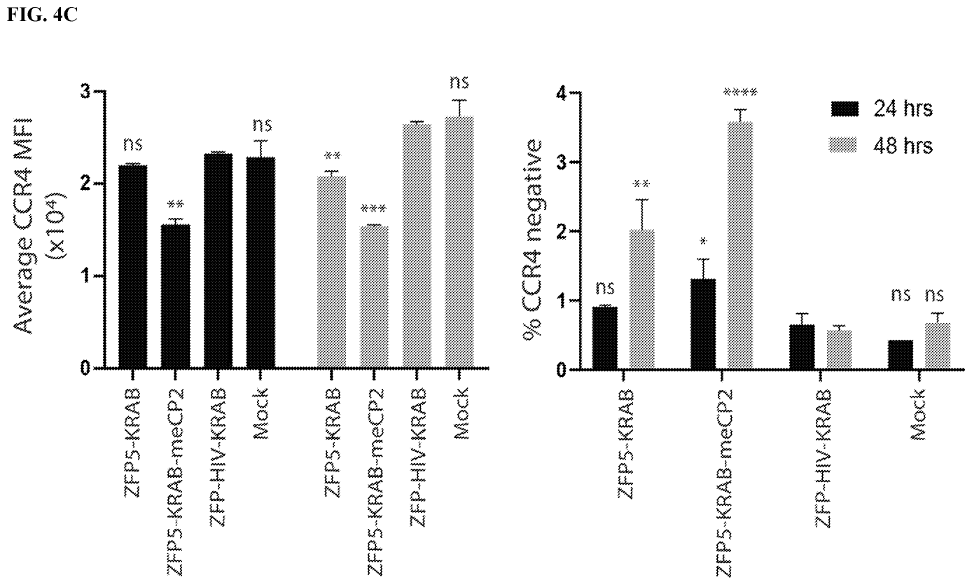

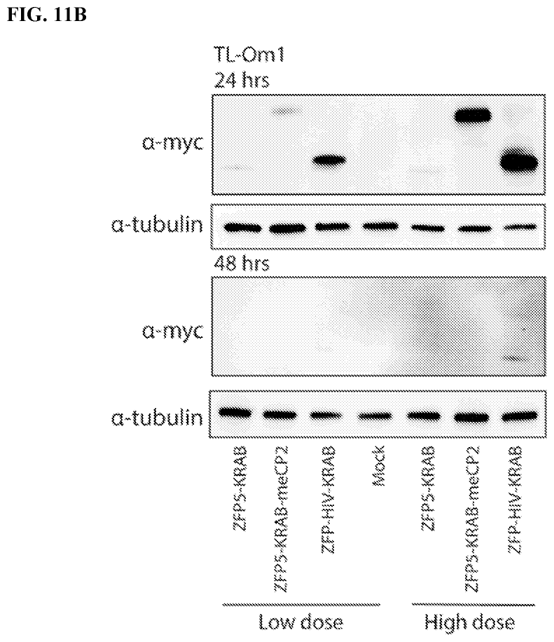

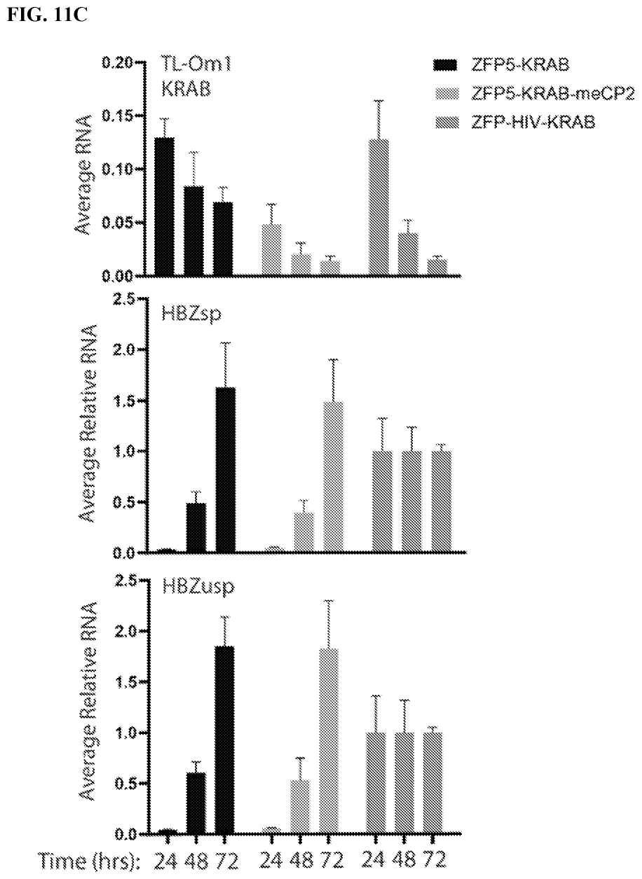

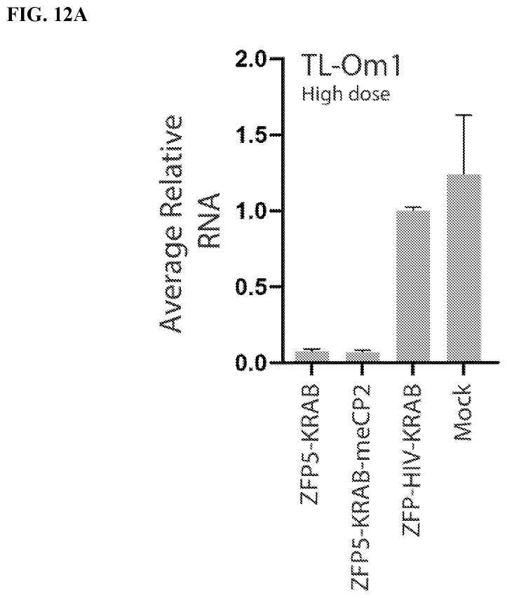

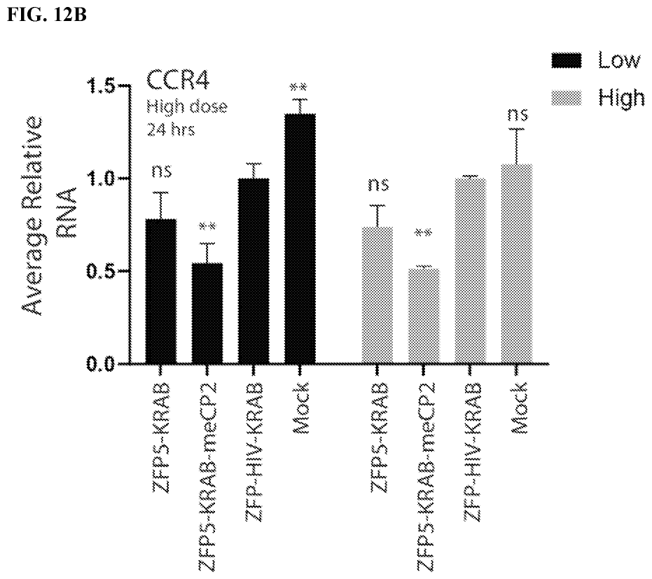

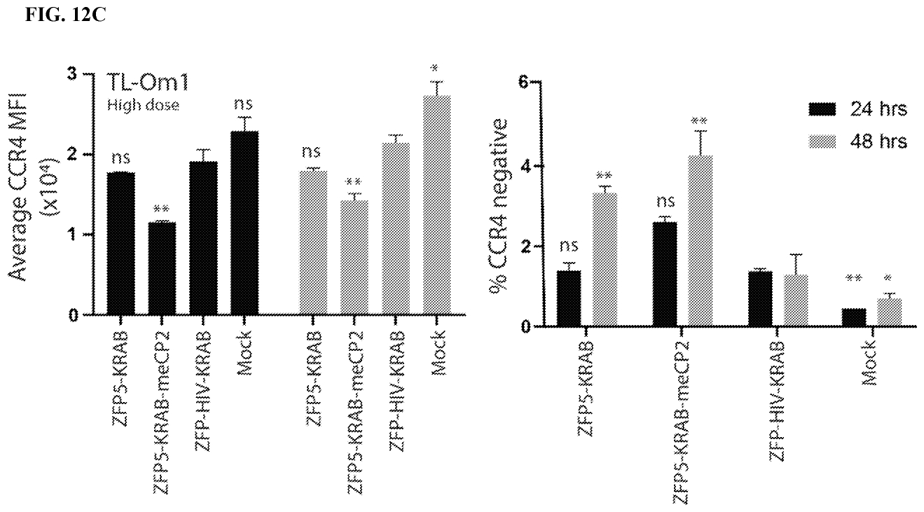

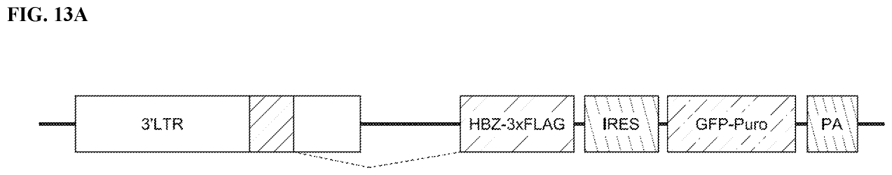

Next, the effect the ZFP5 repressors had on HBZ expression in TL-Om1 cells assessed. The ZFP5-KRAB and ZFP5-KRAB-meCP2 mRNA treated cells showed a comparable reduction in HBZ RNA levels (FIG. 4A and FIG. 12A). As expected, the detected ZFP5 repressor mRNA and protein rapidly reduced when measured over a 72 hr or 48 hr period, respectively, (FIGS. 11A-11C) and the declined in ZFP mRNA was mirrored by a concordant increase in HBZ RNA levels (FIG. 11C), confirming the ZFPs were affecting HBZ expression within its genomic context.

The HBZ RNA and protein affects a number of host genes in ATL and both upregulate surface receptor CCR4 expression (11). Interestingly, the CCR4 mRNA levels were significantly reduced to about 50% at 24 hrs but only in the ZFP5-KRAB-meCP2 treated cells (FIG. 4B). Even though CCR4 mRNA levels were re-established at 48 hrs, the amount of surface CCR4 detected by flow cytometry was reduced at 24 and 48 hrs (FIG. 4C). Increasing the amount of ZFP mRNA to the ‘high’ dose did not improve the reduction of HBZ or CCR4 levels (FIGS. 12A-12C).

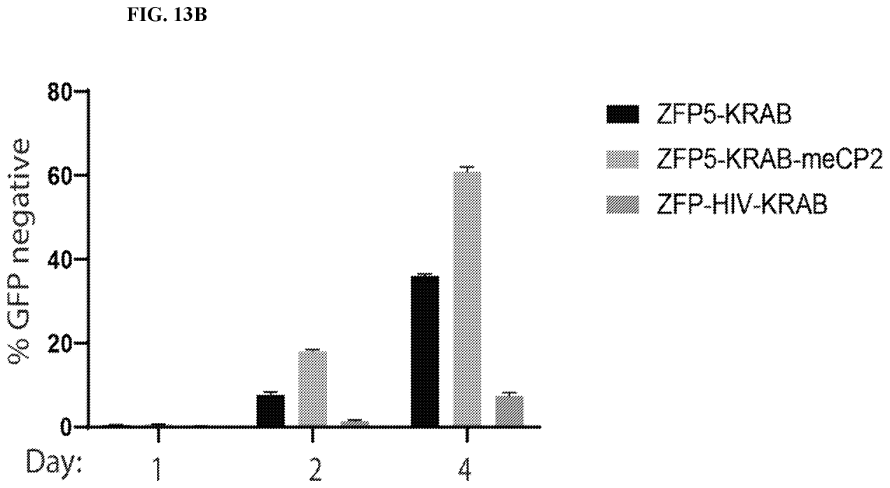

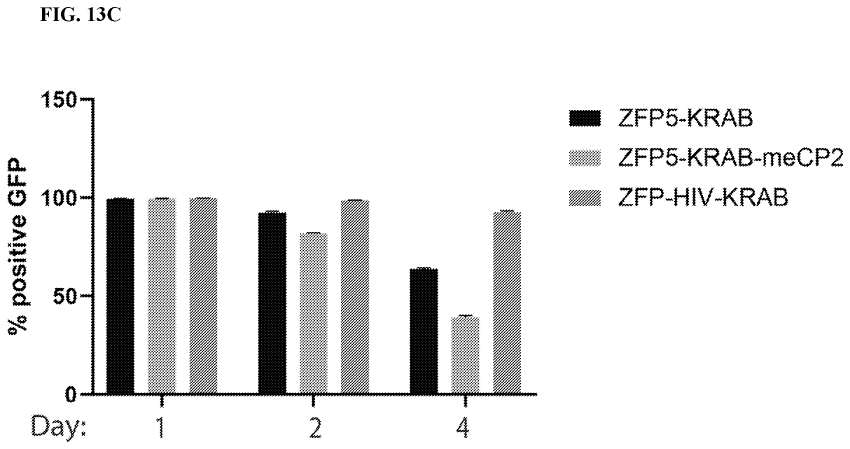

The ZFP5-KRAB and ZFP5-KRAB-meCP2 showed comparable levels of HBZ suppression in the TL-Om1 cells (FIG. 4A), but only ZFP5-KRAB-meCP2 was able to affect CCR4 levels (FIGS. 4B and 4C), suggesting that the ZFP5-KRAB-meCP2 was a more potent effector. In light of this observation, we surmised that the anti-proliferative effects were masking the extent of HBZ suppression. To assess the anti-HBZ effects of the ZFPs in the absence of proliferative factors, the ZFP mRNAs were electroporated into a Jurkat cell line engineered with an LTR-HBZ with an in-frame GFP reporter (FIG. 13A). In the absence of confounding anti-proliferative effects, the ZFP5-KRAB-meCP2 had a higher level of GFP suppression than the ZFP5-KRAB, demonstrating the ZFP5-KRAB-meCP2 was a more potent repressor (FIG. 13B). Overall, these data demonstrate that the ZFPs reduced HBZ mRNAs levels in TL-Om1 cells, and the ZFP5-KRAB-meCP2 was a more potent effector that significantly affects downstream HBZ-induced gene expression.

### the ZFP Repressors Cause Cell Cycle Arrest and Activate Apoptotic Pathways

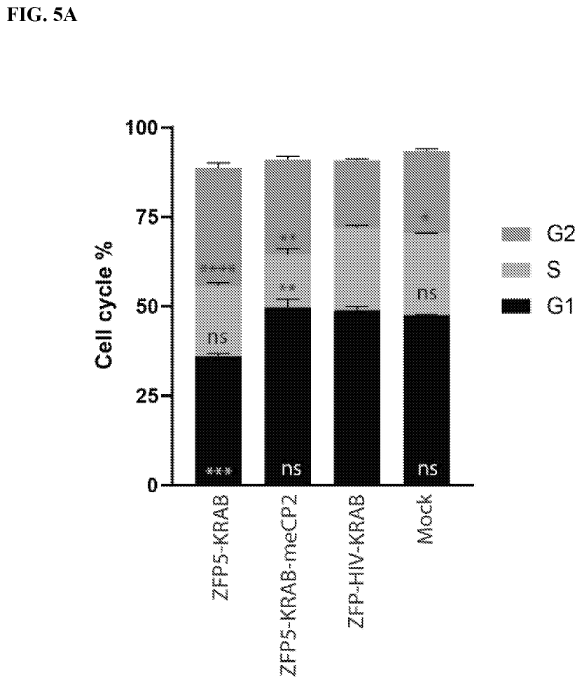

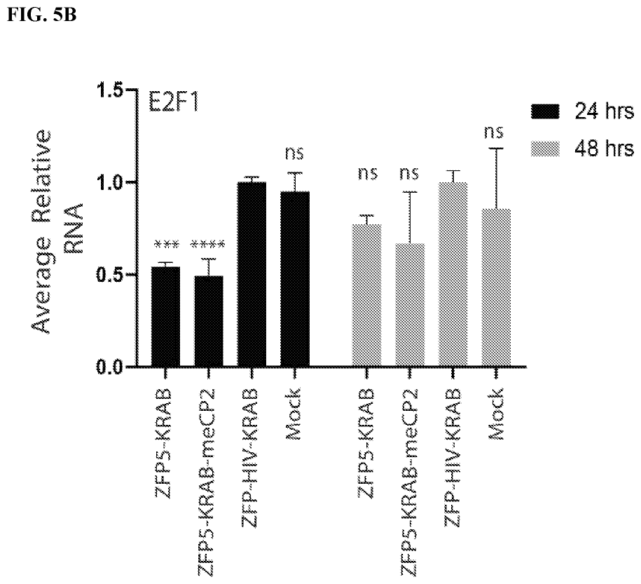

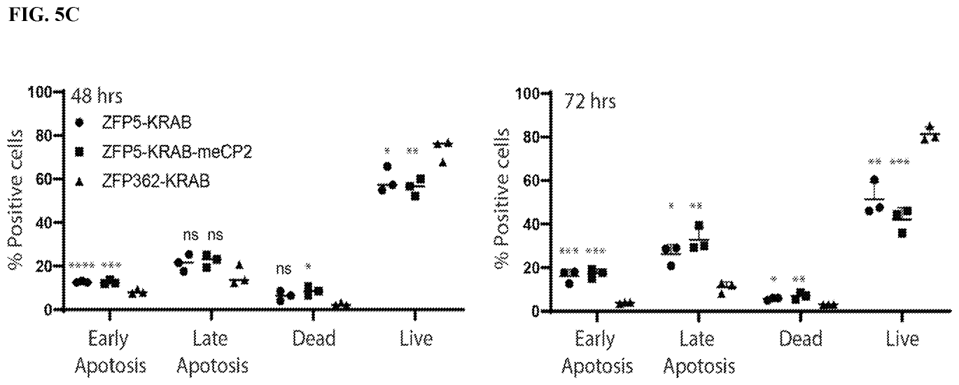

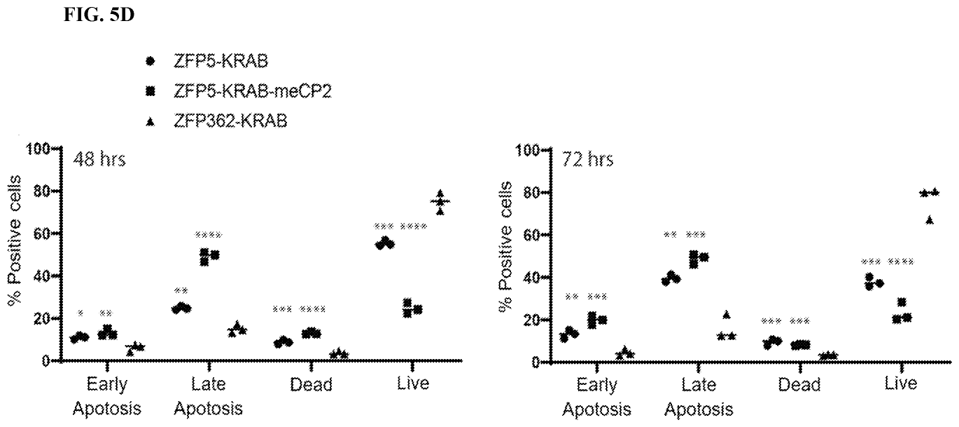

To better understand the mechanisms behind the anti-proliferative effects, a cell cycle arrest assay was performed. At 24 hrs post-electroporation, the ZFP5-KRAB was able to cause an increase in G2 phase and a reduction in G1, suggesting the inhibition of HBZ was causing G2 arrest in the TL-Om1 cells (FIG. 5A). Notably, the ZFP5-KRAB-meCP2 resulted in a different arrest profile, resulting in a likewise increase in G2 phase, although to a lesser extent than the ZFP5-KRAB, and a reduction of cells in S phase. The HBZ RNA is known to upregulate the transcription factor E2F1, which is a well-known driver of cell cycle progression (27). The levels of E2F1 mRNA were reduced at 24 hrs in the ZFP treated TL-Om1 cells (FIG. 5B), further demonstrating the ZFPs were affecting cell cycle factors induced by HBZ.

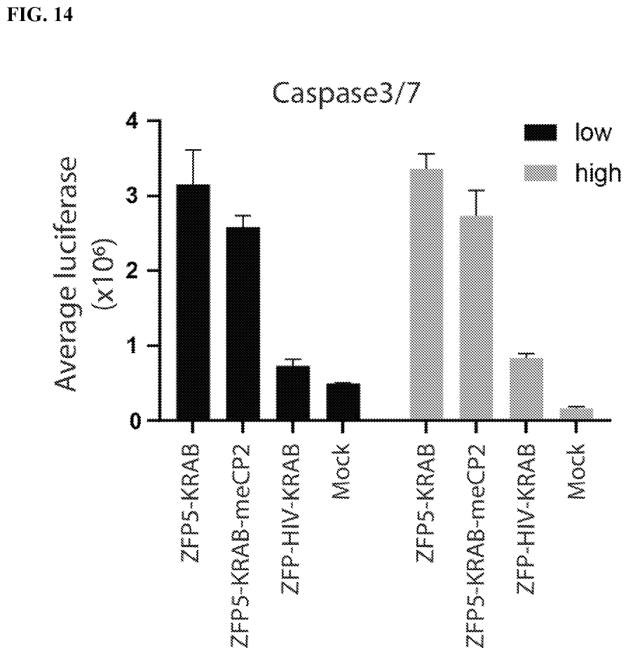

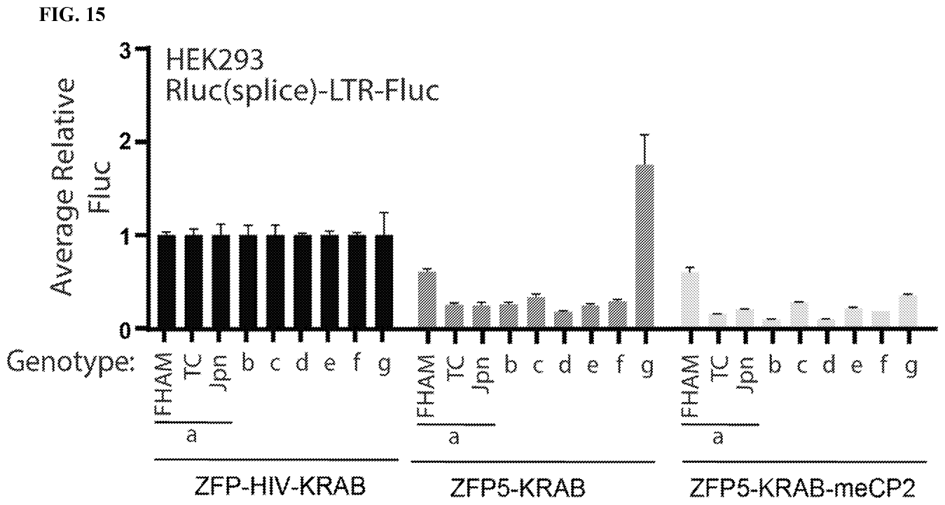

Induction of apoptosis by the ZFPs was then assessed. When determining the activation caspase 3/7 activity, the ZFP repressors induced activity in the TL-Om1 cells to comparable levels even when using a ‘low’ or ‘high’ dose (FIG. 14). Annexin V/PI staining revealed that at the ‘low’ dose, both ZFP repressors induced a modest but equitable induction of late-stage apoptosis at 48 and 72 hrs (FIG. 5C). However, at the ‘high’ dose, TL-Om1 cells receiving the ZFP-KRAB-meCP2, strongly induced late-stage apoptosis at 48 hrs compared to the ZFP5-KRAB (FIG. 5D), which was comparable at the 72-hr time point. Collectively, these data demonstrate the anti-HBZ ZFPs induced anti-proliferative effects are operative through cell cycle arrest and the induction of apoptosis in a ATL cell line.

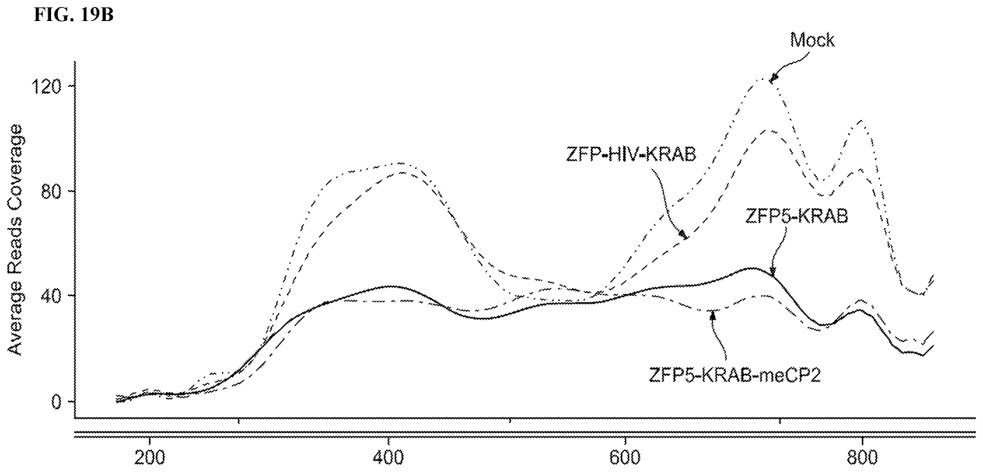

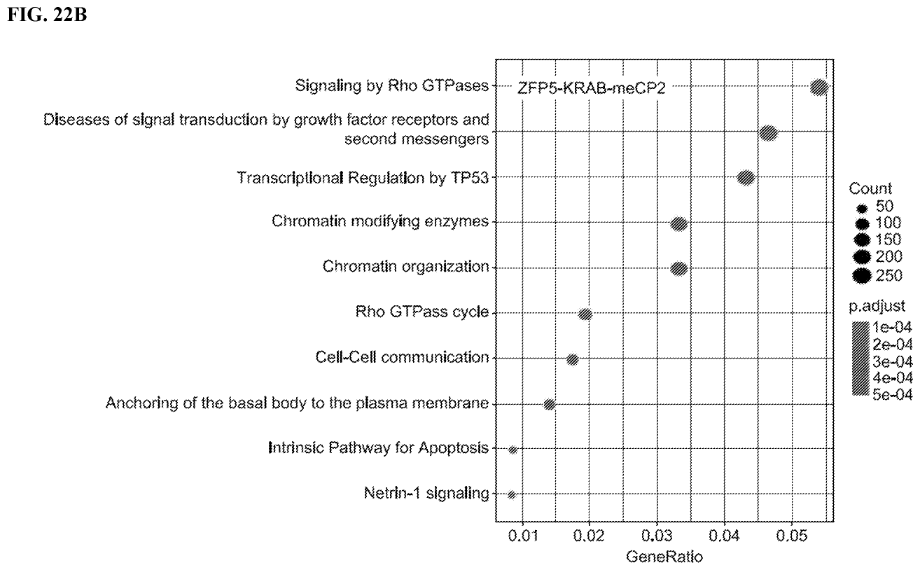

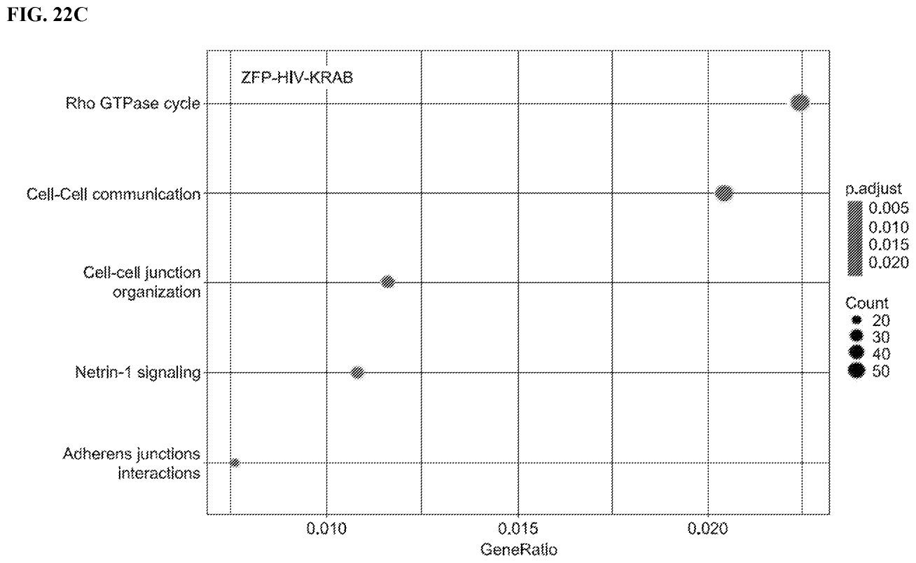

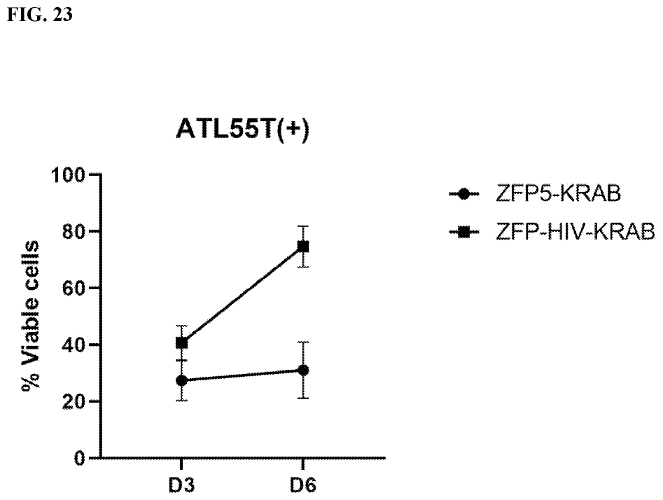

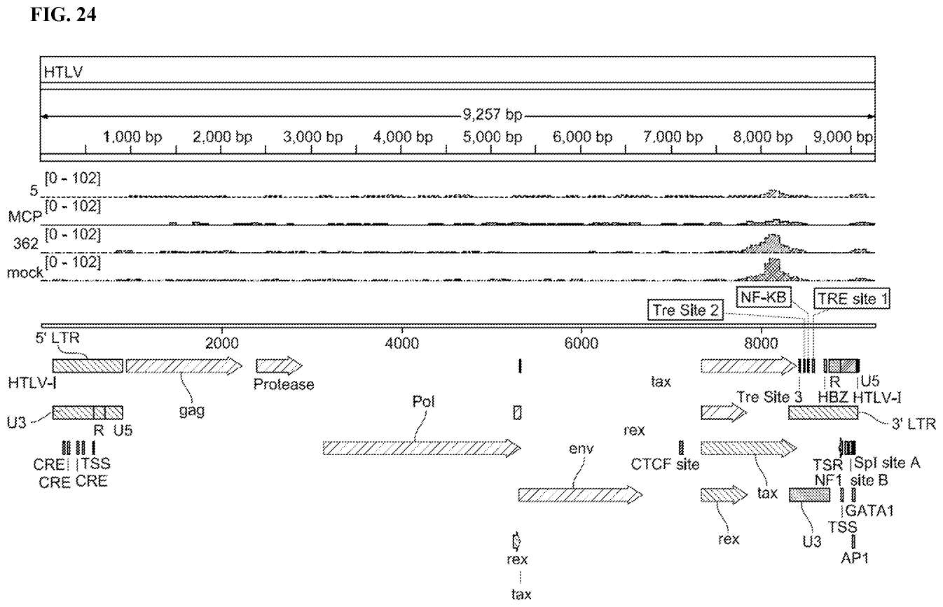

To demonstrate a mechanism of chromatin remodelling at the LTR by the anti-HTLV ZFPs, treated TL-Om1 cells were subjected to ATAC-seq (32). Briefly, chromatin is exposed to Tn5 transposase and euchromatin regions at transcriptional active genomic sites are more accessible to transposase tagmentation. Treatment with the ZFP5-KRAB or ZFP5-KRAB-meCP2 resulted in reduced reads across the HTLV-I genome (FIG. 19A) and a reduction in nucleosome-free regions in the LTR (FIG. 19B). Furthermore, pathway analysis was performed for differential chromatin accessibility across TSS sites. P53 is functionally inhibited by HBZ and a top hit was genes associated with p53 transcription regulation in the ZFP treated samples, which was not observed in the ZFP-HIV-KRAB treated cells (FIGS. 22A-22C), suggesting anti-HBZ ZFPs are affecting genes downstream of p53.

### Anti-HBZ ZFP Repressive Activity is Conserved Across HTLV-I Genotypes

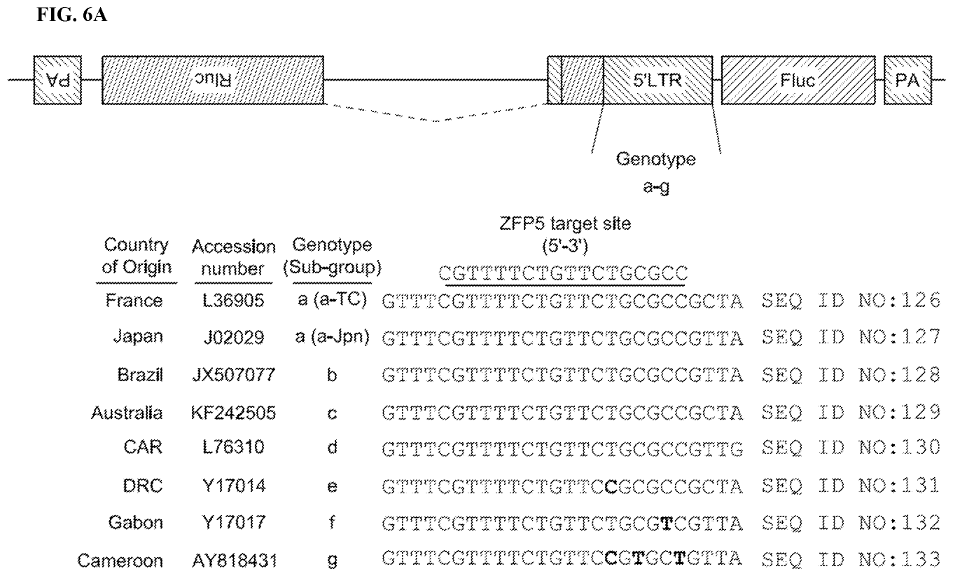

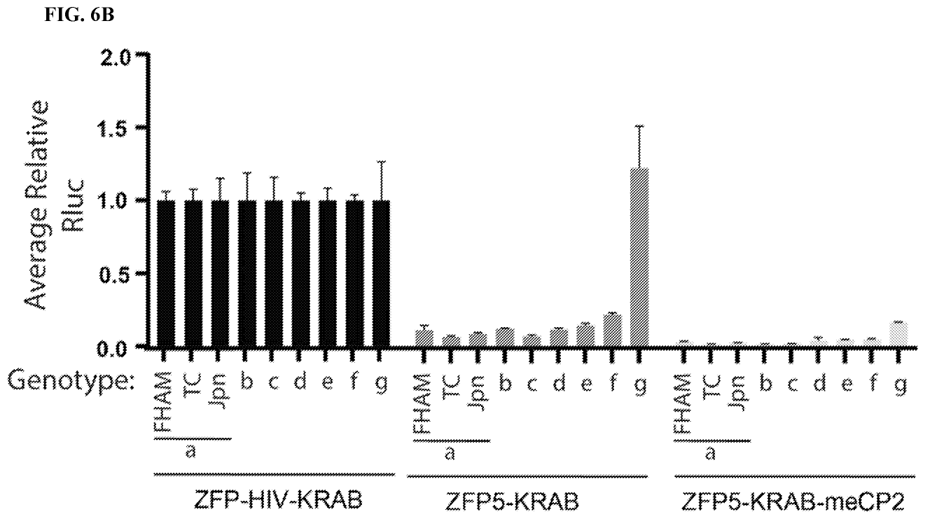

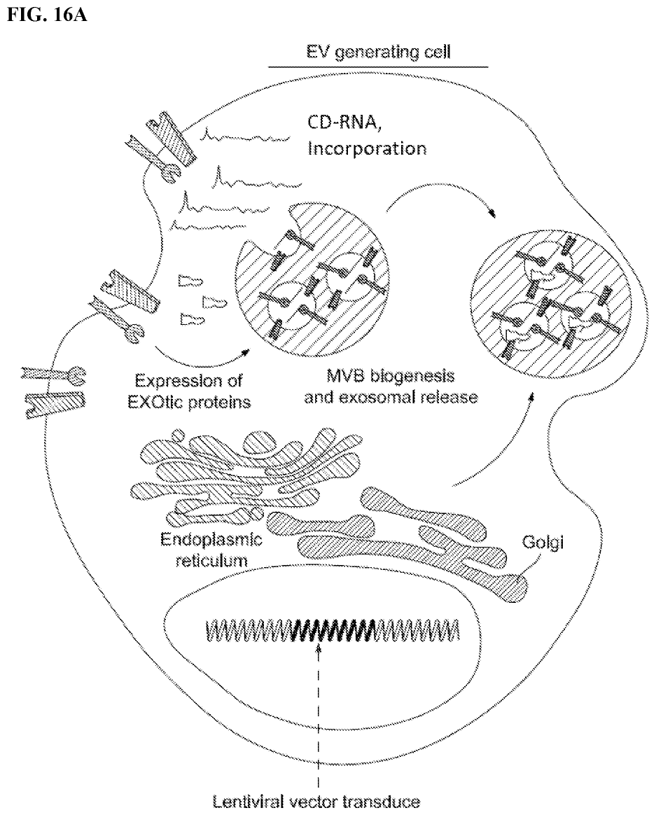

Lastly, the ZFPs were designed to target conserved sites within the LTR to ensure activity against a wide-range of HTLV-1 genotypes. The reference LTR sequence of each global circulating genotype (a-g) was inserted upstream of the HBZ start site in the spliced Rluc luciferase reporter vector (FIG. 6A). The ZFP5 target site is fully conserved within genotypes a-d, single mismatches in genotypes e and f, and a triple mismatch in genotype g. The ZFP5 expression vectors were transfected into HEK293 cells with the spliced Rluc luciferase reporter vectors of each genotype, and the ZFP5-KRAB successfully knocked down each genotype, except for the triple mismatch genotype g (FIG. 6B and FIG. 15). The ZFP5-KRAB-meCP2 inhibited luciferase expression from all genotypes. These data suggest that the ZFPs should affect HBZ expression in a wide range of circulating HTLV-I genotypes.

### Discussion

Current approved treatments for ATL have limited improvements on patient survival, and ATL is considered refractory to chemotherapy and radiation therapy, promoting the development of novel therapeutics. Here we describe a novel molecular therapy against a potential gene driver of ATL, the anti-sense HBZ gene, which is functional against the LTR of a broad range of HTLV-I genotypes. Other knockdown studies have shown that a reduction in HBZ results in reduced proliferation in the TL-Om1 cells as well as a number of in vitro HTLV-I transformed cells (MT-1, SLB-1, PBLACH) (13,14), suggesting that the anti-HBZ ZFP repressors will affect a wide range of ATL samples.

A zinc-finger nuclease that introduce mutations into the LTR through nuclease activity has been shown to reduced HTLV-I associated tumor growth in vitro and in vivo (28). However, no further characterization of the mechanism of inhibition was performed. In the knockdown studies, reduced proliferation in HTLV-I cell lines was observed (13,14), but no reduction in viability (13). The ZFP repressors showed a rapid and strong induction of late-stage apoptosis and, at the ‘high’ dose, the ZFP5-KRAB-meCP2 resulted in a stark reduction in viability (FIGS. 3A-3B and 5A-5D). This difference in observation may reflect the potency of HBZ inhibition, where previous studies knocking down the HBZ RNA and protein levels was limited, and may be insufficient to induce cell death (13). However, some of the cell lines in these studies also expressed a functional Tax oncogene, which may affect anti-proliferative effects and additional studies will be needed to determine the threshold of ATL oncogene suppression required to induce cell death.

Still, it is unclear why ZFP5-KRAB-meCP2 reduced viability at the ‘high’ dose as caspase 3/7 activation was similar to the ZFP5-KRAB (FIG. 14), but a more potent and rapid induction of late-stage apoptosis was observed (FIG. 5D). Furthermore, the ZFP5-KRAB-meCP2 caused S-phase arrest which has been linked to apoptosis, and these observations may suggest this modality is more effective at committing the TL-Om1 cells to programmed death. Furthermore, the ZFP5-KRAB-meCP2 at the ‘high’ dose was the only system that substantially reduced viability, (FIG. 3B), and affected the downstream HBZ factor, CCR4 (FIGS. 4A-4C), suggesting a possible threshold for reversing HBZ-induced factors involved in maintaining the tumor state. The HBZ protein has proapoptotic function while the HBZ RNA has pro-survival effects (10), and this apparent threshold may support the ‘oncogenic shock’ model for this viral oncogene (29), where the reduction of the oncogene's pro-survival signals are outbalanced by the proapoptotic signals, committing the cell to a death pathway. Further studies elucidating this mechanism would assist in a more rational design of anti-HBZ modalities.

An alternative explanation may be warranted. The ZFP5-KRAB-meCP2 was selected as the meCP2 component may elicit epigenetic changes at the target promoter (30), allowing for sustained, if not permanent, silencing. The ‘high’ dose ZFP5-KRAB-meCP2 may elicit a sustained suppressive effect on HBZ, resulting in cell death. Further studies should explore this possibility, and, if so, epigenetic modulators, like those developed for ‘block and lock’ strategies for HIV (17,31), could be applied to the inhibition of HBZ as an ATL treatment approach. Regardless whether the effect was through potency or duration, the unique observation presented here suggests that the ablation of HBZ expression may be a viable means to eliminate HBZ-driven malignances.

HBZ has been implicated in a wide range of pathological features of ATL. The upregulation of CCR4 is known to enhance ATL proliferation and trafficking (11), especially migration to the skin (2). A reduction in CCR4 surface levels was observed when treating the cells with the anti-HBZ ZFPs, which may reduce HBZ-mediated pro-migratory and proliferative effects. Furthermore, the HBZ protein is associated with bone degeneration through the RANKL/c-Fos pathway (32), and the HBZ RNA is known to augment Survivin (10), a factor involved in chemoresistance and a feature of ATL (33,34). Therefore, targeting HBZ with the ZFP repressors may be a means to modify a spectrum of ATL disease features.

HTLV-I has been associated with another disorder, HTLV-I associated myelopathy/tropical spastic paraparesis (HAM/TSP), which is a progressive, chronic neurological disorder that has been associated with HBZ and Tax expression (35). Furthermore, there are currently no therapeutics that can suppress Tax-mediated productive infection in active HTLV-I. The ZFP5 did affect 5′LTR activity in reporter assays (FIG. 2A), however, we observed no significant suppression of Tax transcripts in the ATL55T(+) cells, demonstrating the repressive activity of the ZFPs is 3′ LTR specific. Still, novel ZFP repressors specifically designed to inhibit the 5′ LTR could be developed to affect Tax expression, an important factor in active infection and HAM/TSP.

Delivery of gene therapies remains a challenge within the field. Although viral vectors, such as T-cell tropic adeno-associated viral vectors (AAVs), may be an option, the sustained expression of the ZFPs in potential off-target tissues through systemic administration would not be advisable and antibody responses to the vector would preclude repeat dosing. There has been significant interest in the current development of mRNA and lipid nanoparticle (LNP) formulations because of the success of the COVID-19 LNP-based vaccine. Currently, T-cell delivery in vivo with LNPs is limited; however, there has been recent success with T-cell delivery in vivo (36), and the combination of the ZFP mRNA with an LNP formulation to target ATL cells could be an approach. More innovative solutions could explore extracellular vesicles (EVs) as an emerging delivery platform. EVs are a broad group of small, membraned nano-size products derived from the cell, which are biocompatible and non-immunogenic, and are being developed as a delivery system for therapeutic cargo (37). Recent work has demonstrated that ZFP activators can be transferred to recipient cells to activate an endogenous gene (38) as well as deliver a ZFP repressor targeted to HIV's LTR resulting in epigenetic repression of HIV after systematic administration in a humanized mouse model (17). Therefore, potential platforms compatible with systemic administration are available that could be a viable, druggable approach for clinical application of this novel modality.

In conclusion, described here is a novel ZFP repressor that can target the HTLV-I LTR and suppress the HBZ gene, resulting in the reduced proliferation of a patient derived ATL cell line. These data not only add to the growing body of evidence establishing HBZ a molecular driver and potential target in ATL, but encourages the further development of this modality to potentially treat HTLV-I associated malignancies.

### Example 2: EV Delivery of a Zinc Finger Protein to Direct Killing of Human T-Cell Leukemia Virus Type 1 Transformed Cancer Cells

### Introduction

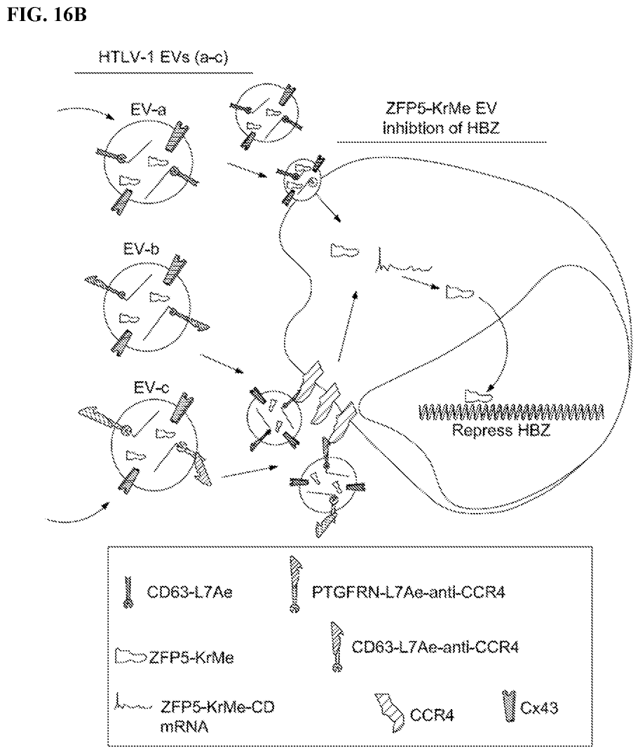

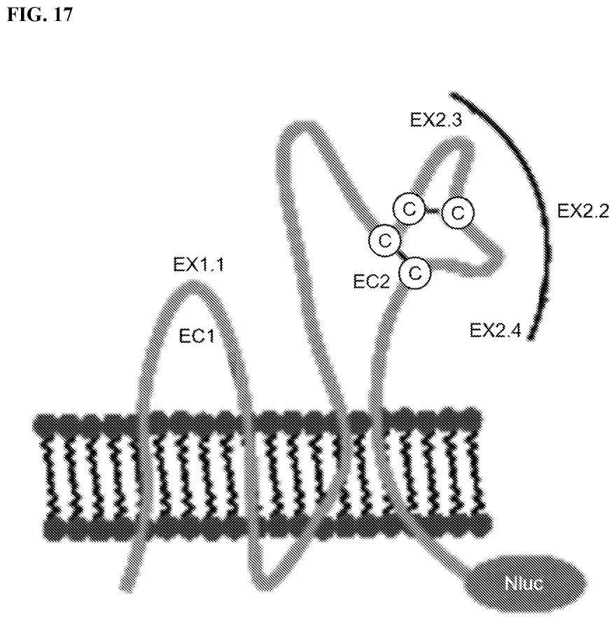

HTLV-1 infects T-cells (Yoshie, 2008 #4489) and the persistent expression of the HTLV-1 HBZ gene plays a part in the oncogenic transformation and maintenance of HTLV-1-infected cells in vivo, while also inducing increased CCR4 expression known to augment disease pathology (Matsuoka, 2011 #4488). A methodology that can target the specific inhibition of HBZ can lead to a loss of those cells transformed by HTLV-1 and presumably a cure for HTLV-1 associated disease. We show that HTLV-1 transformed T-cells can be specifically targeted and killed by a newly developed anti-HTLV HBZ gene targeted zinc finger protein repressor containing a fusion of KRAB and meCP2 epigenetic regulatory proteins (ZFP5-KrMe) delivered to virus transformed CCR4 over-expressing T-cells by targeted extracellular vesicles. We develop and characterize a highly innovative next-generation genetic therapy approach whereby human cells are engineered to produce anti-HTLV-1 ZFP packaged extracellular vesicles (EVs) for targeted killing of HTLV-1 transformed T-cells (FIGS. 16A and 16B).

### Approaches

An approach is used that combines the use of endogenous cell-derived EVs to deliver a anti-HBZ repressor to target and kill HTLV-1 transformed T-cells. We develop and characterize HTLV-1 HBZ directed ZFP containing EVs with and without a surface expressed anti-CCR4 antibody (Mogamulizuma)(Moore, 2020 #4451), which will target the effector EVs to CCR4 expressing T-cells (FIGS. 16A-16B). This technology allows for the conversion of any cell into exosome factories, containing the packaging of any desired RNA, by incorporating a CD63 fusion with the archaeal ribosomal protein L7Ae, which specifically binds to the C/D box RNA structure (Kojima, 2018 #3639). The resultant CD63-L7ae fusion binds those RNAs containing the C/D box embedded into the 3′-untranslated region (3′-UTR) of the candidate RNA, which results in the packaging of the desired RNA into the exosomes. The approach envisioned here utilizes ex vivo cell-derived EVs packaged with our newly developed HBZ specific Zinc Finger protein ZFP5-Me to target and kill HTLV-1 provirus infected cells by targeted epigenetic repression of HBZ (FIGS. 16A and 16B).

### Zinc Finger Repression of HBZ Results in Specific Death and Loss of HTLV-1 ATL Cell Line Viability.

We screened 9 ZFPs fused to the KRAB epigenetic repressor targeting vector with an LTR-driven HBZ gene, the gene required for oncogenic addiction in HTLV-1 transformed cells (Zhao, 2016 #4442), and found two candidate ZFPs, ZFP3 and ZFP5 which potently repressed HBZ RNA and protein (FIGS. 2A-2C). Both ZFP3 and ZFP5 mRNA and recombinant protein were readily detected in the treated cells. The levels of HBZ repression by ZFP3 and ZFP5 correlated with the reduction of viability in an ATL patient-derived cell line, TL-Om1 cells. Notably, ZFP5 was able to reduce proliferation of the HBZ-driven TL-Om1 cells for 19 days. To determine if a methylation-based inhibitor is more effective against HTLV-1 HBZ, we generated a modified ZFP5-KRAB to contain the methyl CpG binding protein 2 (meCP2). Notably, the ZFP5-KRAB-meCP2 outperformed ZFP5-KRAB and robustly repressed TL-Om1 cell proliferation and viability for 21 days.

There are many genotypes of HTLV-1. To determine how ubiquitous the targeting ZFP5 is across these genotypes, we developed an HBZ spliced luciferase reporter, expressed by LTRs from every genotypes a-g (FIGS. 6B, FIG. 15). We found that ZFP5-KRAB can repress all of the variants on the planet, except the triple mismatched Cameroon variant (genotype g), but ZFP5-KRAB-meCP2 can repress all known variants (FIGS. 6B, FIG. 15).

Collectively, these data demonstrate the ZFP5 fused with either KRAB or KRAB-meCP2 are robust inhibitors of HTLV-1 HBZ expression and induce cell death in HTLV-1 transformed cells. Engineered EVs contain and deliver these ZFP repressors of HBZ to HTLV-1 transformed CCR4 over-expressing cells. These data prove that we have the therapeutic modality necessary to target and inhibit HBZ expression, which is compatible with our validated ZFP delivery platform, in order to kill transformed oncogenic cells.

### Receptor Targeted EVs

Exosomes produced from the EXOtic system containing ZFP5-KRAB-meCP2 transcripts are developed to specifically target and kill HTLV-1 transformed cells. An antibody targeted to CCR4 (Mogamulizuma)(Moore, 2020 #4451) can be embedded onto the surface of the EVs to target the EVs specifically to high CCR4 expressing T-cells. EVs alone can be taken up by cells in a non-specific manner, but may be taken up by cells similar to their origin (23). One means to bias EV uptake to a particular cell type is by generating EVs that have a specific receptor agonist, single-chain fragment variable (scFv) or nanobodies, embedded into the extracellular membrane of the CD63 EV-associated protein. Towards this goal, we first determined the optimal extracellular loop and position within CD63 to embed the targeting protein. Results show that one optimal extra-cellular loop in CD63 to embed antibodies is loop 2 (EC2) in the Ex2.4 configuration.

Development of Stable Lentiviral Transduced HEK293 Cells Expressing ZFP5-KRAB-meCP2-CD mRNA Packaged EVs

EVs packaged with ZFP5-KRAB-meCP2 are generated by fusing the CD RNA binding domain from the EXOtic system (7) to the 3′ end of each gene generate ZFP5-KRAB-meCP2-CD and cloning these genes along with Connexion 43 (Cnx43) into the pHIV7GFP lentiviral vector containing CD63-L7ae; described by our group in (8). The resultant lentiviral vectors are generated and titered initially on HEK293 cells and used to make stable (pHIV7-EXOtic-ZFP5-KRAB-meCP2-CD; EV-a) (FIG. 16B) expressing HEK293 cells. The EVs (EV-a, FIG. 16B) generated from these stable cell lines are characterized for size, charge and numbers of EVs generated using the IZON qNano, Nanoparticle Tracking Analysis (NTA), and transmission electron microscopy (TEM), as was done by our group in (8). The relative numbers of ZFP5-KRAB-meCP2 packaged per EV using ddPCR are determined as described in (26), whereby the virus targeted gene (ZFP5-KRAB-meCP2) and a reference gene (RPP30) are measured and copy number is determined by calculating the ratio concentrations of the target to reference gene. Specific primer-probe pairs for each lentiviral vector and the reference gene are designed and the Bio-Rad QX200™ ddPCR system is utilized. Further, it is imperative to determine the content of the EVs, including the presence of any recombinant ZFP5-KRAB-meCP2-CD packaged into the resultant EVs, as our previous studies have found both mRNA and protein packaged in EVs (8). The EV protein content is determined by western blot for the various known EV markers and the presence of recombinant ZFP5-KRAB-meCP2 determined by anti-myc (ZFP5 contains a myc tag) and validated by LC-MS (COH fee for service core facility). Collectively, these assays allow for the quantification of both mRNA and protein content of the resultant EVs (EV-a, FIG. 16B) generated from stable HEK293 producer cells.

### Determination of ZFP5-KRAB-meCP2 EVs Repression of HBZ Expression and Ability to Kill HTLV-1 Infected Cells.

To determine the ability of these stable transduced cells to produce EVs capable of inhibiting HBZ gene expression and killing HTLV-1 infected cells, the resultant ZFP5-KRAB-meCP2 or control nLuc EVs producing transduced 293HEK cells, are either (1) co-cultured using a transwell culture approach (27) with HBZ reporter cells, or (2) added to HTLV-1 infected TL-Om1 cells in an EV-concentration dependent manner (ranging from 0 EVs/cell to 3×10\hat{ }5 EVs/cell) and ability to kill cells determined by direct cell counts, fluorescence activated cell sorting for markers of cell death and apoptosis (BCL-2, CD95, and Caspase 3/7; BioRad FACS panel), and viability. Collectively, these studies determine the ability of stable transduced HEK293 cells to produce EV-a (FIG. 16B) and if the resultant EVs are functional in repressing HBZ expression and killing HTLV-1 infected cells in vitro.

Exosome production may further be enhanced (˜10× fold) using chemically defined EV boost from RoosterBio® (RoosterBio Inc.). Further, repression of CHMP4C and VPS4B by RNAi can bolster EV production (23). Thus, shRNAs to CMP4C and VPS4B may be engineered into the resultant lentiviral vectors.

Development and Testing of CCR4 scFv Containing EVs

CCR4 transformed oncogenic T-cells exhibit high CCR4 expression that is driven by the action of HBZ gene expression (Sugata, 2016 #4445). This allows for using CCR4 as a receptor to target therapeutic agents to HTLV-1 transformed T-cells. Various EV membrane proteins can be developed containing antibodies, nanobodies and single chain fragment variable (scFv) fragments (FIG. 17). Mogamulizuma is an anti-CCR4 antibody (Moore, 2020 #4451) that can target HTLV-1 infected CCR4 over-expressing cells. Two membrane fused EVs; EV-b containing the anti-CCR4 scFv Mogamulizuma fused to PTGFRN (ZFP5-KrMe-PTscR4) and EV-c containing the anti-CCR4 Mogamulizuma fused to CD63 (ZFP5-KrMe-CD63-R4) (FIG. 16B) are developed. While surface expression of the CCR4 targeted antibody facilitates targeting and uptake into CCR4 expressing T-cells, the EVs will also be taken up by non-CCR4 expressing cells. While one may be concerned that the non-HTLV-1 transformed cells will be killed when they non-specifically take up the respective EVs, we did not observe any killing in various preliminary studies in HEK293 cells by the action of ZFP5-KRAB-meCP2, indicating that non-specific uptake of the various EVs will most likely not prove problematic.

CCR4 scFv containing EVs (ZFP5-KrMe-PTscR4 and ZFP5-KrMe-CD63-R4) are generated and contrasted with ZFP5-KRAB-meCP2 and cell Nanoluc packaged EV controls. Both the control Nanoluc packaged EVs (nLuc) and ZFP5-KRAB-meCP2 packaged EVs (EV-b, FIG. 16B) are generated to contain surface expressed CCR4 scFv by incorporating the previously reported CCR4 scFv (Han, 2012 #4447) into PTGFRN (ZFP5-KrMe-PTscR4) and CD63 (ZFP5-KrMe-CD63-R4). Notably, PTGFRN has been shown to tolerate scFvs (Dooley, 2021 #4446) and we show here that the CD63 Ex2.4 locus can tolerate antibody and nanobody fusions (FIG. 17). The putative advantage to EV-b and EV-c is that these EVs should be capable of not only targeting CCR4 receptor expressing T-cells but also be able to deliver the HBZ repressive ZFP5-KRAB-meCP2 to kill viral transformed T-cells.

Lentiviral transduced stable EV-a producing cells are transduced with the pcDNA3.1 vector expressing either the PTGFRN-anti-CCR4 or the CD63-anti-CCR4 fusion proteins and puromycin select to generate the new stable EV-b and EV-c EVs, HEK293 producer cells. The EVs generated from these cells are characterized, relative to control HEK293 cell and nLuc packaged EVs, for size, charge and numbers of EVs generated using the IZON qNano, Nanoparticle Tracking Analysis (NTA), and transmission electron microscopy (TEM) and determine the packaging efficiency of ZFP5-KRAB-meCP2 in each targeted EV. The relative incorporation of anti-CCR4 scFv into each EV is determined. Further, the ability of these EVs to bind and be taken up by CCR4 expressing cells using an innovative CCR4-uptake assay is completed, whereby we measure nLuc activity using FACS, as described in (Theodoraki, 2021 #4441). These studies allow for a molecular characterization of the respective EVs.

To determine the ability of the resultant EVs (EV-b and EV-c, FIG. 16B) to target and kill HTLV-1 transformed cells, CCR4 expressing TL-Om1 cells (Ferenczi, 2002 #4452) are be exposed, in varying concentrations (ranging from 0 EVs/cell to 3.0×10≡exosomes/cell. The exosome exposed cells will be assessed for metabolism (AlamarBlue assay), cell viability (trypan staining) and cell survival by direct cell count. The EV treated cells are characterized for CCR4 expression by FACS. To determine the relative killing of HTLV-1 transformed cells by those EV developed here, the EV treated cells are assessed using an apoptosis and caspase assay as described in (Kabakov, 2018 #4462) as well as western blot analysis to determine repression of HBZ and determination of p53 activation (Nakagawa, 2014 #4464). These studies determine the ability of the various stable HEK293 EV producing cell generated EVs (EV-a, EV-b and EV-c) to deliver functional ZFP5-KrMe and target and specifically kill CCR4 expressing cells as well as provide insights into the mode of cell death resulting from EV treatment.

### the Effects of CCR4 Mutations on Anti-CCR4 EV Cell Binding.

The chemokine receptor CCR4 has two natural ligand agonists, MDC (CCL22) and TARC (CCL17). Binding of these agonists to CCR4 are known to induce cellular chemotaxis also CCR4 receptor internalization (Ajram, 2014 #4454). However, Mogamulizuma binds the N-terminus of CCR4 but does not induce internalization (Duvic, 2015 #4463). Moreover, roughly one third of ATLs accumulate mutations in CCR4 which stabilize it on the surface and reduced cycling (Nakagawa, 2014 #4464)(Duvic, 2015 #4463). To determine what extent CCR4 directed EVs can target the various CCR4 stabilizing mutations which are commonly found in HTLV-1 infected T-cells, Jurkat cells, which are inherently CCR4 negative, are generated to overexpress wildtype CCR4 and those known mutants CCR4 (Nakagawa, 2014 #4464). Uptake of the various EVs is tested on these cells. nLuc expression is assessed following treatment with the various EVs (FIG. 16B). These studies delineate the ability of the CCR4 directed EVs to function in the various CCR4 mutational backgrounds.

### Characterization of EV Secretome and Genomic Payloads.

EVs have been used clinically (9), however each cell generated EV contains contents of the producer cell line. HEK293 cells are engineered to constitutively express the PTGFRN or CD63-anti-CCR4 fusions and package ZFP5-KRAB-meCP2, it will be important to understand to what extent engineered EVs modify the endogenous EV pathways including both the respective secretome and nucleic acid content of the EVs. To determine the incorporation and relative expression of CD63-anti-CCR4, PTGFRN-anti-CCR4 and ZFP5-KRAB-meCP2 into these EVs and the relative nucleic acid signatures in the HTLV-1 directed EVs compared to parental cell EV controls EVs are isolated (Shrivastava, 2021 #4449), and RNA and DNA high-throughput genomic sequencing is completed. Genomic networks that are differentially modulated from the treatment of various cells with exosomes are determined (38, 16). The protein content (secretome) of the EVs using LC-MS based analysis (Multi-omics) is used to determine any unique proteins packaged into the various EVs. Collectively, these studies provide a better understanding of those EV those RNAs and proteins packaged into EVs, and determine those EV associated membrane proteins.

### Ev Biodistribution.

To determine the biodistribution of EVs, EVs packaged with NanoLuc (nLuc) Luciferase and TRDye 800-labeled are generated. EV-a, EV-b and EV-c with nLuc from the EXOtic system (7) are characterized, as nLuc can be readily used for in vivo imaging (Shrivastava, 2021 #4449). The nLuc/TRDye 800-labeled EV-a-nLuc and EV-c-nLuc are injected RO (range between ˜20-100 billion exosomes per injection) into NOD SCID ß2m (NSC-ß2m) mice treated apriori with HTLV-1 transformed TL-Om1 cells in matrigel and the distribution of EVs determined in the TL-Om1 tumour cell injection site as well as in the brain, spleen, lymph nodes, GALT and bone marrow at 4 hrs, 24 hrs and 1-week post-injection by qRT-PCR for nLuc, HBZ and immunohistochemical staining of the various tissues (Shrivastava, 2021 #4449). These data inform as the biodistribution, persistence and dosage required for the studies outlined in A.3.3.

### Characterization Intravenous Administered Anti-HTLV-1 EVs in HTLV-1 Infected NOD SCID ß2m Mouse.

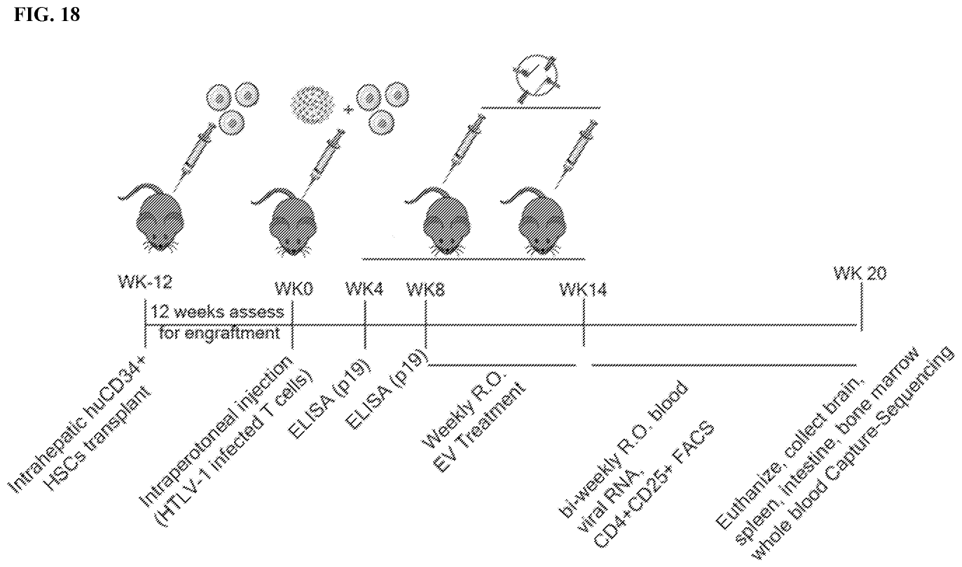

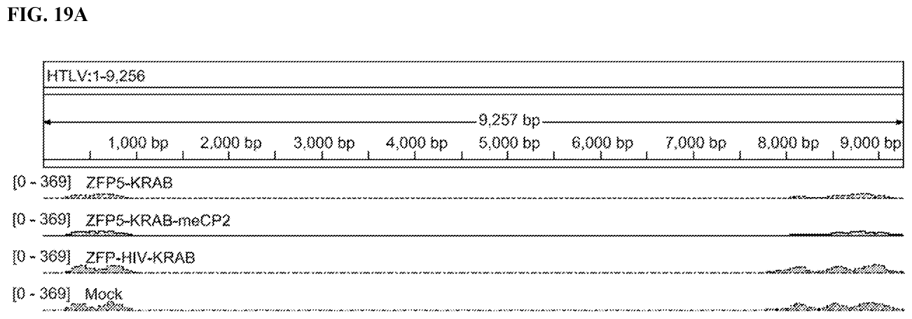

The ability of the anti-HTLV-1 EVs to target and kill HTLV-1 transformed T cells in vivo is determined using the using humanized NSC-ß2m mice infected with HTLV-1 (Van Duyne, 2009 #4457)(Banerjee, 2010 #4456). The NSC-ß2m mice are inoculated with ex vivo HTLV-1 infected patient derived T-cells (MOI-5.0) (FIG. 18). To evaluate the in vivo efficacy of the anti-HTLV-1 EVs and the approach proposed here (FIGS. 16A-16B), a total of 70 HTLV-1-infected humanized NSG mice (5M/5F) per group are injected retro-orbital venous sinus (R.O.), which is considered synonymous with intravenous in humans, with 80 billion exosomes (EV-a, b, c and control EVs derived from stable anti-HTLV-1 EV producing HEK293 cells (FIG. 18) (refer to vertebrate animal section). Virus infected untreated mice alone also serve as a control. On week 0, 12 weeks post-CD34 engraftment, the mice are treated with matched HTLV-1 infected CD4+ T-cells and then monitored for 4 weeks for viral infection by ELISA and qRT-PCR for viral RNAs in T-cells collected from the blood (FIG. 18). Following successful infection, the mice are treated weekly for 6 weeks with R.O. administered EVs (80-120 billion EVs/mouse)(Shrivastava, 2021 #4449). Following the EV treatment and on bi-weekly basis, from week 14-18, 100 μl of blood will be collected and huCD45⁺, CD4⁺CD25⁺ and CD8⁺ populations determined by flow cytometry. ZFP5-KRAB-meCP2 and viral RNAs are also measured from the isolated blood by quantitative qRT-PCR. Notably a shift to CD4+-CD25+ T-cells by FACS is routinely observed in HTLV-1-mediated ATL (Zimmerman, 2010 #4458). At the termination of the experiment intracardiac perfusion with PBS solution containing sodium nitrate and heparin is carried out to remove blood from capillaries, tissues collected and the genomic DNA from brain, spleen, bone marrow isolated and processed and the relative integrated remaining HTLV-1 variants determined by capture sequencing for integrated virus, as described in (Katsuya, 2019 #4459). Additional analysis includes immunohistochemistry of brain and lymphoid tissues for HTLV-1 p19 antigen, the development of CD4+ T-cell lymphoma by assessment of atypical lymphocytes containing lobulated nuclei resembling ATL-specific flower cells and flow cytometry carried out for cell surface markers (e.g., hCD45, CD3, CD4+CD25+, CD14, CCR5, CCR4, and HTLV-1 HBZ) and qRT-PCR carried out for HTLV-1 RNA and EV-delivered RNAs (ZFP5-KRAB-meCP2). These data are critical in the assessment of the efficacy of the approach outlined here in vivo and serve as a proof of concept regarding the overall approach and to what extent the engineered EVs facilitate the targeted killing of HTLV-1 infected cells in vivo.

### REFERENCES

1. Hausen, H. z. (1991) Viruses in Human Cancers. 254, 1167-1173.
2. Yoshie, 0. (2005) Expression of CCR4 in adult T-cell leukemia. Leukemia & lymphoma, 46, 185-190.
3. Adrienne, A. P., Paul, A. F., Olivier, H., Juan, C. R., Brady, E. B., Juliana, P., Farooq, W., Tatyana, F., Graham, P. T., Ahmed, S. et al. (2019) Mogamulizumab versus investigator's choice of chemotherapy regimen in relapsed/refractory adult T-cell leukemia/lymphoma. Haematologica, 104, 993-1003.
4. Sakamoto, Y., Ishida, T., Masaki, A., Murase, T., Yonekura, K., Tashiro, Y., Tokunaga, M., Utsunomiya, A., Ito, A., Kusumoto, S. et al. (2018) CCR4 mutations associated with superior outcome of adult T-cell leukemia/lymphoma under mogamulizumab treatment. Blood, 132, 758-761.
5. Giam, C. Z. and Jeang, K. T. (2007) HTLV-1 Tax and adult T-cell leukemia. Front Biosci, 12, 1496-1507.
6. Takeda, S., Maeda, M., Morikawa, S., Taniguchi, Y., Yasunaga, J.-i., Nosaka, K., Tanaka, Y. and Matsuoka, M. (2004) Genetic and epigenetic inactivation of tax gene in adult T-cell leukemia cells. Int. J Cancer, 109, 559-567.
7. Tanaka-Nakanishi, A., Yasunaga, J.-i., Takai, K. and Matsuoka, M. (2014) HTLV-1 bZIP Factor Suppresses Apoptosis by Attenuating the Function of FoxO3a and Altering Its Localization. Cancer Research, 74, 188-200.
8. Vernin, C., Thenoz, M., Pinatel, C., Gessain, A., Gout, O., Delfau-Larue, M.-H., Nazaret, N., Legras-Lachuer, C., Wattel, E. and Mortreux, F. (2014) HTLV-1 bZIP Factor HBZ Promotes Cell Proliferation and Genetic Instability by Activating OncomiRs. Cancer Research, 74, 6082-6093.
9. Satou, Y., Yasunaga, J.-i., Zhao, T., Yoshida, M., Miyazato, P., Takai, K., Shimizu, K., Ohshima, K., Green, P. L., Ohkura, N. et al. (2011) HTLV-1 bZIP Factor Induces T-Cell Lymphoma and Systemic Inflammation In Vivo. PLOS Pathogens, 7, e1001274.
10. Mitobe, Y., Yasunaga, J.-i., Furuta, R. and Matsuoka, M. (2015) HTLV-1 bZIP Factor RNA and Protein Impart Distinct Functions on T-cell Proliferation and Survival. Cancer Research, 75, 4143-4152.
11. Sugata, K., Yasunaga, J.-i., Kinosada, H., Mitobe, Y., Furuta, R., Mahgoub, M., Onishi, C., Nakashima, K., Ohshima, K. and Matsuoka, M. (2016) HTLV-1 Viral Factor HBZ Induces CCR4 to Promote T-cell Migration and Proliferation. Cancer Research, 76, 5068.
12. Kataoka, K., Nagata, Y., Kitanaka, A., Shiraishi, Y., Shimamura, T., Yasunaga, J.-i., Totoki, Y., Chiba, K., Sato-Otsubo, A., Nagae, G. et al. (2015) Integrated molecular analysis of adult T cell leukemia/lymphoma. Nature Genetics, 47, 1304-1315.
13. Arnold, J., Zimmerman, B., Li, M., Lairmore, M. D. and Green, P. L. (2008) Human T-cell leukemia virus type-1 antisense-encoded gene, Hbz, promotes T-lymphocyte proliferation. Blood, 112, 3788-3797.
14. Satou, Y., Yasunaga, J.-i., Yoshida, M. and Matsuoka, M. (2006) HTLV-I basic leucine zipper factor gene mRNA supports proliferation of adult T cell leukemia cells. Proceedings of the National Academy of Sciences of the United States of America, 103, 720-725.
15. Papworth, M., Kolasinska, P. and Minczuk, M. (2006) Designer zinc-finger proteins and their applications. Gene, 366, 27-38.
16. Mandell, J. G. and Barbas, C. F., III. (2006) Zinc Finger Tools: custom DNA-binding domains for transcription factors and nucleases. Nucleic Acids Research, 34, W516-W523.
17. Shrivastava, S., Ray, R. M., Holguin, L., Echavarria, L., Grepo, N., Scott, T. A., Burnett, J. and Morris, K. V. (2021) Exosome-mediated stable epigenetic repression of HIV-1. Nature Communications, 12, 5541.
18. Kuramitsu, M., Okuma, K., Yamagishi, M., Yamochi, T., Firouzi, S., Momose, H., Mizukami, T., Takizawa, K., Araki, K., Sugamura, K. et al. (2015) Identification of TL-Om1, an Adult T-Cell Leukemia (ATL) Cell Line, as Reference Material for Quantitative PCR for Human T-Lymphotropic Virus 1. Journal of Clinical Microbiology, 53, 587-596.
19. Mandell, J. G. and Barbas, C. F., III. (2006) Zinc Finger Tools: custom DNA-binding domains for transcription factors and nucleases. Nucleic Acids Res, 34, W516-W523.
20. Urrutia, R. (2003) KRAB-containing zinc-finger repressor proteins. Genome biology, 4, 231-231.
21. Scott, T. A., O'Meally, D., Grepo, N. A., Soemardy, C., Lazar, D. C., Zheng, Y., Weinberg, M. S., Planelles, V. and Morris, K. V. (2021) Broadly active zinc finger protein-guided transcriptional activation of HIV-1. Molecular Therapy—Methods & Clinical Development, 20, 18-29.
22. Sugamura, K., Fujii, M., Kannagi, M., Sakitani, M., Takeuchi, M. and Hinuma, Y. (1984) Cell surface phenotypes and expression of viral antigens of various human cell lines carrying human T-cell leukemia virus. International Journal of Cancer, 34, 221-228.
23. Tanaka, Y., Mizuguchi, M., Takahashi, Y., Fujii, H., Tanaka, R., Fukushima, T., Tomoyose, T., Ansari, A. A. and Nakamura, M. (2015) Human T-cell leukemia virus type-I Tax induces the expression of CD83 on T cells. Retrovirology, 12, 56.
24. Koiwa, T., Hamano-Usami, A., Ishida, T., Okayama, A., Yamaguchi, K., Kamihira, S. and Watanabe, T. (2002) 5′-long terminal repeat-selective CpG methylation of latent human T-cell leukemia virus type 1 provirus in vitro and in vivo. Journal of virology, 76, 9389-9397.
25. Alerasool, N., Segal, D., Lee, H. and Taipale, M. (2020) An efficient KRAB domain for CRISPRi applications in human cells. Nature Methods, 17, 1093-1096.
26. Yeo, N.C., Chavez, A., Lance-Byrne, A., Chan, Y., Menn, D., Milanova, D., Kuo, C.-C., Guo, X., Sharma, S., Tung, A. et al. (2018) An enhanced CRISPR repressor for targeted mammalian gene regulation. Nature methods, 15, 611-616.
27. Kawatsuki, A., Yasunaga, J. I., Mitobe, Y., Green, P. L. and Matsuoka, M. (2016) HTLV-1 bZIP factor protein targets the Rb/E2F-1 pathway to promote proliferation and apoptosis of primary CD4(+) T cells. Oncogene, 35, 4509-4517.
28. Tanaka, A., Takeda, S., Kariya, R., Matsuda, K., Urano, E., Okada, S. and Komano, J. (2013) A novel therapeutic molecule against HTLV-1 infection targeting provirus. Leukemia, 27, 1621-1627.
29. Sharma, S. V., Fischbach, M. A., Haber, D. A. and Settleman, J. (2006) “Oncogenic Shock”: Explaining Oncogene Addiction through Differential Signal Attenuation. Clinical cancer research, 12, 4392s-4395s.
30. Fuks, F., Hurd, P. J., Wolf, D., Nan, X., Bird, A. P. and Kouzarides, T. (2003) The Methyl-CpG-binding Protein MeCP2 Links DNA Methylation to Histone Methylation*. Journal of Biological Chemistry, 278, 4035-4040.
31. Vansant, G., Bruggemans, A., Janssens, J. and Debyser, Z. (2020) Block-And-Lock Strategies to Cure HIV Infection. Viruses, 12, 84.
32. Xiang, J., Rauch, D. A., Huey, D. D., Panfil, A. R., Cheng, X., Esser, A. K., Su, X., Harding, J. C., Xu, Y., Fox, G. C. et al. (2019) HTLV-1 viral oncogene HBZ drives bone destruction in adult T cell leukemia. JCI Insight, 4, e128713.
33. Garg, H., Suri, P., Gupta, J. C., Talwar, G. P. and Dubey, S. (2016) Survivin: a unique target for tumor therapy. Cancer Cell International, 16, 49.
34. El Hajj, H., Tsukasaki, K., Cheminant, M., Bazarbachi, A., Watanabe, T. and Hermine, O. (2020) Novel Treatments of Adult T Cell Leukemia Lymphoma. Front Microbiology, 11.
35. Enose-Akahata, Y., Vellucci, A. and Jacobson, S. (2017) Role of HTLV-1 Tax and HBZ in the Pathogenesis of HAM/TSP. Front Microbiol, 8, 2563.
36. Rurik, J. G., Tombicz, I., Yadegari, A., Fernández, P. O. M., Shewale, S. V., Li, L., Kimura, T., Soliman, O. Y., Papp, T. E., Tam, Y. K. et al. (2022) CAR T cells produced in vivo to treat cardiac injury. Science, 375, 91-96.
37. O'Brien, K., Breyne, K., Ughetto, S., Laurent, L. C. and Breakefield, X. O. (2020) RNA delivery by extracellular vesicles in mammalian cells and its applications. Nature Reviews Molecular Cell Biology, 21, 585-606.
38. Villamizar, O., Waters, S. A., Scott, T., Grepo, N., Jaffe, A. and Morris, K. V. (2021) Mesenchymal Stem Cell exosome delivered Zinc Finger Protein activation of cystic fibrosis transmembrane conductance regulator. J Extracell Vesicle, 10, e12053.

### INFORMAL SEQUENCE LISTING

TABLE 1
Sequences of zinc finger domains

| Vector | Amino acid sequence | SEQ ID NO |
|---|---|---|
| HTLV- | LEPGEKPYKCPECGKSFSRNDTLTEHQRTHTGEKPYKCPECGKSFSSRRTCR | 1 |
| ZFP-2 | AHQRTHTGEKPYKCPECGKSFSRNDALTEHQRTHTGEKPYKCPECGKSFS | |
| | QSSNLVRHQRTHTGEKPYKCPECGKSFSTKNSLTEHQRTHTGEKPYKCPEC | |
| | GKSFSTKNSLTEHQRTHTGKKTS | |
| HTLV- | LEPGEKPYKCPECGKSFSRADNLTEHQRTHTGEKPYKCPECGKSFSTSGSLV | 2 |
| ZFP-3 | RHQRTHTGEKPYKCPECGKSFSQSGDLRRHQRTHTGEKPYKCPECGKSFS | |
| | DPGNLVRHQRTHTGEKPYKCPECGKSFSRSDDLVRHQRTHTGEKPYKCPE | |
| | CGKSFSRTDTLRDHQRTHTGKKTS | |
| HTLV- | LEPGEKPYKCPECGKSFSTKNSLTEHQRTHTGEKPYKCPECGKSFSRSDKLV | 3 |
| ZFP-4 | RHQRTHTGEKPYKCPECGKSFSDKKDLTRHQRTHTGEKPYKCPECGKSFSR | |
| | SDNLVRHQRTHTGEKPYKCPECGKSFSDPGALVRHQRTHTGEKPYKCPEC | |
| | GKSFSRADNLTEHQRTHTGKKTS | |
| HTLV- | LEPGEKPYKCPECGKSFSRTDTLRDHQRTHTGEKPYKCPECGKSFSQRANL | 4 |
| ZFP-5 | RAHQRTHTGEKPYKCPECGKSFSRADNLTEHQRTHTGEKPYKCPECGKSFS | |
| | QSSNLVRHQRTHTGEKPYKCPECGKSFSQSGDLRRHQRTHTGEKPYKCPE | |
| | CGKSFSDPGHLVRHQRTHTGKKTS | |
| HTLV- | LEPGEKPYKCPECGKSFSRNDTLTEHQRTHTGEKPYKCPECGKSFSRKDNL | 5 |
| ZFP-6 | KNHQRTHTGEKPYKCPECGKSFSDCRDLARHQRTHTGEKPYKCPECGKSF | |
| | SRSDHLTTHQRTHTGEKPYKCPECGKSFSRNDTLTEHQRTHTGEKPYKCPE | |
| | CGKSFSRSDHLTTHQRTHTGKKTS | |
| HTLV- | LEPGEKPYKCPECGKSFSTHLDLIRHQRTHTGEKPYKCPECGKSFSQRAHLE | 6 |
| ZFP-7 | RHQRTHTGEKPYKCPECGKSFSTSGHLVRHQRTHTGEKPYKCPECGKSFSR | |
| | SDHLTNHQRTHTGEKPYKCPECGKSFSQSSNLVRHQRTHTGEKPYKCPEC | |
| | GKSFSRSDHLTNHQRTHTGKKTS | |
| HTLV- | LEPGEKPYKCPECGKSFSRADNLTEHQRTHTGEKPYKCPECGKSFSQSGHL | 7 |
| ZFP-8 | TEHQRTHTGEKPYKCPECGKSFSRADNLTEHQRTHTGEKPYKCPECGKSFS | |
| | ERSHLREHQRTHTGEKPYKCPECGKSFSRSDKLTEHQRTHTGEKPYKCPEC | |
| | GKSFSTSGELVRHQRTHTGKKTS | |
| HTLV- | LEPGEKPYKCPECGKSFSDPGHLVRHQRTHTGEKPYKCPECGKSFSDCRDL | 8 |
| ZFP-9 | ARHQRTHTGEKPYKCPECGKSFSQSGNLTEHQRTHTGEKPYKCPECGKSFS | |
| | DPGHLVRHQRTHTGEKPYKCPECGKSFSRSDELVRHQRTHTGEKPYKCPE | |
| | CGKSFSDCRDLARHQRTHTGKKTS | |
| HTLV- | LEPGEKPYKCPECGKSFSRSDKLTEHQRTHTGEKPYKCPECGKSFSRSDHLT | 9 |
| ZFP-10 | THQRTHTGEKPYKCPECGKSFSQRAHLERHQRTHTGEKPYKCPECGKSFSS | |
| | RRTCRAHQRTHTGEKPYKCPECGKSFSRSDKLTEHQRTHTGEKPYKCPECG | |
| | KSFSDSGNLRVHQRTHTGKKTS | |

TABLE 2
Sequences of proteins including zinc finger domains

| Vector | Amino acid sequence | SEQ ID NO |
|---|---|---|
| HTLV-ZFP-KRAB-2 | MISEFGSGAPGRKKRRQRRRVDLEQKLISEEDLLKRPAATKK | 10 |
| | AGQAKKKKLEPGEKPYKCPECGKSFSRNDTLTEHQRTHTGEK | |
| | PYKCPECGKSFSSRRTCRAHQRTHTGEKPYKCPECGKSFSRND | |
| | ALTEHQRTHTGEKPYKCPECGKSFSQSSNLVRHQRTHTGEKP | |
| | YKCPECGKSFSTKNSLTEHQRTHTGEKPYKCPECGKSFSTKNS | |
| | LTEHQRTHTGKKTSSAGGGGSGGGGSGGGGSGRTLVTFKD | |
| | VFVDFTREEWKLLDTAQQIVYRNVMLENYKNLVSLGYQLTKP | |
| | DVILRLEKGEEPWLV | |
| HTLV-ZFP-KRAB-3 | MISEFGSGAPGRKKRRQRRRVDLEQKLISEEDLLKRPAATKK | 11 |
| | AGQAKKKKLEPGEKPYKCPECGKSFSRADNLTEHQRTHTGEK | |
| | PYKCPECGKSFSTSGSLVRHQRTHTGEKPYKCPECGKSFSQSG | |
| | DLRRHQRTHTGEKPYKCPECGKSFSDPGNLVRHQRTHTGEK | |
| | PYKCPECGKSFSRSDDLVRHQRTHTGEKPYKCPECGKSFSRTD | |
| | TLRDHQRTHTGKKTSSAGGGGSGGGGSGGGGSGRTLVTFK | |
| | DVFVDFTREEWKLLDTAQQIVYRNVMLENYKNLVSLGYQLTK | |
| | PDVILRLEKGEEPWLV | |
| HTLV-ZFP-KRAB-4 | MISEFGSGAPGRKKRRQRRRVDLEQKLISEEDLLKRPAATKK | 12 |
| | AGQAKKKKLEPGEKPYKCPECGKSFSTKNSLTEHQRTHTGEK | |
| | PYKCPECGKSFSRSDKLVRHQRTHTGEKPYKCPECGKSFSDKK | |
| | DLTRHQRTHTGEKPYKCPECGKSFSRSDNLVRHQRTHTGEKP | |
| | YKCPECGKSFSDPGALVRHQRTHTGEKPYKCPECGKSFSRAD | |
| | NLTEHQRTHTGKKTSSAGGGGSGGGGSGGGGSGRTLVTFK | |
| | DVFVDFTREEWKLLDTAQQIVYRNVMLENYKNLVSLGYQLTK | |
| | PDVILRLEKGEEPWLV | |
| HTLV-ZFP-KRAB-5 | MISEFGSGAPGRKKRRQRRRVDLEQKLISEEDLLKRPAATKK | 13 |
| | AGQAKKKKLEPGEKPYKCPECGKSFSRTDTLRDHQRTHTGEK | |
| | PYKCPECGKSFSQRANLRAHQRTHTGEKPYKCPECGKSFSRA | |
| | DNLTEHQRTHTGEKPYKCPECGKSFSQSSNLVRHQRTHTGEK | |
| | PYKCPECGKSFSQSGDLRRHQRTHTGEKPYKCPECGKSFSDP | |
| | GHLVRHQRTHTGKKTSSAGGGGSGGGGSGGGGSGRTLVTF | |
| | KDVFVDFTREEWKLLDTAQQIVYRNVMLENYKNLVSLGYQL | |
| | TKPDVILRLEKGEEPWLV | |
| HTLV-ZFP-KRAB-6 | MISEFGSGAPGRKKRRQRRRVDLEQKLISEEDLLKRPAATKK | 14 |
| | AGQAKKKKLEPGEKPYKCPECGKSFSRNDTLTEHQRTHTGEK | |
| | PYKCPECGKSFSRKDNLKNHQRTHTGEKPYKCPECGKSFSDC | |
| | RDLARHQRTHTGEKPYKCPECGKSFSRSDHLTTHQRTHTGEK | |
| | PYKCPECGKSFSRNDTLTEHQRTHTGEKPYKCPECGKSFSRSD | |
| | HLTTHQRTHTGKKTSSAGGGGSGGGGSGGGGSGRTLVTFK | |
| | DVFVDFTREEWKLLDTAQQIVYRNVMLENYKNLVSLGYQLTK | |
| | PDVILRLEKGEEPWLV | |
| HTLV-ZFP-KRAB-7 | MISEFGSGAPGRKKRRQRRRVDLEQKLISEEDLLKRPAATKK | 15 |
| | AGQAKKKKLEPGEKPYKCPECGKSFSTHLDLIRHQRTHTGEK | |
| | PYKCPECGKSFSQRAHLERHQRTHTGEKPYKCPECGKSFSTSG | |
| | HLVRHQRTHTGEKPYKCPECGKSFSRSDHLTNHQRTHTGEKP | |
| | YKCPECGKSFSQSSNLVRHQRTHTGEKPYKCPECGKSFSRSDH | |
| | LTNHQRTHTGKKTSSAGGGGSGGGGSGGGGSGRTLVTFKD | |
| | VFVDFTREEWKLLDTAQQIVYRNVMLENYKNLVSLGYQLTKP | |
| | DVILRLEKGEEPWLV | |
| HTLV-ZFP-KRAB-8 | MISEFGSGAPGRKKRRQRRRVDLEQKLISEEDLLKRPAATKK | 16 |
| | AGQAKKKKLEPGEKPYKCPECGKSFSRADNLTEHQRTHTGEK | |
| | PYKCPECGKSFSQSGHLTEHQRTHTGEKPYKCPECGKSFSRAD | |
| | NLTEHQRTHTGEKPYKCPECGKSFSERSHLREHQRTHTGEKP | |
| | YKCPECGKSFSRSDKLTEHQRTHTGEKPYKCPECGKSFSTSGEL | |
| | VRHQRTHTGKKTSSAGGGGSGGGGSGGGGSGRTLVTFKDV | |
| | FVDFTREEWKLLDTAQQIVYRNVMLENYKNLVSLGYQLTKPD | |
| | VILRLEKGEEPWLV | |
| HTLV-ZFP-KRAB-9 | MISEFGSGAPGRKKRRQRRRVDLEQKLISEEDLLKRPAATKK | 17 |
| | AGQAKKKKLEPGEKPYKCPECGKSFSDPGHLVRHQRTHTGE | |
| | KPYKCPECGKSFSDCRDLARHQRTHTGEKPYKCPECGKSFSQS | |
| | GNLTEHQRTHTGEKPYKCPECGKSFSDPGHLVRHQRTHTGE | |
| | KPYKCPECGKSFSRSDELVRHQRTHTGEKPYKCPECGKSFSDC | |
| | RDLARHQRTHTGKKTSSAGGGGSGGGGSGGGGSGRTLVTF | |
| | KDVFVDFTREEWKLLDTAQQIVYRNVMLENYKNLVSLGYQLT | |
| | KPDVILRLEKGEEPWLV | |
| HTLV-ZFP-KRAB-10 | MISEFGSGAPGRKKRRQRRRVDLEQKLISEEDLLKRPAATKK | 18 |
| | AGQAKKKKLEPGEKPYKCPECGKSFSRSDKLTEHQRTHTGEK | |
| | PYKCPECGKSFSRSDHLTTHQRTHTGEKPYKCPECGKSFSQRA | |
| | HLERHQRTHTGEKPYKCPECGKSFSSRRTCRAHQRTHTGEKP | |
| | YKCPECGKSFSRSDKLTEHQRTHTGEKPYKCPECGKSFSDSGN | |
| | LRVHQRTHTGKKTSSAGGGGSGGGGSGGGGSGRTLVTFKD | |
| | VFVDFTREEWKLLDTAQQIVYRNVMLENYKNLVSLGYQLTKP | |
| | DVILRLEKGEEPWLV | |
| HTLV-ZFP-3 | MISEFGSGAPGRKKRRQRRRVDLEQKLISEEDLLKRPAATKK | 19 |
| | AGQAKKKKLEPGEKPYKCPECGKSFSRADNLTEHQRTHTGEK | |
| | PYKCPECGKSFSTSGSLVRHQRTHTGEKPYKCPECGKSFSQSG | |
| | DLRRHQRTHTGEKPYKCPECGKSFSDPGNLVRHQRTHTGEK | |
| | PYKCPECGKSFSRSDDLVRHQRTHTGEKPYKCPECGKSFSRTD | |
| | TLRDHQRTHTGKKTS | |
| HTLV-ZFP-5 | MISEFGSGAPGRKKRRQRRRVDLEQKLISEEDLLKRPAATKK | 20 |
| | AGQAKKKKLEPGEKPYKCPECGKSFSRTDTLRDHQRTHTGEK | |
| | PYKCPECGKSFSQRANLRAHQRTHTGEKPYKCPECGKSFSRA | |
| | DNLTEHQRTHTGEKPYKCPECGKSFSQSSNLVRHQRTHTGEK | |
| | PYKCPECGKSFSQSGDLRRHQRTHTGEKPYKCPECGKSFSDP | |
| | GHLVRHQRTHTGKKTS | |
| HTLV-ZFP- | MISEFGSGAPGRKKRRQRRRVDLEQKLISEEDLLKRPAATKK | 21 |
| KRAB(ZIM3)-5 | AGQAKKKKLEPGEKPYKCPECGKSFSRTDTLRDHQRTHTGEK | |
| | PYKCPECGKSFSQRANLRAHQRTHTGEKPYKCPECGKSFSRA | |
| | DNLTEHQRTHTGEKPYKCPECGKSFSQSSNLVRHQRTHTGEK | |
| | PYKCPECGKSFSQSGDLRRHQRTHTGEKPYKCPECGKSFSDP | |
| | GHLVRHQRTHTGKKTSSAGGGGSGGGGSGGGGSGMGRVT | |
| | FEDVTVNFTQGEWQRLNPEQRNLYRDVMLENYSNLVSVGQ | |
| | GETTKPDVILRLEQGKEPWLEEEEVLGSGRAEKNGDIGGQIW | |
| | KPKDVKESL | |
| HTLV-ZFP-KRAB- | MISEFGSGAPGRKKRRQRRRVDLEQKLISEEDLLKRPAATKK | 22 |
| meCP2-5 | AGQAKKKKLEPGEKPYKCPECGKSFSRTDTLRDHQRTHTGEK | |
| | PYKCPECGKSFSQRANLRAHQRTHTGEKPYKCPECGKSFSRA | |
| | DNLTEHQRTHTGEKPYKCPECGKSFSQSSNLVRHQRTHTGEK | |
| | PYKCPECGKSFSQSGDLRRHQRTHTGEKPYKCPECGKSFSDP | |
| | GHLVRHQRTHTGKKTSSAGGGGSGGGGSGGGGSGRTLVTF | |
| | KDVFVDFTREEWKLLDTAQQIVYRNVMLENYKNLVSLGYQLT | |
| | KPDVILRLEKGEEPWLVASSPKKKRKVEASVQVKRVLEKSPGK | |
| | LLVKMPFQASPGGKGEGGGATTSAQVMVIKRPGRKRKAEA | |
| | DPQAIPKKRGRKPGSVVAAAAAEAKKKAVKESSIRSVQETVLP | |
| | IKKRKTRETVSIEVKEVVKPLLVSTLGEKSGKGLKTCKSPGRKSK | |
| | ESSPKGRSSSASSPPKKEHHHHHHHAESPKAPMPLLPPPPPP | |
| | EPQSSEDPISPPEPQDLSSSICKEEKMPRAGSLESDGCPKEPAK | |
| | TQPMVAAAATTTTTTTTTVAEKYKHRGEGERKDIVSSSMPRP | |
| | NREEPVDSRTPVTERVSEF | |
| HTLV-ZFP-PAM-5 | MISEFGSGAPGRKKRRQRRRVDLEQKLISEEDLLKRPAATKK | 23 |
| | AGQAKKKKLEPGEKPYKCPECGKSFSRTDTLRDHQRTHTGEK | |
| | PYKCPECGKSFSQRANLRAHQRTHTGEKPYKCPECGKSFSRA | |
| | DNLTEHQRTHTGEKPYKCPECGKSFSQSSNLVRHQRTHTGEK | |
| | PYKCPECGKSFSQSGDLRRHQRTHTGEKPYKCPECGKSFSDP | |
| | GHLVRHQRTHTGKKTSSAGGGGSGGGGSGGGGSGGIGELV | |
| | WGKLRGFSWWPGRIVSWWMTGRSRAAEGTRWVMWFG | |
| | DGKFSVVCVEKLMPLSSFCSAFHQATYNKQPMYRKAIYEVLQ | |
| | VASSRAGKLFPVCHDSDESDTAKAVEVONKPMIEWALGGFQ | |
| | PSGPKGLEPINSRSSGSEVRQKCRNIEDICISCGSLNVTLEHPLF | |
| | VGGMCQNCKNCFLECAYQYDDDGYQSYCTICCGGREVLMC | |
| | GNNNCCRCFCVECVDLLVGPGAAQAAIKEDPWNCYMCGHK | |
| | GTYGLLRRREDWPSRLQMFFANNHDQEFDPPKVYPPVPAEK | |
| | RKPIRVLSLFDGIATGLLVLKDLGIQVDRYIASEVCEDSITVGM | |
| | VRHQGKIMYVGDVRSVTQKHIQEWGPFDLVIGGSPCNDLSI | |
| | VNPARKGLYEGTGRLFFEFYRLLHDARPKEGDDRPFFWLFEN | |
| | WVAMGVSDKRDISRFLESNPVMIDAKEVSAAHRARYFWGNL | |
| | PGMNRPLASTVNDKLELQECLEHGRIAKFSKVRTITTRSNSIK | |
| | QGKDQHFPVFMNEKEDILWCTEMERVFGFPVHYTDVSNMS | |
| | RLARQRLLGRSWSVPVIRHLFAPLKEYFACV | |

Bold: Tat peptide; Underline: myc-tag; Bold underlined: NLS; Underlined italics: ZFP; Bold italics: linker; italics: repressor domains

TABLE 3
Target sequences within the HTLV-ILTR

| | Target sequence in HTLV-I | SEQ |
|---|---|---|
| Vector | LTR (5′-3′) | ID NO |
| HTLV-ZFP-2 | CCTCCTGAACTGCGTCCG | 24 |
| HTLV-ZFP-3 | ACGGCGGACGCAGTTCAG | 25 |
| HTLV-ZFP-4 | CAGGTCGAGACCGGGCCT | 26 |
| HTLV-ZFP-5 | GGCGCAGAACAGAAAACG | 27 |
| HTLV-ZFP-6 | TGGCCGTGGGCCAAGCCG | 28 |
| HTLV-ZFP-7 | AGGGAAAGGGGTGGAACT | 29 |
| HTLV-ZFP-8 | GCTCGGAGCCAGCGACAG | 30 |
| HTLV-ZFP-9 | GCCGTGGGCCAAGCCGGC | 31 |
| HTLV-ZFP-10 | AACCGGCGTGGATGGCGG | 32 |

TABLE 4
Target sequences within the HTLV-1 LTR and associated zinc finger
domain recognition helix regions

| | | | | | | | Recognition |
|---|---|---|---|---|---|---|---|
| | | SEQ | | | | | Helix |
| ZFN | | ID | | | Triplets | SEQ ID | Region |
| name | Target sequence | NO | Finger | Position | (5′-3′) | NO | (N-C) |
| HTLV- | 5′-CCTCCTGAACTGCGTCCG-3′ | 24 | F1 | 19 | CCG | 33 | RNDTLTE |
| ZFP-2 | | | F2 | 47 | CGT | 34 | SRRTCRA |
| | | | F3 | 75 | CTG | 35 | RNDALTE |
| | | | F4 | 103 | GAA | 36 | QSSNLVR |
| | | | F5 | 131 | CCT | 37 | TKNSLTE |
| | | | F6 | 159 | CCT | 38 | TKNSLTE |
| HTLV- | 5′-ACGGCGGACGCAGTTCAG-3′ | 25 | F1 | 19 | CAG | 39 | RADNLTE |
| ZFP-3 | | | F2 | 47 | GTT | 40 | TSGSLVR |
| | | | F3 | 75 | GCA | 41 | QSGDLRR |
| | | | F4 | 103 | GAC | 42 | DPGNLVR |
| | | | F5 | 131 | GCG | 43 | RSDDLVR |
| | | | F6 | 159 | ACG | 44 | RTDTLRD |
| HTLV- | 5′-CAGGTCGAGACCGGGCCT-3′ | 26 | F1 | 19 | CCT | 45 | TKNSLTE |
| ZFP-4 | | | F2 | 47 | GGG | 46 | RSDKLVR |
| | | | F3 | 75 | ACC | 47 | DKKDLTR |
| | | | F4 | 103 | GAG | 48 | RSDNLVR |
| | | | F5 | 131 | GTC | 49 | DPGALVR |
| | | | F6 | 159 | CAG | 50 | RADNLTE |
| HTLV- | 5′-GGCGCAGAACAGAAAACG-3′ | 27 | F1 | 19 | ACG | 51 | RTDTLRD |
| ZFP-5 | | | F2 | 47 | AAA | 52 | QRANLRA |
| | | | F3 | 75 | CAG | 53 | RADNLTE |
| | | | F4 | 103 | GAA | 54 | QSSNLVR |
| | | | F5 | 131 | GCA | 55 | QSGDLRR |
| | | | F6 | 159 | GGC | 56 | DPGHLVR |
| HTLV- | 5′-TGGCCGTGGGCCAAGCCG-3′ | 28 | F1 | 19 | CCG | 57 | RNDTLTE |
| ZFP-6 | | | F2 | 47 | AAG | 58 | RKDNLKN |
| | | | F3 | 75 | GCC | 59 | DCRDLAR |
| | | | F4 | 103 | TGG | 60 | RSDHLTT |
| | | | F5 | 131 | CCG | 61 | RNDTLTE |
| | | | F6 | 159 | TGG | 62 | RSDHLTT |
| HTLV- | 5′-AGGGAAAGGGGTGGAACT-3′ | 29 | F1 | 19 | ACT | 63 | THLDLIR |
| ZFP-7 | | | F2 | 47 | GGA | 64 | QRAHLER |
| | | | F3 | 75 | GGT | 65 | TSGHLVR |
| | | | F4 | 103 | AGG | 66 | RSDHLTN |
| | | | F5 | 131 | GAA | 67 | QSSNLVR |
| | | | F6 | 159 | AGG | 68 | RSDHLTN |
| HTLV- | 5′-GCTCGGAGCCAGCGACAG-3′ | 30 | F1 | 19 | CAG | 69 | RADNLTE |
| ZFP-8 | | | F2 | 47 | CGA | 70 | QSGHLTE |
| | | | F3 | 75 | CAG | 71 | RADNLTE |
| | | | F4 | 103 | AGC | 72 | ERSHLRE |
| | | | F5 | 131 | CGG | 73 | RSDKLTE |
| | | | F6 | 159 | GCT | 74 | TSGELVR |
| HTLV- | 5′-GCCGTGGGCCAAGCCGGC-3′ | 31 | F1 | 19 | GGC | 75 | DPGHLVR |
| ZFP-9 | | | F2 | 47 | GCC | 76 | DCRDLAR |
| | | | F3 | 75 | CAA | 77 | QSGNLTE |
| | | | F4 | 103 | GGC | 78 | DPGHLVR |
| | | | F5 | 131 | GTG | 79 | RSDELVR |
| | | | F6 | 159 | GCC | 80 | DCRDLAR |
| HTLV- | 5′-AACCGGCGTGGATGGCGG-3′ | 32 | F1 | 19 | CGG | 81 | RSDKLTE |
| ZFP- | | | F2 | 47 | TGG | 82 | RSDHLTT |
| 10 | | | F3 | 75 | GGA | 83 | QRAHLER |
| | | | F4 | 103 | CGT | 84 | SRRTCRA |
| | | | F5 | 131 | CGG | 85 | RSDKLTE |
| | | | F6 | 159 | AAC | 86 | DSGNLRV |

TABLE 5
Primer and cloning sequences

| qPCR | | SEQ ID |
|---|---|---|
| oligomers | Sequence (5′-3′) | NO |
| CCR4 F | GAAGAACAAGGCGGTGAAGA | 87 |
| CCR4 R | GGGTCTCTAGGAAGAGCACTAT | 88 |
| E2F1 qPCR F | GACCTGGAAACTGACCATCA | 89 |
| E2F1 qPCR R | GGTCTCATAGCGTGACTTCTC | 90 |
| KRAB qPCR F | CTTGACACTGCCCAACAGAT | 91 |
| KRAB qPCR R | ACCTGAGAATAACGTCTGGTTTAG | 92 |
| NLS qPCR F | GCTGGACAGGCTAAGAAGAAG | 93 |
| ZFP-3 qPCR R | CGTATGGGTTCGCTGATGT | 94 |
| ZFP-5 qPCR R | TTGATGATCGCGCAGTGT | 95 |
| GAPDH F | CTCTGCTCCTCCTGTTCGAC | 96 |
| GAPDH R | TTAAAAGCAGCCCTGGTGAC | 97 |
| HBZ spliced | GTTCAGGAGGCACCACAG | 98 |
| qPCR F | | |
| HBZ spliced | ACAGGCAAGCATCGAAACA | 99 |
| qPCR R | | |
| HBZ | CGGACGCAGTTCAGGAG | 100 |
| unspliced | | |
| qPCR F | | |
| HBZ | AAAGCGTGGAGACAGTTCAG | 101 |
| unspliced | | |
| qPCR R | | |
| Clonging | | SEQ ID |
| oligomers | Sequence (5′-3′) | NO |
| pcDNA-HBZ F | GGCAAGGCTTGACCGACAATTGGTGTACTAAGTTTCTCTCCTGGA | 102 |
| | GAGTGCTATA | |
| pcDNA-HBZ- | TTAAACGGGCCCTCTAGACTCGAGCTATTGCAACCACATCGCCTCCA | 103 |
| R | | |
| Myc-F | CTGATCTCAGAGGAGGACCTGC | 104 |
| ZFP5-R | TGGACTAGTGGATCCGAGCTCGGTACCTCAGCTCGTTTTCTT | 105 |
| | CCCCGTGTG | |
| ZFP5-PAM-F | ACACGGGGAAGAAAACGAGCAGCGCTGGAGGAGGTGGAA | 106 |
| | GCGGAG | |
| ZFP5-PAM-R | TGGACTAGTGGATCCGAGCTCGGTACCCTATCAAACGCACGCG | 107 |
| | AAGTACTC | |

TABLE 6

| | | Accession | SEQ |
|---|---|---|---|
| gBlock | Sequence (5′-3′) | number | ID NO |
| HTLV-15′ | GTACGGGCCAGATATACGCGTTTGACAATGACCATGAGCCCCAAATA | LC192515 | 108 |
| LTR | TCCCCCGGGGGCTTAGAGCCTCCCAGTGAAAAACATTTCCGAGAAAC | | |
| LC192515.1 | AGAAGTCTGAAAAGGTCAGGGCCCAGACTAAGGCTCTGACGTCTCCC | | |
| | CCCGGAGGGACAGCTCAGCACCGGCTCAGGCTAGGCCCTGACGTGTC | | |
| | CCCCTGAAGACAAATCATAAGCTCAGACCTCCGGGAAGCCACCGGAA | | |
| | CCACCCATTTCCTCCCCATGTTTGTCAAGCCACCCTCAGGCGTTGACGA | | |
| | CAACCCCTCACCTCAAAAAACTTTTCATGGCACGCATATGGCTGAATA | | |
| | AACTAACAGGAGTCTATAAAAGCGTGGAGACAGTTCAGGAGGGGGC | | |
| | TCGCATCTCTCCTTCACGCGCCCGCCGCCTTACCTGAGGCCGCCATCC | | |
| | ACGCCGGTTGAGTCGCGTTCTGCCGCCTCCCGCCTGTGGTGCCTCCTG | | |
| | AACTGCGTCCGCCGTCTAGGTAAGTTTAGAGCTCAGGTCGAGACCGG | | |
| | GCCTTTGTCCGGCGCTCCCTTGGAGCCTACCTAGACTCAGCCGGCTCT | | |
| | CCACGCTTTGCCTGACCCTGCTTGCTCGACTCCGCGTCTTTGTTTCGTT | | |
| | TTCTGTTCTGCGCCGCTACAGATCGAAAGTTCCACCCCTTTCCCTTTCA | | |
| | TTCACGACTGACTGCCGGCTTGGCCCACGGCCAAGTACCGGCGACTCT | | |
| | GTTGGCTCGGAGCCAGCGACAGCCCATTCTATAGCACTCTCCAGGAG | | |
| | AGAAACTTAGTACACACTCTGGCTAACTAGAGAACCCACTGCTTACTG | | |
| | GCTTATCGAAATTAATACGACTCACTATAGGGAGACCCAAGCTGGCTA | | |
| | GCGCCACCATGGAAGACG | | |
| Rluc-HBZ | TCGCTCTTGATCAGGGCGATATCCTCCTCGATGTCAGGCCACTCGTCC | LC192515 | 109 |
| splice | CAGGACTCGATCACGTCCACGACACTCTCAGCATGGACGATGGCCTT | | |
| LC192515 | GATCTTGTCTTGGTGCTCGTAGGAGTAGTGAAAGGCCAGACAAGCCC | | |
| | CCCAGTCGTGGCCCACAAAGATGATTTTCTTTGGAAGGTTCAGCAGCT | | |
| | CGAACCAAGCGGTGAGGTACTTGTAGTGATCCAGGAGGCGATATGA | | |
| | GCCATTCCCGCTCTTGCCGGACTTACCCATTCCGATCAGATCAGGGAT | | |
| | GATGCATCTAGCCACGGGCTCGATGTGAGGCACGACGTGCCTCCACA | | |
| | GGTAGCTGGAGGCAGCGTTACCATGCAGAAAAATCACGGCGTTCTCG | | |
| | GCGTGCTTCTCGGAATCATAGTAGTTGATGAAGGAGTCCAGCACGTT | | |
| | CATTTGCTTGCAGCGAGCCCACCACTGAGGCCCAGTGATCATGCGTTT | | |
| | GCGTTGCTCGGGGTCGTACACCTTGGAAGCCCCTACAGATACAAAGT | | |
| | TAACCATGCTTATTATCAGCCCACTTCCCAGGGTTTGGACAGAGTCTTC | | |
| | TTTTCGGATACCCAGTCTACGTGTTTGGAGACTGTGTACAAGGCGACT | | |
| | GGTGCCCCATCTCTGGGGGACTATGTTCGGCCCGCCTACATCGTCACG | | |
| | CCCTACTGGCCACCTGTCCAGAGCATCAGATCACCTGGGACCCCATCG | | |
| | ATGGACGCGTTATCGGCTCAGCTCTACAGTTCCTTATCCCTCGACTCCC | | |
| | CTCCTTCCCCACCCAGAGAACCTCTAAGACCCTCAAGGTCCTTACCCCG | | |
| | CCAATCACTCATACGACCCCCAACATTCCACCCTCCTTCCTCCAGGCCA | | |
| | TGCGCAAATACTCCCCCTTCCGAAATGGATACATGGAACCCACCCTTG | | |
| | GGCAGCACCTCCCAACCCTGTCTTTTCCAGACCCCGGACTCCGGCCCC | | |
| | AAAACCTGTACACCCTCTGGGGAGGCTCCGTTGTCTGCATGTACCTCT | | |
| | ACCAGCTTTCCCCCCCCATCACCTGGCCCCTCCTGCCCCACGTGATTTT | | |
| | TTGCCACCCCGGCCAGCTCGGGGCCTTCCTCACCAATGTTCCCTACAA | | |
| | GCGAATAGAAGAACTCCTCTATAAAATTTCCCTTACCACAGGGGCCCT | | |
| | AATAATTCTACCCGAAGACTGTTTGCCCACCACCCTTTTCCAGCCTGTT | | |
| | AGAGCACCCGTCACGCTGACAGCCTGGCAAAACGGCCTCCTTCCGTTC | | |
| | CACTCAACCCTCACCACTCCAGGCCTTATTTGGACATTTACCGATGGCA | | |
| | CGCCTATGATTTCCGGGCCCTGCCCTAAAGATGGCCAGCCATCTTTAG | | |
| | TACTACAGTCCTCCTCCTTTATATTTCACAAATTTCAAACCAAGGCCTA | | |
| | CCACCCCTCATTTCTACTCTCACACGGCCTCATACAGTACTCTTCCTTTC | | |
| | ATAATTTACATCTCCTGTTTGAAGAATACACCAACATCCCCATTTCTCT | | |
| | ACTTTTTAACGAAAAAGAGGCAGATGACAATGACCATGAGCCCC | | |
| HTLV-aAa- | CTCAAAAAACTTTTCATGGCACGCATATGGCTGAATAAACTAACAGGA | L36905 | 110 |
| TC | GTCTATAAAAGCGTGGAGACAGTTCAGGAGGGGGCTCGCATCTCTCC | | |
| France | TTCACGCGCCCGCCGCCTTACCTGAGGCCGCCATCCACGCCGGTTGAG | | |
| L36905 | TCGCGTTCTGCCGCCTCCCGCCTGTGGTGCCTCCTGAACTGCGTCCGC | | |
| | CGTCTAGGTAAGTTTAAAGCTCAGGTCGAGACCGGGCCTTTGTCCGG | | |
| | CGCTCCCTTGGAGCCTACCTAGACTCAGCCGGCTCTCCACGCTTTGCC | | |
| | TGACCCTGCTTGCTCAACTCTGCGTCTTTGTTTCGTTTTCTGTTCTGCGC | | |
| | CGCTACAGATCGAAAGTTCCACCCCTTTCCCTTTCATTCACGACTGACT | | |
| | GCCGGCTTGGCCCACGGCCAAGTACCGGCGACTCCGTTGGCTCGGAG | | |
| | CCAGCGACAGCCCATTCTATAGCACTCTCCAGGAGAGAAACTTAGTAC | | |
| | ACAGTCTCTGGCTAACTAGAGAACCCACTGCTTACTGGCTTATCGAAA | | |
| | TTAATACGACTCACTATAGGGAGACCCAAGCTGGCTAGCGCCACCAT | | |
| | GGAAGACG | | |
| HTLV-bAB- | CTCAAAAAACTTTTCATGGCACGCATATGGCTGAATAAACTAACAGGA | J02029 | 111 |
| Japanese | GTCTATAAAAGCGTGGAGACAGTTCAGGAGGGGGCTCGCATCTCTCC | | |
| (a-Jpn) | TTCACGCGCCCGCCGCCTTACCTGAGGCCGCCATCCACGCCGGTTGAG | | |
| ATK Japan | TCGCGTTCTGCCGCCTCCCGCCTGTGGTGCCTCCTGAACTGCGTCCGC | | |
| J02029 | CGTCTAGGTAAGTTTAAAGCTCAGGTCGAGACCGGGCCTTTGTCCGG | | |
| | CGCTCCCTTGGAGCCTACCTAGACTCAGCCGGCTCTCCACGCTTTGCC | | |
| | TGACCCTGCTTGCTCAACTCTACGTCTTTGTTTCGTTTTCTGTTCTGCGC | | |
| | CGTTACAGATCGAAAGTTCCACCCCTTTCCCTTTCATTCACGACTGACT | | |
| | GCCGGCTTGGCCCACGGCCAAGTACCGGCGACTCCGTTGGCTCGGAG | | |
| | CCAGCGACAGCCCATCCTATAGCACTCTCAGGAGAGAAATTTAGTACA | | |
| | CATAGTTGGAGGTAGCTCTGGCTAACTAGAGAACCCACTGCTTACTG | | |
| | GCTTATCGAAATTAATACGACTCACTATAGGGAGACCCAAGCTGGCTA | | |
| | GCGCCACCATGGAAGACG | | |
| HTLV-B | CTCAAAAAACTTTTCATGGCACGCATATGGCTGAATAAACTAACAGGA | JX507077 | 112 |
| HTLV-1 | GTCTATAAAAGCGTGGAGACAGTTCAGGAGGGGGCTCGCATCTCTCC | | |
| Central | TTCACGCGCCCGCCGCCTTACCTGAGGCCGCCATCCACGCCGGTTGAG | | |
| African | TCGCGTTCTGCCGCCTCCCGCCTGTGGTGCCTCCTGAACTGCGTCCGC | | |
| JX507077 | CGTCTAGGTAAGTTTAGAGCTCAGGTCGAGACCGGGCCTTTGTCCGG | | |
| | CGCTCCCTTAGAGCCTACCTAGACTCAGCCGGCTCTCCACGCTTTGCCT | | |
| | GACCCTGTTTGCTCACCTCTACGTCTTTGTTTCGTTTTCTGTTCTGCGCC | | |
| | GTTACAGATCGAAAGTTCCACCCCTTTCCCTTTCATTCACGACTGACTG | | |
| | CCGGCTTGGCCCACGGCCAAGTACCGGCGACTTTACTGGCTCGGAGC | | |
| | CAGCGACAGCCTATTCTATAGCACTCTCCAGGAGAGAAATTCAGTACA | | |
| | CACTCTGGCTAACTAGAGAACCCACTGCTTACTGGCTTATCGAAATTA | | |
| | ATACGACTCACTATAGGGAGACCCAAGCTGGCTAGCGCCACCATGGA | | |
| | AGACG | | |
| HTLV-C1 | CTCAAAAAACTTTTCATGGCACGCATATGGCTGAATAAACTAACAGGA | KF242505 | 113 |
| Australia | GTCTATAAAAGCGTGGAGACAGTTCAGGAGGGGGCTCGCATCTCTCC | | |
| Aus-DF | TTCACGCGCCCGCCGCCTTACCTGAGGCCGCCATCCACGCCGGTTGAG | | |
| Australia | TCGCGTTCTGCCGCCTCCCGCCTGTGGTGCCTCCTGAACTGCGTCCGC | | |
| KF242505 | CGTCTAGGTAAGTTCGGAGCTCAGGTCGAGACCGGGCCTTTGTCCGG | | |
| | CGCTCCCTTAGAGCCCACTTAGATTCGGTCGGCTCTCCACGCTTTGCCT | | |
| | GACCCTGCTTGCTCAACTCCACGTCTTTGTTTCGTTTTCTGTTCTGCGCC | | |
| | GCTACCGATCGAAAGTTCCGCCCCTTTCCCTTTCATTCACGTCTGACTG | | |
| | CCGGCTTGGCCCACGGCCAAGCACCGGCACCCTTACTGGCTCGGAGC | | |
| | CAGCGACAGCCCATTCTATACCTCTCTCCAGGAGAGAGACATAGAAC | | |
| | ACACTCTGGCTAACTAGAGAACCCACTGCTTACTGGCTTATCGAAATT | | |
| | AATACGACTCACTATAGGGAGACCCAAGCTGGCTAGCGCCACCATGG | | |
| | AAGACG | | |
| HTLV-D d | CTCAAAAAACTTTTCATGGCACGCATATGGCTGAATAAACTAACAGGA | L76310 | 114 |
| Pyg19a | GTCTATAAAAGCGTGGAGACAGTTCAGGAGGGGGCTCGCATCTCTCC | | |
| CAR | TTCACGCGCCCGCCGCCTTACCTGAGGCCGCCATCCACGCCGGTCGAG | | |
| L76310 | TCGCGTCCTGCCGCCTCCCGCCTGTGGTGCCTCCTGAACTGCGCCCGC | | |
| | CGTCTAGGTAAGTTTAGAGCTCAGGTCGAGACCGGGCCTTTGTCCGG | | |
| | TGCTCCCTTAGAGCCTACCTAGACTCAATCGGCTCTCCACGCTTTGCCT | | |
| | GACCCTGATTGCTCGCCTCTACGTCTTTGTTTCGTTTTCTGTTCTGCGCC | | |
| | GTTGCGAATCGAAAGTTCCACCCCTTTCCCTTTCGTTCACGACAGACTG | | |
| | CCTGCTTGCCCACGGCCAAGTACCAGCGACTCTGCTGGCTCGGAGCC | | |
| | AGCGACAGCCTATTCCATAGCACTCTCCAGGAGAGAAATTTAGTACAC | | |
| | ACTCTGGCTAACTAGAGAACCCACTGCTTACTGGCTTATCGAAATTAA | | |
| | TACGACTCACTATAGGGAGACCCAAGCTGGCTAGCGCCACCATGGAA | | |
| | GACG | | |
| HTLV-E e | CTCAAAAAACTTTTCATGGCACGCATATGGCTGAATAAACTAACAGGA | Y17014 | 115 |
| Efe1a DRC | GTCTATAAAAGCGTGGAGACAGTTCAGGAGGGGGCTCGCATCTCTCC | | |
| Y17014 | TTCACGCGCCCGCCGCCTTACCTGAGGCCGCCATCCACGCCGGTTGAG | | |
| | TCGCGTTCTGTCGCCTCCCGCCTGTGGTGCCTCCTGAACTGCGTCCGC | | |
| | CGTCTAGGTAAGTTTAGAGCTCAGGTCGAGACCGGGCCTTTGTCCGG | | |
| | CGCTCCCTTAGAGCCTACCTAGACTCAGCCGGCTCTCCACGCTTTGCCT | | |
| | GACCCTGCTTGCTCAACTCTACGTCTTTGTTTCGTTTTCTGTTCCGCGCC | | |
| | GCTACAGATCAAAAGTTCCACCCCTTTCCCTTTCATTCACGACTGACTG | | |
| | CCGGCTTGGCCCACGGCCAGGCACCGGCGACTTTACTGGCTCGGAGC | | |
| | CAGTGACAGCCCATTCCATAGCACTCTCCAGGAGAGAAATTTAGTACA | | |
| | CACTCTGGCTAACTAGAGAACCCACTGCTTACTGGCTTATCGAAATTA | | |
| | ATACGACTCACTATAGGGAGACCCAAGCTGGCTAGCGCCACCATGGA | | |
| | AGACG | | |
| HTLV-F f | CTCAAAAAACTTTTCATGGCACGCATATGGCTGAATAAACTAACAGGA | Y17017 | 116 |
| Lib2a | GTCTATAAAAGCGTGGAGACAGTTCAGGAGGGGGCTCGCATCTCTCC | | |
| Gabon | TTCACGCGCCCGCCGCCTTACCTGAGGCCGCCATCCACGCCGGTTGAG | | |
| Y17017 | TCGCGTTCTGCCGCCTCCCGCCCGTGGTGCCTCCTGAACTGCGTTCGC | | |
| | CGTCTAGGTAAGCTTAGAGCTCAGGTCGAGACCGGGCCTTTGTCCGG | | |
| | CGCTCCCTTAGAGCCTTCCTAGACTCAGCCGGCTCTCCACGCTTTGCCT | | |
| | GACCCTGCTTGCTCAAACTCTACGTCTTTGTTTCGTTTTCTGTTCTGCGT | | |
| | CGTTACAGATCGAAAGTTCCACCCCTTTCCCTTTCATTCACGACTGACT | | |
| | GCCGGCTTGGCCCACGGCCAGGTACCGGCGACTTTACTGGCTCGGAG | | |
| | CCAGCGACAGCCTATTCTATAGCGCTCTCCAGGAGAGAAACTTAGTAC | | |
| | ACACTCTGGCTAACTAGAGAACCCACTGCTTACTGGCTTATCGAAATT | | |
| | AATACGACTCACTATAGGGAGACCCAAGCTGGCTAGCGCCACCATGG | | |
| | AAGACG | | |
| HTLV-G g | CTCAAAAAACTTTTCATGGCACGCATATGGCTGAATAAACTAACAGGA | AY818431 | 117 |
| 2656NDa | GTCTATAAAAGCGTGGAGACAGTTCAGGAGGGGGCTCGCATCTCTCC | | |
| Cameroon | TTCACGCGCCCGCCGCCTTACCTGAGGCCGCCATCCACGCCGGTTGAG | | |
| AY818431 | TCGCGTTCTGCCGCCTCCCGCCTGTGGTGCCTCCTGAACTGCGTCCGC | | |
| | CGTCTAGGTAAGTTTAGAGCTCAGGTCGAGACCGGGCCTTTGTCCGG | | |
| | CGCTCCCTTAGAGCCTACCTAGACTCAGCCGGCTCTCCACGCTTTGCCT | | |
| | GACCCTGCTTGTTCAACTCTACGTCTTTGTTTCGTTTTCTGTTCCGTGCT | | |
| | GTTACAAATCGAAAGTTCCACCCCTTTCCCTTTCGTTCACGACTGACTG | | |
| | CCGGCTTGGCCCACGGCCAAGTGCCGGCGACTTTACTGGCTCGGAGC | | |
| | CAGTAACAGCCTATTCTATAACACTCTCCAGGAGAGAAATTTAGTACG | | |
| | TAACAACAAGTCTCTGGCTAACTAGAGAACCCACTGCTTACTGGCTTA | | |
| | TCGAAATTAATACGACTCACTATAGGGAGACCCAAGCTGGCTAGCGC | | |
| | CACCATGGAAGACG | | |
| 3xFLAG | CTATCCTTAGAAGAGGAAAGCCGCGGCCGGCTGCGACGGGGCCCTCC | | 118 |
| tag | AGGGGAGAAAGCGCCACCTCGCGGGGAAACGCATCGTGATCGGCAG | | |
| | CGACGGGCTGAGGAGAAGAGGAAGCGAAAAAAAGAGCGGGAGAAA | | |
| | GAGGAGGAAAAGCAGATTGCTGAGTATTTGAAAAGGAAGGAAGAG | | |
| | GAGAAGGCACGGCGCAGGAGGCGGGCGGAGAAGAAGGCCGCTGAC | | |
| | GTCGCCCGGAGAAAGCAGGAAGAGCAGGAGCGCCGTGAGCGCAAG | | |
| | TGGAGACAAGGGGCTGAGAAGGCGAAACAGCATAGTGCTAGGAAA | | |
| | GAAAAAATGCAGGAGTTGGGGATTGATGGCTATACTAGACAGTTGG | | |
| | AAGGCGAGGTGGAGTCCTTGGAGGCTGAACGGAGGAAGTTGCTGCA | | |
| | GGAGAAGGAGGATTTAATGGGAGAGGTTAATTATTGGCAGGGGAGG | | |
| | CTGGAGGCGATGTGGTTGCAAGGCAGTGGCGACTACAAAGACCATG | | |
| | ACGGTGATTATAAAGATCATGACATCGATTACAAGGATGACGATGAC | | |
| | AAGTAGCTCGAGTCTAGAGGGCCCGTTTAA | | |
| IRES-GFP- | GACATCGATTACAAGGATGACGATGACAAGTAGCCCCTCTCCCTCCCC | | 119 |
| Puro | CCCCCCTAACGTTACTGGCCGAAGCCGCTTGGAATAAGGCCGGTGTG | | |
| | CGTTTGTCTATATGTTATTTTCCACCATATTGCCGTCTTTTGGCAATGT | | |
| | GAGGGCCCGGAAACCTGGCCCTGTCTTCTTGACGAGCATTCCTAGGG | | |
| | GTCTTTCCCCTCTCGCCAAAGGAATGCAAGGTCTGTTGAATGTCGTGA | | |
| | AGGAAGCAGTTCCTCTGGAAGCTTCTTGAAGACAAACAACGTCTGTA | | |
| | GCGACCCTTTGCAGGCAGCGGAACCCCCCACCTGGCGACAGGTGCCT | | |
| | CTGCGGCCAAAAGCCACGTGTATAAGATACACCTGCAAAGGCGGCAC | | |
| | AACCCCAGTGCCACGTTGTGAGTTGGATAGTTGTGGAAAGAGTCAAA | | |
| | TGGCTCTCCTCAAGCGTATTCAACAAGGGGCTGAAGGATGCCCAGAA | | |
| | GGTACCCCATTGTATGGGATCTGATCTGGGGCCTCGGTACACATGCTT | | |
| | TACATGTGTTTAGTCGAGGTTAAAAAAACGTCTAGGCCCCCCGAACCA | | |
| | CGGGGACGTGGTTTTCCTTTGAAAAACACGATGATAATATGGCCACA | | |
| | ACCATGGTGAGCAAGGGCGAGGAGCTGTTCACCGGGGTGGTGCCCA | | |
| | TCCTGGTCGAGCTGGACGGCGACGTAAACGGCCACAAGTTCAGCGTG | | |
| | TCCGGCGAGGGCGAGGGCGATGCCACCTACGGCAAGCTGACCCTGA | | |
| | AGTTCATCTGCACCACCGGCAAGCTGCCCGTGCCCTGGCCCACCCTCG | | |
| | TGACCACCCTGACCTACGGCGTGCAGTGCTTCAGCCGCTACCCCGACC | | |
| | ACATGAAGCAGCACGACTTCTTCAAGTCCGCCATGCCCGAAGGCTAC | | |
| | GTCCAGGAGCGCACCATCTTCTTCAAGGACGACGGCAACTACAAGAC | | |
| | CCGCGCCGAGGTGAAGTTCGAGGGCGACACCCTGGTGAACCGCATC | | |
| | GAGCTGAAGGGCATCGACTTCAAGGAGGACGGCAACATCCTGGGGC | | |
| | ACAAGCTGGAGTACAACTACAACAGCCACAACGTCTATATCATGGCC | | |
| | GACAAGCAGAAGAACGGCATCAAGGTGAACTTCAAGATCCGCCACAA | | |
| | CATCGAGGACGGCAGCGTGCAGCTCGCCGACCACTACCAGCAGAACA | | |
| | CCCCCATCGGCGACGGCCCCGTGCTGCTGCCCGACAACCACTACCTGA | | |
| | GCACCCAGTCCGCCCTGAGCAAAGACCCCAACGAGAAGCGCGATCAC | | |
| | ATGGTCCTGCTGGAGTTCGTGACCGCCGCCGGGATCACTCTCGGCAT | | |
| | GGACGAGCTGTACAAGTCCGGACTCAGATCTAGGAGACGACCTTCCA | | |
| | TGACCGAGTACAAGCCCACGGTGCGCCTCGCCACCCGCGACGACGTC | | |
| | CCCCGGGCCGTACGCACCCTCGCCGCCGCGTTCGCCGACTACCCCGCC | | |
| | ACGCGCCACACCGTCGACCCGGACCGCCACATCGAGCGGGTCACCGA | | |
| | GCTGCAAGAACTCTTCCTCACGCGCGTCGGGCTCGACATCGGCAAGG | | |
| | TGTGGGTCGCGGACGACGGCGCCGCGGTGGCGGTCTGGACCACGCC | | |
| | GGAGAGCGTCGAAGCGGGGGCGGTGTTCGCCGAGATCGGCCCGCGC | | |
| | ATGGCCGAGTTGAGCGGTTCCCGGCTGGCCGCGCAGCAACAGATGG | | |
| | AAGGCCTCCTGGCGCCGCACCGGCCCAAGGAGCCCGCGTGGTTCCTG | | |
| | GCCACCGTCGGCGTCTCGCCCGACCACCAGGGCAAGGGTCTGGGCAG | | |
| | CGCCGTCGTGCTCCCCGGAGTGGAGGCGGCCGAGCGCGCCGGGGTG | | |
| | CCCGCCTTCCTGGAGACCTCCGCGCCCCGCAACCTCCCCTTCTACGAG | | |
| | CGGCTCGGCTTCACCGTCACCGCCGACGTCGAGGTGCCCGAAGGACC | | |
| | GCGCACCTGGTGCATGACCCGCAAGCCCGGTGCGGGATCCACCGGAT | | |
| | CTAGATAAACCGGTCTCGAGTCTAGAGGGCCCGTTT | | |

(Tat domain sequence)
SEQ ID NO: 120
GRKKRRQRRR
(nucleoplasmin NLS sequence)
SEQ ID NO: 121
KRPAATKKAGQAKKKK
(Myc sequence)
SEQ ID NO: 122
EQKLISEEDL
(KRAB domain sequence)
SEQ ID NO: 123
RTLVTFKDVFVDFTREEWKLLDTAQQIVYRNVMLENYKNLVSLGYQLTK
PDVILRLEKGEEPWLV
(SV40 NLS sequence)
SEQ ID NO: 124
PKKKRKV
(meCP2 sequence)
SEQ ID NO: 125
VQVKRVLEKSPGKLLVKMPFQASPGGKGEGGGATTSAQVMVIKRPGRKR
KAEADPQAIPKKRGRKPGSVVAAAAAEAKKKAVKESSIRSVQETVLPIK
KRKTRETVSIEVKEVVKPLLVSTLGEKSGKGLKTCKSPGRKSKESSPKG
RSSSASSPPKKEHHHHHHHAESPKAPMPLLPPPPPPEPQSSEDPISPPE
PQDLSSSICKEEKMPRAGSLESDGCPKEPAKTQPMVAAAATTTTTTTTT
VAEKYKHRGEGERKDIVSSSMPRPNREEPVDSRTPVTERVS
(HTLV-a (a-TC) France L36905)
SEQ ID NO: 126
GTTTCGTTTTCTGTTCTGCGCCGCTA
(HTLV-a (a-Jpn) Japan J02029)
SEQ ID NO: 127
GTTTCGTTTTCTGTTCTGCGCCGTTA
(HTLV-b Brazil JX507077)
SEQ ID NO: 128
GTTTCGTTTTCTGTTCTGCGCCGTTA
(HTLV-c Australia KF242505)
SEQ ID NO: 129
GTTTCGTTTTCTGTTCTGCGCCGCTA
(HTLV-d CAR L76310)
SEQ ID NO: 130
GTTTCGTTTTCTGTTCTGCGCCGTTG
(HTLV-e DRC Y17014)
SEQ ID NO: 131
GTTTCGTTTTCTGTTCCGCGCCGCTA
(HTLV-f Gabon Y17017)
SEQ ID NO: 132
GTTTCGTTTTCTGTTCTGCGTCGTTA
(HTLV-g Cameroon AY818431)
SEQ ID NO: 133
GTTTCGTTTTCTGTTCCGTGCTGTTA

What is claimed is:

1. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:27.

2. The protein of claim 1, wherein the sequence within the HTLV-1 LTR comprises the sequence of SEQ ID NO:27.

3. The protein of claim 1, wherein the sequence within the HTLV-1 LTR is operably linked to a nucleic acid encoding HTLV-1 bZIP factor (HBZ).

4. The protein of claim 1, wherein the zinc finger domain comprises six zinc finger recognition helix regions designated F1 to F6, wherein F1 comprises SEQ ID NO:51, F2 comprises SEQ ID NO:52, F3 comprises SEQ ID NO:53, F4 comprises SEQ ID NO:54, F5 comprises SEQ ID NO:55 and F6 comprises SEQ ID NO:56.

5. The protein of claim 1, wherein the zinc finger domain comprises a sequence having at least 75% sequence identity to SEQ ID NO:4.

6. The protein of claim 5, wherein the zinc finger domain comprises the sequence of SEQ ID NO:4.

7. The protein of claim 1, wherein the protein further comprises a transcriptional repressor.

8. The protein of claim 7, wherein the transcriptional repressor comprises a Kruppel associated box (KRAB) domain, methyl CpG binding protein 2 (meCP2), a DNA methyltransferase (DNMT) domain, or combinations thereof.

9. The protein of claim 8, wherein the transcriptional repressor comprises a KRAB domain.

10. The protein of claim 8, wherein the transcriptional repressor comprises a KRAB domain and meCP2.

11. The protein of claim 1, further comprising a nuclear localization signal, a Tat domain, a Myc tag, or combinations thereof.

12. The protein of claim 1, comprising a sequence having at least 75% sequence identity to SEQ ID NO:13, 20, 21, 22, or 23.

13. The protein of claim 12, comprising the sequence of SEQ ID NO:13, 20, 21, 22, or 23.

14. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:25.

15. The protein of claim 14, wherein the sequence within the HTLV-1 LTR comprises the sequence of SEQ ID NO:25.

16. The protein of claim 14, wherein the sequence within the HTLV-1 LTR is operably linked to a nucleic acid encoding HTLV-1 bZIP factor (HBZ).

17. The protein of claim 14, wherein the zinc finger domain comprises six zinc finger recognition helix regions designated F1 to F6, wherein F1 comprises SEQ ID NO:39, F2 comprises SEQ ID NO:40, F3 comprises SEQ ID NO:41, F4 comprises SEQ ID NO:42, F5 comprises SEQ ID NO:43 and F6 comprises SEQ ID NO:44.

18. The protein of claim 14, wherein the zinc finger domain comprises a sequence having at least 75% sequence identity to SEQ ID NO:2.

19. The protein of claim 18, wherein the zinc finger domain comprises the sequence of SEQ ID NO:2.

20. The protein of claim 14, wherein the protein further comprises a transcriptional repressor.

21. The protein of claim 20, wherein the transcriptional repressor comprises a Kruppel associated box (KRAB) domain, methyl CpG binding protein 2 (meCP2), a DNA methyltransferase (DNMT) domain, or combinations thereof.

22. The protein of claim 21, wherein the transcriptional repressor comprises a KRAB domain.

23. The protein of claim 21, wherein the transcriptional repressor comprises a KRAB domain and mcCP2.

24. The protein of claim 14, further comprising a nuclear localization signal, a Tat domain, a Myc tag, or combinations thereof.

25. The protein of claim 14, comprising a sequence having at least 75% sequence identity to SEQ ID NO:11 or 19.

26. The protein of claim 25, comprising the sequence of SEQ ID NO:11 or 19.

27. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:28.

28. The protein of claim 27, wherein the sequence within the HTLV-1 LTR comprises SEQ ID NO:28.

29. The protein of claim 27, wherein the sequence within the HTLV-1 LTR is operably linked to a nucleic acid encoding HTLV-1 bZIP factor (HBZ).

30. The protein of claim 27, wherein the zinc finger domain comprises six zinc finger recognition helix regions designated F1 to F6, wherein F1 comprises SEQ ID NO:57, F2 comprises SEQ ID NO:58, F3 comprises SEQ ID NO:59, F4 comprises SEQ ID NO:60, F5 comprises SEQ ID NO:61 and F6 comprises SEQ ID NO:62.

31. The protein of claim 27, wherein the zinc finger domain comprises a sequence having at least 75% sequence identity to SEQ ID NO:5.

32. The protein of claim 31, wherein the zinc finger domain comprises the sequence of SEQ ID NO:5.

33. The protein of claim 27, wherein the protein further comprises a transcriptional repressor.

34. The protein of claim 33, wherein the transcriptional repressor comprises a Kruppel associated box (KRAB) domain, methyl CpG binding protein 2 (meCP2), a DNA methyltransferase (DNMT) domain, or combinations thereof.

35. The protein of claim 34, wherein the transcriptional repressor comprises a KRAB domain.

36. The protein of claim 34, wherein the transcriptional repressor comprises a KRAB domain and meCP2.

37. The protein of claim 27, further comprising a nuclear localization signal, a Tat domain, a Myc tag, or combinations thereof.

38. The protein of claim 27, comprising a sequence having at least 75% sequence identity to SEQ ID NO:14.

39. The protein of claim 38, comprising the sequence of SEQ ID NO:14.

40. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:32.

41. The protein of claim 40, wherein the sequence within the HTLV-1 LTR comprises the sequence of SEQ ID NO:32.

42. The protein of claim 40, wherein the sequence within the HTLV-1 LTR is operably linked to a nucleic acid encoding HTLV-1 bZIP factor (HBZ).

43. The protein of claim 40, wherein the zinc finger domain comprises six zinc finger recognition helix regions designated F1 to F6, wherein F1 comprises SEQ ID NO:81, F2 comprises SEQ ID NO:82, F3 comprises SEQ ID NO:83, F4 comprises SEQ ID NO:84, F5 comprises SEQ ID NO:85 and F6 comprises SEQ ID NO:86.

44. The protein of claim 40, wherein the zinc finger domain comprises a sequence having at least 75% sequence identity to SEQ ID NO:9.

45. The protein of claim 44, wherein the zinc finger domain comprises the sequence of SEQ ID NO:9.

46. The protein of claim 40, wherein the protein further comprises a transcriptional repressor.

47. The protein of claim 46, wherein the transcriptional repressor comprises a Kruppel associated box (KRAB) domain, methyl CpG binding protein 2 (meCP2), a DNA methyltransferase (DNMT) domain, or combinations thereof.

48. The protein of claim 47, wherein the transcriptional repressor comprises a KRAB domain.

49. The protein of claim 47, wherein the transcriptional repressor comprises a KRAB domain and meCP2.

50. The protein of claim 40, further comprising a nuclear localization signal, a Tat domain, a Myc tag, or combinations thereof.

51. The protein of claim 40, comprising a sequence having at least 75% sequence identity to SEQ ID NO:18.

52. The protein of claim 51, comprising the sequence of SEQ ID NO:18.

53. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:31.

54. The protein of claim 53, wherein the sequence within the HTLV-1 LTR comprises the sequence of SEQ ID NO:31.

55. The protein of claim 53, wherein the sequence within the HTLV-1 LTR is operably linked to a nucleic acid encoding HTLV-1 bZIP factor (HBZ).

56. The protein of claim 53, wherein the zinc finger domain comprises six zinc finger recognition helix regions designated F1 to F6, wherein F1 comprises SEQ ID NO:75, F2 comprises SEQ ID NO:76, F3 comprises SEQ ID NO:77, F4 comprises SEQ ID NO:78, F5 comprises SEQ ID NO:79 and F6 comprises SEQ ID NO:80.

57. The protein of claim 53, wherein the zinc finger domain comprises a sequence having at least 75% sequence identity to SEQ ID NO:8.

58. The protein of claim 57, wherein the zinc finger domain comprises the sequence of SEQ ID NO:8.

59. The protein of claim 53, wherein the protein further comprises a transcriptional repressor.

60. The protein of claim 59, wherein the transcriptional repressor comprises a Kruppel associated box (KRAB) domain, methyl CpG binding protein 2 (meCP2), a DNA methyltransferase (DNMT) domain, or combinations thereof.

61. The protein of claim 60, wherein the transcriptional repressor comprises a KRAB domain.

62. The protein of claim 60, wherein the transcriptional repressor comprises a KRAB domain and meCP2.

63. The protein of claim 53, further comprising a nuclear localization signal, a Tat domain, a Myc tag, or combinations thereof.

64. The protein of claim 53, comprising a sequence having at least 75% sequence identity to SEQ ID NO:17.

65. The protein of claim 64, comprising the sequence of SEQ ID NO:17.

66. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:30.

67. The protein of claim 66, wherein the sequence within the HTLV-1 LTR comprises the sequence of SEQ ID NO:30.

68. The protein of claim 66, wherein the sequence within the HTLV-1 LTR is operably linked to a nucleic acid encoding HTLV-1 bZIP factor (HBZ).

69. The protein of claim 66, wherein the zinc finger domain comprises six zinc finger recognition helix regions designated F1 to F6, wherein F1 comprises SEQ ID NO:69, F2 comprises SEQ ID NO:70, F3 comprises SEQ ID NO:71, F4 comprises SEQ ID NO:72, F5 comprises SEQ ID NO:73 and F6 comprises SEQ ID NO:74.

70. The protein of claim 66, wherein the zinc finger domain comprises a sequence having at least 75% sequence identity to SEQ ID NO:7.

71. The protein of claim 70, wherein the zinc finger domain comprises the sequence of SEQ ID NO:7.

72. The protein of claim 66, wherein the protein further comprises a transcriptional repressor.

73. The protein of claim 72, wherein the transcriptional repressor comprises a Kruppel associated box (KRAB) domain, methyl CpG binding protein 2 (meCP2), a DNA methyltransferase (DNMT) domain, or combinations thereof.

74. The protein of claim 73, wherein the transcriptional repressor comprises a KRAB domain.

75. The protein of claim 73, wherein the transcriptional repressor comprises a KRAB domain and meCP2.

76. The protein of claim 66, further comprising a nuclear localization signal, a Tat domain, a Myc tag, or combinations thereof.

77. The protein of claim 66, comprising a sequence having at least 75% sequence identity to SEQ ID NO:16.

78. The protein of claim 77, comprising the sequence of SEQ ID NO:16.

79. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:24.

80. The protein of claim 79, wherein the sequence within the HTLV-1 LTR comprises the sequence of SEQ ID NO:24.

81. The protein of claim 79, wherein the sequence within the HTLV-1 LTR is operably linked to a nucleic acid encoding HTLV-1 bZIP factor (HBZ).

82. The protein of claim 79, wherein the zinc finger domain comprises six zinc finger recognition helix regions designated F1 to F6, wherein F1 comprises SEQ ID NO:33, F2 comprises SEQ ID NO:34, F3 comprises SEQ ID NO:35, F4 comprises SEQ ID NO:36, F5 comprises SEQ ID NO:37 and F6 comprises SEQ ID NO:38.

83. The protein of claim 79, wherein the zinc finger domain comprises a sequence having at least 75% sequence identity to SEQ ID NO:1.

84. The protein of claim 83, wherein the zinc finger domain comprises the sequence of SEQ ID NO:1.

85. The protein of claim 79, wherein the protein further comprises a transcriptional repressor.

86. The protein of claim 85, wherein the transcriptional repressor comprises a Kruppel associated box (KRAB) domain, methyl CpG binding protein 2 (meCP2), a DNA methyltransferase (DNMT) domain, or combinations thereof.

87. The protein of claim 86, wherein the transcriptional repressor comprises a KRAB domain.

88. The protein of claim 86, wherein the transcriptional repressor comprises a KRAB domain and meCP2.

89. The protein of claim 79, further comprising a nuclear localization signal, a Tat domain, a Myc tag, or combinations thereof.

90. The protein of claim 79, comprising a sequence having at least 75% sequence identity to SEQ ID NO:10.

91. The protein of claim 90, comprising the sequence of SEQ ID NO:10.

92. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:26.

93. The protein of claim 92, wherein the sequence within the HTLV-1 LTR comprises the sequence of SEQ ID NO:26.

94. The protein of claim 92, wherein the sequence within the HTLV-1 LTR is operably linked to a nucleic acid encoding HTLV-1 bZIP factor (HBZ).

95. The protein of claim 92, wherein the zinc finger domain comprises six zinc finger recognition helix regions designated F1 to F6, wherein F1 comprises SEQ ID NO:45, F2 comprises SEQ ID NO:46, F3 comprises SEQ ID NO:47, F4 comprises SEQ ID NO:48, F5 comprises SEQ ID NO:49 and F6 comprises SEQ ID NO:50.

96. The protein of claim 92, wherein the zinc finger domain comprises a sequence having at least 75% sequence identity to SEQ ID NO:3.

97. The protein of claim 96, wherein the zinc finger domain comprises the sequence of SEQ ID NO:3.

98. The protein of claim 92, wherein the protein further comprises a transcriptional repressor.

99. The protein of claim 98, wherein the transcriptional repressor comprises a Kruppel associated box (KRAB) domain, methyl CpG binding protein 2 (meCP2), a DNA methyltransferase (DNMT) domain, or combinations thereof.

100. The protein of claim 99, wherein the transcriptional repressor comprises a KRAB domain.

101. The protein of claim 99, wherein the transcriptional repressor comprises a KRAB domain and meCP2.

102. The protein of claim 92, further comprising a nuclear localization signal, a Tat domain, a Myc tag, or combinations thereof.

103. The protein of claim 92, comprising a sequence having at least 75% sequence identity to SEQ ID NO:12.

104. The protein of claim 103, comprising the sequence of SEQ ID NO:12.

105. A protein comprising a zinc finger domain capable of binding a sequence within a long terminal repeat (LTR) of Human T-cell lymphotropic virus type 1 (HTLV-1), wherein the sequence has at least 75% sequence identity to SEQ ID NO:29.

106. The protein of claim 105, wherein the sequence within the HTLV-1 LTR comprises the sequence of SEQ ID NO:29.

107. The protein of claim 105, wherein the sequence within the HTLV-1 LTR is operably linked to a nucleic acid encoding HTLV-1 bZIP factor (HBZ).

108. The protein of claim 105, wherein the zinc finger domain comprises six zinc finger recognition helix regions designated F1 to F6, wherein F1 comprises SEQ ID NO:63, F2 comprises SEQ ID NO:64, F3 comprises SEQ ID NO:65, F4 comprises SEQ ID NO:66, F5 comprises SEQ ID NO:67 and F6 comprises SEQ ID NO:68.

109. The protein of claim 105, wherein the zinc finger domain comprises a sequence having at least 75% sequence identity to SEQ ID NO:6.

110. The protein of claim 109, wherein the zinc finger domain comprises the sequence of SEQ ID NO:6.

111. The protein of claim 105, wherein the protein further comprises a transcriptional repressor.

112. The protein of claim 111, wherein the transcriptional repressor comprises a Kruppel associated box (KRAB) domain, methyl CpG binding protein 2 (meCP2), a DNA methyltransferase (DNMT) domain, or combinations thereof.

113. The protein of claim 112, wherein the transcriptional repressor comprises a KRAB domain.

114. The protein of claim 112, wherein the transcriptional repressor comprises a KRAB domain and meCP2.

115. The protein of claim 105, further comprising a nuclear localization signal, a Tat domain, a Myc tag, or combinations thereof.

116. The protein of claim 105, comprising a sequence having at least 75% sequence identity to SEQ ID NO:15.

117. The protein of claim 116, comprising the sequence of SEQ ID NO:15.

118. A nucleic acid encoding the protein of claim 1.

119. A vector comprising the nucleic acid of claim 118.

120. An extracellular vesicle (EV) comprising a nucleic acid encoding the protein of claim 1.

121. The EV of claim 120, wherein the EV further comprises an EV membrane-associated protein and an oncogenic T-cell targeting protein.

122. The EV of claim 121, wherein the EV membrane-associated protein is CD63 or PTGFRN.

123. The EV of claim 121, wherein the oncogenic T-cell targeting protein is an anti-CCR4 antibody or fragment thereof.

124. The EV of claim 121, wherein the oncogenic T-cell targeting protein is fused to an extracellular portion of the EV membrane-associated protein.

125. A pharmaceutical composition comprising the protein of claim 1, the nucleic acid of claim 118, the vector of claim 119, or the EV of claim 120.

126. A cell comprising the protein of claim 1, the nucleic acid of claim 118, the vector of claim 119, or the EV of claim 120.

127. The cell of claim 126, wherein the cell is an oncogenic T-cell.

128. The cell of claim 127, wherein the oncogenic T-cell is an adult T-cell leukemia cell or an adult T-cell lymphoma cell.

129. A method of treating a human T-cell lymphotropic virus type 1 (HTLV-1) associated disease in a subject in need thereof, comprising administering to the subject an effective amount of the protein of claim 1, the nucleic acid of claim 118, the vector of claim 119, or the EV of claim 120.

130. The method of claim 129, wherein the HTLV-1 associated disease is adult T-cell leukemia, adult T-cell lymphoma, HTLV-1 associated myelopathy, tropical spastic paraparesis, or HTLV-1 infection.

131. The method of claim 130, wherein the HTLV-1 associated disease is adult T-cell leukemia.

132. The method of claim 130, wherein the HTLV-1 associated disease is adult T-cell lymphoma.


SEQUENCE LISTING (133 sequences)

SEQ ID NO: 1 - protein, 176 aa, synthetic construct
LEPGEKPYKCPECGKSFSRNDTLTEHQRTHTGEKPYKCPECGKSFSSRRTCRAHQRTHTGEKPYKCPECGKSFSRNDALTEHQRTHTGEKPYKCPECGKSFSQSSNLVRHQRTHTGEKPYKCPECGKSFSTKNSLTEHQRTHTGEKPYKCPECGKSFSTKNSLTEHQRTHTGKKTS

SEQ ID NO: 2 - protein, 176 aa, synthetic construct
LEPGEKPYKCPECGKSFSRADNLTEHQRTHTGEKPYKCPECGKSFSTSGSLVRHQRTHTGEKPYKCPECGKSFSQSGDLRRHQRTHTGEKPYKCPECGKSFSDPGNLVRHQRTHTGEKPYKCPECGKSFSRSDDLVRHQRTHTGEKPYKCPECGKSFSRTDTLRDHQRTHTGKKTS

SEQ ID NO: 3 - protein, 176 aa, synthetic construct
LEPGEKPYKCPECGKSFSTKNSLTEHQRTHTGEKPYKCPECGKSFSRSDKLVRHQRTHTGEKPYKCPECGKSFSDKKDLTRHQRTHTGEKPYKCPECGKSFSRSDNLVRHQRTHTGEKPYKCPECGKSFSDPGALVRHQRTHTGEKPYKCPECGKSFSRADNLTEHQRTHTGKKTS

SEQ ID NO: 4 - protein, 176 aa, synthetic construct
LEPGEKPYKCPECGKSFSRTDTLRDHQRTHTGEKPYKCPECGKSFSQRANLRAHQRTHTGEKPYKCPECGKSFSRADNLTEHQRTHTGEKPYKCPECGKSFSQSSNLVRHQRTHTGEKPYKCPECGKSFSQSGDLRRHQRTHTGEKPYKCPECGKSFSDPGHLVRHQRTHTGKKTS

SEQ ID NO: 5 - protein, 176 aa, synthetic construct
LEPGEKPYKCPECGKSFSRNDTLTEHQRTHTGEKPYKCPECGKSFSRKDNLKNHQRTHTGEKPYKCPECGKSFSDCRDLARHQRTHTGEKPYKCPECGKSFSRSDHLTTHQRTHTGEKPYKCPECGKSFSRNDTLTEHQRTHTGEKPYKCPECGKSFSRSDHLTTHQRTHTGKKTS

SEQ ID NO: 6 - protein, 176 aa, synthetic construct
LEPGEKPYKCPECGKSFSTHLDLIRHQRTHTGEKPYKCPECGKSFSQRAHLERHQRTHTGEKPYKCPECGKSFSTSGHLVRHQRTHTGEKPYKCPECGKSFSRSDHLTNHQRTHTGEKPYKCPECGKSFSQSSNLVRHQRTHTGEKPYKCPECGKSFSRSDHLTNHQRTHTGKKTS

SEQ ID NO: 7 - protein, 176 aa, synthetic construct
LEPGEKPYKCPECGKSFSRADNLTEHQRTHTGEKPYKCPECGKSFSQSGHLTEHQRTHTGEKPYKCPECGKSFSRADNLTEHQRTHTGEKPYKCPECGKSFSERSHLREHQRTHTGEKPYKCPECGKSFSRSDKLTEHQRTHTGEKPYKCPECGKSFSTSGELVRHQRTHTGKKTS

SEQ ID NO: 8 - protein, 176 aa, synthetic construct
LEPGEKPYKCPECGKSFSDPGHLVRHQRTHTGEKPYKCPECGKSFSDCRDLARHQRTHTGEKPYKCPECGKSFSQSGNLTEHQRTHTGEKPYKCPECGKSFSDPGHLVRHQRTHTGEKPYKCPECGKSFSRSDELVRHQRTHTGEKPYKCPECGKSFSDCRDLARHQRTHTGKKTS

SEQ ID NO: 9 - protein, 176 aa, synthetic construct
LEPGEKPYKCPECGKSFSRSDKLTEHQRTHTGEKPYKCPECGKSFSRSDHLTTHQRTHTGEKPYKCPECGKSFSQRAHLERHQRTHTGEKPYKCPECGKSFSSRRTCRAHQRTHTGEKPYKCPECGKSFSRSDKLTEHQRTHTGEKPYKCPECGKSFSDSGNLRVHQRTHTGKKTS

SEQ ID NO: 10 - protein, 309 aa, synthetic construct
MISEFGSGAPGRKKRRQRRRVDLEQKLISEEDLLKRPAATKKAGQAKKKKLEPGEKPYKCPECGKSFSRNDTLTEHQRTHTGEKPYKCPECGKSFSSRRTCRAHQRTHTGEKPYKCPECGKSFSRNDALTEHQRTHTGEKPYKCPECGKSFSQSSNLVRHQRTHTGEKPYKCPECGKSFSTKNSLTEHQRTHTGEKPYKCPECGKSFSTKNSLTEHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 11 - protein, 309 aa, synthetic construct
MISEFGSGAPGRKKRRQRRRVDLEQKLISEEDLLKRPAATKKAGQAKKKKLEPGEKPYKCPECGKSFSRADNLTEHQRTHTGEKPYKCPECGKSFSTSGSLVRHQRTHTGEKPYKCPECGKSFSQSGDLRRHQRTHTGEKPYKCPECGKSFSDPGNLVRHQRTHTGEKPYKCPECGKSFSRSDDLVRHQRTHTGEKPYKCPECGKSFSRTDTLRDHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 12 - protein, 309 aa, synthetic construct
MISEFGSGAPGRKKRRQRRRVDLEQKLISEEDLLKRPAATKKAGQAKKKKLEPGEKPYKCPECGKSFSTKNSLTEHQRTHTGEKPYKCPECGKSFSRSDKLVRHQRTHTGEKPYKCPECGKSFSDKKDLTRHQRTHTGEKPYKCPECGKSFSRSDNLVRHQRTHTGEKPYKCPECGKSFSDPGALVRHQRTHTGEKPYKCPECGKSFSRADNLTEHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 13 - protein, 309 aa, synthetic construct
MISEFGSGAPGRKKRRQRRRVDLEQKLISEEDLLKRPAATKKAGQAKKKKLEPGEKPYKCPECGKSFSRTDTLRDHQRTHTGEKPYKCPECGKSFSQRANLRAHQRTHTGEKPYKCPECGKSFSRADNLTEHQRTHTGEKPYKCPECGKSFSQSSNLVRHQRTHTGEKPYKCPECGKSFSQSGDLRRHQRTHTGEKPYKCPECGKSFSDPGHLVRHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 14 - protein, 309 aa, synthetic construct
MISEFGSGAPGRKKRRQRRRVDLEQKLISEEDLLKRPAATKKAGQAKKKKLEPGEKPYKCPECGKSFSRNDTLTEHQRTHTGEKPYKCPECGKSFSRKDNLKNHQRTHTGEKPYKCPECGKSFSDCRDLARHQRTHTGEKPYKCPECGKSFSRSDHLTTHQRTHTGEKPYKCPECGKSFSRNDTLTEHQRTHTGEKPYKCPECGKSFSRSDHLTTHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 15 - protein, 309 aa, synthetic construct
MISEFGSGAPGRKKRRQRRRVDLEQKLISEEDLLKRPAATKKAGQAKKKKLEPGEKPYKCPECGKSFSTHLDLIRHQRTHTGEKPYKCPECGKSFSQRAHLERHQRTHTGEKPYKCPECGKSFSTSGHLVRHQRTHTGEKPYKCPECGKSFSRSDHLTNHQRTHTGEKPYKCPECGKSFSQSSNLVRHQRTHTGEKPYKCPECGKSFSRSDHLTNHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 16 - protein, 309 aa, synthetic construct
MISEFGSGAPGRKKRRQRRRVDLEQKLISEEDLLKRPAATKKAGQAKKKKLEPGEKPYKCPECGKSFSRADNLTEHQRTHTGEKPYKCPECGKSFSQSGHLTEHQRTHTGEKPYKCPECGKSFSRADNLTEHQRTHTGEKPYKCPECGKSFSERSHLREHQRTHTGEKPYKCPECGKSFSRSDKLTEHQRTHTGEKPYKCPECGKSFSTSGELVRHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 17 - protein, 309 aa, synthetic construct
MISEFGSGAPGRKKRRQRRRVDLEQKLISEEDLLKRPAATKKAGQAKKKKLEPGEKPYKCPECGKSFSDPGHLVRHQRTHTGEKPYKCPECGKSFSDCRDLARHQRTHTGEKPYKCPECGKSFSQSGNLTEHQRTHTGEKPYKCPECGKSFSDPGHLVRHQRTHTGEKPYKCPECGKSFSRSDELVRHQRTHTGEKPYKCPECGKSFSDCRDLARHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 18 - protein, 309 aa, synthetic construct
MISEFGSGAPGRKKRRQRRRVDLEQKLISEEDLLKRPAATKKAGQAKKKKLEPGEKPYKCPECGKSFSRSDKLTEHQRTHTGEKPYKCPECGKSFSRSDHLTTHQRTHTGEKPYKCPECGKSFSQRAHLERHQRTHTGEKPYKCPECGKSFSSRRTCRAHQRTHTGEKPYKCPECGKSFSRSDKLTEHQRTHTGEKPYKCPECGKSFSDSGNLRVHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLV

SEQ ID NO: 19 - protein, 226 aa, synthetic construct
MISEFGSGAPGRKKRRQRRRVDLEQKLISEEDLLKRPAATKKAGQAKKKKLEPGEKPYKCPECGKSFSRADNLTEHQRTHTGEKPYKCPECGKSFSTSGSLVRHQRTHTGEKPYKCPECGKSFSQSGDLRRHQRTHTGEKPYKCPECGKSFSDPGNLVRHQRTHTGEKPYKCPECGKSFSRSDDLVRHQRTHTGEKPYKCPECGKSFSRTDTLRDHQRTHTGKKTS

SEQ ID NO: 20 - protein, 226 aa, synthetic construct
MISEFGSGAPGRKKRRQRRRVDLEQKLISEEDLLKRPAATKKAGQAKKKKLEPGEKPYKCPECGKSFSRTDTLRDHQRTHTGEKPYKCPECGKSFSQRANLRAHQRTHTGEKPYKCPECGKSFSRADNLTEHQRTHTGEKPYKCPECGKSFSQSSNLVRHQRTHTGEKPYKCPECGKSFSQSGDLRRHQRTHTGEKPYKCPECGKSFSDPGHLVRHQRTHTGKKTS

SEQ ID NO: 21 - protein, 340 aa, synthetic construct
MISEFGSGAPGRKKRRQRRRVDLEQKLISEEDLLKRPAATKKAGQAKKKKLEPGEKPYKCPECGKSFSRTDTLRDHQRTHTGEKPYKCPECGKSFSQRANLRAHQRTHTGEKPYKCPECGKSFSRADNLTEHQRTHTGEKPYKCPECGKSFSQSSNLVRHQRTHTGEKPYKCPECGKSFSQSGDLRRHQRTHTGEKPYKCPECGKSFSDPGHLVRHQRTHTGKKTSSAGGGGSGGGGSGGGGSGMGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 22 - protein, 610 aa, synthetic construct
MISEFGSGAPGRKKRRQRRRVDLEQKLISEEDLLKRPAATKKAGQAKKKKLEPGEKPYKCPECGKSFSRTDTLRDHQRTHTGEKPYKCPECGKSFSQRANLRAHQRTHTGEKPYKCPECGKSFSRADNLTEHQRTHTGEKPYKCPECGKSFSQSSNLVRHQRTHTGEKPYKCPECGKSFSQSGDLRRHQRTHTGEKPYKCPECGKSFSDPGHLVRHQRTHTGKKTSSAGGGGSGGGGSGGGGSGRTLVTFKDVFVDFTREEWKLLDTAQQIVYRNVMLENYKNLVSLGYQLTKPDVILRLEKGEEPWLVASSPKKKRKVEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSEF

SEQ ID NO: 23 - protein, 817 aa, synthetic construct
MISEFGSGAPGRKKRRQRRRVDLEQKLISEEDLLKRPAATKKAGQAKKKKLEPGEKPYKCPECGKSFSRTDTLRDHQRTHTGEKPYKCPECGKSFSQRANLRAHQRTHTGEKPYKCPECGKSFSRADNLTEHQRTHTGEKPYKCPECGKSFSQSSNLVRHQRTHTGEKPYKCPECGKSFSQSGDLRRHQRTHTGEKPYKCPECGKSFSDPGHLVRHQRTHTGKKTSSAGGGGSGGGGSGGGGSGGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPINSRSSGSEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 24 - DNA, 18 bp, human T-cell lymphotropic virus type 1
  source 1..18: mol_type = genomic DNA
cctcctgaactgcgtccg

SEQ ID NO: 25 - DNA, 18 bp, human T-cell lymphotropic virus type 1
  source 1..18: mol_type = genomic DNA
acggcggacgcagttcag

SEQ ID NO: 26 - DNA, 18 bp, human T-cell lymphotropic virus type 1
  source 1..18: mol_type = genomic DNA
caggtcgagaccgggcct

SEQ ID NO: 27 - DNA, 18 bp, human T-cell lymphotropic virus type 1
  source 1..18: mol_type = genomic DNA
ggcgcagaacagaaaacg

SEQ ID NO: 28 - DNA, 18 bp, human T-cell lymphotropic virus type 1
  source 1..18: mol_type = genomic DNA
tggccgtgggccaagccg

SEQ ID NO: 29 - DNA, 18 bp, human T-cell lymphotropic virus type 1
  source 1..18: mol_type = genomic DNA
agggaaaggggtggaact

SEQ ID NO: 30 - DNA, 18 bp, human T-cell lymphotropic virus type 1
  source 1..18: mol_type = genomic DNA
gctcggagccagcgacag

SEQ ID NO: 31 - DNA, 18 bp, human T-cell lymphotropic virus type 1
  source 1..18: mol_type = genomic DNA
gccgtgggccaagccggc

SEQ ID NO: 32 - DNA, 18 bp, human T-cell lymphotropic virus type 1
  source 1..18: mol_type = genomic DNA
aaccggcgtggatggcgg

SEQ ID NO: 33 - protein, 7 aa, synthetic construct
RNDTLTE

SEQ ID NO: 34 - protein, 7 aa, synthetic construct
SRRTCRA

SEQ ID NO: 35 - protein, 7 aa, synthetic construct
RNDALTE

SEQ ID NO: 36 - protein, 7 aa, synthetic construct
QSSNLVR

SEQ ID NO: 37 - protein, 7 aa, synthetic construct
TKNSLTE

SEQ ID NO: 38 - protein, 7 aa, synthetic construct
TKNSLTE

SEQ ID NO: 39 - protein, 7 aa, synthetic construct
RADNLTE

SEQ ID NO: 40 - protein, 7 aa, synthetic construct
TSGSLVR

SEQ ID NO: 41 - protein, 7 aa, synthetic construct
QSGDLRR

SEQ ID NO: 42 - protein, 7 aa, synthetic construct
DPGNLVR

SEQ ID NO: 43 - protein, 7 aa, synthetic construct
RSDDLVR

SEQ ID NO: 44 - protein, 7 aa, synthetic construct
RTDTLRD

SEQ ID NO: 45 - protein, 7 aa, synthetic construct
TKNSLTE

SEQ ID NO: 46 - protein, 7 aa, synthetic construct
RSDKLVR

SEQ ID NO: 47 - protein, 7 aa, synthetic construct
DKKDLTR

SEQ ID NO: 48 - protein, 7 aa, synthetic construct
RSDNLVR

SEQ ID NO: 49 - protein, 7 aa, synthetic construct
DPGALVR

SEQ ID NO: 50 - protein, 7 aa, synthetic construct
RADNLTE

SEQ ID NO: 51 - protein, 7 aa, synthetic construct
RTDTLRD

SEQ ID NO: 52 - protein, 7 aa, synthetic construct
QRANLRA

SEQ ID NO: 53 - protein, 7 aa, synthetic construct
RADNLTE

SEQ ID NO: 54 - protein, 7 aa, unidentified
QSSNLVR

SEQ ID NO: 55 - protein, 7 aa, synthetic construct
QSGDLRR

SEQ ID NO: 56 - protein, 7 aa, synthetic construct
DPGHLVR

SEQ ID NO: 57 - protein, 7 aa, synthetic construct
RNDTLTE

SEQ ID NO: 58 - protein, 7 aa, synthetic construct
RKDNLKN

SEQ ID NO: 59 - protein, 7 aa, synthetic construct
DCRDLAR

SEQ ID NO: 60 - protein, 7 aa, synthetic construct
RSDHLTT

SEQ ID NO: 61 - protein, 7 aa, synthetic construct
RNDTLTE

SEQ ID NO: 62 - protein, 7 aa, synthetic construct
RSDHLTT

SEQ ID NO: 63 - protein, 7 aa, synthetic construct
THLDLIR

SEQ ID NO: 64 - protein, 7 aa, synthetic construct
QRAHLER

SEQ ID NO: 65 - protein, 7 aa, synthetic construct
TSGHLVR

SEQ ID NO: 66 - protein, 7 aa, synthetic construct
RSDHLTN

SEQ ID NO: 67 - protein, 7 aa, synthetic construct
QSSNLVR

SEQ ID NO: 68 - protein, 7 aa, synthetic construct
RSDHLTN

SEQ ID NO: 69 - protein, 7 aa, synthetic construct
RADNLTE

SEQ ID NO: 70 - protein, 7 aa, synthetic construct
QSGHLTE

SEQ ID NO: 71 - protein, 7 aa, synthetic construct
RADNLTE

SEQ ID NO: 72 - protein, 7 aa, synthetic construct
ERSHLRE

SEQ ID NO: 73 - protein, 7 aa, synthetic construct
RSDKLTE

SEQ ID NO: 74 - protein, 7 aa, synthetic construct
TSGELVR

SEQ ID NO: 75 - protein, 7 aa, synthetic construct
DPGHLVR

SEQ ID NO: 76 - protein, 7 aa, synthetic construct
DCRDLAR

SEQ ID NO: 77 - protein, 7 aa, synthetic construct
QSGNLTE

SEQ ID NO: 78 - protein, 7 aa, synthetic construct
DPGHLVR

SEQ ID NO: 79 - protein, 7 aa, synthetic construct
RSDELVR

SEQ ID NO: 80 - protein, 7 aa, synthetic construct
DCRDLAR

SEQ ID NO: 81 - protein, 7 aa, synthetic construct
RSDKLTE

SEQ ID NO: 82 - protein, 7 aa, synthetic construct
RSDHLTT

SEQ ID NO: 83 - protein, 7 aa, synthetic construct
QRAHLER

SEQ ID NO: 84 - protein, 7 aa, synthetic construct
SRRTCRA

SEQ ID NO: 85 - protein, 7 aa, synthetic construct
RSDKLTE

SEQ ID NO: 86 - protein, 7 aa, synthetic construct
DSGNLRV

SEQ ID NO: 87 - DNA, 20 bp, synthetic construct
  source 1..20: mol_type = other DNA
gaagaacaaggcggtgaaga

SEQ ID NO: 88 - DNA, 22 bp, synthetic construct
  source 1..22: mol_type = other DNA
gggtctctaggaagagcactat

SEQ ID NO: 89 - DNA, 20 bp, synthetic construct
  source 1..20: mol_type = other DNA
gacctggaaactgaccatca

SEQ ID NO: 90 - DNA, 21 bp, synthetic construct
  source 1..21: mol_type = other DNA
ggtctcatagcgtgacttctc

SEQ ID NO: 91 - DNA, 20 bp, synthetic construct
  source 1..20: mol_type = other DNA
cttgacactgcccaacagat

SEQ ID NO: 92 - DNA, 24 bp, synthetic construct
  source 1..24: mol_type = other DNA
acctgagaataacgtctggtttag

SEQ ID NO: 93 - DNA, 21 bp, synthetic construct
  source 1..21: mol_type = other DNA
gctggacaggctaagaagaag

SEQ ID NO: 94 - DNA, 19 bp, synthetic construct
  source 1..19: mol_type = other DNA
cgtatgggttcgctgatgt

SEQ ID NO: 95 - DNA, 18 bp, synthetic construct
  source 1..18: mol_type = other DNA
ttgatgatcgcgcagtgt

SEQ ID NO: 96 - DNA, 20 bp, synthetic construct
  source 1..20: mol_type = other DNA
ctctgctcctcctgttcgac

SEQ ID NO: 97 - DNA, 20 bp, synthetic construct
  source 1..20: mol_type = other DNA
ttaaaagcagccctggtgac

SEQ ID NO: 98 - DNA, 18 bp, synthetic construct
  source 1..18: mol_type = other DNA
gttcaggaggcaccacag

SEQ ID NO: 99 - DNA, 19 bp, synthetic construct
  source 1..19: mol_type = other DNA
acaggcaagcatcgaaaca

SEQ ID NO: 100 - DNA, 17 bp, synthetic construct
  source 1..17: mol_type = other DNA
cggacgcagttcaggag

SEQ ID NO: 101 - DNA, 20 bp, synthetic construct
  source 1..20: mol_type = other DNA
aaagcgtggagacagttcag

SEQ ID NO: 102 - DNA, 55 bp, synthetic construct
  source 1..55: mol_type = other DNA
ggcaaggcttgaccgacaattggtgtactaagtttctctcctggagagtgctata

SEQ ID NO: 103 - DNA, 47 bp, synthetic construct
  source 1..47: mol_type = other DNA
ttaaacgggccctctagactcgagctattgcaaccacatcgcctcca

SEQ ID NO: 104 - DNA, 22 bp, synthetic construct
  source 1..22: mol_type = other DNA
ctgatctcagaggaggacctgc

SEQ ID NO: 105 - DNA, 51 bp, synthetic construct
  source 1..51: mol_type = other DNA
tggactagtggatccgagctcggtacctcagctcgttttcttccccgtgtg

SEQ ID NO: 106 - DNA, 45 bp, synthetic construct
  source 1..45: mol_type = other DNA
acacggggaagaaaacgagcagcgctggaggaggtggaagcggag

SEQ ID NO: 107 - DNA, 51 bp, synthetic construct
  source 1..51: mol_type = other DNA
tggactagtggatccgagctcggtaccctatcaaacgcacgcgaagtactc

SEQ ID NO: 108 - DNA, 876 bp, human T-cell lymphotropic virus type 1
  source 1..876: mol_type = genomic DNA
gtacgggccagatatacgcgtttgacaatgaccatgagccccaaatatcccccgggggcttagagcctcccagtgaaaaacatttccgagaaacagaagtctgaaaaggtcagggcccagactaaggctctgacgtctccccccggagggacagctcagcaccggctcaggctaggccctgacgtgtccccctgaagacaaatcataagctcagacctccgggaagccaccggaaccacccatttcctccccatgtttgtcaagccaccctcaggcgttgacgacaacccctcacctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgccgcctcccgcctgtggtgcctcctgaactgcgtccgccgtctaggtaagtttagagctcaggtcgagaccgggcctttgtccggcgctcccttggagcctacctagactcagccggctctccacgctttgcctgaccctgcttgctcgactccgcgtctttgtttcgttttctgttctgcgccgctacagatcgaaagttccacccctttccctttcattcacgactgactgccggcttggcccacggccaagtaccggcgactctgttggctcggagccagcgacagcccattctatagcactctccaggagagaaacttagtacacactctggctaactagagaacccactgcttactggcttatcgaaattaatacgactcactatagggagacccaagctggctagcgccaccatggaagacg

SEQ ID NO: 109 - DNA, 1535 bp, human T-cell lymphotropic virus type 1
  source 1..1535: mol_type = genomic DNA
tcgctcttgatcagggcgatatcctcctcgatgtcaggccactcgtcccaggactcgatcacgtccacgacactctcagcatggacgatggccttgatcttgtcttggtgctcgtaggagtagtgaaaggccagacaagccccccagtcgtggcccacaaagatgattttctttggaaggttcagcagctcgaaccaagcggtgaggtacttgtagtgatccaggaggcgatatgagccattcccgctcttgccggacttacccattccgatcagatcagggatgatgcatctagccacgggctcgatgtgaggcacgacgtgcctccacaggtagctggaggcagcgttaccatgcagaaaaatcacggcgttctcggcgtgcttctcggaatcatagtagttgatgaaggagtccagcacgttcatttgcttgcagcgagcccaccactgaggcccagtgatcatgcgtttgcgttgctcggggtcgtacaccttggaagcccctacagatacaaagttaaccatgcttattatcagcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctctaagaccctcaaggtccttaccccgccaatcactcatacgacccccaacattccaccctccttcctccaggccatgcgcaaatactcccccttccgaaatggatacatggaacccacccttgggcagcacctcccaaccctgtcttttccagaccccggactccggccccaaaacctgtacaccctctggggaggctccgttgtctgcatgtacctctaccagctttccccccccatcacctggcccctcctgccccacgtgattttttgccaccccggccagctcggggccttcctcaccaatgttccctacaagcgaatagaagaactcctctataaaatttcccttaccacaggggccctaataattctacccgaagactgtttgcccaccacccttttccagcctgttagagcacccgtcacgctgacagcctggcaaaacggcctccttccgttccactcaaccctcaccactccaggccttatttggacatttaccgatggcacgcctatgatttccgggccctgccctaaagatggccagccatctttagtactacagtcctcctcctttatatttcacaaatttcaaaccaaggcctaccacccctcatttctactctcacacggcctcatacagtactcttcctttcataatttacatctcctgtttgaagaatacaccaacatccccatttctctactttttaacgaaaaagaggcagatgacaatgaccatgagcccc

SEQ ID NO: 110 - DNA, 583 bp, human T-cell lymphotropic virus type 1
  source 1..583: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctgcgtctttgtttcgttttctgttctgcgccgctacagatcgaaagttccacccctttccctttcattcacgactgactgccggcttggcccacggccaagtaccggcgactccgttggctcggagccagcgacagcccattctatagcactctccaggagagaaacttagtacacagtctctggctaactagagaacccactgcttactggcttatcgaaattaatacgactcactatagggagacccaagctggctagcgccaccatggaagacg

SEQ ID NO: 111 - DNA, 593 bp, human T-cell lymphotropic virus type 1
  source 1..593: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcgaaagttccacccctttccctttcattcacgactgactgccggcttggcccacggccaagtaccggcgactccgttggctcggagccagcgacagcccatcctatagcactctcaggagagaaatttagtacacatagttggaggtagctctggctaactagagaacccactgcttactggcttatcgaaattaatacgactcactatagggagacccaagctggctagcgccaccatggaagacg

SEQ ID NO: 112 - DNA, 581 bp, human T-cell lymphotropic virus type 1
  source 1..581: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgccgcctcccgcctgtggtgcctcctgaactgcgtccgccgtctaggtaagtttagagctcaggtcgagaccgggcctttgtccggcgctcccttagagcctacctagactcagccggctctccacgctttgcctgaccctgtttgctcacctctacgtctttgtttcgttttctgttctgcgccgttacagatcgaaagttccacccctttccctttcattcacgactgactgccggcttggcccacggccaagtaccggcgactttactggctcggagccagcgacagcctattctatagcactctccaggagagaaattcagtacacactctggctaactagagaacccactgcttactggcttatcgaaattaatacgactcactatagggagacccaagctggctagcgccaccatggaagacg

SEQ ID NO: 113 - DNA, 581 bp, human T-cell lymphotropic virus type 1
  source 1..581: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgccgcctcccgcctgtggtgcctcctgaactgcgtccgccgtctaggtaagttcggagctcaggtcgagaccgggcctttgtccggcgctcccttagagcccacttagattcggtcggctctccacgctttgcctgaccctgcttgctcaactccacgtctttgtttcgttttctgttctgcgccgctaccgatcgaaagttccgcccctttccctttcattcacgtctgactgccggcttggcccacggccaagcaccggcacccttactggctcggagccagcgacagcccattctatacctctctccaggagagagacatagaacacactctggctaactagagaacccactgcttactggcttatcgaaattaatacgactcactatagggagacccaagctggctagcgccaccatggaagacg

SEQ ID NO: 114 - DNA, 580 bp, human T-cell lymphotropic virus type 1
  source 1..580: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggtcgagtcgcgtcctgccgcctcccgcctgtggtgcctcctgaactgcgcccgccgtctaggtaagtttagagctcaggtcgagaccgggcctttgtccggtgctcccttagagcctacctagactcaatcggctctccacgctttgcctgaccctgattgctcgcctctacgtctttgtttcgttttctgttctgcgccgttgcgaatcgaaagttccacccctttccctttcgttcacgacagactgcctgcttgcccacggccaagtaccagcgactctgctggctcggagccagcgacagcctattccatagcactctccaggagagaaatttagtacacactctggctaactagagaacccactgcttactggcttatcgaaattaatacgactcactatagggagacccaagctggctagcgccaccatggaagacg

SEQ ID NO: 115 - DNA, 581 bp, human T-cell lymphotropic virus type 1
  source 1..581: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgtcgcctcccgcctgtggtgcctcctgaactgcgtccgccgtctaggtaagtttagagctcaggtcgagaccgggcctttgtccggcgctcccttagagcctacctagactcagccggctctccacgctttgcctgaccctgcttgctcaactctacgtctttgtttcgttttctgttccgcgccgctacagatcaaaagttccacccctttccctttcattcacgactgactgccggcttggcccacggccaggcaccggcgactttactggctcggagccagtgacagcccattccatagcactctccaggagagaaatttagtacacactctggctaactagagaacccactgcttactggcttatcgaaattaatacgactcactatagggagacccaagctggctagcgccaccatggaagacg

SEQ ID NO: 116 - DNA, 582 bp, human T-cell lymphotropic virus type 1
  source 1..582: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgccgcctcccgcccgtggtgcctcctgaactgcgttcgccgtctaggtaagcttagagctcaggtcgagaccgggcctttgtccggcgctcccttagagccttcctagactcagccggctctccacgctttgcctgaccctgcttgctcaaactctacgtctttgtttcgttttctgttctgcgtcgttacagatcgaaagttccacccctttccctttcattcacgactgactgccggcttggcccacggccaggtaccggcgactttactggctcggagccagcgacagcctattctatagcgctctccaggagagaaacttagtacacactctggctaactagagaacccactgcttactggcttatcgaaattaatacgactcactatagggagacccaagctggctagcgccaccatggaagacg

SEQ ID NO: 117 - DNA, 590 bp, human T-cell lymphotropic virus type 1
  source 1..590: mol_type = genomic DNA
ctcaaaaaacttttcatggcacgcatatggctgaataaactaacaggagtctataaaagcgtggagacagttcaggagggggctcgcatctctccttcacgcgcccgccgccttacctgaggccgccatccacgccggttgagtcgcgttctgccgcctcccgcctgtggtgcctcctgaactgcgtccgccgtctaggtaagtttagagctcaggtcgagaccgggcctttgtccggcgctcccttagagcctacctagactcagccggctctccacgctttgcctgaccctgcttgttcaactctacgtctttgtttcgttttctgttccgtgctgttacaaatcgaaagttccacccctttccctttcgttcacgactgactgccggcttggcccacggccaagtgccggcgactttactggctcggagccagtaacagcctattctataacactctccaggagagaaatttagtacgtaacaacaagtctctggctaactagagaacccactgcttactggcttatcgaaattaatacgactcactatagggagacccaagctggctagcgccaccatggaagacg

SEQ ID NO: 118 - DNA, 579 bp, synthetic construct
  source 1..579: mol_type = other DNA
ctatccttagaagaggaaagccgcggccggctgcgacggggccctccaggggagaaagcgccacctcgcggggaaacgcatcgtgatcggcagcgacgggctgaggagaagaggaagcgaaaaaaagagcgggagaaagaggaggaaaagcagattgctgagtatttgaaaaggaaggaagaggagaaggcacggcgcaggaggcgggcggagaagaaggccgctgacgtcgcccggagaaagcaggaagagcaggagcgccgtgagcgcaagtggagacaaggggctgagaaggcgaaacagcatagtgctaggaaagaaaaaatgcaggagttggggattgatggctatactagacagttggaaggcgaggtggagtccttggaggctgaacggaggaagttgctgcaggagaaggaggatttaatgggagaggttaattattggcaggggaggctggaggcgatgtggttgcaaggcagtggcgactacaaagaccatgacggtgattataaagatcatgacatcgattacaaggatgacgatgacaagtagctcgagtctagagggcccgtttaa

SEQ ID NO: 119 - DNA, 2013 bp, synthetic construct
  source 1..2013: mol_type = other DNA
gacatcgattacaaggatgacgatgacaagtagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcgggatccaccggatctagataaaccggtctcgagtctagagggcccgttt

SEQ ID NO: 120 - protein, 10 aa, synthetic construct
GRKKRRQRRR

SEQ ID NO: 121 - protein, 16 aa, synthetic construct
KRPAATKKAGQAKKKK

SEQ ID NO: 122 - protein, 10 aa, synthetic construct
EQKLISEEDL

SEQ ID NO: 123 - protein, 65 aa, synthetic construct
RTLVTFKDVFVDFTREEWKLLDTAQQIVYRNVMLENYKNLVSLGYQLTKPDVILRLEKGEEPWLV

SEQ ID NO: 124 - protein, 7 aa, synthetic construct
PKKKRKV

SEQ ID NO: 125 - protein, 286 aa, synthetic construct
VQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 126 - DNA, 26 bp, human T-cell lymphotropic virus type 1
  source 1..26: mol_type = genomic DNA
gtttcgttttctgttctgcgccgcta

SEQ ID NO: 127 - DNA, 26 bp, human T-cell lymphotropic virus type 1
  source 1..26: mol_type = genomic DNA
gtttcgttttctgttctgcgccgtta

SEQ ID NO: 128 - DNA, 26 bp, human T-cell lymphotropic virus type 1
  source 1..26: mol_type = genomic DNA
gtttcgttttctgttctgcgccgtta

SEQ ID NO: 129 - DNA, 26 bp, human T-cell lymphotropic virus type 1
  source 1..26: mol_type = genomic DNA
gtttcgttttctgttctgcgccgcta

SEQ ID NO: 130 - DNA, 26 bp, human T-cell lymphotropic virus type 1
  source 1..26: mol_type = genomic DNA
gtttcgttttctgttctgcgccgttg

SEQ ID NO: 131 - DNA, 26 bp, human T-cell lymphotropic virus type 1
  source 1..26: mol_type = genomic DNA
gtttcgttttctgttccgcgccgcta

SEQ ID NO: 132 - DNA, 26 bp, human T-cell lymphotropic virus type 1
  source 1..26: mol_type = genomic DNA
gtttcgttttctgttctgcgtcgtta

SEQ ID NO: 133 - DNA, 26 bp, human T-cell lymphotropic virus type 1
  source 1..26: mol_type = genomic DNA
gtttcgttttctgttccgtgctgtta